(19) United States
(12) Patent Application Publication
(10) Pub. No.: US 2023/0406937 A1
(43) Pub. Date: Dec. 21, 2023

(54) MULTISPECIFIC ANTIBODIES FOR TARGETING CD28 AND PD-L1 AND METHODS OF USE THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US)

(21) Appl. No.: 18/249,886

(22) PCT Filed: Oct. 29, 2021
(86) PCT No.: PCT/US2021/057384

§ 371 Date: Apr. 20, 2023

Related U.S. Application Data
(60) Provisional application No. 63/189,843, filed on May 18, 2021.
(60) Provisional application No. 63/187,690, filed on May 12, 2021.
(60) Provisional application No. 63/187,699, filed on May 12, 2021.
(60) Provisional application No. 63/187,719, filed on May 12, 2021.
(60) Provisional application No. 63/141,268, filed on Jan. 25, 2021.
(60) Provisional application No. 63/123,329, filed on Dec. 9, 2020.
(60) Provisional application No. 63/123,327, filed on Dec. 9, 2020.
(60) Provisional application No. 63/107,942, filed on Oct. 30, 2020.

(51) Int. Cl. C07K 16/28

(52) U.S. Cl. CPC C07K 16/2827; C07K 16/2818; C07K 2317/31; C07K 2317/622; A61K 2039/505

(57) ABSTRACT

Provided herein are antibodies that selectively bind to CD28 and PD-L1, pharmaceutical compositions thereof, as well as nucleic acids, and methods of use, and methods for making and discovering the same.

### CROSS-REFERENCE

This application claims the benefit of U.S. Provisional Application No. 63/107,942, filed Oct. 30, 2020, U.S. Provisional Application No. 63/141,268, filed Jan. 25, 2021, U.S. Provisional Application No. 63/189,843, filed May 18, 2021, U.S. Provisional Application No. 63/123,327, filed Dec. 9, 2020, U.S. Provisional Application No. 63/187,719, filed May 12, 2021, U.S. Provisional Application No. 63/123,329, filed Dec. 9, 2020, U.S. Provisional Application No. 63/187,699, filed May 12, 2021, U.S. Provisional Application No. 63/187,690, filed May 12, 2021, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 27, 2021, is named 52426-726_601_SL.txt and is 483,304 bytes in size.

### SUMMARY

Disclosed herein, in certain embodiments, are multispecific antibodies comprising a CD28 binding domain and a PD-L1 binding domain, wherein when the CD28 binding domain is a single chain variable fragment (scFv), then the PD-L1 binding domain is not a scFv. In some instances, the multispecific antibody is according to the following formula:

A-L-B (Formula I)

wherein A comprises the CD28 binding domain; B comprises the PD-L1 binding domain; and L comprises a linker that connects A to B. In some instances, the CD28 binding domain comprises a single chain variable fragment, a single domain antibody, a Fab, or a Fab′. In some instances, the CD28 binding domain comprises the single chain variable fragment. In some instances, the CD28 binding domain comprises the single domain antibody. In some instances, the CD28 binding domain comprises the Fab or the Fab′. In some instances, the PD-L1 binding domain comprises a single domain antibody, a Fab, or a Fab′. In some instances, the PD-L1 binding domain comprises the Fab or the Fab′. In some instances, the PD-L1 binding domain comprises the Fab or the Fab′ and the CD28 binding domain comprises the single chain variable fragment. In some instances, the PD-L1 binding domain that comprises the Fab or the Fab′ comprises a Fab heavy chain polypeptide comprising a Fab heavy chain variable domain and a Fab light chain polypeptide comprising a Fab light chain variable domain. In some instances, the CD28 binding domain that comprises the single chain variable fragment comprises a scFv heavy chain variable domain and a scFv light chain variable domain. In some instances, the linker connects the C-terminus of A to an N-terminus of B. In some instances, the linker connects the N-terminus of A to a C-terminus of B. In some instances, the linker connects the C-terminus of A to the N-terminus of the Fab heavy chain polypeptide. In some instances, the linker connects the N-terminus of A to the C-terminus of the Fab heavy chain polypeptide. In some instances, the linker connects the C-terminus of A to the N-terminus of the Fab light chain polypeptide. In some instances, the linker connects the N-terminus of A to the C-terminus of the Fab light chain polypeptide. In some instances, the linker connects the Fab light chain polypeptide to the scFv light chain variable domain. In some instances, the linker connects the Fab light chain polypeptide to the scFv heavy chain variable domain. In some instances, the linker connects the Fab heavy chain polypeptide to the scFv light chain variable domain. In some instances, the linker connects the Fab heavy chain polypeptide to the scFv heavy chain variable domain. In some instances, the linker connects the Fab light chain polypeptide to the N-terminus of the scFv light chain variable domain. In some instances, the linker connects the Fab light chain polypeptide to the C-terminus of the scFv light chain variable domain. In some instances, the linker connects the Fab light chain polypeptide to the N-terminus of the scFv heavy chain variable domain. In some instances, the linker connects the Fab light chain polypeptide to the C-terminus of the scFv heavy chain variable domain. In some instances, the linker connects the Fab heavy chain polypeptide to the N-terminus of the scFv light chain variable domain. In some instances, the linker connects the Fab heavy chain polypeptide to the C-terminus of the scFv light chain variable domain. In some instances, the linker connects the Fab heavy chain polypeptide to the N-terminus of the scFv heavy chain variable domain. In some instances, the linker connects the Fab heavy chain polypeptide to the C-terminus of the scFv heavy chain variable domain. In some instances, the linker is at least 5 amino acids in length. In some instances, the linker is no more than 30 amino acids in length. In some instances, the linker is at least 5 amino acids and no more than 30 amino acids in length. In some instances, the linker is 5 amino acids in length. In some instances, the linker is 15 amino acids in length. In some instances, the linker is selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some instances, L has a formula comprising (G₂S)ₙ (SEQ ID NO: 233), wherein n is an integer from 1 to 3. In some instances, the linker comprises an amino acid sequence of SEQ ID NO: 18 (GGGGSGGGGSGGGGS) or SEQ ID NO: 19 (GGGGS). In some instances, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some instances, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3. In some instances, the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12; HC-CDR1: SEQ ID NO: 24; HC-CDR2: SEQ ID NO: 25; HC-CDR3: SEQ ID NO: 26; HC-CDR1: SEQ ID NO: 27; HC-CDR2: SEQ ID NO: 28; HC-CDR3: SEQ ID NO: 29; or HC-CDR1: SEQ ID NO: 30; HC-CDR2: SEQ ID NO: 31; HC-CDR3: SEQ ID NO: 32 and wherein said CDRs comprise from 0-2 amino acid modifications in at least one of said HC-CDR1, HC-CDR2, or HC-CDR3. In some instances, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise:LC-CDR1: SEQ ID NO: 13; LC-CDR2: SEQ ID NO: 14; and LC-CDR3: SEQ ID NO: 15; LC-CDR1: SEQ ID NO: 33; LC-CDR2: SEQ ID NO: 34; and LC-CDR3: SEQ ID NO: 35; LC-CDR1: SEQ ID NO: 36; LC-CDR2: SEQ ID NO: 37; and LC-CDR3: SEQ ID NO: 38; or LC-CDR1: SEQ ID NO: 39; LC-CDR2: SEQ ID NO: 40; and LC-CDR3: SEQ ID NO: 41 and wherein the CDRs comprise from 0-2 amino acid modifications in at least one of said LC-CDR1, LC-CDR2, or LC-CDR3. In some instances, the scFv heavy chain variable domain comprises an amino acid sequence that has at least 80% sequence identity to the amino acid sequence according to SEQ ID NO: 7. In some instances, the scFv heavy chain variable domain comprises an amino acid sequence of at least 75 consecutive amino acid residues of SEQ ID NO: 7 In some instances, the scFv heavy chain variable domain comprises an amino acid sequence of at least 110 consecutive amino acid residues of SEQ ID NO: 7. In some instances, the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7. In some instances, the scFv heavy chain variable domain comprises an amino acid sequence according to SEQ ID NO: 7. In some instances, the scFv light chain variable domain comprises an amino acid sequence that has at least 80% sequence identity to the amino acid sequence according to SEQ ID NO: 8. In some instances, the scFv light chain variable domain comprises an amino acid sequence of at least 75 consecutive amino acid residues of SEQ ID NO: 8. In some instances, the scFv light chain variable domain comprises an amino acid sequence of at least 100 consecutive amino acid residues of SEQ ID NO: 8. In some instances, the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some instances, the scFv light chain variable domain comprises an amino acid sequence according to SEQ ID NO: 8. In some instances, the scFv comprises an amino acid sequence that has at least 80% sequence identity to the amino acid sequence according to SEQ ID NO: 9. In some instances, the scFv comprises an amino acid sequence of at least 175 consecutive amino acid residues of SEQ ID NO: 9. In some instances, the scFv comprises an amino acid sequence of at least 210 consecutive amino acid residues of SEQ ID NO: 9. In some instances, the scFv comprises an amino acid sequence of at least 210 consecutive amino acid residues of SEQ ID NO: 9 and has at least 80% sequence identity to the at least 210 consecutive amino acid residues of SEQ ID NO: 9. In some instances, the scFv comprises an amino acid sequence according to SEQ ID NO: 9. In some instances, the Fab heavy chain polypeptide comprises an amino acid sequence that has at least 80% sequence identity to the amino acid sequence according to SEQ ID NO: 17, 43, 45, or 47. In some instances, the Fab heavy chain polypeptide comprises an amino acid sequence of at least 175 consecutive amino acid residues of SEQ ID NO: 17, 43, 45, or 47. In some instances, the Fab heavy chain polypeptide comprises an amino acid sequence of at least 215 consecutive amino acid residues of SEQ ID NO: 17, 43, 45, or 47. In some instances, the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17, 43, 45, or 47. In some instances, the Fab heavy chain polypeptide comprises an amino acid sequence according to SEQ ID NO: 17, 43, 45, or 47. In some instances, the Fab light chain polypeptide comprises an amino acid sequence that has at least 80% sequence identity to the amino acid sequence according to SEQ ID NO: 16, 42, 44, or 46. In some instances, the Fab light chain polypeptide comprises an amino acid sequence of at least 175 consecutive amino acid residues of SEQ ID NO: 16, 42, 44, or 46. In some instances, the Fab light chain polypeptide comprises an amino acid sequence of at least 200 consecutive amino acid residues of SEQ ID NO: 16, 42, 44, or 46. In some instances, the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16, 42, 44, or 46. In some instances, the Fab light chain polypeptide comprises an amino acid sequence according to SEQ ID NO: 16, 42, 44, or 46. In some instances, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21. In some instances, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21. In some instances, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21. In some instances, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21. In some instances, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22. In some instances, the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

Disclosed herein, in certain embodiments, are pharmaceutical compositions comprising: the multispecific antibody described herein; and a pharmaceutically acceptable excipient.

Disclosed herein, in certain embodiments, are isolated recombinant nucleic acid molecules encoding a polypeptide of the multispecific antibody described herein.

Disclosed herein, in certain embodiments, are methods of treating cancer in a subject in need thereof comprising administering to the subject the multispecific antibody described herein or the pharmaceutical composition described herein. In some instances, the multispecific antibody is administered to the subject as a single agent therapy. In some instances, the multispecific antibody is not administered as a part of a treatment regimen with a second therapeutic agent. In some instances, the multispecific antibody is not administered as a part of a treatment regimen with a second therapeutic agent comprising a tumor binding domain. In some instances, the multispecific antibody is not administered as a part of a treatment regimen with a second therapeutic agent comprising an anti-CD19 antibody. In some instances, the multispecific antibody is not administered as a part of a treatment regimen with a second therapeutic agent comprising an antibody that has an anti-CD19 binding domain and an anti-CD3 binding domain. In some instances, the multispecific antibody induces T cell mediated cytotoxicity of tumor cells. In some instances, the administering to the subject of the multispecific antibody is sufficient to reduce or eliminate the cancer as compared to a baseline measurement of the cancer taken from the subject prior to the administering of the multispecific antibody. In some instances, the reduction is at least about 1-fold, 5-fold, 10-fold, 20-fold, 40-fold, 60-fold, 80-fold, or up to about 100-fold. In some instances, the cancer is a hematological malignancy.

In some instances, the cancer is leukemia or lymphoma. In some instances, the cancer is lymphoma, and wherein the lymphoma is B-cell lymphoma. In some instances, the cancer is a solid tumor. In some instances, the solid tumor expresses PD-L1. In some instances, the solid tumor is sarcoma, breast cancer, lung cancer, or carcinoma. In some instances, the solid tumor is lung cancer, and wherein the lung cancer is non-small cell lung cancer. Disclosed herein are methods of treating cancer in a subject in need thereof comprising administering to the subject a multispecific antibody that comprises a CD28 binding domain and a PD-L1 binding domain wherein the multispecific antibody that comprises the CD28 binding domain and the PD-L1 binding domain is not administered as part of a treatment regimen with another multispecific antibody that targets a cancer antigen different from PD-L1 or CD28. In some embodiments, the multispecific antibody that comprises the CD28 binding domain and the PD-L1 binding domain is administered to the subject as a single agent therapy. In some embodiments, the multispecific antibody that comprises the CD28 binding domain and the PD-L1 binding domain comprises an IgG framework, an IgA framework, an IgE framework, or an IgM framework. In some embodiments, the CD28 binding domain comprises a single chain variable fragment, a single domain antibody, a Fab, or a Fab′. In some embodiments, the PD-L1 binding domain comprises a single chain variable fragment, a single domain antibody, a Fab, or a Fab′. In some embodiments, the PD-L1 binding domain comprises a single chain variable fragment and the CD28 binding domain comprises a single chain variable fragment. In some embodiments, the CD28 binding domain comprises an anti-CD28 light chain polypeptide. In some embodiments, the anti-CD28 light chain polypeptide comprises a variable domain of an IgG1, IgG2, IgG3, or IgG4 light chain. In some embodiments, the CD28 binding domain comprises an anti-CD28 heavy chain polypeptide. In some embodiments, the anti-CD28 heavy chain polypeptide comprises a variable domain of an IgG1, IgG2, IgG3, or IgG4 heavy chain. In some embodiments, the PD-L1 binding domain comprises an anti-PD-L1 light chain polypeptide. In some embodiments, the anti-PD-L1 light chain polypeptide comprises a variable domain of an IgG1, IgG2, IgG3, or IgG4 light chain. In some embodiments, the PD-L1 binding domain comprises an anti-PD-L1 heavy chain polypeptide. In some embodiments, the anti-PD-L1 heavy chain polypeptide comprises a variable domain of an IgG1, IgG2, IgG3, or IgG4 heavy chain. In some embodiments, the multispecific antibody further comprises a fragment crystallizable (Fc) region. In some embodiments, the Fc region comprises an IgG CH2 domain and an IgG CH3 domain. In some embodiments, the Fc region comprises a heterodimeric Fc region. In some embodiments, the Fc region comprises at least one amino acid modification that increases the half-life of the multispecific antibody. In some embodiments, the Fc region comprises at least one amino acid modification that modulates its interaction with an Fc receptor. In some embodiments, the Fc region comprises at least one amino acid modification that increases binding of the Fc region to an Fc receptor. In some embodiments, the Fc region comprises at least one amino acid modification that decreases glycosylation of the Fe region. In some embodiments, the modification is an amino acid substitution, deletion, or addition. In some embodiments, the modification is an amino acid substitution. In some embodiments, the at least one amino acid modification that decreases glycosylation of the Fc region comprises an amino acid substitution at a position corresponding to position N297 of human IgG1, wherein the numbering is according to the EU index of Kabat. In some embodiments, the Fc region is afucosylated. In some embodiments, the anti-CD28 light chain polypeptide comprises complementarity determining regions (CDRs): LC-CDR1, LC-CDR2, and LC-CDR3, wherein the LC-CDR1, the LC-CDR2, and the LC-CDR3 the anti-CD28 light chain polypeptide: LC-CDR1: SEQ ID NO: 4; LC-CDR2: SEQ ID NO: 5; and LC-CDR3: SEQ ID NO: 6 and wherein the CDRs comprise from 0-2 amino acid modifications in at least one of said LC-CDR1, LC-CDR2, or LC-CDR3. In some embodiments, the anti-CD28 heavy chain polypeptide comprises complementarity determining regions (CDRs): HC-CDR1, HC-CDR2, and HC-CDR3, wherein the HC-CDR1, the HC-CDR2, and the HC-CDR3 of the anti-CD28 heavy chain polypeptide comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 13; LC-CDR2: SEQ ID NO: 14; and LC-CDR3: SEQ ID NO: 15; LC-CDR1: SEQ ID NO: 33; LC-CDR2: SEQ ID NO: 34; and LC-CDR3: SEQ ID NO: 35; LC-CDR1: SEQ ID NO: 36; LC-CDR2: SEQ ID NO: 37; and LC-CDR3: SEQ ID NO: 38; or LC-CDR1: SEQ ID NO: 39; LC-CDR2: SEQ ID NO: 40; and LC-CDR3: SEQ ID NO: 41;and wherein the CDRs comprise from 0-2 amino acid modifications in at least one of said LC-CDR1, LC-CDR2, or LC-CDR3. In some embodiments, the anti-PD-L1 heavy chain polypeptide the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 10; HC-CDR2: SEQ ID NO: 11; HC-CDR3: SEQ ID NO: 12; HC-CDR1: SEQ ID NO: 24; HC-CDR2: SEQ ID NO: 25; HC-CDR3: SEQ ID NO: 26; HC-CDR1: SEQ ID NO: 27; HC-CDR2: SEQ ID NO: 28; HC-CDR3: SEQ ID NO: 29; or HC-CDR1: SEQ ID NO: 30; HC-CDR2: SEQ ID NO: 31; HC-CDR3: SEQ ID NO: 32 and wherein said CDRs comprise from 0-2 amino acid modifications in at least one of said HC-CDR1, HC-CDR2, or HC-CDR3. In some embodiments, the multispecific antibody induces T cell mediated cytotoxicity of tumor cells. In some embodiments, the administering to the subject of the multispecific antibody is sufficient to reduce or eliminate the cancer as compared to a baseline measurement of the cancer taken from the subject prior to the administering of the multispecific antibody. In some embodiments, the reduction is at least about 1-fold, 5-fold, 10-fold, 20-fold, 40-fold, 60-fold, 80-fold, or up to about 100-fold. In some embodiments, the cancer is a hematological malignancy. In some embodiments, the cancer is leukemia or lymphoma. In some embodiments, the cancer is lymphoma, and wherein the lymphoma is B-cell lymphoma.

In some embodiments, the cancer is a solid tumor. In some embodiments, the solid tumor expresses PD-L1. In some embodiments, the solid tumor is sarcoma, breast cancer, lung cancer, or carcinoma. In some embodiments, the solid tumor is lung cancer, and wherein the lung cancer is non-small cell lung cancer.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B illustrate exemplary schemas of PDL1 x CD28 multispecific antibodies.

FIG. 2 illustrates an exemplary schema of the in vitro immune cell activation assays using target coated beads.

FIG. 3A illustrates a graph of IFNy cytokine release from PBMCs cultured with single agent PDL1 x CD28 multispecific Ab-1.

FIG. 3B illustrates a graph of TNFα cytokine release from PBMCs cultured with single agent PDL1 x CD28 multispecific Ab-1.

FIG. 3C illustrates a graph of IL-2 cytokine release from PBMCs cultured with single agent PDL1 x CD28 multispecific Ab-1.

FIG. 4A illustrates a graph of the number of live immune cells overtime in response to PBMC co-cultured with PDL1 target coated beads and Ab-1.

FIG. 4B illustrates a graph of the number of live CD3+ cells overtime in response to PBMC co-cultured with PDL1 target coated beads and Ab-1.

FIG. 4C illustrates a graph of the number of live CD4+ cells over time in response to PBMC co-cultured with PDL1 target coated beads and Ab-1.

FIG. 4D illustrates a graph of the number of live CD8+ cells over time in response to PBMC co-culture with PDL1 target coated beads and Ab-1.

FIG. 5A illustrates binding kinetics of Ab-1 to biotinylated human PD-L1.

FIG. 5B illustrates binding kinetics of anti-PD-L1 Fab 1 to biotinylated human PD-L1.

FIG. 6A illustrates binding kinetics of Ab-1 to biotinylated human CD28.

FIG. 6B illustrates binding kinetics of Ab-2 to biotinylated human CD28.

FIG. 7A illustrates binding kinetics of Ab-1 to human PD-L1 Fc.

FIG. 7B illustrates binding kinetics of Ab-1 to cynomolgus monkey PD-L1 Fc.

FIG. 8A illustrates binding of Ab-1 to Ab-6 to PD-L1 as measured by ELISA.

FIG. 8B illustrates binding of Ab-1 to Ab-6 to CD28 as measured by ELISA.

FIG. 9A illustrates a cartoon configuration of a multispecific antibody that targets CD28 and PD-L1 that is administered in combination with a T cell engager that targets a tumor associated antigen such as TROP2 and CD3 of T cell.

FIGS. 9B-9D illustrate immune cell activation as measured by cytokine release after co-culture of target coated beads and PBMCs and administration of Ab-1 and the combination of Ab-1 and TCE-1 an anti-TROP2 x CD3 T cell engager.

FIGS. 9E-9H illustrate immune cell proliferation after co-culture of target coated beads and PBMCs and administration of Ab-1 and the combination of Ab-1 and TCE-1 an anti-TROP2 x CD3 T cell engager.

FIGS. 91-9K illustrate polypeptide complexes of different orientation harboring different PD-L1 binding domains (Ab-1 through Ab-8) activate PBMCs as measured by cytokine release in combination with a T cell engager (TCE-3) against non-immunogenic beads coated with tumor associated antigen and PD-L1.

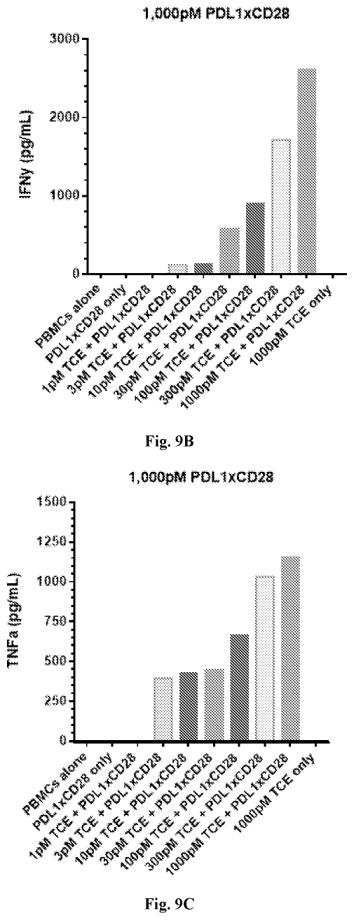

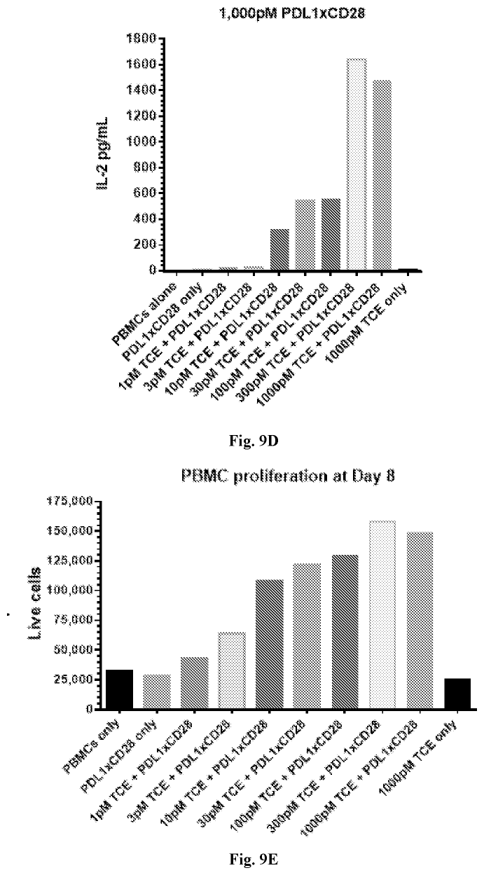

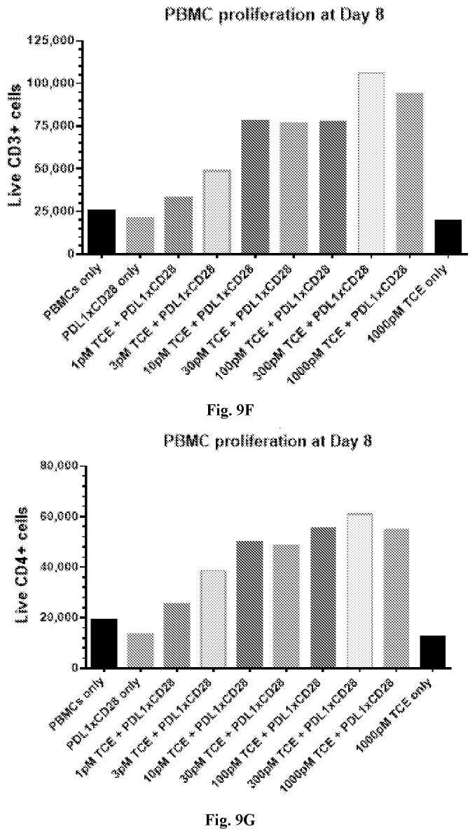

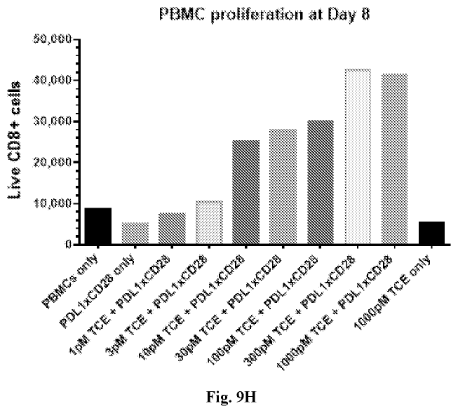

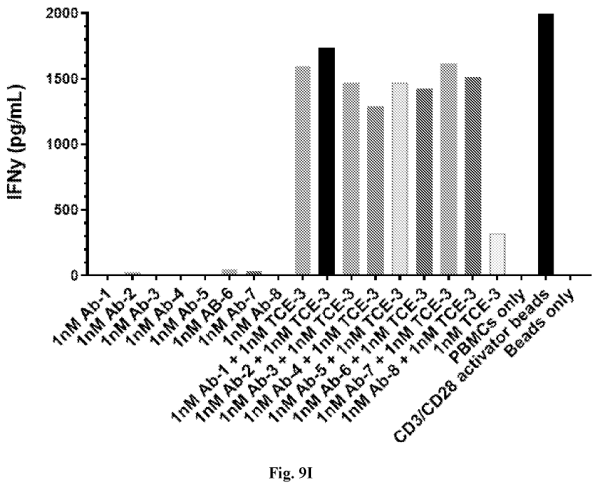

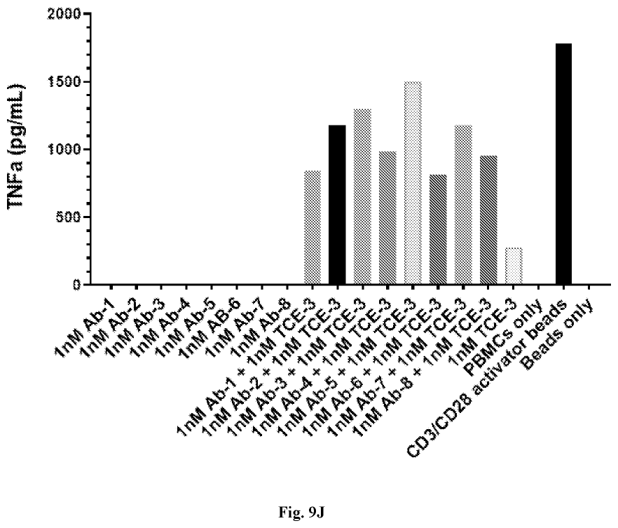

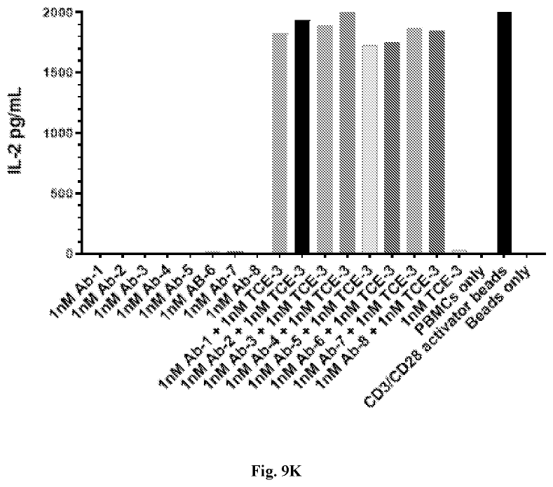

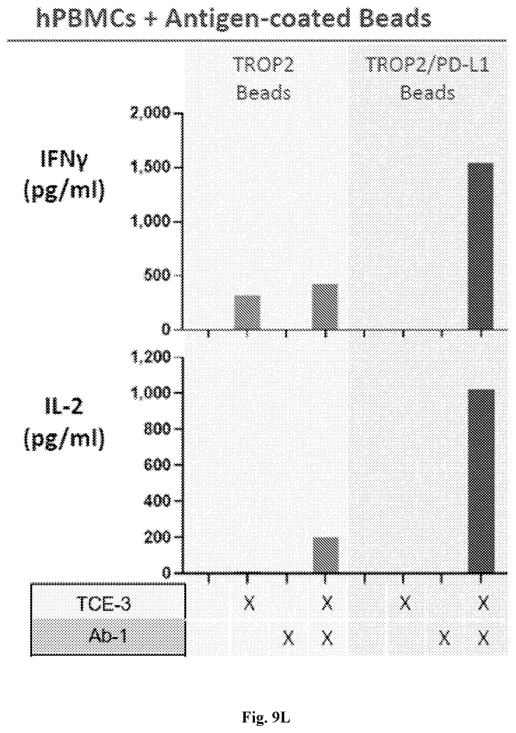

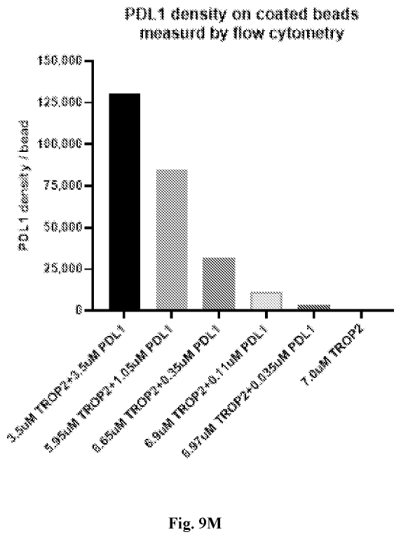

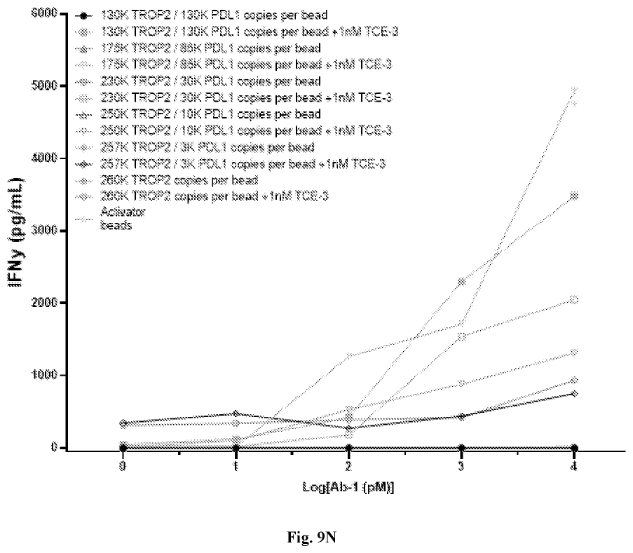

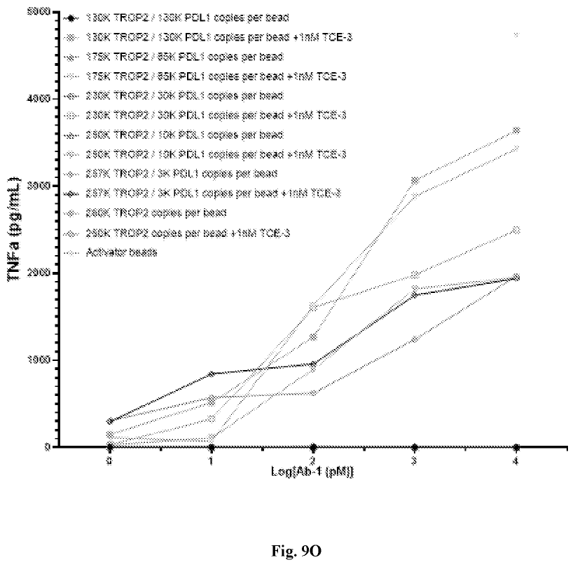

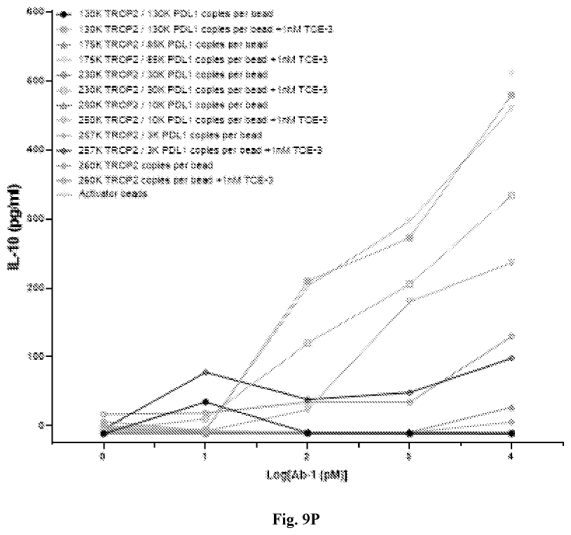

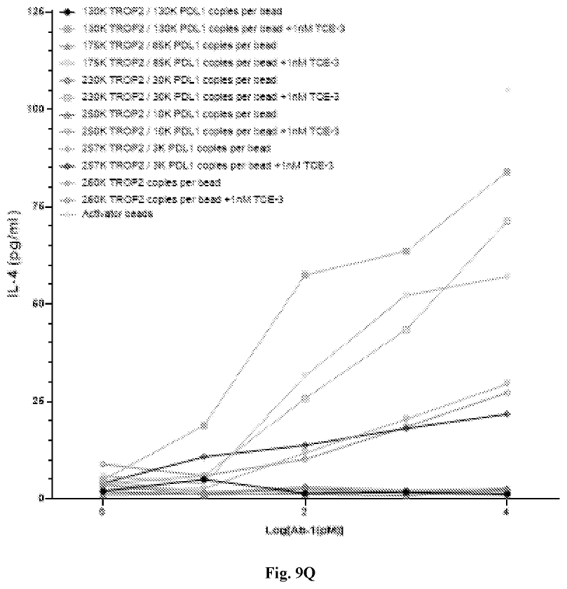

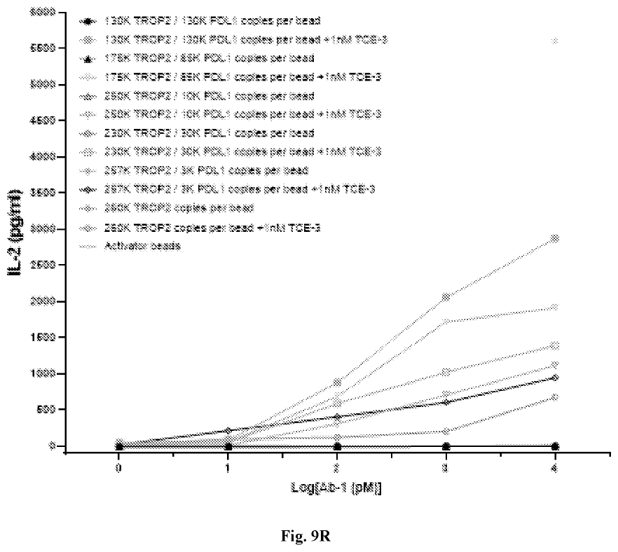

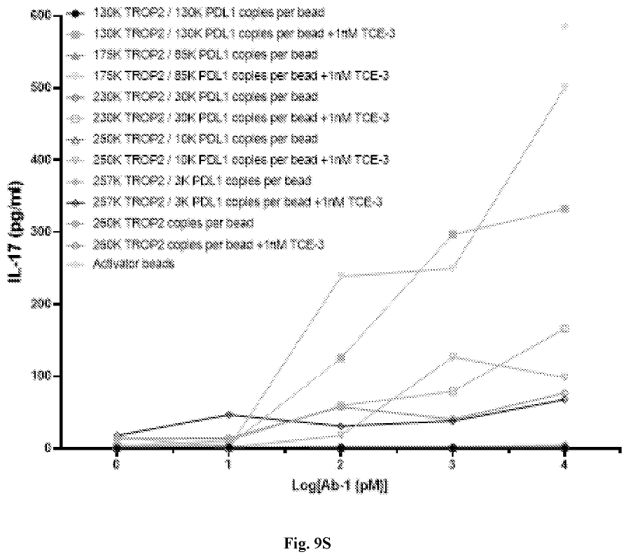

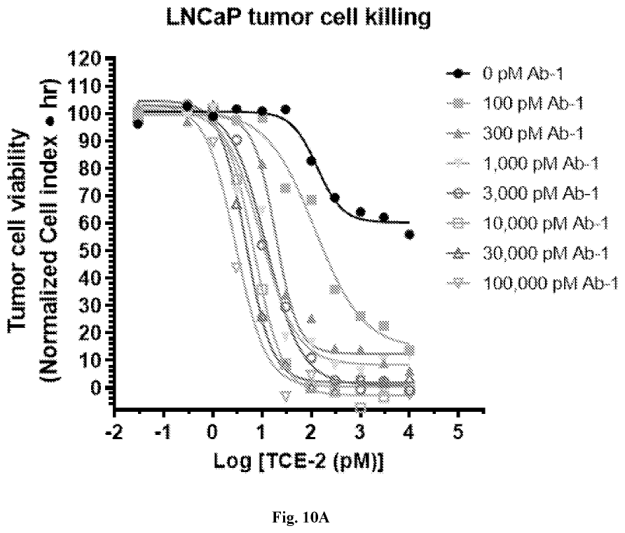

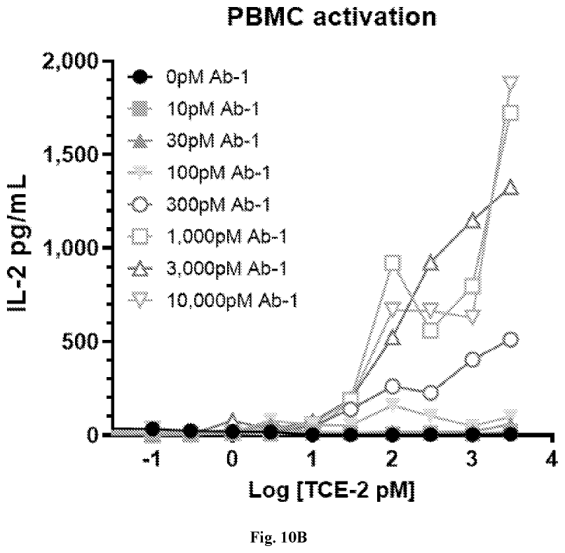

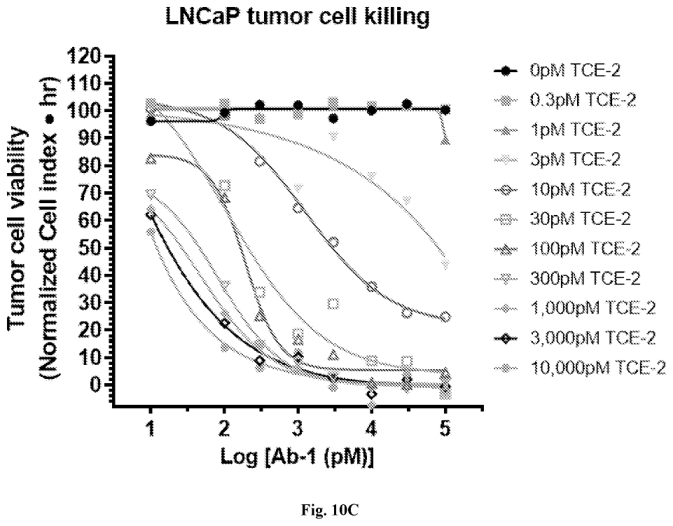

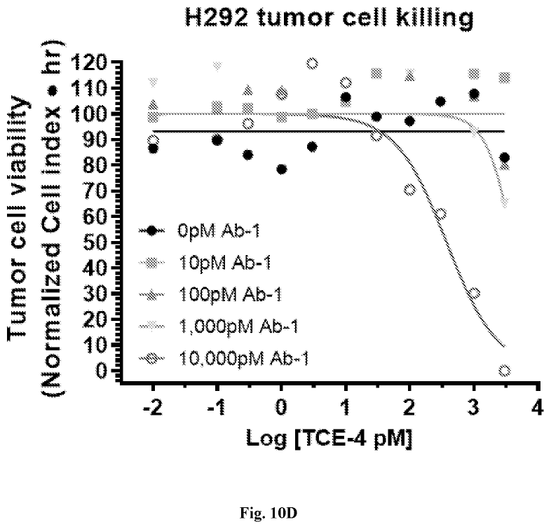

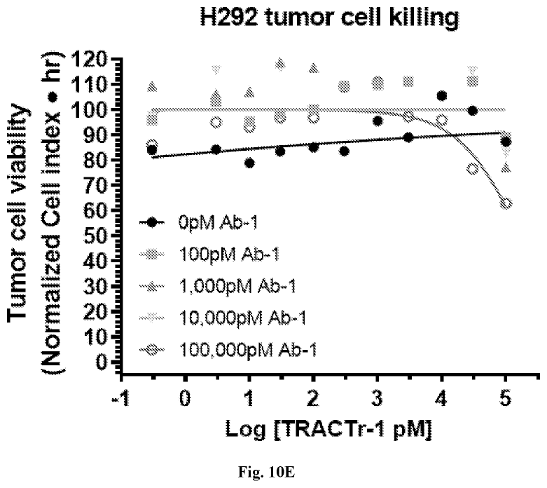

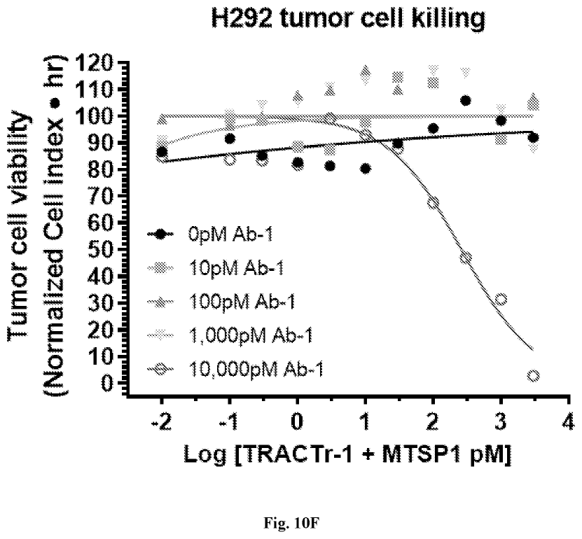

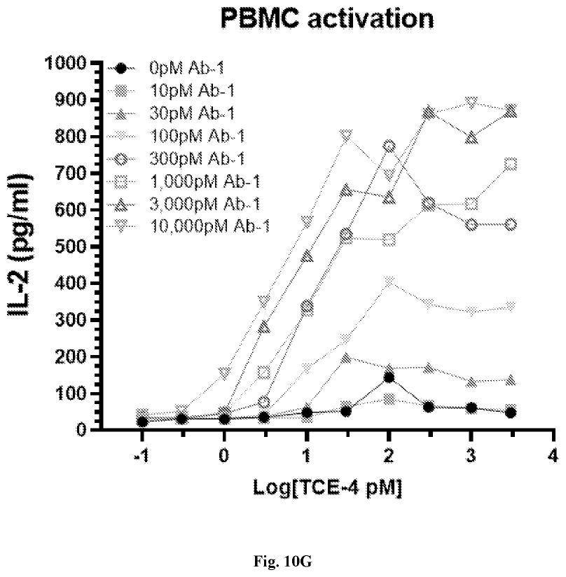

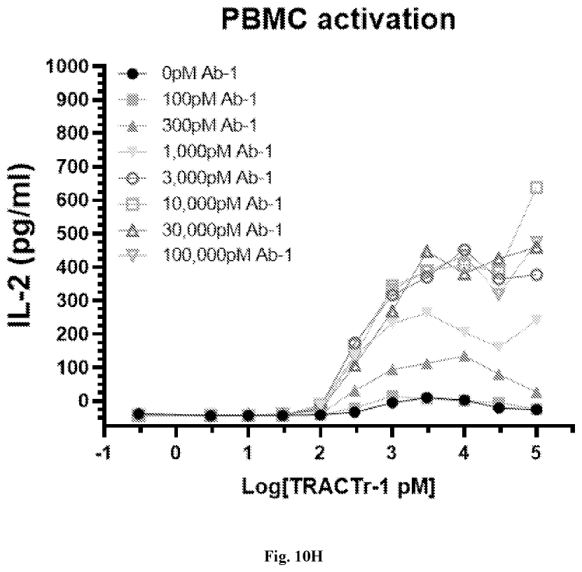

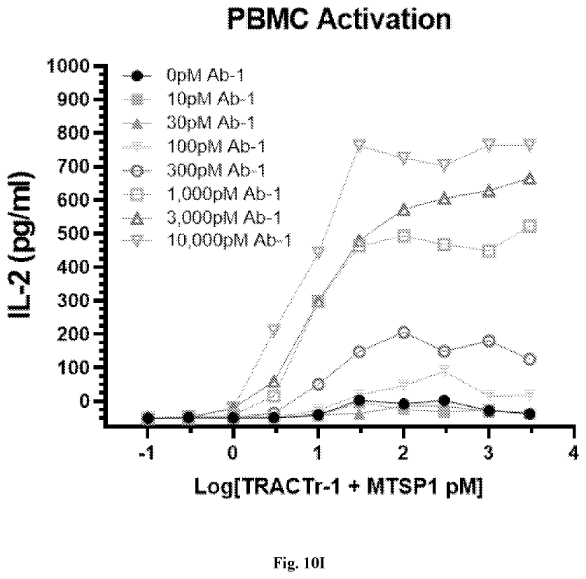

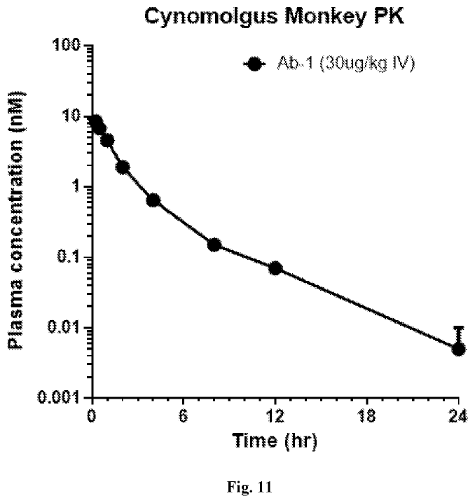

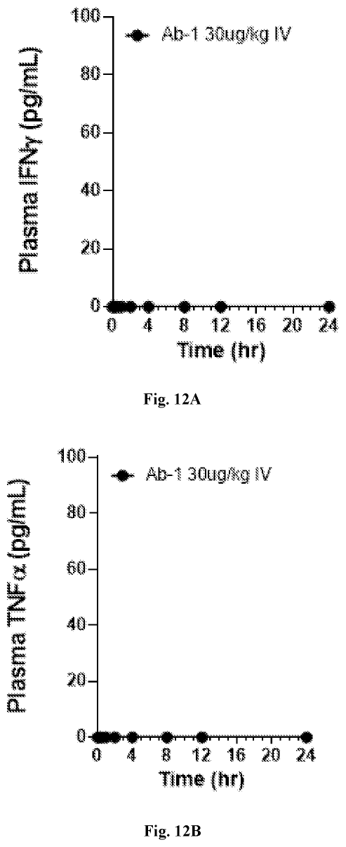

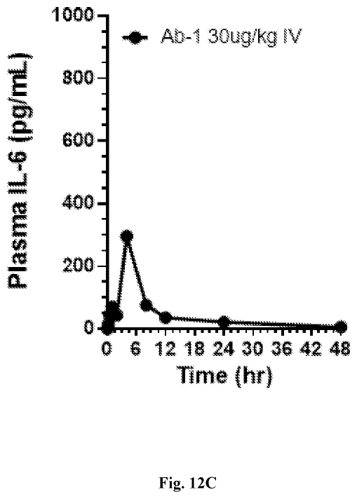

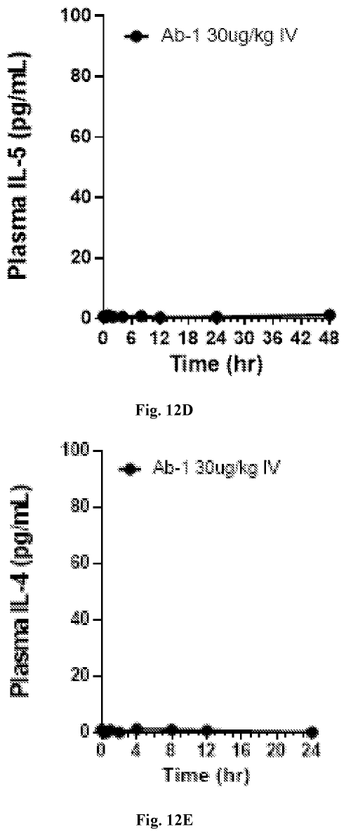

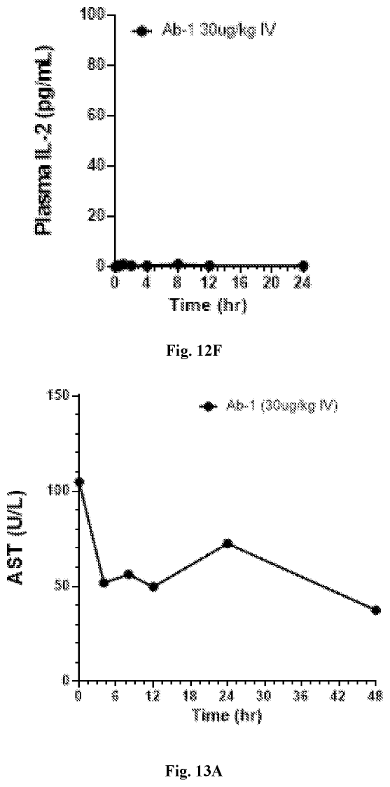

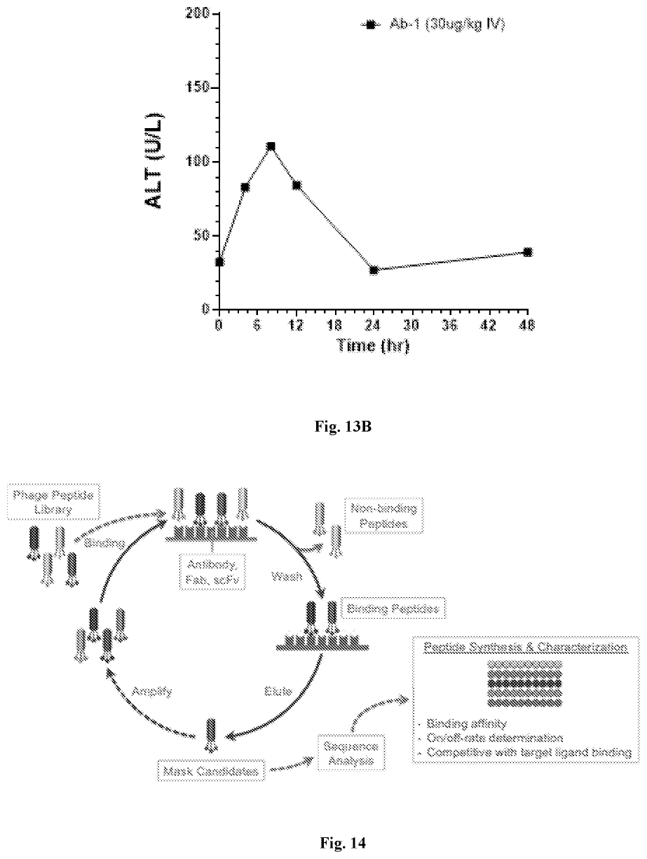

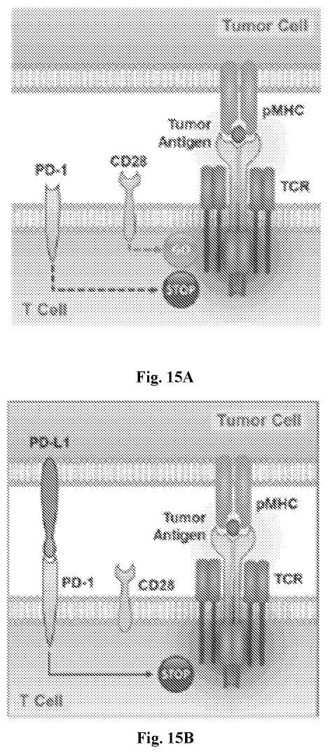

FIG. 9L illustrates polypeptide complex mediated activation of PBMCs in combination with a T cell engager.

FIG. 9M-9S illustrate polypeptide complex mediated activation of PBMCs in combination with a T cell engager is PDL1 density dependent.

FIGS. 10A-10C illustrate results of an in vitro tumor cell killing assay using the LNCaP PDL1 positive tumor cell line in which Ab-1 and TCE-2 are co-administered in the presence of human PBMCs. In vitro tumor cell killing and PBMC activation measured by cytokine release is synergized when Ab-1 is combined with an anti-PSMA x CD3 T cell engager, TCE-2.

FIG. 10D illustrates a graph of PBMC mediated in vitro killing of H292 tumor cells using Ab-1 and TCE-4.

FIG. 10E illustrates a graph of PBMC mediated in vitro killing of H292 tumor cells using Ab-1 and TRACTr-1.

FIG. 10F illustrates a graph of PBMC mediated in vitro killing of H292 tumor cells using Ab-1 and TRACTr-pre-treated with MTSP1.

FIG. 10G illustrates a graph of IL-2 cytokine release from PBMCs co-cultured with H292 cells, Ab-1, and TCE-4.

FIG. 10H illustrates a graph of IL-2 cytokine release from PBMCs co-cultured with H292 cells, Ab-1 and TRACTr-1.

FIG. 10I illustrates a graph of IL-2 cytokine release from PBMCs co-cultured with H292 cells Ab-1, and TRACTr-1 pre-treated with MTSP1.

FIG. 11 illustrates pharmacokinetics of Ab-1 in cynomolgus monkey after a single IV bolus injection.

FIG. 12A-12F illustrate cytokine release in cynomolgus monkey after a single IV bolus injection of Ab-1.

FIG. 13A-13B illustrate serum liver enzymes in cynomolgus monkey after a single IV bolus injection of Ab-1.

FIG. 14 illustrates a schematic for identifying P1 or P2 peptides that can be attached to the PD-L1 and CD28 multispecific antibodies for selective activation in tumor microenvironments. The schematic illustrates a directed evolution and phage display technology to identify peptides that block antigen recognition by antigen binding domains.

FIGS. 15A-15C exemplify schematics for the multispecific antibodies comprising a CD28 binding domain and a PD-L1 binding domain described herein can increase activation as a monotherapy. FIG. 15A shows a T cell attacking a tumor cell, and the potential secondary signals are the PD-1 (stop) and CD28 (go).

FIG. 15B shows how a tumor deactivates T cells through PD-L1. The PD-L1/PD-1 triggers the stop signal while the CD28 (go) signaling is not activated. FIG. 15C shows a reactivated T cell in which the multispecific antibody with a PD-L1 binding domain and CD28 binding domain reactivate the T cell by converting the PD-L1/PD-1 stop signal to a CD28 go signal.

FIG. 16 exemplifies a schematic of a multispecific antibody comprising a CD28 binding domain and a PD-L1 binding domain in combination with a T cell engager.

### DETAILED DESCRIPTION

Bispecific antibodies for redirecting T cells for mediating cancer cell killing have shown promise in both pre-clinical and in clinical studies. Efficient T cell activation has been obtained with single chain variable fragments (scFv), notably the Bispecific T-cell Engagers (BiTEs) format, in which one scFv targets a tumor cell antigen, and the other scFv targets an epitope such as CD3 that is involved in T cell activation. One such example of a BiTE is blinatumomab that targets CD19 and CD3 which has been approved in Europe and the United States for treatment of chemotherapy-resistant CD19+ B cell acute lymphoblastic leukemia. Despite advances with T cell engagers such as blinatumomab some patients respond poorly to treatment even if the patient expresses the tumor antigen for reasons that are not entirely understood.

Strategies for increasing T cell cytotoxicity of T cell engagers have been explored through co-administration with a second antibody that targets the co-inhibitory immune checkpoint programmed death-ligand 1 (PD-L1) and/or CD28. CD28 is a protein expressed on T cells that provide co-stimulatory signals required for T cell activation and survival. It is known that stimulatory signaling through CD28 in combinations with BiTEs increase T cell-induced tumor cell cytotoxicity. However, central to obtaining T cell mediated cytotoxicity of tumor cells in prior studies required the presence of a BiTE that has a tumor binding domain, such as an anti-CD19 antibody, and a CD3 binding domain, while single agent administration of an anti-CD28 and anti-PD-L1 in a scFv-scFv format was found to not induce T cell mediated cytotoxicity against tumor cells.

Activation of T cells is a highly regulated process that typically requires two signaling events for full functionality: the first signal is initiated upon binding of the MHC-antigen complex, which helps distinguish “self” from “non-self” to the T cell receptor (TCR) and the second signal through activation of a costimulatory receptor. While the first recognition signal activates a T cell and triggers T cell mediated toxicity of the recognized cell, if the T cell does not receive a second costimulatory signal it can lead to T cell tolerance whereby the T cells continue to recognize the tumor antigen but do not mount an immune response against the tumor cell. The second costimulatory signal prevents T cell tolerance, and further activates the T cell to enhance T cell cytotoxicity towards the targeted cell.

Multispecific antibodies comprising a CD28 binding domain and PD-L1 binding domain as described herein are designed to act both as an antagonist of PD-L1 and a conditional agonist of C28. While CD28 agonism has shown some clinical promise, the efficacy seen with this approach has been limited due to dose-limiting toxicities that result from systemic activation of CD28. The multispecific antibodies comprising a CD28 binding domain and PD-L1 binding domain, described herein, are designed to conditionally agonize CD28 only in the presence of PD-L1, which is often overexpressed by tumors to avoid T cell mediated killing. In addition, engagement of PD-L1 is designed to block PD-1 binding and provide checkpoint inhibition. This combination provides a mechanism of action that enhances anti-tumor responses and limits the systemic toxicity of CD28 agonism. As shown in FIG. 11, studies of multispecific antibodies described herein demonstrate a lack of systemic immune system activation, as evidenced by the lack of cytokine release. Despite unprecedented clinical response rates, most patients fail to respond to therapies targeting PD-1 and PD-L1, which is due in part because T cells require costimulation for full functionality. As such, checkpoint inhibition alone is likely insufficient to fully enable the immune system to attack a tumor. Further benefit can be derived by the addition of the multispecific antibodies as described herein.

Disclosed herein are antibodies that bind specifically to PD-L1 and CD28 which are able to induce T cell mediated cytotoxicity of tumor cells as a single agent (FIG. 15C) or in combination with a T cell engager (FIG. 16). Significantly, such antibodies that target PD-L1 and CD28 are able to induce T cell mediated cytotoxicity of tumor cells as a single agent, even when not administered with a second agent that specifically targets a tumor cell antigen as exemplified in the schematics of FIGS. 15A-15C. Such antibodies that bind specifically to PD-L1 and CD28 are not in a scFv-scFv format.

Disclosed herein are multispecific antibodies that comprise a CD28 binding domain and a PD-L1 binding domain, wherein when the CD28 binding domain is a single chain variable fragment (scFv), then the PD-L1 binding domain is not a scFv. In some embodiments, the multispecific antibody is according to the formula:

A-L-B (Formula I)

wherein A comprises the CD28 binding domain; B comprises the PD-L1 binding domain; and L comprises a linker that connects A to B. In some embodiments, the multispecific antibody comprises the formula:

A-L-B (Formula I)

wherein A comprises the CD28 binding domain; B comprises the PD-L1 binding domain; and L comprises a linker that connects A to B. In some embodiments, the multispecific antibody comprises the formula:

A-L-B (Formula I)

wherein A is the CD28 binding domain; B is the PD-L1 binding domain; and L is the linker that connects A to B. In some embodiments, the multispecific antibody is according to the formula:

A-L-B (Formula I)

wherein A is the CD28 binding domain; B is the PD-L1 binding domain; and L is the linker that connects A to B.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

In some instances, the CDRs of an antibody are determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273 :927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabatnumbering scheme as 35 A and 35B) (CDRl), amino acid positions 50 to 65 (CDR2), and amino acid positions 95 to 102 (CDR3). Using the Kabat numbering system, CDRs within an antibody light chain molecule are typically present at amino acid positions 24 to 34 (CDRl), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

The term “multispecific” means that the antibody is able to specifically bind to two or more distinct antigenic determinants for example two or more binding sites each formed by a pair of an antibody heavy chain variable domain (VH) and an antibody light chain variable domain (VL), or in the case of a single domain antibody a single variable domain, binding to different antigens.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### CD28 Binding Domains

Disclosed herein are multispecific antibodies that comprise a CD28 binding domain. In some embodiments, the CD28 binding domain comprises an antibody or antigen binding fragment. In some embodiments, the antibody or antigen binding fragment is a monoclonal antibody. In some embodiments, the antibody or antigen binding fragment is a human antibody, a murine antibody, a humanized antibody, or a chimeric antibody. In some embodiments, antibody or antigen binding fragment that binds specifically to CD28 comprises an anti-CD28 heavy chain polypeptide and an anti-CD28 light chain polypeptide.

In some embodiments, the anti-CD28 heavy chain polypeptide comprises an anti-CD28 heavy chain variable domain. In some embodiments, the anti-CD28 heavy chain variable domain comprises an IgG framework, an IgA framework, an IgE framework, or an IgM framework. In some embodiments, the anti-CD28 heavy chain variable domain comprises a variable domain of an IgG1, IgG2, IgG3, or IgG4 heavy chain. In some embodiments, the anti-CD28 light chain polypeptide comprises an anti-CD28 light chain variable domain. In some embodiments, the anti-CD28 light chain variable domain comprises an IgG framework, an IgA framework, an IgE framework, or an IgM framework. In some embodiments, the anti-CD28 light chain variable domain comprises a variable domain of an IgG1, IgG2, IgG3, or IgG4 light chain.

In some embodiments the CD28 binding domain comprises a single chain variable fragment, a single domain antibody, a Fab, or a Fab′. In some embodiments, the CD28 binding domain comprises the single chain variable fragment. In some embodiments, the CD28 binding domain comprises the single domain antibody. In some embodiments, the CD28 binding domain comprises the Fab or the Fab′. In some embodiments, the CD28 binding domain that comprises the single chain variable fragment comprises a scFv heavy chain variable domain and a scFv light chain variable domain. In some embodiments, the anti-CD28 heavy chain polypeptide comprises the scFv heavy chain variable domain. In some embodiments, the anti-CD28 light chain polypeptide comprises the scFv light chain variable domain.

In some embodiments, the anti-CD28 heavy chain polypeptide comprises complementarity determining regions (CDRs): HC-CDR1, HC-CDR2, and HC-CDR3, wherein the HC-CDR1, the HC-CDR2, and the HC-CDR3 of the anti-CD28 heavy chain polypeptide comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some embodiments, the anti-CD28 heavy chain polypeptide comprises complementarity determining regions (CDRs): HC-CDR1, HC-CDR2, and HC-CDR3, wherein the HC-CDR1, the HC-CDR2, and the HC-CDR3 of the anti-CD28 heavy chain polypeptide comprise: HC-CDR1: SEQ ID NO: 1; HC-CDR2: SEQ ID NO: 2; HC-CDR3: SEQ ID NO: 3.

In some embodiments, the anti-CD28 light chain polypeptide comprises complementarity determining regions (CDRs): LC-CDR1, LC-CDR2, and LC-CDR3, wherein the LC-CDR1, the LC-CDR2, and the LC-CDR3 the anti-CD28 light chain polypeptide: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3. In some embodiments, the anti-CD28 light chain polypeptide comprises complementarity determining regions (CDRs): LC-CDR1, LC-CDR2, and LC-CDR3, wherein the LC-CDR1, the LC-CDR2, and the LC-CDR3 of the anti-CD28 light chain polypeptide: LC-CDR1: SEQ ID NO: 4; LC-CDR2: SEQ ID NO: 5; and LC-CDR3: SEQ ID NO: 6.

In some embodiments, anti-CD28 heavy chain polypeptide comprises complementarity determining regions (CDRs): HC-CDR1, HC-CDR2, and HC-CDR3, wherein the HC-CDR1, the HC-CDR2, and the HC-CDR3 of the anti-CD28 heavy chain polypeptide comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3; and the anti-CD28 light chain polypeptide comprises complementarity determining regions (CDRs): LC-CDR1, LC-CDR2, and LC-CDR3, wherein the LC-CDR1, the LC-CDR2, and the LC-CDR3 of the anti-CD28 light chain polypeptide comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3.

In some embodiments, the anti-CD28 heavy chain polypeptide comprises complementarity determining regions (CDRs): HC-CDR1, HC-CDR2, and HC-CDR3, wherein the HC-CDR1, the HC-CDR2, and the HC-CDR3 of the anti-CD28 heavy chain polypeptide comprise: HC-CDR1: SEQ ID NO: 1; HC-CDR2: SEQ ID NO: 2; HC-CDR3: SEQ ID NO: 3; and the anti-CD28 light chain polypeptide comprises complementarity determining regions (CDRs): LC-CDR1, LC-CDR2, and LC-CDR3, wherein the LC-CDR1, the LC-CDR2, and the LC-CDR3 of the anti-CD28 light chain polypeptide comprise: LC-CDR1: SEQ ID NO: 4; LC-CDR2: SEQ ID NO: 5; and LC-CDR3: SEQ ID NO: 6.

TABLE 1
anti-CD28 heavy chain polypeptide complemen-
tarity determining regions (CDR)s as
determined by IMGT definition.

| | Amino Acid | SEQ |
|---|---|---|
| Construct | Sequence | ID |
| Description | (N to C) | NO: |
| anti-CD28: HC: CDR1 | GYTFTSYY | 1 |
| anti-CD28: HC: CDR2 | IYPGNVNT | 2 |
| anti-CD28: HC: CDR3 | TRSHYGLDWNFDV | 3 |

TABLE 2
anti-CD28 light chain polypeptide complemen-
tarity determining regions (CDR)s as
determined by IMGT definition.

| | Amino Acid | SEQ |
|---|---|---|
| Construct | Sequence | ID |
| Description | (N to C) | NO: |
| anti-CD28: LC: CDR1 | QNIYVW | 4 |
| anti-CD28: LC: CDR2 | KA | 5 |
| anti-CD28: LC: CDR3 | QQGQTYPYT | 6 |

In some embodiments, the scFv heavy chain variable domain comprises an amino acid sequence that has at least 70% sequence identity to the amino acid sequence according to SEQ ID NO: 7. In some embodiments, the scFv heavy chain variable domain comprises an amino acid sequence that has at least 80% sequence identity to the amino acid sequence according to SEQ ID NO: 7. In some embodiments, the scFv heavy chain variable domain comprises an amino acid sequence that has at least 85% sequence identity to the amino acid sequence according to SEQ ID NO: 7. In some embodiments, the scFv heavy chain variable domain comprises an amino acid sequence that has at least 90% sequence identity to the amino acid sequence according to SEQ ID NO: 7. In some embodiments, the scFv heavy chain variable domain comprises an amino acid sequence that has at least 95% sequence identity to the amino acid sequence according to SEQ ID NO: 7. In some embodiments, the scFv heavy chain variable domain comprises an amino acid sequence that has at least 96% sequence identity to the amino acid sequence according to SEQ ID NO: 7. In some embodiments, the scFv heavy chain variable domain comprises an amino acid sequence that has at least 97% sequence identity to the amino acid sequence according to SEQ ID NO: 7. In some embodiments, the scFv heavy chain variable domain comprises an amino acid sequence that has at least 98% sequence identity to the amino acid sequence according to SEQ ID NO: 7. In some embodiments, the scFv heavy chain variable domain comprises an amino acid sequence that has at least 99% sequence identity to the amino acid sequence according to SEQ ID NO: 7. In some embodiments, the scFv heavy chain variable domain comprises an amino acid sequence according to SEQ ID NO: 7.

In some embodiments, the scFv heavy chain variable domain comprises an amino acid sequence of at least 75 consecutive amino acid residues of SEQ ID NO: 7. In some embodiments, the scFv heavy chain variable domain comprises an amino acid sequence of at least 100 consecutive amino acid residues of SEQ ID NO: 7 In some embodiments, the scFv heavy chain variable domain comprises an amino acid sequence of at least 105 consecutive amino acid residues of SEQ ID NO: 7 In some embodiments, the scFv heavy chain variable domain comprises an amino acid sequence of at least 110 consecutive amino acid residues of SEQ ID NO: 7. In some embodiments, the scFv heavy chain variable domain comprises an amino acid sequence of at least 115 consecutive amino acid residues of SEQ ID NO: 7.

In some embodiments, the scFv heavy chain variable domain comprises an amino acid sequence of at least 75 consecutive amino acid residues of SEQ ID NO: 7, and has at least 80% sequence identity to the at least 75 consecutive amino acid residues of SEQ ID NO: 7. In some embodiments, the scFv heavy chain variable domain comprises an amino acid sequence of at least 100 consecutive amino acid residues of SEQ ID NO: 7, and has at least 80% sequence identity to the at least 100 consecutive amino acid residues of SEQ ID NO: 7. In some embodiments, the scFv heavy chain variable domain comprises an amino acid sequence of at least 105 consecutive amino acid residues of SEQ ID NO: 7, and has at least 80% sequence identity to the at least 105 consecutive amino acid residues of SEQ ID NO: 7. In some embodiments, the scFv heavy chain variable domain comprises an amino acid sequence of at least 110 consecutive amino acid residues of SEQ ID NO: 7, and has at least 80% sequence identity to the at least 110 consecutive amino acid residues of SEQ ID NO: 7. In some embodiments, the scFv heavy chain variable domain comprises an amino acid sequence of at least 115 consecutive amino acid residues of SEQ ID NO: 7, and has at least 80% sequence identity to the at least 115 consecutive amino acid residues of SEQ ID NO: 7.

In some embodiments, the scFv heavy chain variable domain comprises an amino acid sequence of at least 75 consecutive amino acid residues of SEQ ID NO: 7, and has at least 90% sequence identity to the at least 75 consecutive amino acid residues of SEQ ID NO: 7. In some embodiments, the scFv heavy chain variable domain comprises an amino acid sequence of at least 100 consecutive amino acid residues of SEQ ID NO: 7, and has at least 90% sequence identity to the at least 100 consecutive amino acid residues of SEQ ID NO: 7. In some embodiments, the scFv heavy chain variable domain comprises an amino acid sequence of at least 105 consecutive amino acid residues of SEQ ID NO: 7, and has at least 90% sequence identity to the at least 105 consecutive amino acid residues of SEQ ID NO: 7. In some embodiments, the scFv heavy chain variable domain comprises an amino acid sequence of at least 110 consecutive amino acid residues of SEQ ID NO: 7, and has at least 90% sequence identity to the at least 110 consecutive amino acid residues of SEQ ID NO: 7. In some embodiments, the scFv heavy chain variable domain comprises an amino acid sequence of at least 115 consecutive amino acid residues of SEQ ID NO: 7, and has at least 90% sequence identity to the at least 115 consecutive amino acid residues of SEQ ID NO: 7.

In some embodiments, the scFv heavy chain variable domain comprises an amino acid sequence of at least 75 consecutive amino acid residues of SEQ ID NO: 7, and has at least 95% sequence identity to the at least 75 consecutive amino acid residues of SEQ ID NO: 7. In some embodiments, the scFv heavy chain variable domain comprises an amino acid sequence of at least 100 consecutive amino acid residues of SEQ ID NO: 7, and has at least 95% sequence identity to the at least 100 consecutive amino acid residues of SEQ ID NO: 7. In some embodiments, the scFv heavy chain variable domain comprises an amino acid sequence of at least 105 consecutive amino acid residues of SEQ ID NO: 7, and has at least 95% sequence identity to the at least 105 consecutive amino acid residues of SEQ ID NO: 7. In some embodiments, the scFv heavy chain variable domain comprises an amino acid sequence of at least 110 consecutive amino acid residues of SEQ ID NO: 7, and has at least 95% sequence identity to the at least 110 consecutive amino acid residues of SEQ ID NO: 7. In some embodiments, the scFv heavy chain variable domain comprises an amino acid sequence of at least 115 consecutive amino acid residues of SEQ ID NO: 7, and has at least 95% sequence identity to the at least 115 consecutive amino acid residues of SEQ ID NO: 7.

In some embodiments, the scFv heavy chain variable domain comprises an amino acid sequence of at least 75 consecutive amino acid residues of SEQ ID NO: 7, and has at least 99% sequence identity to the at least 75 consecutive amino acid residues of SEQ ID NO: 7. In some embodiments, the scFv heavy chain variable domain comprises an amino acid sequence of at least 100 consecutive amino acid residues of SEQ ID NO: 7, and has at least 99% sequence identity to the at least 100 consecutive amino acid residues of SEQ ID NO: 7. In some embodiments, the scFv heavy chain variable domain comprises an amino acid sequence of at least 105 consecutive amino acid residues of SEQ ID NO: 7, and has at least 99% sequence identity to the at least 105 consecutive amino acid residues of SEQ ID NO: 7. In some embodiments, the scFv heavy chain variable domain comprises an amino acid sequence of at least 110 consecutive amino acid residues of SEQ ID NO: 7, and has at least 99% sequence identity to the at least 110 consecutive amino acid residues of SEQ ID NO: 7. In some embodiments, the scFv heavy chain variable domain comprises an amino acid sequence of at least 115 consecutive amino acid residues of SEQ ID NO: 7, and has at least 99% sequence identity to the at least 115 consecutive amino acid residues of SEQ ID NO: 7.

In some embodiments, the scFv light chain variable domain comprises an amino acid sequence that has at least 70% sequence identity to the amino acid sequence according to SEQ ID NO: 8. In some embodiments, the scFv light chain variable domain comprises an amino acid sequence that has at least 80% sequence identity to the amino acid sequence according to SEQ ID NO: 8. In some embodiments, the scFv light chain variable domain comprises an amino acid sequence that has at least 85% sequence identity to the amino acid sequence according to SEQ ID NO: 8. In some embodiments, the scFv light chain variable domain comprises an amino acid sequence that has at least 90% sequence identity to the amino acid sequence according to SEQ ID NO: 8. In some embodiments, the scFv light chain variable domain comprises an amino acid sequence that has at least 95% sequence identity to the amino acid sequence according to SEQ ID NO: 8. In some embodiments, the scFv light chain variable domain comprises an amino acid sequence that has at least 96% sequence identity to the amino acid sequence according to SEQ ID NO: 8. In some embodiments, the scFv light chain variable domain comprises an amino acid sequence that has at least 97% sequence identity to the amino acid sequence according to SEQ ID NO: 8. In some embodiments, the scFv light chain variable domain comprises an amino acid sequence that has at least 98% sequence identity to the amino acid sequence according to SEQ ID NO: 8. In some embodiments, the scFv light chain variable domain comprises an amino acid sequence that has at least 99% sequence identity to the amino acid sequence according to SEQ ID NO: 8. In some embodiments, the scFv light chain variable domain comprises an amino acid sequence according to SEQ ID NO: 8.

In some embodiments, the scFv light chain variable domain comprises an amino acid sequence of at least 75 consecutive amino acid residues of SEQ ID NO: 8. In some embodiments, the scFv light chain variable domain comprises an amino acid sequence of at least 80 consecutive amino acid residues of SEQ ID NO: 8. In some embodiments, the scFv light chain variable domain comprises an amino acid sequence of at least 90 consecutive amino acid residues of SEQ ID NO: 8. In some embodiments, the scFv light chain variable domain comprises an amino acid sequence of at least 95 consecutive amino acid residues of SEQ ID NO: 8. In some embodiments, the scFv light chain variable domain comprises an amino acid sequence of at least 100 consecutive amino acid residues of SEQ ID NO: 8. In some embodiments, the scFv light chain variable domain comprises an amino acid sequence of at least 105 consecutive amino acid residues of SEQ ID NO: 8.

In some embodiments, the scFv light chain variable domain comprises an amino acid sequence of at least 75 consecutive amino acid residues of SEQ ID NO: 8, and has at least 80% sequence identity to the at least 75 consecutive amino acid residues of SEQ ID NO: 8. In some embodiments, the scFv light chain variable domain comprises an amino acid sequence of at least 80 consecutive amino acid residues of SEQ ID NO: 8, and has at least 80% sequence identity to the at least 80 consecutive amino acid residues of SEQ ID NO: 8. In some embodiments, the scFv light chain variable domain comprises an amino acid sequence of at least 90 consecutive amino acid residues of SEQ ID NO: 8, and has at least 80% sequence identity to the at least 90 consecutive amino acid residues of SEQ ID NO: 8. In some embodiments, the scFv light chain variable domain comprises an amino acid sequence of at least 95 consecutive amino acid residues of SEQ ID NO: 8, and has at least 80% sequence identity to the at least 95 consecutive amino acid residues of SEQ ID NO: 8. In some embodiments, the scFv light chain variable domain comprises an amino acid sequence of at least 100 consecutive amino acid residues of SEQ ID NO: 8, and has at least 80% sequence identity to the at least 100 consecutive amino acid residues of SEQ ID NO: 8. In some embodiments, the scFv light chain variable domain comprises an amino acid sequence of at least 105 consecutive amino acid residues of SEQ ID NO: 8, and has at least 80% sequence identity to the at least 105 consecutive amino acid residues of SEQ ID NO: 8.

In some embodiments, the scFv light chain variable domain comprises an amino acid sequence of at least 75 consecutive amino acid residues of SEQ ID NO: 8, and has at least 90% sequence identity to the at least 75 consecutive amino acid residues of SEQ ID NO: 8. In some embodiments, the scFv light chain variable domain comprises an amino acid sequence of at least 80 consecutive amino acid residues of SEQ ID NO: 8, and has at least 90% sequence identity to the at least 80 consecutive amino acid residues of SEQ ID NO: 8. In some embodiments, the scFv light chain variable domain comprises an amino acid sequence of at least 90 consecutive amino acid residues of SEQ ID NO: 8, and has at least 90% sequence identity to the at least 90 consecutive amino acid residues of SEQ ID NO: 8. In some embodiments, the scFv light chain variable domain comprises an amino acid sequence of at least 95 consecutive amino acid residues of SEQ ID NO: 8, and has at least 90% sequence identity to the at least 95 consecutive amino acid residues of SEQ ID NO: 8. In some embodiments, the scFv light chain variable domain comprises an amino acid sequence of at least 100 consecutive amino acid residues of SEQ ID NO: 8, and has at least 90% sequence identity to the at least 100 consecutive amino acid residues of SEQ ID NO: 8. In some embodiments, the scFv light chain variable domain comprises an amino acid sequence of at least 105 consecutive amino acid residues of SEQ ID NO: 8, and has at least 90% sequence identity to the at least 105 consecutive amino acid residues of SEQ ID NO: 8.

In some embodiments, the scFv light chain variable domain comprises an amino acid sequence of at least 75 consecutive amino acid residues of SEQ ID NO: 8, and has at least 95% sequence identity to the at least 75 consecutive amino acid residues of SEQ ID NO: 8. In some embodiments, the scFv light chain variable domain comprises an amino acid sequence of at least 80 consecutive amino acid residues of SEQ ID NO: 8, and has at least 95% sequence identity to the at least 80 consecutive amino acid residues of SEQ ID NO: 8. In some embodiments, the scFv light chain variable domain comprises an amino acid sequence of at least 90 consecutive amino acid residues of SEQ ID NO: 8, and has at least 95% sequence identity to the at least 90 consecutive amino acid residues of SEQ ID NO: 8. In some embodiments, the scFv light chain variable domain comprises an amino acid sequence of at least 95 consecutive amino acid residues of SEQ ID NO: 8, and has at least 95% sequence identity to the at least 95 consecutive amino acid residues of SEQ ID NO: 8. In some embodiments, the scFv light chain variable domain comprises an amino acid sequence of at least 100 consecutive amino acid residues of SEQ ID NO: 8, and has at least 95% sequence identity to the at least 100 consecutive amino acid residues of SEQ ID NO: 8. In some embodiments, the scFv light chain variable domain comprises an amino acid sequence of at least 105 consecutive amino acid residues of SEQ ID NO: 8, and has at least 95% sequence identity to the at least 105 consecutive amino acid residues of SEQ ID NO: 8.

In some embodiments, the scFv light chain variable domain comprises an amino acid sequence of at least 75 consecutive amino acid residues of SEQ ID NO: 8, and has at least 99% sequence identity to the at least 75 consecutive amino acid residues of SEQ ID NO: 8. In some embodiments, the scFv light chain variable domain comprises an amino acid sequence of at least 80 consecutive amino acid residues of SEQ ID NO: 8, and has at least 99% sequence identity to the at least 80 consecutive amino acid residues of SEQ ID NO: 8. In some embodiments, the scFv light chain variable domain comprises an amino acid sequence of at least 90 consecutive amino acid residues of SEQ ID NO: 8, and has at least 99% sequence identity to the at least 90 consecutive amino acid residues of SEQ ID NO: 8. In some embodiments, the scFv light chain variable domain comprises an amino acid sequence of at least 95 consecutive amino acid residues of SEQ ID NO: 8, and has at least 99% sequence identity to the at least 95 consecutive amino acid residues of SEQ ID NO: 8. In some embodiments, the scFv light chain variable domain comprises an amino acid sequence of at least 100 consecutive amino acid residues of SEQ ID NO: 8, and has at least 99% sequence identity to the at least 100 consecutive amino acid residues of SEQ ID NO: 8. In some embodiments, the scFv light chain variable domain comprises an amino acid sequence of at least 105 consecutive amino acid residues of SEQ ID NO: 8, and has at least 99% sequence identity to the at least 105 consecutive amino acid residues of SEQ ID NO: 8.

In some embodiments, the scFv comprises an amino acid sequence that has at least 70% sequence identity to the amino acid sequence according to SEQ ID NO: 9. In some embodiments, the scFv comprises an amino acid sequence that has at least 80% sequence identity to the amino acid sequence according to SEQ ID NO: 9. In some embodiments, the scFv comprises an amino acid sequence that has at least 85% sequence identity to the amino acid sequence according to SEQ ID NO: 9. In some embodiments, the scFv comprises an amino acid sequence that has at least 90% sequence identity to the amino acid sequence according to SEQ ID NO: 9. In some embodiments, the scFv comprises an amino acid sequence that has at least 91% sequence identity to the amino acid sequence according to SEQ ID NO: 9. In some embodiments, the scFv comprises an amino acid sequence that has at least 92% sequence identity to the amino acid sequence according to SEQ ID NO: 9. In some embodiments, the scFv comprises an amino acid sequence that has at least 93% sequence identity to the amino acid sequence according to SEQ ID NO: 9. In some embodiments, the scFv comprises an amino acid sequence that has at least 94% sequence identity to the amino acid sequence according to SEQ ID NO: 9. In some embodiments, the scFv comprises an amino acid sequence that has at least 95% sequence identity to the amino acid sequence according to SEQ ID NO: 9. In some embodiments, the scFv comprises an amino acid sequence that has at least 96% sequence identity to the amino acid sequence according to SEQ ID NO: 9. In some embodiments, the scFv comprises an amino acid sequence that has at least 97% sequence identity to the amino acid sequence according to SEQ ID NO: 9. In some embodiments, the scFv comprises an amino acid sequence that has at least 98% sequence identity to the amino acid sequence according to SEQ ID NO: 9. In some embodiments, the scFv comprises an amino acid sequence that has at least 99% sequence identity to the amino acid sequence according to SEQ ID NO: 9. In some embodiments, the scFv comprises an amino acid sequence according to SEQ ID NO: 9.

In some embodiments, the scFv comprises an amino acid sequence of at least 175 consecutive amino acid residues of SEQ ID NO: 9. In some embodiments, the scFv comprises an amino acid sequence of at least 190 consecutive amino acid residues of SEQ ID NO: 9. In some embodiments, the scFv comprises an amino acid sequence of at least 200 consecutive amino acid residues of SEQ ID NO: 9.In some embodiments, the scFv comprises an amino acid sequence of at least 205 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 215 consecutive amino acid residues of SEQ ID NO: 9.

In some embodiments, the scFv comprises an amino acid sequence of at least 175 consecutive amino acid residues of SEQ ID NO: 9, and has at least 80% sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 80% sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 80% sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 80% sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 80% sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 80% sequence identity to the at least 215 consecutive amino acid residues of SEQ ID NO: 9.

In some embodiments, the scFv comprises an amino acid sequence of at least 175 consecutive amino acid residues of SEQ ID NO: 9, and has at least 90% sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 90% sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 90% sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 90% sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 90% sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 90% sequence identity to the at least 215 consecutive amino acid residues of SEQ ID NO: 9.

In some embodiments, the scFv comprises an amino acid sequence of at least 175 consecutive amino acid residues of SEQ ID NO: 9, and has at least 95% sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 95% sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 95% sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 95% sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 95% sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 95 sequence identity to the at least 215 consecutive amino acid residues of SEQ ID NO: 9.

In some embodiments, the scFv comprises an amino acid sequence of at least 175 consecutive amino acid residues of SEQ ID NO: 9, and has at least 99% o sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 99% o sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 99% o sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 99% o sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 99% o sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 99% o sequence identity to the at least 215 consecutive amino acid residues of SEQ ID NO: 9.

TABLE 3
anti-CD28 light chain variable domain, heavy
chain variable domain sequences, and full
length sequence. CDR sequences are underlined
and were determined using IMGT definition.

| | Amino Acid | SEQ |
|---|---|---|
| Construct | Sequence | ID |
| Description | (N to C) | NO: |
| anti-CD28: | QVQLVQSGAEVKKPGASVKV | 7 |
| HC | SCKASGYTFTSYYIHWVRQAP | |
| | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSS | |
| anti-CD28: | DIQMTQSPSSLSASVGDRVTIT | 8 |
| LC | CHASQNIYVWLNWYQQKPG | |
| | KAPKLLIYKASNLHTGVPSRFS | |
| | GSGSGTDFTLTISSLQPEDFAT | |
| | YYCQQGQTYPYTFGGGTKVE | |
| | IK | |
| Anti-CD28 | QVQLVQSGAEVKKPGASVKV | 9 |
| scFv (VH - | SCKASGYTFTSYYIHWVRQAP | |
| linker 1 - | GQGLEWIGSIYPGNVNTNYNE | |
| VL) | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIK | |

In some embodiments, the CD28 binding domain comprises complementarity determining regions (CDRs): HC-CDR1, HC-CDR2, and HC-CDR3, wherein the HC-CDR1, the HC-CDR2, and the HC-CDR3 of the CD28 binding domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3. In some embodiments, the CD28 binding domain comprises complementarity determining regions (CDRs): HC-CDR1, HC-CDR2, and HC-CDR3, wherein the HC-CDR1, the HC-CDR2, and the HC-CDR3 of the CD28 binding domain comprise: HC-CDR1: SEQ ID NO: 1; HC-CDR2: SEQ ID NO: 2; HC-CDR3: SEQ ID NO: 3.

In some embodiments, the CD28 binding domain comprises complementarity determining regions (CDRs): LC-CDR1, LC-CDR2, and LC-CDR3, wherein the LC-CDR1, the LC-CDR2, and the LC-CDR3 the anti-CD28 light chain polypeptide: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3. In some embodiments, the CD28 binding domain comprises complementarity determining regions (CDRs): LC-CDR1, LC-CDR2, and LC-CDR3, wherein the LC-CDR1, the LC-CDR2, and the LC-CDR3 of the anti-CD28 light chain polypeptide: LC-CDR1: SEQ ID NO: 4; LC-CDR2: SEQ ID NO: 5; and LC-CDR3: SEQ ID NO: 6.

In some embodiments, CD28 binding domain comprises complementarity determining regions (CDRs): HC-CDR1, HC-CDR2, and HC-CDR3, wherein the HC-CDR1, the HC-CDR2, and the HC-CDR3 of the CD28 binding domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3; and the CD28 binding domain comprises complementarity determining regions (CDRs): LC-CDR1, LC-CDR2, and LC-CDR3, wherein the LC-CDR1, the LC-CDR2, and the LC-CDR3 of the CD28 binding domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3.

In some embodiments, the CD28 binding domain comprises complementarity determining regions (CDRs): HC-CDR1, HC-CDR2, and HC-CDR3, wherein the HC-CDR1, the HC-CDR2, and the HC-CDR3 of the CD28 binding domain comprise: HC-CDR1: SEQ ID NO: 1; HC-CDR2: SEQ ID NO: 2; HC-CDR3: SEQ ID NO: 3; and the CD28 binding domain comprises complementarity determining regions (CDRs): LC-CDR1, LC-CDR2, and LC-CDR3, wherein the LC-CDR1, the LC-CDR2, and the LC-CDR3 of the CD28 binding domain comprise: LC-CDR1: SEQ ID NO: 4; LC-CDR2: SEQ ID NO: 5; and LC-CDR3: SEQ ID NO: 6.

In some embodiments, the CD28 binding domain comprises an amino acid sequence that has at least 70% sequence identity to the amino acid sequence according to SEQ ID NO: 7. In some embodiments, the CD28 binding domain comprises an amino acid sequence that has at least 80% sequence identity to the amino acid sequence according to SEQ ID NO: 7. In some embodiments, the CD28 binding domain comprises an amino acid sequence that has at least 85% sequence identity to the amino acid sequence according to SEQ ID NO: 7. In some embodiments, the CD28 binding domain comprises an amino acid sequence that has at least 90% sequence identity to the amino acid sequence according to SEQ ID NO: 7. In some embodiments, the CD28 binding domain comprises an amino acid sequence that has at least 95% sequence identity to the amino acid sequence according to SEQ ID NO: 7. In some embodiments, the CD28 binding domain comprises an amino acid sequence that has at least 96% sequence identity to the amino acid sequence according to SEQ ID NO: 7. In some embodiments, the CD28 binding domain comprises an amino acid sequence that has at least 97% sequence identity to the amino acid sequence according to SEQ ID NO: 7. In some embodiments, the CD28 binding domain comprises an amino acid sequence that has at least 98% sequence identity to the amino acid sequence according to SEQ ID NO: 7. In some embodiments, the CD28 binding domain comprises an amino acid sequence that has at least 99% sequence identity to the amino acid sequence according to SEQ ID NO: 7. In some embodiments, the CD28 binding domain comprises an amino acid sequence according to SEQ ID NO: 7.

In some embodiments, the CD28 binding domain comprises an amino acid sequence of at least 75 consecutive amino acid residues of SEQ ID NO: 7. In some embodiments, the CD28 binding domain comprises an amino acid sequence of at least 100 consecutive amino acid residues of SEQ ID NO: 7 In some embodiments, the CD28 binding domain comprises an amino acid sequence of at least 105 consecutive amino acid residues of SEQ ID NO: 7 In some embodiments, the CD28 binding domain comprises an amino acid sequence of at least 110 consecutive amino acid residues of SEQ ID NO: 7. In some embodiments, the CD28 binding domain comprises an amino acid sequence of at least 115 consecutive amino acid residues of SEQ ID NO: 7.

In some embodiments, the CD28 binding domain comprises an amino acid sequence of at least 75 consecutive amino acid residues of SEQ ID NO: 7, and has at least 80% sequence identity to the at least 75 consecutive amino acid residues of SEQ ID NO: 7. In some embodiments, the CD28 binding domain comprises an amino acid sequence of at least 100 consecutive amino acid residues of SEQ ID NO: 7, and has at least 80% sequence identity to the at least 100 consecutive amino acid residues of SEQ ID NO: 7. In some embodiments, the CD28 binding domain comprises an amino acid sequence of at least 105 consecutive amino acid residues of SEQ ID NO: 7, and has at least 80% sequence identity to the at least 105 consecutive amino acid residues of SEQ ID NO: 7. In some embodiments, the CD28 binding domain comprises an amino acid sequence of at least 110 consecutive amino acid residues of SEQ ID NO: 7, and has at least 80% sequence identity to the at least 110 consecutive amino acid residues of SEQ ID NO: 7. In some embodiments, the CD28 binding domain comprises an amino acid sequence of at least 115 consecutive amino acid residues of SEQ ID NO: 7, and has at least 80% sequence identity to the at least 115 consecutive amino acid residues of SEQ ID NO: 7.

In some embodiments, the CD28 binding domain comprises an amino acid sequence of at least 75 consecutive amino acid residues of SEQ ID NO: 7, and has at least 90% sequence identity to the at least 75 consecutive amino acid residues of SEQ ID NO: 7. In some embodiments, the CD28 binding domain comprises an amino acid sequence of at least 100 consecutive amino acid residues of SEQ ID NO: 7, and has at least 90% sequence identity to the at least 100 consecutive amino acid residues of SEQ ID NO: 7. In some embodiments, the CD28 binding domain comprises an amino acid sequence of at least 105 consecutive amino acid residues of SEQ ID NO: 7, and has at least 90% sequence identity to the at least 105 consecutive amino acid residues of SEQ ID NO: 7. In some embodiments, the CD28 binding domain comprises an amino acid sequence of at least 110 consecutive amino acid residues of SEQ ID NO: 7, and has at least 90% sequence identity to the at least 110 consecutive amino acid residues of SEQ ID NO: 7. In some embodiments, the CD28 binding domain comprises an amino acid sequence of at least 115 consecutive amino acid residues of SEQ ID NO: 7, and has at least 90% sequence identity to the at least 115 consecutive amino acid residues of SEQ ID NO: 7.

In some embodiments, the CD28 binding domain comprises an amino acid sequence of at least 75 consecutive amino acid residues of SEQ ID NO: 7, and has at least 95% sequence identity to the at least 75 consecutive amino acid residues of SEQ ID NO: 7. In some embodiments, the CD28 binding domain comprises an amino acid sequence of at least 100 consecutive amino acid residues of SEQ ID NO: 7, and has at least 95% sequence identity to the at least 100 consecutive amino acid residues of SEQ ID NO: 7. In some embodiments, the CD28 binding domain comprises an amino acid sequence of at least 105 consecutive amino acid residues of SEQ ID NO: 7, and has at least 95% sequence identity to the at least 105 consecutive amino acid residues of SEQ ID NO: 7. In some embodiments, the CD28 binding domain comprises an amino acid sequence of at least 110 consecutive amino acid residues of SEQ ID NO: 7, and has at least 95% sequence identity to the at least 110 consecutive amino acid residues of SEQ ID NO: 7. In some embodiments, the CD28 binding domain comprises an amino acid sequence of at least 115 consecutive amino acid residues of SEQ ID NO: 7, and has at least 95% sequence identity to the at least 115 consecutive amino acid residues of SEQ ID NO: 7.

In some embodiments, the CD28 binding domain comprises an amino acid sequence of at least 75 consecutive amino acid residues of SEQ ID NO: 7, and has at least 99% sequence identity to the at least 75 consecutive amino acid residues of SEQ ID NO: 7. In some embodiments, the CD28 binding domain comprises an amino acid sequence of at least 100 consecutive amino acid residues of SEQ ID NO: 7, and has at least 99% sequence identity to the at least 100 consecutive amino acid residues of SEQ ID NO: 7. In some embodiments, the CD28 binding domain comprises an amino acid sequence of at least 105 consecutive amino acid residues of SEQ ID NO: 7, and has at least 99% sequence identity to the at least 105 consecutive amino acid residues of SEQ ID NO: 7. In some embodiments, the CD28 binding domain comprises an amino acid sequence of at least 110 consecutive amino acid residues of SEQ ID NO: 7, and has at least 99% sequence identity to the at least 110 consecutive amino acid residues of SEQ ID NO: 7. In some embodiments, the CD28 binding domain comprises an amino acid sequence of at least 115 consecutive amino acid residues of SEQ ID NO: 7, and has at least 99% sequence identity to the at least 115 consecutive amino acid residues of SEQ ID NO: 7.

In some embodiments, the CD28 binding domain comprises an amino acid sequence that has at least 70% sequence identity to the amino acid sequence according to SEQ ID NO: 8. In some embodiments, the CD28 binding domain comprises an amino acid sequence that has at least 80% sequence identity to the amino acid sequence according to SEQ ID NO: 8. In some embodiments, the CD28 binding domain comprises an amino acid sequence that has at least 85% sequence identity to the amino acid sequence according to SEQ ID NO: 8. In some embodiments, the CD28 binding domain comprises an amino acid sequence that has at least 90% sequence identity to the amino acid sequence according to SEQ ID NO: 8. In some embodiments, the CD28 binding domain comprises an amino acid sequence that has at least 95% sequence identity to the amino acid sequence according to SEQ ID NO: 8. In some embodiments, the CD28 binding domain comprises an amino acid sequence that has at least 96% sequence identity to the amino acid sequence according to SEQ ID NO: 8. In some embodiments, the CD28 binding domain comprises an amino acid sequence that has at least 97% sequence identity to the amino acid sequence according to SEQ ID NO: 8. In some embodiments, the CD28 binding domain comprises an amino acid sequence that has at least 98% sequence identity to the amino acid sequence according to SEQ ID NO: 8. In some embodiments, the CD28 binding domain comprises an amino acid sequence that has at least 99% sequence identity to the amino acid sequence according to SEQ ID NO: 8. In some embodiments, the CD28 binding domain comprises an amino acid sequence according to SEQ ID NO: 8.

In some embodiments, the CD28 binding domain comprises an amino acid sequence of at least 75 consecutive amino acid residues of SEQ ID NO: 8. In some embodiments, the CD28 binding domain comprises an amino acid sequence of at least 80 consecutive amino acid residues of SEQ ID NO: 8. In some embodiments, the CD28 binding domain comprises an amino acid sequence of at least 90 consecutive amino acid residues of SEQ ID NO: 8. In some embodiments, the CD28 binding domain comprises an amino acid sequence of at least 95 consecutive amino acid residues of SEQ ID NO: 8. In some embodiments, the CD28 binding domain comprises an amino acid sequence of at least 100 consecutive amino acid residues of SEQ ID NO: 8. In some embodiments, the CD28 binding domain comprises an amino acid sequence of at least 105 consecutive amino acid residues of SEQ ID NO: 8.

In some embodiments, the CD28 binding domain comprises an amino acid sequence of at least 75 consecutive amino acid residues of SEQ ID NO: 8, and has at least 80% sequence identity to the at least 75 consecutive amino acid residues of SEQ ID NO: 8. In some embodiments, the CD28 binding domain comprises an amino acid sequence of at least 80 consecutive amino acid residues of SEQ ID NO: 8, and has at least 80% sequence identity to the at least 80 consecutive amino acid residues of SEQ ID NO: 8. In some embodiments, the CD28 binding domain comprises an amino acid sequence of at least 90 consecutive amino acid residues of SEQ ID NO: 8, and has at least 80% sequence identity to the at least 90 consecutive amino acid residues of SEQ ID NO: 8. In some embodiments, the CD28 binding domain comprises an amino acid sequence of at least 95 consecutive amino acid residues of SEQ ID NO: 8, and has at least 80% sequence identity to the at least 95 consecutive amino acid residues of SEQ ID NO: 8. In some embodiments, the CD28 binding domain comprises an amino acid sequence of at least 100 consecutive amino acid residues of SEQ ID NO: 8, and has at least 80% sequence identity to the at least 100 consecutive amino acid residues of SEQ ID NO: 8. In some embodiments, the CD28 binding domain comprises an amino acid sequence of at least 105 consecutive amino acid residues of SEQ ID NO: 8, and has at least 80% sequence identity to the at least 105 consecutive amino acid residues of SEQ ID NO: 8.

In some embodiments, the CD28 binding domain comprises an amino acid sequence of at least 75 consecutive amino acid residues of SEQ ID NO: 8, and has at least 90% sequence identity to the at least 75 consecutive amino acid residues of SEQ ID NO: 8. In some embodiments, the CD28 binding domain comprises an amino acid sequence of at least 80 consecutive amino acid residues of SEQ ID NO: 8, and has at least 90% sequence identity to the at least 80 consecutive amino acid residues of SEQ ID NO: 8. In some embodiments, the CD28 binding domain comprises an amino acid sequence of at least 90 consecutive amino acid residues of SEQ ID NO: 8, and has at least 90% sequence identity to the at least 90 consecutive amino acid residues of SEQ ID NO: 8. In some embodiments, the CD28 binding domain comprises an amino acid sequence of at least 95 consecutive amino acid residues of SEQ ID NO: 8, and has at least 90% sequence identity to the at least 95 consecutive amino acid residues of SEQ ID NO: 8. In some embodiments, the CD28 binding domain comprises an amino acid sequence of at least 100 consecutive amino acid residues of SEQ ID NO: 8, and has at least 90% sequence identity to the at least 100 consecutive amino acid residues of SEQ ID NO: 8. In some embodiments, the CD28 binding domain comprises an amino acid sequence of at least 105 consecutive amino acid residues of SEQ ID NO: 8, and has at least 90% sequence identity to the at least 105 consecutive amino acid residues of SEQ ID NO: 8.

In some embodiments, the CD28 binding domain comprises an amino acid sequence of at least 75 consecutive amino acid residues of SEQ ID NO: 8, and has at least 95% sequence identity to the at least 75 consecutive amino acid residues of SEQ ID NO: 8. In some embodiments, the CD28 binding domain comprises an amino acid sequence of at least 80 consecutive amino acid residues of SEQ ID NO: 8, and has at least 95% sequence identity to the at least 80 consecutive amino acid residues of SEQ ID NO: 8. In some embodiments, the CD28 binding domain comprises an amino acid sequence of at least 90 consecutive amino acid residues of SEQ ID NO: 8, and has at least 95% sequence identity to the at least 90 consecutive amino acid residues of SEQ ID NO: 8.

In some embodiments, the CD28 binding domain comprises an amino acid sequence of at least 95 consecutive amino acid residues of SEQ ID NO: 8, and has at least 95% sequence identity to the at least 95 consecutive amino acid residues of SEQ ID NO: 8. In some embodiments, the CD28 binding domain comprises an amino acid sequence of at least 100 consecutive amino acid residues of SEQ ID NO: 8, and has at least 95% sequence identity to the at least 100 consecutive amino acid residues of SEQ ID NO: 8. In some embodiments, the CD28 binding domain comprises an amino acid sequence of at least 105 consecutive amino acid residues of SEQ ID NO: 8, and has at least 95% sequence identity to the at least 105 consecutive amino acid residues of SEQ ID NO: 8.

In some embodiments, the CD28 binding domain comprises an amino acid sequence of at least 75 consecutive amino acid residues of SEQ ID NO: 8, and has at least 99% sequence identity to the at least 75 consecutive amino acid residues of SEQ ID NO: 8. In some embodiments, the CD28 binding domain comprises an amino acid sequence of at least 80 consecutive amino acid residues of SEQ ID NO: 8, and has at least 99% sequence identity to the at least 80 consecutive amino acid residues of SEQ ID NO: 8. In some embodiments, the CD28 binding domain comprises an amino acid sequence of at least 90 consecutive amino acid residues of SEQ ID NO: 8, and has at least 99% sequence identity to the at least 90 consecutive amino acid residues of SEQ ID NO: 8.

In some embodiments, the CD28 binding domain comprises an amino acid sequence of at least 95 consecutive amino acid residues of SEQ ID NO: 8, and has at least 99% sequence identity to the at least 95 consecutive amino acid residues of SEQ ID NO: 8. In some embodiments, the CD28 binding domain comprises an amino acid sequence of at least 100 consecutive amino acid residues of SEQ ID NO: 8, and has at least 99% sequence identity to the at least 100 consecutive amino acid residues of SEQ ID NO: 8. In some embodiments, the CD28 binding domain comprises an amino acid sequence of at least 105 consecutive amino acid residues of SEQ ID NO: 8, and has at least 99% sequence identity to the at least 105 consecutive amino acid residues of SEQ ID NO: 8.

In some embodiments, the scFv comprises an amino acid sequence that has at least 70% sequence identity to the amino acid sequence according to SEQ ID NO: 9. In some embodiments, the scFv comprises an amino acid sequence that has at least 80% sequence identity to the amino acid sequence according to SEQ ID NO: 9. In some embodiments, the scFv comprises an amino acid sequence that has at least 85% sequence identity to the amino acid sequence according to SEQ ID NO: 9. In some embodiments, the scFv comprises an amino acid sequence that has at least 90% sequence identity to the amino acid sequence according to SEQ ID NO: 9. In some embodiments, the scFv comprises an amino acid sequence that has at least 91% sequence identity to the amino acid sequence according to SEQ ID NO: 9. In some embodiments, the scFv comprises an amino acid sequence that has at least 92% sequence identity to the amino acid sequence according to SEQ ID NO: 9. In some embodiments, the scFv comprises an amino acid sequence that has at least 93% sequence identity to the amino acid sequence according to SEQ ID NO: 9. In some embodiments, the scFv comprises an amino acid sequence that has at least 94% sequence identity to the amino acid sequence according to SEQ ID NO: 9. In some embodiments, the scFv comprises an amino acid sequence that has at least 95% sequence identity to the amino acid sequence according to SEQ ID NO: 9. In some embodiments, the scFv comprises an amino acid sequence that has at least 96% sequence identity to the amino acid sequence according to SEQ ID NO: 9. In some embodiments, the scFv comprises an amino acid sequence that has at least 97% sequence identity to the amino acid sequence according to SEQ ID NO: 9. In some embodiments, the scFv comprises an amino acid sequence that has at least 98% sequence identity to the amino acid sequence according to SEQ ID NO: 9. In some embodiments, the scFv comprises an amino acid sequence that has at least 99% sequence identity to the amino acid sequence according to SEQ ID NO: 9. In some embodiments, the scFv comprises an amino acid sequence according to SEQ ID NO: 9.

In some embodiments, the scFv comprises an amino acid sequence of at least 175 consecutive amino acid residues of SEQ ID NO: 9. In some embodiments, the scFv comprises an amino acid sequence of at least 190 consecutive amino acid residues of SEQ ID NO: 9. In some embodiments, the scFv comprises an amino acid sequence of at least 200 consecutive amino acid residues of SEQ ID NO: 9.In some embodiments, the scFv comprises an amino acid sequence of at least 205 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 215 consecutive amino acid residues of SEQ ID NO: 9.

In some embodiments, the scFv comprises an amino acid sequence of at least 175 consecutive amino acid residues of SEQ ID NO: 9, and has at least 80% sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 80% sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 80% sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 80% sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 80% sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 80% sequence identity to the at least 215 consecutive amino acid residues of SEQ ID NO: 9.

In some embodiments, the scFv comprises an amino acid sequence of at least 175 consecutive amino acid residues of SEQ ID NO: 9, and has at least 90% sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 90% sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 90% sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 90% sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 90% sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 90% sequence identity to the at least 215 consecutive amino acid residues of SEQ ID NO: 9.

In some embodiments, the scFv comprises an amino acid sequence of at least 175 consecutive amino acid residues of SEQ ID NO: 9, and has at least 95% sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 95% sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 95% sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 95% sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 95% sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 95% sequence identity to the at least 215 consecutive amino acid residues of SEQ ID NO: 9.

In some embodiments, the scFv comprises an amino acid sequence of at least 175 consecutive amino acid residues of SEQ ID NO: 9, and has at least 99% sequence identity to the at least 175 consecutive amino acid residues of SEQ ID NO: 9. In some embodiments, the scFv comprises an amino acid sequence of at least 190 consecutive amino acid residues of SEQ ID NO: 9, and has at least 99% sequence identity to the at least 190 consecutive amino acid residues of SEQ ID NO: 9. In some embodiments, the scFv comprises an amino acid sequence of at least 200 consecutive amino acid residues of SEQ ID NO: 9, and has at least 99% sequence identity to the at least 200 consecutive amino acid residues of SEQ ID NO: 9. In some embodiments, the scFv comprises an amino acid sequence of at least 205 consecutive amino acid residues of SEQ ID NO: 9, and has at least 99% sequence identity to the at least 205 consecutive amino acid residues of SEQ ID NO: 9. In some embodiments, the scFv comprises an amino acid sequence of at least 210 consecutive amino acid residues of SEQ ID NO: 9, and has at least 99% sequence identity to the at least 210 consecutive amino acid residues of SEQ ID NO: 9. In some embodiments, the scFv comprises an amino acid sequence of at least 215 consecutive amino acid residues of SEQ ID NO: 9, and has at least 99% sequence identity to the at least 215 consecutive amino acid residues of SEQ ID NO: 9.

### PD-L1 Binding Domains

Disclosed here are multispecific antibodies that comprise a PD-L1 binding domain. In some embodiments, the PD-L1 binding domain comprises an antibody or antigen binding fragment. In some embodiments, the antibody or antigen binding fragment is a monoclonal antibody. In some embodiments, the antibody or antigen binding fragment is a human antibody, a murine antibody, a humanized antibody, or a chimeric antibody. In some embodiments, antibody or antigen binding fragment that binds specifically to PD-L1 comprises an anti-PD-L1 heavy chain polypeptide and an anti-PD-L1 light chain polypeptide.

In some embodiments, the PD-L1 binding domain is derived from BMS-936559. In some embodiments, the PD-L1 binding domain is derived from atezolizumab, durvalumab, and avelumab, CK-301, CS-1001, SHR-1316, CBT-502, envafolimab, or BGB-A333.In some embodiments, the anti-PD-L1 heavy chain polypeptide comprises an anti-PD-L1 heavy chain variable domain. In some embodiments, the anti-PD-L1 heavy chain variable domain comprises an IgG framework, an IgA framework, an IgE framework, or an IgM framework. In some embodiments, the anti-PD-L1 heavy chain variable domain comprises a variable domain of an IgG1, IgG2, IgG3, or IgG4 heavy chain. In some embodiments, the anti-PD-L1 light chain polypeptide comprises an anti-PD-L1 light chain variable domain. In some embodiments, the anti-PD-L1 light chain variable domain comprises an IgG framework, an IgA framework, an IgE framework, or an IgM framework. In some embodiments, the anti-PD-L1 light chain variable domain comprises a variable domain of an IgG1, IgG2, IgG3, or IgG4 light chain.

In some embodiments, the PD-L1 binding domain comprises a single domain antibody, a Fab, or a Fab′. In some embodiments, the PD-L1 binding domain comprises the Fab or the Fab′. In some embodiments, the PD-L1 binding domain comprises the Fab or the Fab′. In some embodiments, the PD-L1 binding domain that comprises the Fab or the Fab′ comprises a Fab heavy chain polypeptide comprising a Fab heavy chain variable domain and a Fab light chain polypeptide comprising a Fab light chain variable domain. In some embodiments, the anti-PD-L1 heavy chain polypeptide comprises the Fab heavy chain polypeptide comprising the Fab heavy chain variable domain. In some embodiments, the anti-PD-L1 light chain polypeptide comprises the Fab light chain polypeptide comprising the Fab light chain variable domain.

In some embodiments, the PD-L1 binding domain comprises the single chain variable fragment. In some embodiments, the PD-L1 binding domain that comprises the single chain variable fragment comprises a scFv heavy chain variable domain and a scFv light chain variable domain. In some embodiments, the anti-PD-L1 heavy chain polypeptide comprises the scFv heavy chain variable domain. In some embodiments, the anti-PD-L1 light chain polypeptide comprises the scFv light chain variable domain.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 10; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3. In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 10; HC-CDR2: SEQ ID NO: 11; HC-CDR3: SEQ ID NO: 12.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of said LC-CDR1, LC-CDR2, or LC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 13; LC-CDR2: SEQ ID NO: 14; and LC-CDR3: SEQ ID NO: 15.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 10; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of said LC-CDR1, LC-CDR2, or LC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 10; HC-CDR2: SEQ ID NO: 11; HC-CDR3: SEQ ID NO: 12; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 13; LC-CDR2: SEQ ID NO: 14; and LC-CDR3: SEQ ID NO: 15.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26, and wherein said CDRs comprise from 0-2 amino acid modifications in at least one of said HC-CDR1, HC-CDR2, or HC-CDR3. In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 33; LC-CDR2: SEQ ID NO: 34; and LC-CDR3: SEQ ID NO: 35, and wherein the CDRs comprise from 0-2 amino acid modifications in at least one of said LC-CDR1, LC-CDR2, or LC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 33; LC-CDR2: SEQ ID NO: 34; and LC-CDR3: SEQ ID NO: 35.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26, and wherein said CDRs comprise from 0-2 amino acid modifications in at least one of said HC-CDR1, HC-CDR2, or HC-CDR3; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 33; LC-CDR2: SEQ ID NO: 34; and LC-CDR3: SEQ ID NO: 35, and wherein the CDRs comprise from 0-2 amino acid modifications in at least one of said LC-CDR1, LC-CDR2, or LC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 33; LC-CDR2: SEQ ID NO: 34; and LC-CDR3: SEQ ID NO: 35.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29, and wherein said CDRs comprise from 0-2 amino acid modifications in at least one of said HC-CDR1, HC-CDR2, or HC-CDR3. In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 36; LC-CDR2: SEQ ID NO: 37; and LC-CDR3: SEQ ID NO: 38, and wherein the CDRs comprise from 0-2 amino acid modifications in at least one of said LC-CDR1, LC-CDR2, or LC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 36; LC-CDR2: SEQ ID NO: 37; and LC-CDR3: SEQ ID NO: 38.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29, and wherein said CDRs comprise from 0-2 amino acid modifications in at least one of said HC-CDR1, HC-CDR2, or HC-CDR3; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 36; LC-CDR2: SEQ ID NO: 37; and LC-CDR3: SEQ ID NO: 38, and wherein the CDRs comprise from 0-2 amino acid modifications in at least one of said LC-CDR1, LC-CDR2, or LC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 36; LC-CDR2: SEQ ID NO: 37; and LC-CDR3: SEQ ID NO: 38.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32, and wherein said CDRs comprise from 0-2 amino acid modifications in at least one of said HC-CDR1, HC-CDR2, or HC-CDR3. In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 39; LC-CDR2: SEQ ID NO: 40; and LC-CDR3: SEQ ID NO: 41, and wherein the CDRs comprise from 0-2 amino acid modifications in at least one of said LC-CDR1, LC-CDR2, or LC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 39; LC-CDR2: SEQ ID NO: 40; and LC-CDR3: SEQ ID NO: 41.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32, and wherein said CDRs comprise from 0-2 amino acid modifications in at least one of said HC-CDR1, HC-CDR2, or HC-CDR3; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 39; LC-CDR2: SEQ ID NO: 40; and LC-CDR3: SEQ ID NO: 41, and wherein the CDRs comprise from 0-2 amino acid modifications in at least one of said LC-CDR1, LC-CDR2, or LC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 39; LC-CDR2: SEQ ID NO: 40; and LC-CDR3: SEQ ID NO: 41.

TABLE 4
anti-PD-L1 heavy chain polypeptide comple-
mentarity determining regions (CDR)s as
determined by IMGT definition.

| | Amino Acid | SEQ |
|---|---|---|
| Construct | Sequence | ID |
| Description | (N to C) | NO: |
| anti-PD-L1 Fab 1: | GDTFSTYA | 10 |
| HC: CDR1 | | |
| anti-PD-L1 Fab 1: | IIPIFGKA | 11 |
| HC: CDR2 | | |
| anti-PD-L1 Fab 1: | ARKFHFVSGSPFGMDV | 12 |
| HC: CDR3 | | |
| anti-PD-L1 Fab 2: | GFTFSDSW | 24 |
| HC: CDR1 | | |
| anti-PD-L1 Fab 2: | ISPYGGST | 25 |
| HC: CDR2 | | |
| anti-PD-L1 Fab 2: | ARRHWPGGFDY | 26 |
| HC: CDR3 | | |
| anti-PD-L1 Fab 3: | GFTFSSYI | 27 |
| HC: CDR1 | | |
| anti-PD-L1 Fab 3: | IYPSGGIT | 28 |
| HC: CDR2 | | |
| anti-PD-L1 Fab 3: | ARIKLGTVTTVDY | 29 |
| HC: CDR3 | | |
| anti-PD-L1 Fab 4: | GFTFSRYW | 30 |
| HC: CDR1 | | |
| anti-PD-L1 Fab 4: | IKQDGSEK | 31 |
| HC: CDR2 | | |
| anti-PD-L1 Fab 4: | AREGGWFGELAFDY | 32 |
| HC: CDR3 | | |

TABLE 5
anti-PD-L1 light chain polypeptide comple-
mentarity determining regions (CDR)s as
determined by IMGT definition.

| | Amino Acid | SEQ |
|---|---|---|
| Construct | Sequence | ID |
| Description | (N to C) | NO: |
| anti-PD-L1 Fab 1: | QSVSSY | 13 |
| LC: CDR1 | | |
| anti-PD-L1 Fab 1: | DA | 14 |
| LC: CDR2 | | |
| anti-PD-L1 Fab 1: | QQRSNWPT | 15 |
| LC: CDR3 | | |
| anti-PD-L1 Fab 2: | QDVSTA | 33 |
| LC: CDR1 | | |
| anti-PD-L1 Fab 2: | SA | 34 |
| LC: CDR2 | | |
| anti-PD-L1 Fab 2: | QQYLYHPAT | 35 |
| LC: CDR3 | | |
| anti-PD-L1 Fab 3: | SSDVGGYNY | 36 |
| LC: CDR1 | | |
| anti-PD-L1 Fab 3: | DV | 37 |
| LC: CDR2 | | |
| anti-PD-L1 Fab 3: | FGTGTKVTVLGQP | 38 |
| LC: CDR3 | | |
| anti-PD-L1 Fab 4: | QRVSSSY | 39 |
| LC: CDR1 | | |
| anti-PD-L1 Fab 4: | DA | 40 |
| LC: CDR2 | | |
| anti-PD-L1 Fab 4: | QQYGSLPWT | 41 |
| LC: CDR3 | | |

In some embodiments, the Fab heavy chain polypeptide comprises an amino acid sequence that has at least 70% o sequence identity to the amino acid sequence according to SEQ ID NO: 17. In some embodiments, the Fab heavy chain polypeptide comprises an amino acid sequence that has at least 80% sequence identity to the amino acid sequence according to SEQ ID NO: 17. In some embodiments, the Fab heavy chain polypeptide comprises an amino acid sequence that has at least 85% o sequence identity to the amino acid sequence according to SEQ ID NO: 17. In some embodiments, the Fab heavy chain polypeptide comprises an amino acid sequence that has at least 9000 sequence identity to the amino acid sequence according to SEQ ID NO: 17. In some embodiments, the Fab heavy chain polypeptide comprises an amino acid sequence that has at least 95% o sequence identity to the amino acid sequence according to SEQ ID NO: 17. In some embodiments, the Fab heavy chain polypeptide comprises an amino acid sequence that has at least 96% sequence identity to the amino acid sequence according to SEQ ID NO: 17. In some embodiments, the Fab heavy chain polypeptide comprises an amino acid sequence that has at least 97% sequence identity to the amino acid sequence according to SEQ ID NO: 17. In some embodiments, the Fab heavy chain polypeptide comprises an amino acid sequence that has at least 98% sequence identity to the amino acid sequence according to SEQ ID NO: 17. In some embodiments, the Fab heavy chain polypeptide comprises an amino acid sequence that has at least 99% sequence identity to the amino acid sequence according to SEQ ID NO: 17. In some embodiments, the Fab heavy chain polypeptide comprises an amino acid sequence according to SEQ ID NO: 17.

In some embodiments, the Fab heavy chain polypeptide comprises an amino acid sequence of at least 175 consecutive amino acid residues of SEQ ID NO: 17. In some embodiments, the Fab heavy chain polypeptide comprises an amino acid sequence of at least 190 consecutive amino acid residues of SEQ ID NO: 17. In some embodiments, the Fab heavy chain polypeptide comprises an amino acid sequence of at least 200 consecutive amino acid residues of SEQ ID NO: 17. In some embodiments, the Fab heavy chain polypeptide comprises an amino acid sequence of at least 210 consecutive amino acid residues of SEQ ID NO: 17. In some embodiments, the Fab heavy chain polypeptide comprises an amino acid sequence of at least 215 consecutive amino acid residues of SEQ ID NO: 17. In some embodiments, the Fab heavy chain polypeptide comprises an amino acid sequence of at least 220 consecutive amino acid residues of SEQ ID NO: 17.

In some embodiments, the Fab heavy chain polypeptide comprises an amino acid sequence of at least 175 consecutive amino acid residues of SEQ ID NO: 17, and has at least 80% sequence identity to the at least 175 consecutive amino acid residues of SEQ ID NO: 17. In some embodiments, the Fab heavy chain polypeptide comprises an amino acid sequence of at least 190 consecutive amino acid residues of SEQ ID NO: 17, and has at least 80% sequence identity to the at least 190 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 80% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 80% sequence identity to the at least 210 consecutive amino acid residues of SEQ ID NO: 17. In some embodiments, the Fab heavy chain polypeptide comprises an amino acid sequence of at least 215 consecutive amino acid residues of SEQ ID NO: 17, and has at least 80% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 80% sequence identity to the at least 220 consecutive amino acid residues of SEQ ID NO: 17.

In some embodiments, the Fab heavy chain polypeptide comprises an amino acid sequence of at least 175 consecutive amino acid residues of SEQ ID NO: 17, and has at least 90% sequence identity to the at least 175 consecutive amino acid residues of SEQ ID NO: 17. In some embodiments, the Fab heavy chain polypeptide comprises an amino acid sequence of at least 190 consecutive amino acid residues of SEQ ID NO: 17, and has at least 90% sequence identity to the at least 190 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 90% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 90% sequence identity to the at least 210 consecutive amino acid residues of SEQ ID NO: 17. In some embodiments, the Fab heavy chain polypeptide comprises an amino acid sequence of at least 215 consecutive amino acid residues of SEQ ID NO: 17, and has at least 90% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 90% sequence identity to the at least 220 consecutive amino acid residues of SEQ ID NO: 17.

In some embodiments, the Fab heavy chain polypeptide comprises an amino acid sequence of at least 175 consecutive amino acid residues of SEQ ID NO: 17, and has at least 95% sequence identity to the at least 175 consecutive amino acid residues of SEQ ID NO: 17. In some embodiments, the Fab heavy chain polypeptide comprises an amino acid sequence of at least 190 consecutive amino acid residues of SEQ ID NO: 17, and has at least 95% sequence identity to the at least 190 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 95% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 95% sequence identity to the at least 210 consecutive amino acid residues of SEQ ID NO: 17. In some embodiments, the Fab heavy chain polypeptide comprises an amino acid sequence of at least 215 consecutive amino acid residues of SEQ ID NO: 17, and has at least 95% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 95% sequence identity to the at least 220 consecutive amino acid residues of SEQ ID NO: 17.

In some embodiments, the Fab heavy chain polypeptide comprises an amino acid sequence of at least 175 consecutive amino acid residues of SEQ ID NO: 17, and has at least 99% sequence identity to the at least 175 consecutive amino acid residues of SEQ ID NO: 17. In some embodiments, the Fab heavy chain polypeptide comprises an amino acid sequence of at least 190 consecutive amino acid residues of SEQ ID NO: 17, and has at least 99% sequence identity to the at least 190 consecutive amino acid residues of SEQ ID NO: 17. In some embodiments, the Fab heavy chain polypeptide comprises an amino acid sequence of at least 200 consecutive amino acid residues of SEQ ID NO: 17, and has at least 99% sequence identity to the at least 200 consecutive amino acid residues of SEQ ID NO: 17. In some embodiments, the Fab heavy chain polypeptide comprises an amino acid sequence of at least 210 consecutive amino acid residues of SEQ ID NO: 17, and has at least 99% sequence identity to the at least 210 consecutive amino acid residues of SEQ ID NO: 17. In some embodiments, the Fab heavy chain polypeptide comprises an amino acid sequence of at least 215 consecutive amino acid residues of SEQ ID NO: 17, and has at least 99% sequence identity to the at least 215 consecutive amino acid residues of SEQ ID NO: 17. In some embodiments, the Fab heavy chain polypeptide comprises an amino acid sequence of at least 220 consecutive amino acid residues of SEQ ID NO: 17, and has at least 99% sequence identity to the at least 220 consecutive amino acid residues of SEQ ID NO: 17.

In some embodiments, the Fab light chain polypeptide comprises an amino acid sequence that has at least 70% sequence identity to the amino acid sequence according to SEQ ID NO: 16. In some embodiments, the Fab light chain polypeptide comprises an amino acid sequence that has at least 80% sequence identity to the amino acid sequence according to SEQ ID NO: 16. In some embodiments, the Fab light chain polypeptide comprises an amino acid sequence that has at least 85% sequence identity to the amino acid sequence according to SEQ ID NO: 16. In some embodiments, the Fab light chain polypeptide comprises an amino acid sequence that has at least 90% sequence identity to the amino acid sequence according to SEQ ID NO: 16. In some embodiments, the Fab light chain polypeptide comprises an amino acid sequence that has at least 95% sequence identity to the amino acid sequence according to SEQ ID NO: 16. In some embodiments, the Fab light chain polypeptide comprises an amino acid sequence that has at least 96% sequence identity to the amino acid sequence according to SEQ ID NO: 16. In some embodiments, the Fab light chain polypeptide comprises an amino acid sequence that has at least 97% sequence identity to the amino acid sequence according to SEQ ID NO: 16. In some embodiments, the Fab light chain polypeptide comprises an amino acid sequence that has at least 98% sequence identity to the amino acid sequence according to SEQ ID NO: 16. In some embodiments, the Fab light chain polypeptide comprises an amino acid sequence that has at least 99% sequence identity to the amino acid sequence according to SEQ ID NO: 16. In some embodiments, the Fab light chain polypeptide comprises an amino acid sequence according to SEQ ID NO: 16.

In some embodiments, the Fab light chain polypeptide comprises an amino acid sequence of at least 175 consecutive amino acid residues of SEQ ID NO: 16. In some embodiments, the Fab light chain polypeptide comprises an amino acid sequence of at least 190 consecutive amino acid residues of SEQ ID NO: 16. In some embodiments, the Fab light chain polypeptide comprises an amino acid sequence of at least 200 consecutive amino acid residues of SEQ ID NO: 16. In some embodiments, the Fab light chain polypeptide comprises an amino acid sequence of at least 205 consecutive amino acid residues of SEQ ID NO: 16. In some embodiments, the Fab light chain polypeptide comprises an amino acid sequence of at least 210 consecutive amino acid residues of SEQ ID NO: 16.

In some embodiments, the Fab light chain polypeptide comprises an amino acid sequence of at least 175 consecutive amino acid residues of SEQ ID NO: 16, and has at least 80% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 80% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 80% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 80% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 80% sequence identity to the at least 210 consecutive amino acid residues of SEQ ID NO: 16.

In some embodiments, the Fab light chain polypeptide comprises an amino acid sequence of at least 175 consecutive amino acid residues of SEQ ID NO: 16, and has at least 90% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 90% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 90% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 90% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 90% sequence identity to the at least 210 consecutive amino acid residues of SEQ ID NO: 16.

In some embodiments, the Fab light chain polypeptide comprises an amino acid sequence of at least 175 consecutive amino acid residues of SEQ ID NO: 16, and has at least 95% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 95% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 95% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 95% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 95% sequence identity to the at least 210 consecutive amino acid residues of SEQ ID NO: 16.

In some embodiments, the Fab light chain polypeptide comprises an amino acid sequence of at least 175 consecutive amino acid residues of SEQ ID NO: 16, and has at least 99% sequence identity to the at least 175 consecutive amino acid residues of SEQ ID NO: 16. In some embodiments, the Fab light chain polypeptide comprises an amino acid sequence of at least 190 consecutive amino acid residues of SEQ ID NO: 16, and has at least 99% sequence identity to the at least 190 consecutive amino acid residues of SEQ ID NO: 16. In some embodiments, the Fab light chain polypeptide comprises an amino acid sequence of at least 200 consecutive amino acid residues of SEQ ID NO: 16, and has at least 99% sequence identity to the at least 200 consecutive amino acid residues of SEQ ID NO: 16. In some embodiments, the Fab light chain polypeptide comprises an amino acid sequence of at least 205 consecutive amino acid residues of SEQ ID NO: 16, and has at least 99% sequence identity to the at least 205 consecutive amino acid residues of SEQ ID NO: 16. In some embodiments, the Fab light chain polypeptide comprises an amino acid sequence of at least 210 consecutive amino acid residues of SEQ ID NO: 16, and has at least 99% sequence identity to the at least 210 consecutive amino acid residues of SEQ ID NO: 16.

In some embodiments, the Fab heavy chain polypeptide comprises an amino acid sequence that has at least 70% sequence identity to the amino acid sequence according to SEQ ID NO: 17; and the Fab light chain polypeptide comprises an amino acid sequence that has at least 70% sequence identity to the amino acid sequence according to SEQ ID NO: 16. In some embodiments, the Fab heavy chain polypeptide comprises an amino acid sequence that has at least 80% sequence identity to the amino acid sequence according to SEQ ID NO: 17; and the Fab light chain polypeptide comprises an amino acid sequence that has at least 80% sequence identity to the amino acid sequence according to SEQ ID NO: 16. In some embodiments, the Fab heavy chain polypeptide comprises an amino acid sequence that has at least 85% sequence identity to the amino acid sequence according to SEQ ID NO: 17; and the Fab light chain polypeptide comprises an amino acid sequence that has at least 85% sequence identity to the amino acid sequence according to SEQ ID NO: 16. In some embodiments, the Fab heavy chain polypeptide comprises an amino acid sequence that has at least 90% sequence identity to the amino acid sequence according to SEQ ID NO: 17; and the Fab light chain polypeptide comprises an amino acid sequence that has at least 90% sequence identity to the amino acid sequence according to SEQ ID NO: 16. In some embodiments, the Fab heavy chain polypeptide comprises an amino acid sequence that has at least 95% sequence identity to the amino acid sequence according to SEQ ID NO: 17; and the Fab light chain polypeptide comprises an amino acid sequence that has at least 95% sequence identity to the amino acid sequence according to SEQ ID NO: 16. In some embodiments, the Fab heavy chain polypeptide comprises an amino acid sequence that has at least 96% sequence identity to the amino acid sequence according to SEQ ID NO: 17; and the Fab light chain polypeptide comprises an amino acid sequence that has at least 96% sequence identity to the amino acid sequence according to SEQ ID NO: 16. In some embodiments, the Fab heavy chain polypeptide comprises an amino acid sequence that has at least 97% sequence identity to the amino acid sequence according to SEQ ID NO: 17; and the Fab light chain polypeptide comprises an amino acid sequence that has at least 97% sequence identity to the amino acid sequence according to SEQ ID NO: 16. In some embodiments, the Fab heavy chain polypeptide comprises an amino acid sequence that has at least 98% sequence identity to the amino acid sequence according to SEQ ID NO: 17; and the Fab light chain polypeptide comprises an amino acid sequence that has at least 98% sequence identity to the amino acid sequence according to SEQ ID NO: 16. In some embodiments, the Fab heavy chain polypeptide comprises an amino acid sequence that has at least 99% sequence identity to the amino acid sequence according to SEQ ID NO: 17; and the Fab light chain polypeptide comprises an amino acid sequence that has at least 99% sequence identity to the amino acid sequence according to SEQ ID NO: 16.

In some embodiments, the Fab heavy chain polypeptide comprises an amino acid sequence that has at least 70% sequence identity to the amino acid sequence according to SEQ ID NO: 43. In some embodiments, the Fab heavy chain polypeptide comprises an amino acid sequence that has at least 80% sequence identity to the amino acid sequence according to SEQ ID NO: 43. In some embodiments, the Fab heavy chain polypeptide comprises an amino acid sequence that has at least 85% sequence identity to the amino acid sequence according to SEQ ID NO: 43. In some embodiments, the Fab heavy chain polypeptide comprises an amino acid sequence that has at least 90% sequence identity to the amino acid sequence according to SEQ ID NO: 43. In some embodiments, the Fab heavy chain polypeptide comprises an amino acid sequence that has at least 95% sequence identity to the amino acid sequence according to SEQ ID NO: 43. In some embodiments, the Fab heavy chain polypeptide comprises an amino acid sequence that has at least 96% sequence identity to the amino acid sequence according to SEQ ID NO: 43. In some embodiments, the Fab heavy chain polypeptide comprises an amino acid sequence that has at least 97% sequence identity to the amino acid sequence according to SEQ ID NO: 43. In some embodiments, the Fab heavy chain polypeptide comprises an amino acid sequence that has at least 98% sequence identity to the amino acid sequence according to SEQ ID NO: 43. In some embodiments, the Fab heavy chain polypeptide comprises an amino acid sequence that has at least 99% sequence identity to the amino acid sequence according to SEQ ID NO: 43. In some embodiments, the Fab heavy chain polypeptide comprises an amino acid sequence according to SEQ ID NO: 43.

In some embodiments, the Fab heavy chain polypeptide comprises an amino acid sequence of at least 175 consecutive amino acid residues of SEQ ID NO: 43, and has at least 95% sequence identity to the at least 175 consecutive amino acid residues of SEQ ID NO: 43. In some embodiments, the Fab heavy chain polypeptide comprises an amino acid sequence of at least 190 consecutive amino acid residues of SEQ ID NO: 43, and has at least 95% sequence identity to the at least 190 consecutive amino acid residues of SEQ ID NO: 43. In some embodiments, the Fab heavy chain polypeptide comprises an amino acid sequence of at least 200 consecutive amino acid residues of SEQ ID NO: 43, and has at least 95% sequence identity to the at least 200 consecutive amino acid residues of SEQ ID NO: 43. In some embodiments, the Fab heavy chain polypeptide comprises an amino acid sequence of at least 210 consecutive amino acid residues of SEQ ID NO: 43, and has at least 95% sequence identity to the at least 210 consecutive amino acid residues of SEQ ID NO: 43. In some embodiments, the Fab heavy chain polypeptide comprises an amino acid sequence of at least 215 consecutive amino acid residues of SEQ ID NO: 43, and has at least 95% sequence identity to the at least 215 consecutive amino acid residues of SEQ ID NO: 43. In some embodiments, the Fab heavy chain polypeptide comprises an amino acid sequence of at least 220 consecutive amino acid residues of SEQ ID NO: 43, and has at least 95% sequence identity to the at least 220 consecutive amino acid residues of SEQ ID NO: 43.

In some embodiments, the Fab light chain polypeptide comprises an amino acid sequence that has at least 70% sequence identity to the amino acid sequence according to SEQ ID NO: 42. In some embodiments, the Fab light chain polypeptide comprises an amino acid sequence that has at least 80% sequence identity to the amino acid sequence according to SEQ ID NO: 42. In some embodiments, the Fab light chain polypeptide comprises an amino acid sequence that has at least 85% sequence identity to the amino acid sequence according to SEQ ID NO: 42. In some embodiments, the Fab light chain polypeptide comprises an amino acid sequence that has at least 90% sequence identity to the amino acid sequence according to SEQ ID NO: 42. In some embodiments, the Fab light chain polypeptide comprises an amino acid sequence that has at least 95% sequence identity to the amino acid sequence according to SEQ ID NO: 42. In some embodiments, the Fab light chain polypeptide comprises an amino acid sequence that has at least 96% sequence identity to the amino acid sequence according to SEQ ID NO: 42. In some embodiments, the Fab light chain polypeptide comprises an amino acid sequence that has at least 97% sequence identity to the amino acid sequence according to SEQ ID NO: 42. In some embodiments, the Fab light chain polypeptide comprises an amino acid sequence that has at least 98% sequence identity to the amino acid sequence according to SEQ ID NO: 42. In some embodiments, the Fab light chain polypeptide comprises an amino acid sequence that has at least 99% sequence identity to the amino acid sequence according to SEQ ID NO: 42. In some embodiments, the Fab light chain polypeptide comprises an amino acid sequence according to SEQ ID NO: 42.

In some embodiments, the Fab light chain polypeptide comprises an amino acid sequence of at least 175 consecutive amino acid residues of SEQ ID NO: 42, and has at least 95% sequence identity to the at least 175 consecutive amino acid residues of SEQ ID NO: 42. In some embodiments, the Fab light chain polypeptide comprises an amino acid sequence of at least 190 consecutive amino acid residues of SEQ ID NO: 42, and has at least 95% sequence identity to the at least 190 consecutive amino acid residues of SEQ ID NO: 42. In some embodiments, the Fab light chain polypeptide comprises an amino acid sequence of at least 200 consecutive amino acid residues of SEQ ID NO: 42, and has at least 95% sequence identity to the at least 200 consecutive amino acid residues of SEQ ID NO: 42. In some embodiments, the Fab light chain polypeptide comprises an amino acid sequence of at least 205 consecutive amino acid residues of SEQ ID NO: 42, and has at least 95% sequence identity to the at least 205 consecutive amino acid residues of SEQ ID NO: 42. In some embodiments, the Fab light chain polypeptide comprises an amino acid sequence of at least 210 consecutive amino acid residues of SEQ ID NO: 42, and has at least 95% sequence identity to the at least 210 consecutive amino acid residues of SEQ ID NO: 42.

In some embodiments, the Fab heavy chain polypeptide comprises an amino acid sequence that has at least 70% sequence identity to the amino acid sequence according to SEQ ID NO: 43; and the Fab light chain polypeptide comprises an amino acid sequence that has at least 70% sequence identity to the amino acid sequence according to SEQ ID NO: 42. In some embodiments, the Fab heavy chain polypeptide comprises an amino acid sequence that has at least 80% sequence identity to the amino acid sequence according to SEQ ID NO: 43; and the Fab light chain polypeptide comprises an amino acid sequence that has at least 80% sequence identity to the amino acid sequence according to SEQ ID NO: 42. In some embodiments, the Fab heavy chain polypeptide comprises an amino acid sequence that has at least 85% sequence identity to the amino acid sequence according to SEQ ID NO: 43; and the Fab light chain polypeptide comprises an amino acid sequence that has at least 85% sequence identity to the amino acid sequence according to SEQ ID NO: 42. In some embodiments, the Fab heavy chain polypeptide comprises an amino acid sequence that has at least 90% sequence identity to the amino acid sequence according to SEQ ID NO: 43; and the Fab light chain polypeptide comprises an amino acid sequence that has at least 90% sequence identity to the amino acid sequence according to SEQ ID NO: 42. In some embodiments, the Fab heavy chain polypeptide comprises an amino acid sequence that has at least 95% sequence identity to the amino acid sequence according to SEQ ID NO: 43; and the Fab light chain polypeptide comprises an amino acid sequence that has at least 95% sequence identity to the amino acid sequence according to SEQ ID NO: 42. In some embodiments, the Fab heavy chain polypeptide comprises an amino acid sequence that has at least 96% sequence identity to the amino acid sequence according to SEQ ID NO: 43; and the Fab light chain polypeptide comprises an amino acid sequence that has at least 96% sequence identity to the amino acid sequence according to SEQ ID NO: 42. In some embodiments, the Fab heavy chain polypeptide comprises an amino acid sequence that has at least 97% sequence identity to the amino acid sequence according to SEQ ID NO: 43; and the Fab light chain polypeptide comprises an amino acid sequence that has at least 97% sequence identity to the amino acid sequence according to SEQ ID NO: 42. In some embodiments, the Fab heavy chain polypeptide comprises an amino acid sequence that has at least 98% sequence identity to the amino acid sequence according to SEQ ID NO: 43; and the Fab light chain polypeptide comprises an amino acid sequence that has at least 98% sequence identity to the amino acid sequence according to SEQ ID NO: 42. In some embodiments, the Fab heavy chain polypeptide comprises an amino acid sequence that has at least 99% sequence identity to the amino acid sequence according to SEQ ID NO: 43; and the Fab light chain polypeptide comprises an amino acid sequence that has at least 99% sequence identity to the amino acid sequence according to SEQ ID NO: 42.

In some embodiments, the Fab heavy chain polypeptide comprises an amino acid sequence that has at least 70% sequence identity to the amino acid sequence according to SEQ ID NO: 45. In some embodiments, the Fab heavy chain polypeptide comprises an amino acid sequence that has at least 80% sequence identity to the amino acid sequence according to SEQ ID NO: 45. In some embodiments, the Fab heavy chain polypeptide comprises an amino acid sequence that has at least 85% sequence identity to the amino acid sequence according to SEQ ID NO: 45. In some embodiments, the Fab heavy chain polypeptide comprises an amino acid sequence that has at least 90% sequence identity to the amino acid sequence according to SEQ ID NO: 45. In some embodiments, the Fab heavy chain polypeptide comprises an amino acid sequence that has at least 95% sequence identity to the amino acid sequence according to SEQ ID NO: 45. In some embodiments, the Fab heavy chain polypeptide comprises an amino acid sequence that has at least 96% sequence identity to the amino acid sequence according to SEQ ID NO: 45. In some embodiments, the Fab heavy chain polypeptide comprises an amino acid sequence that has at least 97% sequence identity to the amino acid sequence according to SEQ ID NO: 45. In some embodiments, the Fab heavy chain polypeptide comprises an amino acid sequence that has at least 98% sequence identity to the amino acid sequence according to SEQ ID NO: 45. In some embodiments, the Fab heavy chain polypeptide comprises an amino acid sequence that has at least 99% sequence identity to the amino acid sequence according to SEQ ID NO: 45. In some embodiments, the Fab heavy chain polypeptide comprises an amino acid sequence according to SEQ ID NO: 45.

In some embodiments, the Fab heavy chain polypeptide comprises an amino acid sequence of at least 175 consecutive amino acid residues of SEQ ID NO: 45, and has at least 95% sequence identity to the at least 175 consecutive amino acid residues of SEQ ID NO: 45. In some embodiments, the Fab heavy chain polypeptide comprises an amino acid sequence of at least 190 consecutive amino acid residues of SEQ ID NO: 45, and has at least 95% sequence identity to the at least 190 consecutive amino acid residues of SEQ ID NO: 45. In some embodiments, the Fab heavy chain polypeptide comprises an amino acid sequence of at least 200 consecutive amino acid residues of SEQ ID NO: 45, and has at least 95% sequence identity to the at least 200 consecutive amino acid residues of SEQ ID NO: 45. In some embodiments, the Fab heavy chain polypeptide comprises an amino acid sequence of at least 210 consecutive amino acid residues of SEQ ID NO: 45, and has at least 95% sequence identity to the at least 210 consecutive amino acid residues of SEQ ID NO: 45. In some embodiments, the Fab heavy chain polypeptide comprises an amino acid sequence of at least 215 consecutive amino acid residues of SEQ ID NO: 45, and has at least 95% sequence identity to the at least 215 consecutive amino acid residues of SEQ ID NO: 45. In some embodiments, the Fab heavy chain polypeptide comprises an amino acid sequence of at least 220 consecutive amino acid residues of SEQ ID NO: 45, and has at least 95% sequence identity to the at least 220 consecutive amino acid residues of SEQ ID NO: 45.

In some embodiments, the Fab light chain polypeptide comprises an amino acid sequence that has at least 70% sequence identity to the amino acid sequence according to SEQ ID NO: 44. In some embodiments, the Fab light chain polypeptide comprises an amino acid sequence that has at least 80% sequence identity to the amino acid sequence according to SEQ ID NO: 44. In some embodiments, the Fab light chain polypeptide comprises an amino acid sequence that has at least 85% sequence identity to the amino acid sequence according to SEQ ID NO: 44. In some embodiments, the Fab light chain polypeptide comprises an amino acid sequence that has at least 90% sequence identity to the amino acid sequence according to SEQ ID NO: 44. In some embodiments, the Fab light chain polypeptide comprises an amino acid sequence that has at least 95% sequence identity to the amino acid sequence according to SEQ ID NO: 44. In some embodiments, the Fab light chain polypeptide comprises an amino acid sequence that has at least 96% sequence identity to the amino acid sequence according to SEQ ID NO: 44. In some embodiments, the Fab light chain polypeptide comprises an amino acid sequence that has at least 97% sequence identity to the amino acid sequence according to SEQ ID NO: 44. In some embodiments, the Fab light chain polypeptide comprises an amino acid sequence that has at least 98% sequence identity to the amino acid sequence according to SEQ ID NO: 44. In some embodiments, the Fab light chain polypeptide comprises an amino acid sequence that has at least 99% sequence identity to the amino acid sequence according to SEQ ID NO: 44. In some embodiments, the Fab light chain polypeptide comprises an amino acid sequence according to SEQ ID NO: 44.

In some embodiments, the Fab light chain polypeptide comprises an amino acid sequence of at least 175 consecutive amino acid residues of SEQ ID NO: 44, and has at least 95% sequence identity to the at least 175 consecutive amino acid residues of SEQ ID NO: 44. In some embodiments, the Fab light chain polypeptide comprises an amino acid sequence of at least 190 consecutive amino acid residues of SEQ ID NO: 44, and has at least 95% sequence identity to the at least 190 consecutive amino acid residues of SEQ ID NO: 44. In some embodiments, the Fab light chain polypeptide comprises an amino acid sequence of at least 200 consecutive amino acid residues of SEQ ID NO: 44, and has at least 95% sequence identity to the at least 200 consecutive amino acid residues of SEQ ID NO: 44. In some embodiments, the Fab light chain polypeptide comprises an amino acid sequence of at least 205 consecutive amino acid residues of SEQ ID NO: 44, and has at least 95% sequence identity to the at least 205 consecutive amino acid residues of SEQ ID NO: 44. In some embodiments, the Fab light chain polypeptide comprises an amino acid sequence of at least 210 consecutive amino acid residues of SEQ ID NO: 44, and has at least 95% sequence identity to the at least 210 consecutive amino acid residues of SEQ ID NO: 44.

In some embodiments, the Fab heavy chain polypeptide comprises an amino acid sequence that has at least 70% sequence identity to the amino acid sequence according to SEQ ID NO: 45; and the Fab light chain polypeptide comprises an amino acid sequence that has at least 70% sequence identity to the amino acid sequence according to SEQ ID NO: 44. In some embodiments, the Fab heavy chain polypeptide comprises an amino acid sequence that has at least 80% sequence identity to the amino acid sequence according to SEQ ID NO: 45; and the Fab light chain polypeptide comprises an amino acid sequence that has at least 80% sequence identity to the amino acid sequence according to SEQ ID NO: 44. In some embodiments, the Fab heavy chain polypeptide comprises an amino acid sequence that has at least 85% sequence identity to the amino acid sequence according to SEQ ID NO: 45; and the Fab light chain polypeptide comprises an amino acid sequence that has at least 85% sequence identity to the amino acid sequence according to SEQ ID NO: 44. In some embodiments, the Fab heavy chain polypeptide comprises an amino acid sequence that has at least 90% sequence identity to the amino acid sequence according to SEQ ID NO: 45; and the Fab light chain polypeptide comprises an amino acid sequence that has at least 90% sequence identity to the amino acid sequence according to SEQ ID NO: 44. In some embodiments, the Fab heavy chain polypeptide comprises an amino acid sequence that has at least 95% sequence identity to the amino acid sequence according to SEQ ID NO: 45; and the Fab light chain polypeptide comprises an amino acid sequence that has at least 95% sequence identity to the amino acid sequence according to SEQ ID NO: 44. In some embodiments, the Fab heavy chain polypeptide comprises an amino acid sequence that has at least 96% sequence identity to the amino acid sequence according to SEQ ID NO: 45; and the Fab light chain polypeptide comprises an amino acid sequence that has at least 96% sequence identity to the amino acid sequence according to SEQ ID NO: 44. In some embodiments, the Fab heavy chain polypeptide comprises an amino acid sequence that has at least 97% sequence identity to the amino acid sequence according to SEQ ID NO: 45; and the Fab light chain polypeptide comprises an amino acid sequence that has at least 97% sequence identity to the amino acid sequence according to SEQ ID NO: 44. In some embodiments, the Fab heavy chain polypeptide comprises an amino acid sequence that has at least 98% sequence identity to the amino acid sequence according to SEQ ID NO: 45; and the Fab light chain polypeptide comprises an amino acid sequence that has at least 98% sequence identity to the amino acid sequence according to SEQ ID NO: 44. In some embodiments, the Fab heavy chain polypeptide comprises an amino acid sequence that has at least 99% sequence identity to the amino acid sequence according to SEQ ID NO: 45; and the Fab light chain polypeptide comprises an amino acid sequence that has at least 99% sequence identity to the amino acid sequence according to SEQ ID NO: 44.

In some embodiments, the Fab heavy chain polypeptide comprises an amino acid sequence that has at least 70% sequence identity to the amino acid sequence according to SEQ ID NO: 47. In some embodiments, the Fab heavy chain polypeptide comprises an amino acid sequence that has at least 80% sequence identity to the amino acid sequence according to SEQ ID NO: 47. In some embodiments, the Fab heavy chain polypeptide comprises an amino acid sequence that has at least 85% sequence identity to the amino acid sequence according to SEQ ID NO: 47. In some embodiments, the Fab heavy chain polypeptide comprises an amino acid sequence that has at least 90% sequence identity to the amino acid sequence according to SEQ ID NO: 47. In some embodiments, the Fab heavy chain polypeptide comprises an amino acid sequence that has at least 95% sequence identity to the amino acid sequence according to SEQ ID NO: 47. In some embodiments, the Fab heavy chain polypeptide comprises an amino acid sequence that has at least 96% sequence identity to the amino acid sequence according to SEQ ID NO: 47. In some embodiments, the Fab heavy chain polypeptide comprises an amino acid sequence that has at least 97% sequence identity to the amino acid sequence according to SEQ ID NO: 47. In some embodiments, the Fab heavy chain polypeptide comprises an amino acid sequence that has at least 98% sequence identity to the amino acid sequence according to SEQ ID NO: 47. In some embodiments, the Fab heavy chain polypeptide comprises an amino acid sequence that has at least 99% sequence identity to the amino acid sequence according to SEQ ID NO: 47. In some embodiments, the Fab heavy chain polypeptide comprises an amino acid sequence according to SEQ ID NO: 47.

In some embodiments, the Fab heavy chain polypeptide comprises an amino acid sequence of at least 175 consecutive amino acid residues of SEQ ID NO: 47, and has at least 95% sequence identity to the at least 175 consecutive amino acid residues of SEQ ID NO: 47. In some embodiments, the Fab heavy chain polypeptide comprises an amino acid sequence of at least 190 consecutive amino acid residues of SEQ ID NO: 47, and has at least 95% sequence identity to the at least 190 consecutive amino acid residues of SEQ ID NO: 47. In some embodiments, the Fab heavy chain polypeptide comprises an amino acid sequence of at least 200 consecutive amino acid residues of SEQ ID NO: 47, and has at least 95% sequence identity to the at least 200 consecutive amino acid residues of SEQ ID NO: 47. In some embodiments, the Fab heavy chain polypeptide comprises an amino acid sequence of at least 210 consecutive amino acid residues of SEQ ID NO: 47, and has at least 95% sequence identity to the at least 210 consecutive amino acid residues of SEQ ID NO: 47. In some embodiments, the Fab heavy chain polypeptide comprises an amino acid sequence of at least 215 consecutive amino acid residues of SEQ ID NO: 47, and has at least 95% sequence identity to the at least 215 consecutive amino acid residues of SEQ ID NO: 47. In some embodiments, the Fab heavy chain polypeptide comprises an amino acid sequence of at least 220 consecutive amino acid residues of SEQ ID NO: 47, and has at least 95% sequence identity to the at least 220 consecutive amino acid residues of SEQ ID NO: 47.

In some embodiments, the Fab light chain polypeptide comprises an amino acid sequence that has at least 70% sequence identity to the amino acid sequence according to SEQ ID NO: 46. In some embodiments, the Fab light chain polypeptide comprises an amino acid sequence that has at least 80% sequence identity to the amino acid sequence according to SEQ ID NO: 46. In some embodiments, the Fab light chain polypeptide comprises an amino acid sequence that has at least 85% sequence identity to the amino acid sequence according to SEQ ID NO: 46. In some embodiments, the Fab light chain polypeptide comprises an amino acid sequence that has at least 90% sequence identity to the amino acid sequence according to SEQ ID NO: 46. In some embodiments, the Fab light chain polypeptide comprises an amino acid sequence that has at least 95% sequence identity to the amino acid sequence according to SEQ ID NO: 46. In some embodiments, the Fab light chain polypeptide comprises an amino acid sequence that has at least 96% sequence identity to the amino acid sequence according to SEQ ID NO: 46. In some embodiments, the Fab light chain polypeptide comprises an amino acid sequence that has at least 97% sequence identity to the amino acid sequence according to SEQ ID NO: 46. In some embodiments, the Fab light chain polypeptide comprises an amino acid sequence that has at least 98% sequence identity to the amino acid sequence according to SEQ ID NO: 46. In some embodiments, the Fab light chain polypeptide comprises an amino acid sequence that has at least 99% sequence identity to the amino acid sequence according to SEQ ID NO: 46. In some embodiments, the Fab light chain polypeptide comprises an amino acid sequence according to SEQ ID NO: 46.

In some embodiments, the Fab light chain polypeptide comprises an amino acid sequence of at least 175 consecutive amino acid residues of SEQ ID NO: 46, and has at least 95% sequence identity to the at least 175 consecutive amino acid residues of SEQ ID NO: 46. In some embodiments, the Fab light chain polypeptide comprises an amino acid sequence of at least 190 consecutive amino acid residues of SEQ ID NO: 46, and has at least 95% sequence identity to the at least 190 consecutive amino acid residues of SEQ ID NO: 46. In some embodiments, the Fab light chain polypeptide comprises an amino acid sequence of at least 200 consecutive amino acid residues of SEQ ID NO: 46, and has at least 95% sequence identity to the at least 200 consecutive amino acid residues of SEQ ID NO: 46. In some embodiments, the Fab light chain polypeptide comprises an amino acid sequence of at least 205 consecutive amino acid residues of SEQ ID NO: 46, and has at least 95% sequence identity to the at least 205 consecutive amino acid residues of SEQ ID NO: 46. In some embodiments, the Fab light chain polypeptide comprises an amino acid sequence of at least 210 consecutive amino acid residues of SEQ ID NO: 46, and has at least 95% sequence identity to the at least 210 consecutive amino acid residues of SEQ ID NO: 46.

In some embodiments, the Fab heavy chain polypeptide comprises an amino acid sequence that has at least 70% sequence identity to the amino acid sequence according to SEQ ID NO: 47; and the Fab light chain polypeptide comprises an amino acid sequence that has at least 70% sequence identity to the amino acid sequence according to SEQ ID NO: 46. In some embodiments, the Fab heavy chain polypeptide comprises an amino acid sequence that has at least 80% sequence identity to the amino acid sequence according to SEQ ID NO: 47; and the Fab light chain polypeptide comprises an amino acid sequence that has at least 80% sequence identity to the amino acid sequence according to SEQ ID NO: 46. In some embodiments, the Fab heavy chain polypeptide comprises an amino acid sequence that has at least 85% sequence identity to the amino acid sequence according to SEQ ID NO: 47; and the Fab light chain polypeptide comprises an amino acid sequence that has at least 85% sequence identity to the amino acid sequence according to SEQ ID NO: 46. In some embodiments, the Fab heavy chain polypeptide comprises an amino acid sequence that has at least 90% sequence identity to the amino acid sequence according to SEQ ID NO: 47; and the Fab light chain polypeptide comprises an amino acid sequence that has at least 90% sequence identity to the amino acid sequence according to SEQ ID NO: 46. In some embodiments, the Fab heavy chain polypeptide comprises an amino acid sequence that has at least 95% sequence identity to the amino acid sequence according to SEQ ID NO: 47; and the Fab light chain polypeptide comprises an amino acid sequence that has at least 95% sequence identity to the amino acid sequence according to SEQ ID NO: 46. In some embodiments, the Fab heavy chain polypeptide comprises an amino acid sequence that has at least 96% sequence identity to the amino acid sequence according to SEQ ID NO: 47; and the Fab light chain polypeptide comprises an amino acid sequence that has at least 96% sequence identity to the amino acid sequence according to SEQ ID NO: 46. In some embodiments, the Fab heavy chain polypeptide comprises an amino acid sequence that has at least 97% sequence identity to the amino acid sequence according to SEQ ID NO: 47; and the Fab light chain polypeptide comprises an amino acid sequence that has at least 97% sequence identity to the amino acid sequence according to SEQ ID NO: 46. In some embodiments, the Fab heavy chain polypeptide comprises an amino acid sequence that has at least 98% sequence identity to the amino acid sequence according to SEQ ID NO: 47; and the Fab light chain polypeptide comprises an amino acid sequence that has at least 98% sequence identity to the amino acid sequence according to SEQ ID NO: 46. In some embodiments, the Fab heavy chain polypeptide comprises an amino acid sequence that has at least 99% sequence identity to the amino acid sequence according to SEQ ID NO: 47; and the Fab light chain polypeptide comprises an amino acid sequence that has at least 99% sequence identity to the amino acid sequence according to SEQ ID NO: 46.

TABLE 6
anti-PD-L1 Fab light chain polypeptide and Fab heavy chain
polypeptide sequences. CDR sequences are underlined and were
determined using IMGT definition

| | Amino Acid Sequence | SEQ ID |
|---|---|---|
| Construct Description | (N to C) | NO: |
| anti-PD-L1 Fab 1: LC | EIVLTQSPATLSLSPGERATLSC | 16 |
| | RASQSVSSYLAWYQQKPGQA | |
| | PRLLIYDASNRATGIPARFSGS | |
| | GSGTDFTLTISSLEPEDFAVYY | |
| | CQQRSNWPTFGQGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| anti-PD-L1 Fab 1: HC | QVQLVQSGAEVKKPGSSVKVS | 17 |
| | CKTSGDTFSTYAISWVRQAPG | |
| | QGLEWMGGIIPIFGKAHYAQ | |
| | KFQGRVTITADESTSTAYMEL | |
| | SSLRSEDTAVYFCARKFHFVS | |
| | GSPFGMDVWGQGTTVTVSSA | |
| | STKGPSVFPLAPSSKSTSGGTA | |
| | ALGCLVKDYFPEPVTVSWNSG | |
| | ALTSGVHTFPAVLQSSGLYSLS | |
| | SVVTVPSSSLGTQTYICNVNH | |
| | KPSNTKVDKKVEPKSC | |
| anti-PD-L1 Fab 2: LC | DIQMTQSPSSLSASVGDRVTIT | 42 |
| | CRASQDVSTAVAWYQQKPGK | |
| | APKLLIYSASFLYSGVPSRFSG | |
| | SGSGTDFTLTISSLQPEDFATY | |
| | YCQQYLYHPATFGQGTKVEI | |
| | KRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| anti-PD-L1 Fab 2: HC | EVQLVESGGGLVQPGGSLRLS | 43 |
| | CAASGFTFSDSWIHWVRQAP | |
| | GKGLEWVAWISPYGGSTYYA | |
| | DSVKGRFTISADTSKNTAYLQ | |
| | MNSLRAEDTAVYYCARRHW | |
| | PGGFDYWGQGTLVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKKVEPKSC | |
| anti-PD-L1 Fab 3: LC | QSALTQPASVSGSPGQSITISCT | 44 |
| | GTSSDVGGYNYVSWYQQHPG | |
| | KAPKLMIYDVSNRPSGVSNRF | |
| | SGSKSGNTASLTISGLQAEDEA | |
| | DYYCSSYTSSSTRVFGTGTKV | |
| | TVLGQPKANPTVTLFPPSSEEL | |
| | QANKATLVCLISDFYPGAVTV | |
| | AWKADGSPVKAGVETTKPSK | |
| | QSNNKYAASSYLSLTPEQWKS | |
| | HRSYSCQVTHEGSTVEKTVAP | |
| | TECS | |
| anti-PD-L1 Fab 3: HC | EVQLLESGGGLVQPGGSLRLS | 45 |
| | CAASGFTFSSYIMMWVRQAP | |
| | GKGLEWVSSIYPSGGITFYAD | |
| | TVKGRFTISRDNSKNTLYLQM | |
| | NSLRAEDTAVYYCARIKLGT | |
| | VTTVDYWGQGTLVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKKVEPKSC | |
| anti-PD-L1 Fab 4: LC | EIVLTQSPGTLSLSPGERATLSC | 46 |
| | RASQRVSSSYLAWYQQKPGQ | |
| | APRLLIYDASSRATGIPDRFSG | |
| | SGSGTDFTLTISRLEPEDFAVY | |
| | YCQQYGSLPWTFGQGTKVEI | |
| | KRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| anti-PD-L1 Fab 4: HC | EVQLVESGGGLVQPGGSLRLS | 47 |
| | CAASGFTFSRYWMSWVRQAP | |
| | GKGLEWVANIKQDGSEKYYV | |
| | DSVKGRFTISRDNAKNSLYLQ | |
| | MNSLRAEDTAVYYCAREGG | |
| | WFGELAFDYWGQGTLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKRVEPKSC | |

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 10; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3. In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 10; HC-CDR2: SEQ ID NO: 11; HC-CDR3: SEQ ID NO: 12.

In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise:LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of said LC-CDR1, LC-CDR2, or LC-CDR3. In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise:LC-CDR1: SEQ ID NO: 13; LC-CDR2: SEQ ID NO: 14; and LC-CDR3: SEQ ID NO: 15.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 10; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of said LC-CDR1, LC-CDR2, or LC-CDR3.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 10; HC-CDR2: SEQ ID NO: 11; HC-CDR3: SEQ ID NO: 12; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 13; LC-CDR2: SEQ ID NO: 14; and LC-CDR3: SEQ ID NO: 15.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 24; HC-CDR2: SEQ ID NO: 25; HC-CDR3: SEQ ID NO: 26, and wherein said CDRs comprise from 0-2 amino acid modifications in at least one of said HC-CDR1, HC-CDR2, or HC-CDR3. In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 24; HC-CDR2: SEQ ID NO: 25; HC-CDR3: SEQ ID NO: 26.

In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise:LC-CDR1: SEQ ID NO: 33; LC-CDR2: SEQ ID NO: 34; and LC-CDR3: SEQ ID NO: 35, and wherein the CDRs comprise from 0-2 amino acid modifications in at least one of said LC-CDR1, LC-CDR2, or LC-CDR3. In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise:LC-CDR1: SEQ ID NO: 33; LC-CDR2: SEQ ID NO: 34; and LC-CDR3: SEQ ID NO: 35.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 24; HC-CDR2: SEQ ID NO: 25; HC-CDR3: SEQ ID NO: 26, and wherein said CDRs comprise from 0-2 amino acid modifications in at least one of said HC-CDR1, HC-CDR2, or HC-CDR3; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 33; LC-CDR2: SEQ ID NO: 34; and LC-CDR3: SEQ ID NO: 35, and wherein the CDRs comprise from 0-2 amino acid modifications in at least one of said LC-CDR1, LC-CDR2, or LC-CDR3.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 24; HC-CDR2: SEQ ID NO: 25; HC-CDR3: SEQ ID NO: 26; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 33; LC-CDR2: SEQ ID NO: 34; and LC-CDR3: SEQ ID NO: 35.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 27; HC-CDR2: SEQ ID NO: 28; HC-CDR3: SEQ ID NO: 29, and wherein said CDRs comprise from 0-2 amino acid modifications in at least one of said HC-CDR1, HC-CDR2, or HC-CDR3. In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 27; HC-CDR2: SEQ ID NO: 28; HC-CDR3: SEQ ID NO: 29.

In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise:LC-CDR1: SEQ ID NO: 36; LC-CDR2: SEQ ID NO: 37; and LC-CDR3: SEQ ID NO: 38, and wherein the CDRs comprise from 0-2 amino acid modifications in at least one of said LC-CDR1, LC-CDR2, or LC-CDR3. In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise:LC-CDR1: SEQ ID NO: 36; LC-CDR2: SEQ ID NO: 37; and LC-CDR3: SEQ ID NO: 38.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 27; HC-CDR2: SEQ ID NO: 28; HC-CDR3: SEQ ID NO: 29, and wherein said CDRs comprise from 0-2 amino acid modifications in at least one of said HC-CDR1, HC-CDR2, or HC-CDR3; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 36; LC-CDR2: SEQ ID NO: 37; and LC-CDR3: SEQ ID NO: 38, and wherein the CDRs comprise from 0-2 amino acid modifications in at least one of said LC-CDR1, LC-CDR2, or LC-CDR3.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 27; HC-CDR2: SEQ ID NO: 28; HC-CDR3: SEQ ID NO: 29; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 36; LC-CDR2: SEQ ID NO: 37; and LC-CDR3: SEQ ID NO: 38.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 30; HC-CDR2: SEQ ID NO: 31; HC-CDR3: SEQ ID NO: 32, and wherein said CDRs comprise from 0-2 amino acid modifications in at least one of said HC-CDR1, HC-CDR2, or HC-CDR3. In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 30; HC-CDR2: SEQ ID NO: 31; HC-CDR3: SEQ ID NO: 32.

In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 39; LC-CDR2: SEQ ID NO: 40; and LC-CDR3: SEQ ID NO: 41, and wherein the CDRs comprise from 0-2 amino acid modifications in at least one of said LC-CDR1, LC-CDR2, or LC-CDR3. In some embodiments, the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise:LC-CDR1: SEQ ID NO: 39; LC-CDR2: SEQ ID NO: 40; and LC-CDR3: SEQ ID NO: 41.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 30; HC-CDR2: SEQ ID NO: 31; HC-CDR3: SEQ ID NO: 32, and wherein said CDRs comprise from 0-2 amino acid modifications in at least one of said HC-CDR1, HC-CDR2, or HC-CDR3; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 39; LC-CDR2: SEQ ID NO: 40; and LC-CDR3: SEQ ID NO: 41, and wherein the CDRs comprise from 0-2 amino acid modifications in at least one of said LC-CDR1, LC-CDR2, or LC-CDR3.

In some embodiments, the PD-L1 binding domain comprises complementarity determining region (CDRs): HC-CDR1, HC-CDR2, and HC-CDR3, wherein the HC-CDR1, the HC-CDR2, and the HC-CDR3 of the PD-L1 binding domain comprises: HC-CDR1: SEQ ID NO: 30; HC-CDR2: SEQ ID NO: 31; HC-CDR3: SEQ ID NO: 32; and the PD-L1 binding domain comprises complementarity determining regions (CDRs): LC-CDR1, LC-CDR2, and LC-CDR3, wherein the LC-CDR1, the LC-CDR2, and the LC-CDR3 of the PD-L1 binding domain comprise: LC-CDR1: SEQ ID NO: 39; LC-CDR2: SEQ ID NO: 40; and LC-CDR3: SEQ ID NO: 41.

In some embodiments, the PD-L1 binding domain comprises an amino acid sequence that has at least 70% sequence identity to the amino acid sequence according to SEQ ID NO: 17. In some embodiments, the PD-L1 binding domain comprises an amino acid sequence that has at least 80% sequence identity to the amino acid sequence according to SEQ ID NO: 17. In some embodiments, the PD-L1 binding domain comprises an amino acid sequence that has at least 85% sequence identity to the amino acid sequence according to SEQ ID NO: 17. In some embodiments, the PD-L1 binding domain comprises an amino acid sequence that has at least 90% sequence identity to the amino acid sequence according to SEQ ID NO: 17. In some embodiments, the PD-L1 binding domain comprises an amino acid sequence that has at least 95% sequence identity to the amino acid sequence according to SEQ ID NO: 17. In some embodiments, the PD-L1 binding domain comprises an amino acid sequence that has at least 96% sequence identity to the amino acid sequence according to SEQ ID NO: 17. In some embodiments, the PD-L1 binding domain comprises an amino acid sequence that has at least 97% sequence identity to the amino acid sequence according to SEQ ID NO: 17. In some embodiments, the PD-L1 binding domain comprises an amino acid sequence that has at least 98% sequence identity to the amino acid sequence according to SEQ ID NO: 17. In some embodiments, the PD-L1 binding domain comprises an amino acid sequence that has at least 99% sequence identity to the amino acid sequence according to SEQ ID NO: 17. In some embodiments, the PD-L1 binding domain comprises an amino acid sequence according to SEQ ID NO: 17.

In some embodiments, the PD-L1 binding domain comprises an amino acid sequence of at least 175 consecutive amino acid residues of SEQ ID NO: 17. In some embodiments, the PD-L1 binding domain comprises an amino acid sequence of at least 190 consecutive amino acid residues of SEQ ID NO: 17. In some embodiments, the PD-L1 binding domain comprises an amino acid sequence of at least 200 consecutive amino acid residues of SEQ ID NO: 17. In some embodiments, the PD-L1 binding domain comprises an amino acid sequence of at least 210 consecutive amino acid residues of SEQ ID NO: 17. In some embodiments, the PD-L1 binding domain comprises an amino acid sequence of at least 215 consecutive amino acid residues of SEQ ID NO: 17. In some embodiments, the PD-L1 binding domain comprises an amino acid sequence of at least 220 consecutive amino acid residues of SEQ ID NO: 17.

In some embodiments, the PD-L1 binding domain comprises an amino acid sequence of at least 175 consecutive amino acid residues of SEQ ID NO: 17, and has at least 80% sequence identity to the at least 175 consecutive amino acid residues of SEQ ID NO: 17. In some embodiments, the PD-L1 binding domain comprises an amino acid sequence of at least 190 consecutive amino acid residues of SEQ ID NO: 17, and has at least 80% sequence identity to the at least 190 consecutive amino acid residues of SEQ ID NO: 17. In some embodiments, the PD-L1 binding domain comprises an amino acid sequence of at least 200 consecutive amino acid residues of SEQ ID NO: 17, and has at least 80% sequence identity to the at least 200 consecutive amino acid residues of SEQ ID NO: 17. In some embodiments, the PD-L1 binding domain comprises an amino acid sequence of at least 210 consecutive amino acid residues of SEQ ID NO: 17, and has at least 80% sequence identity to the at least 210 consecutive amino acid residues of SEQ ID NO: 17.

In some embodiments, the PD-L1 binding domain comprises an amino acid sequence of at least 215 consecutive amino acid residues of SEQ ID NO: 17, and has at least 80% sequence identity to the at least 215 consecutive amino acid residues of SEQ ID NO: 17. In some embodiments, the PD-L1 binding domain comprises an amino acid sequence of at least 220 consecutive amino acid residues of SEQ ID NO: 17, and has at least 80% sequence identity to the at least 220 consecutive amino acid residues of SEQ ID NO: 17.

In some embodiments, the PD-L1 binding domain comprises an amino acid sequence of at least 175 consecutive amino acid residues of SEQ ID NO: 17, and has at least 90% sequence identity to the at least 175 consecutive amino acid residues of SEQ ID NO: 17. In some embodiments, the PD-L1 binding domain comprises an amino acid sequence of at least 190 consecutive amino acid residues of SEQ ID NO: 17, and has at least 90% sequence identity to the at least 190 consecutive amino acid residues of SEQ ID NO: 17. In some embodiments, the PD-L1 binding domain comprises an amino acid sequence of at least 200 consecutive amino acid residues of SEQ ID NO: 17, and has at least 90% sequence identity to the at least 200 consecutive amino acid residues of SEQ ID NO: 17. In some embodiments, the PD-L1 binding domain comprises an amino acid sequence of at least 210 consecutive amino acid residues of SEQ ID NO: 17, and has at least 90% sequence identity to the at least 210 consecutive amino acid residues of SEQ ID NO: 17.

In some embodiments, the PD-L1 binding domain comprises an amino acid sequence of at least 215 consecutive amino acid residues of SEQ ID NO: 17, and has at least 90% sequence identity to the at least 215 consecutive amino acid residues of SEQ ID NO: 17. In some embodiments, the PD-L1 binding domain comprises an amino acid sequence of at least 220 consecutive amino acid residues of SEQ ID NO: 17, and has at least 90% sequence identity to the at least 220 consecutive amino acid residues of SEQ ID NO: 17.

In some embodiments, the PD-L1 binding domain comprises an amino acid sequence of at least 175 consecutive amino acid residues of SEQ ID NO: 17, and has at least 95% sequence identity to the at least 175 consecutive amino acid residues of SEQ ID NO: 17. In some embodiments, the PD-L1 binding domain comprises an amino acid sequence of at least 190 consecutive amino acid residues of SEQ ID NO: 17, and has at least 95% sequence identity to the at least 190 consecutive amino acid residues of SEQ ID NO: 17. In some embodiments, the PD-L1 binding domain comprises an amino acid sequence of at least 200 consecutive amino acid residues of SEQ ID NO: 17, and has at least 95% sequence identity to the at least 200 consecutive amino acid residues of SEQ ID NO: 17. In some embodiments, the PD-L1 binding domain comprises an amino acid sequence of at least 210 consecutive amino acid residues of SEQ ID NO: 17, and has at least 95% sequence identity to the at least 210 consecutive amino acid residues of SEQ ID NO: 17.

In some embodiments, the PD-L1 binding domain comprises an amino acid sequence of at least 215 consecutive amino acid residues of SEQ ID NO: 17, and has at least 95% sequence identity to the at least 215 consecutive amino acid residues of SEQ ID NO: 17. In some embodiments, the PD-L1 binding domain comprises an amino acid sequence of at least 220 consecutive amino acid residues of SEQ ID NO: 17, and has at least 95% sequence identity to the at least 220 consecutive amino acid residues of SEQ ID NO: 17.

In some embodiments, the PD-L1 binding domain comprises an amino acid sequence of at least 175 consecutive amino acid residues of SEQ ID NO: 17, and has at least 99% sequence identity to the at least 175 consecutive amino acid residues of SEQ ID NO: 17. In some embodiments, the PD-L1 binding domain comprises an amino acid sequence of at least 190 consecutive amino acid residues of SEQ ID NO: 17, and has at least 99% sequence identity to the at least 190 consecutive amino acid residues of SEQ ID NO: 17. In some embodiments, the PD-L1 binding domain comprises an amino acid sequence of at least 200 consecutive amino acid residues of SEQ ID NO: 17, and has at least 99% sequence identity to the at least 200 consecutive amino acid residues of SEQ ID NO: 17. In some embodiments, the PD-L1 binding domain comprises an amino acid sequence of at least 210 consecutive amino acid residues of SEQ ID NO: 17, and has at least 99% sequence identity to the at least 210 consecutive amino acid residues of SEQ ID NO: 17.

In some embodiments, the PD-L1 binding domain comprises an amino acid sequence of at least 215 consecutive amino acid residues of SEQ ID NO: 17, and has at least 99% sequence identity to the at least 215 consecutive amino acid residues of SEQ ID NO: 17. In some embodiments, the PD-L1 binding domain comprises an amino acid sequence of at least 220 consecutive amino acid residues of SEQ ID NO: 17, and has at least 99% sequence identity to the at least 220 consecutive amino acid residues of SEQ ID NO: 17.

In some embodiments, the PD-L1 binding domain comprises an amino acid sequence that has at least 70% sequence identity to the amino acid sequence according to SEQ ID NO: 16. In some embodiments, the PD-L1 binding domain comprises an amino acid sequence that has at least 80% sequence identity to the amino acid sequence according to SEQ ID NO: 16. In some embodiments, the PD-L1 binding domain comprises an amino acid sequence that has at least 85% sequence identity to the amino acid sequence according to SEQ ID NO: 16. In some embodiments, the PD-L1 binding domain comprises an amino acid sequence that has at least 90% sequence identity to the amino acid sequence according to SEQ ID NO: 16. In some embodiments, the PD-L1 binding domain comprises an amino acid sequence that has at least 95% sequence identity to the amino acid sequence according to SEQ ID NO: 16. In some embodiments, the PD-L1 binding domain comprises an amino acid sequence that has at least 96% sequence identity to the amino acid sequence according to SEQ ID NO: 16. In some embodiments, the PD-L1 binding domain comprises an amino acid sequence that has at least 97% sequence identity to the amino acid sequence according to SEQ ID NO: 16. In some embodiments, the PD-L1 binding domain comprises an amino acid sequence that has at least 98% sequence identity to the amino acid sequence according to SEQ ID NO: 16. In some embodiments, the PD-L1 binding domain comprises an amino acid sequence that has at least 99% sequence identity to the amino acid sequence according to SEQ ID NO: 16. In some embodiments, the PD-L1 binding domain comprises an amino acid sequence according to SEQ ID NO: 16.

In some embodiments, the PD-L1 binding domain comprises an amino acid sequence of at least 175 consecutive amino acid residues of SEQ ID NO: 16. In some embodiments, the PD-L1 binding domain comprises an amino acid sequence of at least 190 consecutive amino acid residues of SEQ ID NO: 16. In some embodiments, the PD-L1 binding domain comprises an amino acid sequence of at least 200 consecutive amino acid residues of SEQ ID NO: 16. In some embodiments, the PD-L1 binding domain comprises an amino acid sequence of at least 205 consecutive amino acid residues of SEQ ID NO: 16. In some embodiments, the PD-L1 binding domain comprises an amino acid sequence of at least 210 consecutive amino acid residues of SEQ ID NO: 16.

In some embodiments, the PD-L1 binding domain comprises an amino acid sequence of at least 175 consecutive amino acid residues of SEQ ID NO: 16, and has at least 80% sequence identity to the at least 175 consecutive amino acid residues of SEQ ID NO: 16. In some embodiments, the PD-L1 binding domain comprises an amino acid sequence of at least 190 consecutive amino acid residues of SEQ ID NO: 16, and has at least 80% sequence identity to the at least 190 consecutive amino acid residues of SEQ ID NO: 16. In some embodiments, the PD-L1 binding domain comprises an amino acid sequence of at least 200 consecutive amino acid residues of SEQ ID NO: 16, and has at least 80% sequence identity to the at least 200 consecutive amino acid residues of SEQ ID NO: 16. In some embodiments, the PD-L1 binding domain comprises an amino acid sequence of at least 205 consecutive amino acid residues of SEQ ID NO: 16, and has at least 80% sequence identity to the at least 205 consecutive amino acid residues of SEQ ID NO: 16. In some embodiments, the PD-L1 binding domain comprises an amino acid sequence of at least 210 consecutive amino acid residues of SEQ ID NO: 16, and has at least 80% sequence identity to the at least 210 consecutive amino acid residues of SEQ ID NO: 16.

In some embodiments, the PD-L1 binding domain comprises an amino acid sequence of at least 175 consecutive amino acid residues of SEQ ID NO: 16, and has at least 90% sequence identity to the at least 175 consecutive amino acid residues of SEQ ID NO: 16. In some embodiments, the PD-L1 binding domain comprises an amino acid sequence of at least 190 consecutive amino acid residues of SEQ ID NO: 16, and has at least 90% sequence identity to the at least 190 consecutive amino acid residues of SEQ ID NO: 16. In some embodiments, the PD-L1 binding domain comprises an amino acid sequence of at least 200 consecutive amino acid residues of SEQ ID NO: 16, and has at least 90% sequence identity to the at least 200 consecutive amino acid residues of SEQ ID NO: 16. In some embodiments, the PD-L1 binding domain comprises an amino acid sequence of at least 205 consecutive amino acid residues of SEQ ID NO: 16, and has at least 90% sequence identity to the at least 205 consecutive amino acid residues of SEQ ID NO: 16. In some embodiments, the PD-L1 binding domain comprises an amino acid sequence of at least 210 consecutive amino acid residues of SEQ ID NO: 16, and has at least 90% sequence identity to the at least 210 consecutive amino acid residues of SEQ ID NO: 16.

In some embodiments, the PD-L1 binding domain comprises an amino acid sequence of at least 175 consecutive amino acid residues of SEQ ID NO: 16, and has at least 95% sequence identity to the at least 175 consecutive amino acid residues of SEQ ID NO: 16. In some embodiments, the PD-L1 binding domain comprises an amino acid sequence of at least 190 consecutive amino acid residues of SEQ ID NO: 16, and has at least 95% sequence identity to the at least 190 consecutive amino acid residues of SEQ ID NO: 16. In some embodiments, the PD-L1 binding domain comprises an amino acid sequence of at least 200 consecutive amino acid residues of SEQ ID NO: 16, and has at least 95% sequence identity to the at least 200 consecutive amino acid residues of SEQ ID NO: 16. In some embodiments, the PD-L1 binding domain comprises an amino acid sequence of at least 205 consecutive amino acid residues of SEQ ID NO: 16, and has at least 95% sequence identity to the at least 205 consecutive amino acid residues of SEQ ID NO: 16. In some embodiments, the PD-L1 binding domain comprises an amino acid sequence of at least 210 consecutive amino acid residues of SEQ ID NO: 16, and has at least 95% sequence identity to the at least 210 consecutive amino acid residues of SEQ ID NO: 16.

In some embodiments, the PD-L1 binding domain comprises an amino acid sequence of at least 175 consecutive amino acid residues of SEQ ID NO: 16, and has at least 99% sequence identity to the at least 175 consecutive amino acid residues of SEQ ID NO: 16. In some embodiments, the PD-L1 binding domain comprises an amino acid sequence of at least 190 consecutive amino acid residues of SEQ ID NO: 16, and has at least 99% sequence identity to the at least 190 consecutive amino acid residues of SEQ ID NO: 16. In some embodiments, the PD-L1 binding domain comprises an amino acid sequence of at least 200 consecutive amino acid residues of SEQ ID NO: 16, and has at least 99% sequence identity to the at least 200 consecutive amino acid residues of SEQ ID NO: 16. In some embodiments, the PD-L1 binding domain comprises an amino acid sequence of at least 205 consecutive amino acid residues of SEQ ID NO: 16, and has at least 99% sequence identity to the at least 205 consecutive amino acid residues of SEQ ID NO: 16. In some embodiments, the PD-L1 binding domain comprises an amino acid sequence of at least 210 consecutive amino acid residues of SEQ ID NO: 16, and has at least 99% sequence identity to the at least 210 consecutive amino acid residues of SEQ ID NO: 16.

In some embodiments, the PD-L1 binding domain comprises an amino acid sequence that has at least 70% sequence identity to the amino acid sequence according to SEQ ID NO: 17; and the PD-L1 binding domain comprises an amino acid sequence that has at least 70% sequence identity to the amino acid sequence according to SEQ ID NO: 16. In some embodiments, the PD-L1 binding domain comprises an amino acid sequence that has at least 80% sequence identity to the amino acid sequence according to SEQ ID NO: 17; and the PD-L1 binding domain comprises an amino acid sequence that has at least 80% sequence identity to the amino acid sequence according to SEQ ID NO: 16. In some embodiments, the PD-L1 binding domain comprises an amino acid sequence that has at least 85% sequence identity to the amino acid sequence according to SEQ ID NO: 17; and the PD-L1 binding domain comprises an amino acid sequence that has at least 85% sequence identity to the amino acid sequence according to SEQ ID NO: 16. In some embodiments, the PD-L1 binding domain comprises an amino acid sequence that has at least 90% sequence identity to the amino acid sequence according to SEQ ID NO: 17; and the PD-L1 binding domain comprises an amino acid sequence that has at least 90% sequence identity to the amino acid sequence according to SEQ ID NO: 16. In some embodiments, the PD-L1 binding domain comprises an amino acid sequence that has at least 95% sequence identity to the amino acid sequence according to SEQ ID NO: 17; and the PD-L1 binding domain comprises an amino acid sequence that has at least 95% sequence identity to the amino acid sequence according to SEQ ID NO: 16. In some embodiments, the PD-L1 binding domain comprises an amino acid sequence that has at least 96% sequence identity to the amino acid sequence according to SEQ ID NO: 17; and the PD-L1 binding domain comprises an amino acid sequence that has at least 96% sequence identity to the amino acid sequence according to SEQ ID NO: 16. In some embodiments, the PD-L1 binding domain comprises an amino acid sequence that has at least 97% sequence identity to the amino acid sequence according to SEQ ID NO: 17; and the PD-L1 binding domain comprises an amino acid sequence that has at least 97% sequence identity to the amino acid sequence according to SEQ ID NO: 16. In some embodiments, the PD-L1 binding domain comprises an amino acid sequence that has at least 98% sequence identity to the amino acid sequence according to SEQ ID NO: 17; and the PD-L1 binding domain comprises an amino acid sequence that has at least 98% sequence identity to the amino acid sequence according to SEQ ID NO: 16. In some embodiments, the PD-L1 binding domain comprises an amino acid sequence that has at least 99% sequence identity to the amino acid sequence according to SEQ ID NO: 17; and the PD-L1 binding domain comprises an amino acid sequence that has at least 99% sequence identity to the amino acid sequence according to SEQ ID NO: 16.

In some embodiments, the PD-L1 binding domain comprises an amino acid sequence that has at least 70% sequence identity to the amino acid sequence according to SEQ ID NO: 43. In some embodiments, the PD-L1 binding domain comprises an amino acid sequence that has at least 80% sequence identity to the amino acid sequence according to SEQ ID NO: 43. In some embodiments, the PD-L1 binding domain comprises an amino acid sequence that has at least 85% sequence identity to the amino acid sequence according to SEQ ID NO: 43. In some embodiments, the PD-L1 binding domain comprises an amino acid sequence that has at least 90% sequence identity to the amino acid sequence according to SEQ ID NO: 43. In some embodiments, the PD-L1 binding domain comprises an amino acid sequence that has at least 95% sequence identity to the amino acid sequence according to SEQ ID NO: 43. In some embodiments, the PD-L1 binding domain comprises an amino acid sequence that has at least 96% sequence identity to the amino acid sequence according to SEQ ID NO: 43. In some embodiments, the PD-L1 binding domain comprises an amino acid sequence that has at least 97% sequence identity to the amino acid sequence according to SEQ ID NO: 43. In some embodiments, the PD-L1 binding domain comprises an amino acid sequence that has at least 98% sequence identity to the amino acid sequence according to SEQ ID NO: 43. In some embodiments, the PD-L1 binding domain comprises an amino acid sequence that has at least 99% sequence identity to the amino acid sequence according to SEQ ID NO: 43. In some embodiments, the PD-L1 binding domain comprises an amino acid sequence according to SEQ ID NO: 43.

In some embodiments, the PD-L1 binding domain comprises an amino acid sequence of at least 175 consecutive amino acid residues of SEQ ID NO: 43, and has at least 95% sequence identity to the at least 175 consecutive amino acid residues of SEQ ID NO: 43. In some embodiments, the PD-L1 binding domain comprises an amino acid sequence of at least 190 consecutive amino acid residues of SEQ ID NO: 43, and has at least 95% sequence identity to the at least 190 consecutive amino acid residues of SEQ ID NO: 43. In some embodiments, the PD-L1 binding domain comprises an amino acid sequence of at least 200 consecutive amino acid residues of SEQ ID NO: 43, and has at least 95% sequence identity to the at least 200 consecutive amino acid residues of SEQ ID NO: 43. In some embodiments, the PD-L1 binding domain comprises an amino acid sequence of at least 210 consecutive amino acid residues of SEQ ID NO: 43, and has at least 95% sequence identity to the at least 210 consecutive amino acid residues of SEQ ID NO: 43.

In some embodiments, the PD-L1 binding domain comprises an amino acid sequence of at least 215 consecutive amino acid residues of SEQ ID NO: 43, and has at least 95% sequence identity to the at least 215 consecutive amino acid residues of SEQ ID NO: 43. In some embodiments, the PD-L1 binding domain comprises an amino acid sequence of at least 220 consecutive amino acid residues of SEQ ID NO: 43, and has at least 95% sequence identity to the at least 220 consecutive amino acid residues of SEQ ID NO: 43.

In some embodiments, the PD-L1 binding domain comprises an amino acid sequence that has at least 70% sequence identity to the amino acid sequence according to SEQ ID NO: 42. In some embodiments, the PD-L1 binding domain comprises an amino acid sequence that has at least 80% sequence identity to the amino acid sequence according to SEQ ID NO: 42. In some embodiments, the PD-L1 binding domain comprises an amino acid sequence that has at least 85% sequence identity to the amino acid sequence according to SEQ ID NO: 42. In some embodiments, the PD-L1 binding domain comprises an amino acid sequence that has at least 90% sequence identity to the amino acid sequence according to SEQ ID NO: 42. In some embodiments, the PD-L1 binding domain comprises an amino acid sequence that has at least 95% sequence identity to the amino acid sequence according to SEQ ID NO: 42. In some embodiments, the PD-L1 binding domain comprises an amino acid sequence that has at least 96% sequence identity to the amino acid sequence according to SEQ ID NO: 42. In some embodiments, the PD-L1 binding domain comprises an amino acid sequence that has at least 97% sequence identity to the amino acid sequence according to SEQ ID NO: 42. In some embodiments, the PD-L1 binding domain comprises an amino acid sequence that has at least 98% sequence identity to the amino acid sequence according to SEQ ID NO: 42. In some embodiments, the PD-L1 binding domain comprises an amino acid sequence that has at least 99% sequence identity to the amino acid sequence according to SEQ ID NO: 42. In some embodiments, the PD-L1 binding domain comprises an amino acid sequence according to SEQ ID NO: 42.

In some embodiments, the PD-L1 binding domain comprises an amino acid sequence of at least 175 consecutive amino acid residues of SEQ ID NO: 42, and has at least 95% sequence identity to the at least 175 consecutive amino acid residues of SEQ ID NO: 42. In some embodiments, the PD-L1 binding domain comprises an amino acid sequence of at least 190 consecutive amino acid residues of SEQ ID NO: 42, and has at least 95% sequence identity to the at least 190 consecutive amino acid residues of SEQ ID NO: 42. In some embodiments, the PD-L1 binding domain comprises an amino acid sequence of at least 200 consecutive amino acid residues of SEQ ID NO: 42, and has at least 95% sequence identity to the at least 200 consecutive amino acid residues of SEQ ID NO: 42. In some embodiments, the PD-L1 binding domain comprises an amino acid sequence of at least 205 consecutive amino acid residues of SEQ ID NO: 42, and has at least 95% sequence identity to the at least 205 consecutive amino acid residues of SEQ ID NO: 42. In some embodiments, the PD-L1 binding domain comprises an amino acid sequence of at least 210 consecutive amino acid residues of SEQ ID NO: 42, and has at least 95% sequence identity to the at least 210 consecutive amino acid residues of SEQ ID NO: 42.

In some embodiments, the PD-L1 binding domain comprises an amino acid sequence that has at least 70% sequence identity to the amino acid sequence according to SEQ ID NO: 43; and the PD-L1 binding domain comprises an amino acid sequence that has at least 70% sequence identity to the amino acid sequence according to SEQ ID NO: 42. In some embodiments, the PD-L1 binding domain comprises an amino acid sequence that has at least 80% sequence identity to the amino acid sequence according to SEQ ID NO: 43; and the PD-L1 binding domain comprises an amino acid sequence that has at least 80% sequence identity to the amino acid sequence according to SEQ ID NO: 42. In some embodiments, the PD-L1 binding domain comprises an amino acid sequence that has at least 85% sequence identity to the amino acid sequence according to SEQ ID NO: 43; and the PD-L1 binding domain comprises an amino acid sequence that has at least 85% sequence identity to the amino acid sequence according to SEQ ID NO: 42. In some embodiments, the PD-L1 binding domain comprises an amino acid sequence that has at least 90% sequence identity to the amino acid sequence according to SEQ ID NO: 43; and the PD-L1 binding domain comprises an amino acid sequence that has at least 90% sequence identity to the amino acid sequence according to SEQ ID NO: 42. In some embodiments, the PD-L1 binding domain comprises an amino acid sequence that has at least 95% sequence identity to the amino acid sequence according to SEQ ID NO: 43; and the PD-L1 binding domain comprises an amino acid sequence that has at least 95% sequence identity to the amino acid sequence according to SEQ ID NO: 42. In some embodiments, the PD-L1 binding domain comprises an amino acid sequence that has at least 96% sequence identity to the amino acid sequence according to SEQ ID NO: 43; and the PD-L1 binding domain comprises an amino acid sequence that has at least 96% sequence identity to the amino acid sequence according to SEQ ID NO: 42. In some embodiments, the PD-L1 binding domain comprises an amino acid sequence that has at least 97% sequence identity to the amino acid sequence according to SEQ ID NO: 43; and the PD-L1 binding domain comprises an amino acid sequence that has at least 97% sequence identity to the amino acid sequence according to SEQ ID NO: 42. In some embodiments, the PD-L1 binding domain comprises an amino acid sequence that has at least 98% sequence identity to the amino acid sequence according to SEQ ID NO: 43; and the PD-L1 binding domain comprises an amino acid sequence that has at least 98% sequence identity to the amino acid sequence according to SEQ ID NO: 42. In some embodiments, the PD-L1 binding domain comprises an amino acid sequence that has at least 99% sequence identity to the amino acid sequence according to SEQ ID NO: 43; and the PD-L1 binding domain comprises an amino acid sequence that has at least 99% sequence identity to the amino acid sequence according to SEQ ID NO: 42.

In some embodiments, the PD-L1 binding domain comprises an amino acid sequence that has at least 70% sequence identity to the amino acid sequence according to SEQ ID NO: 45. In some embodiments, the PD-L1 binding domain comprises an amino acid sequence that has at least 80% sequence identity to the amino acid sequence according to SEQ ID NO: 45. In some embodiments, the PD-L1 binding domain comprises an amino acid sequence that has at least 85% sequence identity to the amino acid sequence according to SEQ ID NO: 45. In some embodiments, the PD-L1 binding domain comprises an amino acid sequence that has at least 90% sequence identity to the amino acid sequence according to SEQ ID NO: 45. In some embodiments, the PD-L1 binding domain comprises an amino acid sequence that has at least 95% sequence identity to the amino acid sequence according to SEQ ID NO: 45. In some embodiments, the PD-L1 binding domain comprises an amino acid sequence that has at least 96% sequence identity to the amino acid sequence according to SEQ ID NO: 45. In some embodiments, the PD-L1 binding domain comprises an amino acid sequence that has at least 97% sequence identity to the amino acid sequence according to SEQ ID NO: 45. In some embodiments, the PD-L1 binding domain comprises an amino acid sequence that has at least 98% sequence identity to the amino acid sequence according to SEQ ID NO: 45. In some embodiments, the PD-L1 binding domain comprises an amino acid sequence that has at least 99% sequence identity to the amino acid sequence according to SEQ ID NO: 45. In some embodiments, the PD-L1 binding domain comprises an amino acid sequence according to SEQ ID NO: 45.

In some embodiments, the PD-L1 binding domain comprises an amino acid sequence of at least 175 consecutive amino acid residues of SEQ ID NO: 45, and has at least 95% sequence identity to the at least 175 consecutive amino acid residues of SEQ ID NO: 45. In some embodiments, the PD-L1 binding domain comprises an amino acid sequence of at least 190 consecutive amino acid residues of SEQ ID NO: 45, and has at least 95% sequence identity to the at least 190 consecutive amino acid residues of SEQ ID NO: 45. In some embodiments, the PD-L1 binding domain comprises an amino acid sequence of at least 200 consecutive amino acid residues of SEQ ID NO: 45, and has at least 95% sequence identity to the at least 200 consecutive amino acid residues of SEQ ID NO: 45. In some embodiments, the PD-L1 binding domain comprises an amino acid sequence of at least 210 consecutive amino acid residues of SEQ ID NO: 45, and has at least 95% sequence identity to the at least 210 consecutive amino acid residues of SEQ ID NO: 45.

In some embodiments, the PD-L1 binding domain comprises an amino acid sequence of at least 215 consecutive amino acid residues of SEQ ID NO: 45, and has at least 95% sequence identity to the at least 215 consecutive amino acid residues of SEQ ID NO: 45. In some embodiments, the PD-L1 binding domain comprises an amino acid sequence of at least 220 consecutive amino acid residues of SEQ ID NO: 45, and has at least 95% sequence identity to the at least 220 consecutive amino acid residues of SEQ ID NO: 45.

In some embodiments, the PD-L1 binding domain comprises an amino acid sequence that has at least 70% sequence identity to the amino acid sequence according to SEQ ID NO: 44. In some embodiments, the PD-L1 binding domain comprises an amino acid sequence that has at least 80% sequence identity to the amino acid sequence according to SEQ ID NO: 44. In some embodiments, the PD-L1 binding domain comprises an amino acid sequence that has at least 85% sequence identity to the amino acid sequence according to SEQ ID NO: 44. In some embodiments, the PD-L1 binding domain comprises an amino acid sequence that has at least 90% sequence identity to the amino acid sequence according to SEQ ID NO: 44. In some embodiments, the PD-L1 binding domain comprises an amino acid sequence that has at least 95% sequence identity to the amino acid sequence according to SEQ ID NO: 44. In some embodiments, the PD-L1 binding domain comprises an amino acid sequence that has at least 96% sequence identity to the amino acid sequence according to SEQ ID NO: 44. In some embodiments, the PD-L1 binding domain comprises an amino acid sequence that has at least 97% sequence identity to the amino acid sequence according to SEQ ID NO: 44. In some embodiments, the PD-L1 binding domain comprises an amino acid sequence that has at least 98% sequence identity to the amino acid sequence according to SEQ ID NO: 44. In some embodiments, the PD-L1 binding domain comprises an amino acid sequence that has at least 99% sequence identity to the amino acid sequence according to SEQ ID NO: 44. In some embodiments, the PD-L1 binding domain comprises an amino acid sequence according to SEQ ID NO: 44.

In some embodiments, the PD-L1 binding domain comprises an amino acid sequence of at least 175 consecutive amino acid residues of SEQ ID NO: 44, and has at least 95% sequence identity to the at least 175 consecutive amino acid residues of SEQ ID NO: 44. In some embodiments, the PD-L1 binding domain comprises an amino acid sequence of at least 190 consecutive amino acid residues of SEQ ID NO: 44, and has at least 95% sequence identity to the at least 190 consecutive amino acid residues of SEQ ID NO: 44. In some embodiments, the PD-L1 binding domain comprises an amino acid sequence of at least 200 consecutive amino acid residues of SEQ ID NO: 44, and has at least 95% sequence identity to the at least 200 consecutive amino acid residues of SEQ ID NO: 44. In some embodiments, the PD-L1 binding domain comprises an amino acid sequence of at least 205 consecutive amino acid residues of SEQ ID NO: 44, and has at least 95% sequence identity to the at least 205 consecutive amino acid residues of SEQ ID NO: 44. In some embodiments, the PD-L1 binding domain comprises an amino acid sequence of at least 210 consecutive amino acid residues of SEQ ID NO: 44, and has at least 95% sequence identity to the at least 210 consecutive amino acid residues of SEQ ID NO: 44.

In some embodiments, the PD-L1 binding domain comprises an amino acid sequence that has at least 70% sequence identity to the amino acid sequence according to SEQ ID NO: 45; and the PD-L1 binding domain comprises an amino acid sequence that has at least 70% sequence identity to the amino acid sequence according to SEQ ID NO: 44. In some embodiments, the PD-L1 binding domain comprises an amino acid sequence that has at least 80% sequence identity to the amino acid sequence according to SEQ ID NO: 45; and the PD-L1 binding domain comprises an amino acid sequence that has at least 80% sequence identity to the amino acid sequence according to SEQ ID NO: 44. In some embodiments, the PD-L1 binding domain comprises an amino acid sequence that has at least 85% sequence identity to the amino acid sequence according to SEQ ID NO: 45; and the PD-L1 binding domain comprises an amino acid sequence that has at least 85% sequence identity to the amino acid sequence according to SEQ ID NO: 44. In some embodiments, the PD-L1 binding domain comprises an amino acid sequence that has at least 90% sequence identity to the amino acid sequence according to SEQ ID NO: 45; and the PD-L1 binding domain comprises an amino acid sequence that has at least 90% sequence identity to the amino acid sequence according to SEQ ID NO: 44. In some embodiments, the PD-L1 binding domain comprises an amino acid sequence that has at least 95% sequence identity to the amino acid sequence according to SEQ ID NO: 45; and the PD-L1 binding domain comprises an amino acid sequence that has at least 95% sequence identity to the amino acid sequence according to SEQ ID NO: 44. In some embodiments, the PD-L1 binding domain comprises an amino acid sequence that has at least 96% sequence identity to the amino acid sequence according to SEQ ID NO: 45; and the PD-L1 binding domain comprises an amino acid sequence that has at least 96% sequence identity to the amino acid sequence according to SEQ ID NO: 44. In some embodiments, the PD-L1 binding domain comprises an amino acid sequence that has at least 97% sequence identity to the amino acid sequence according to SEQ ID NO: 45; and the PD-L1 binding domain comprises an amino acid sequence that has at least 97% sequence identity to the amino acid sequence according to SEQ ID NO: 44. In some embodiments, the PD-L1 binding domain comprises an amino acid sequence that has at least 98% sequence identity to the amino acid sequence according to SEQ ID NO: 45; and the PD-L1 binding domain comprises an amino acid sequence that has at least 98% sequence identity to the amino acid sequence according to SEQ ID NO: 44. In some embodiments, the PD-L1 binding domain comprises an amino acid sequence that has at least 99% sequence identity to the amino acid sequence according to SEQ ID NO: 45; and the PD-L1 binding domain comprises an amino acid sequence that has at least 99% sequence identity to the amino acid sequence according to SEQ ID NO: 44.

In some embodiments, the PD-L1 binding domain comprises an amino acid sequence that has at least 70% sequence identity to the amino acid sequence according to SEQ ID NO: 47. In some embodiments, the PD-L1 binding domain comprises an amino acid sequence that has at least 80% sequence identity to the amino acid sequence according to SEQ ID NO: 47. In some embodiments, the PD-L1 binding domain comprises an amino acid sequence that has at least 85% sequence identity to the amino acid sequence according to SEQ ID NO: 47. In some embodiments, the PD-L1 binding domain comprises an amino acid sequence that has at least 90% sequence identity to the amino acid sequence according to SEQ ID NO: 47. In some embodiments, the PD-L1 binding domain comprises an amino acid sequence that has at least 95% sequence identity to the amino acid sequence according to SEQ ID NO: 47. In some embodiments, the PD-L1 binding domain comprises an amino acid sequence that has at least 96% sequence identity to the amino acid sequence according to SEQ ID NO: 47. In some embodiments, the PD-L1 binding domain comprises an amino acid sequence that has at least 97% sequence identity to the amino acid sequence according to SEQ ID NO: 47. In some embodiments, the PD-L1 binding domain comprises an amino acid sequence that has at least 98% sequence identity to the amino acid sequence according to SEQ ID NO: 47. In some embodiments, the PD-L1 binding domain comprises an amino acid sequence that has at least 99% sequence identity to the amino acid sequence according to SEQ ID NO: 47. In some embodiments, the PD-L1 binding domain comprises an amino acid sequence according to SEQ ID NO: 47.

In some embodiments, the PD-L1 binding domain comprises an amino acid sequence of at least 175 consecutive amino acid residues of SEQ ID NO: 47, and has at least 95% sequence identity to the at least 175 consecutive amino acid residues of SEQ ID NO: 47. In some embodiments, the PD-L1 binding domain comprises an amino acid sequence of at least 190 consecutive amino acid residues of SEQ ID NO: 47, and has at least 95% sequence identity to the at least 190 consecutive amino acid residues of SEQ ID NO: 47. In some embodiments, the PD-L1 binding domain comprises an amino acid sequence of at least 200 consecutive amino acid residues of SEQ ID NO: 47, and has at least 95% sequence identity to the at least 200 consecutive amino acid residues of SEQ ID NO: 47. In some embodiments, the PD-L1 binding domain comprises an amino acid sequence of at least 210 consecutive amino acid residues of SEQ ID NO: 47, and has at least 95% sequence identity to the at least 210 consecutive amino acid residues of SEQ ID NO: 47.

In some embodiments, the PD-L1 binding domain comprises an amino acid sequence of at least 215 consecutive amino acid residues of SEQ ID NO: 47, and has at least 95% sequence identity to the at least 215 consecutive amino acid residues of SEQ ID NO: 47. In some embodiments, the PD-L1 binding domain comprises an amino acid sequence of at least 220 consecutive amino acid residues of SEQ ID NO: 47, and has at least 95% sequence identity to the at least 220 consecutive amino acid residues of SEQ ID NO: 47.

In some embodiments, the PD-L1 binding domain comprises an amino acid sequence that has at least 70% sequence identity to the amino acid sequence according to SEQ ID NO: 46. In some embodiments, the PD-L1 binding domain comprises an amino acid sequence that has at least 80% sequence identity to the amino acid sequence according to SEQ ID NO: 46. In some embodiments, the PD-L1 binding domain comprises an amino acid sequence that has at least 85% sequence identity to the amino acid sequence according to SEQ ID NO: 46. In some embodiments, the PD-L1 binding domain comprises an amino acid sequence that has at least 90% sequence identity to the amino acid sequence according to SEQ ID NO: 46. In some embodiments, the PD-L1 binding domain comprises an amino acid sequence that has at least 95% sequence identity to the amino acid sequence according to SEQ ID NO: 46. In some embodiments, the PD-L1 binding domain comprises an amino acid sequence that has at least 96% sequence identity to the amino acid sequence according to SEQ ID NO: 46. In some embodiments, the PD-L1 binding domain comprises an amino acid sequence that has at least 97% sequence identity to the amino acid sequence according to SEQ ID NO: 46. In some embodiments, the PD-L1 binding domain comprises an amino acid sequence that has at least 98% sequence identity to the amino acid sequence according to SEQ ID NO: 46. In some embodiments, the PD-L1 binding domain comprises an amino acid sequence that has at least 99% sequence identity to the amino acid sequence according to SEQ ID NO: 46. In some embodiments, the PD-L1 binding domain comprises an amino acid sequence according to SEQ ID NO: 46.

In some embodiments, the PD-L1 binding domain comprises an amino acid sequence of at least 175 consecutive amino acid residues of SEQ ID NO: 46, and has at least 95% sequence identity to the at least 175 consecutive amino acid residues of SEQ ID NO: 46. In some embodiments, the PD-L1 binding domain comprises an amino acid sequence of at least 190 consecutive amino acid residues of SEQ ID NO: 46, and has at least 95% sequence identity to the at least 190 consecutive amino acid residues of SEQ ID NO: 46. In some embodiments, the PD-L1 binding domain comprises an amino acid sequence of at least 200 consecutive amino acid residues of SEQ ID NO: 46, and has at least 95% sequence identity to the at least 200 consecutive amino acid residues of SEQ ID NO: 46. In some embodiments, the PD-L1 binding domain comprises an amino acid sequence of at least 205 consecutive amino acid residues of SEQ ID NO: 46, and has at least 95% sequence identity to the at least 205 consecutive amino acid residues of SEQ ID NO: 46. In some embodiments, the PD-L1 binding domain comprises an amino acid sequence of at least 210 consecutive amino acid residues of SEQ ID NO: 46, and has at least 95% sequence identity to the at least 210 consecutive amino acid residues of SEQ ID NO: 46.

In some embodiments, the PD-L1 binding domain comprises an amino acid sequence that has at least 70% sequence identity to the amino acid sequence according to SEQ ID NO: 47; and the PD-L1 binding domain comprises an amino acid sequence that has at least 70% sequence identity to the amino acid sequence according to SEQ ID NO: 46. In some embodiments, the PD-L1 binding domain comprises an amino acid sequence that has at least 80% sequence identity to the amino acid sequence according to SEQ ID NO: 47; and the PD-L1 binding domain comprises an amino acid sequence that has at least 80% sequence identity to the amino acid sequence according to SEQ ID NO: 46. In some embodiments, the PD-L1 binding domain comprises an amino acid sequence that has at least 85% sequence identity to the amino acid sequence according to SEQ ID NO: 47; and the PD-L1 binding domain comprises an amino acid sequence that has at least 85% sequence identity to the amino acid sequence according to SEQ ID NO: 46. In some embodiments, the PD-L1 binding domain comprises an amino acid sequence that has at least 90% sequence identity to the amino acid sequence according to SEQ ID NO: 47; and the PD-L1 binding domain comprises an amino acid sequence that has at least 90% sequence identity to the amino acid sequence according to SEQ ID NO: 46. In some embodiments, the PD-L1 binding domain comprises an amino acid sequence that has at least 95% sequence identity to the amino acid sequence according to SEQ ID NO: 47; and the PD-L1 binding domain comprises an amino acid sequence that has at least 95% sequence identity to the amino acid sequence according to SEQ ID NO: 46. In some embodiments, the PD-L1 binding domain comprises an amino acid sequence that has at least 96% sequence identity to the amino acid sequence according to SEQ ID NO: 47; and the PD-L1 binding domain comprises an amino acid sequence that has at least 96% sequence identity to the amino acid sequence according to SEQ ID NO: 46. In some embodiments, the PD-L1 binding domain comprises an amino acid sequence that has at least 97% sequence identity to the amino acid sequence according to SEQ ID NO: 47; and the PD-L1 binding domain comprises an amino acid sequence that has at least 97% sequence identity to the amino acid sequence according to SEQ ID NO: 46. In some embodiments, the PD-L1 binding domain comprises an amino acid sequence that has at least 98% sequence identity to the amino acid sequence according to SEQ ID NO: 47; and the PD-L1 binding domain comprises an amino acid sequence that has at least 98% sequence identity to the amino acid sequence according to SEQ ID NO: 46. In some embodiments, the PD-L1 binding domain comprises an amino acid sequence that has at least 99% sequence identity to the amino acid sequence according to SEQ ID NO: 47; and the PD-L1 binding domain comprises an amino acid sequence that has at least 99% sequence identity to the amino acid sequence according to SEQ ID NO: 46.

Linker

In some embodiments, the linker is at least 5 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker is 6 amino acids in length. In some embodiments, the linker is 7 amino acids in length. In some embodiments, the linker is 8 amino acids in length. In some embodiments, the linker is 9 amino acids in length. In some embodiments, the linker is 10 amino acids in length. In some embodiments, the linker is 11 amino acids in length. In some embodiments, the linker is 12 amino acids in length. In some embodiments, the linker is 13 amino acids in length. In some embodiments, the linker is 14 amino acids in length. In some embodiments, the linker is 15 amino acids in length. In some embodiments, the linker is 16 amino acids in length. In some embodiments, the linker is 17 amino acids in length. In some embodiments, the linker is 18 amino acids in length. In some embodiments, the linker is 19 amino acids in length. In some embodiments, the linker is 20 amino acids in length. In some embodiments, the linker is 21 amino acids in length. In some embodiments, the linker is 22 amino acids in length. In some embodiments, the linker is 23 amino acids in length. In some embodiments, the linker is 24 amino acids in length. In some embodiments, the linker is 25 amino acids in length. In some embodiments, the linker is 26 amino acids in length. In some embodiments, the linker is 27 amino acids in length. In some embodiments, the linker is 28 amino acids in length. In some embodiments, the linker is 29 amino acids in length. In some embodiments, the linker is 30 amino acids in length. In some embodiments, the linker comprises an amino acid sequence of SEQ ID NO: 18 (GGGGSGGGGSGGGGS) or SEQ ID NO: 19 (GGGGS). In some embodiments, the linker comprises an amino acid sequence of SEQ ID NO: 18 (GGGGSGGGGSGGGGS). In some embodiments, the linker comprises an amino acid sequence of SEQ ID NO: 19 (GGGGS).

In some embodiments, the linker has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the linker has a formula selected from the group consisting of (G₂S)ₙ, (GS)ₙ, (GSGGS)ₙ (SEQ ID NO: 234), (GGGS)ₙ (SEQ ID NO: 235), (GGGGS)ₙ (SEQ ID NO: 236), and (GSSGGS)ₙ (SEQ ID NO: 237), wherein n is an integer of 1. In some embodiments, the linker has a formula selected from the group consisting of (G₂S)ₙ (SEQ ID NO: 233), (GS)ₙ (SEQ ID NO: 238), (GSGGS)ₙ (SEQ ID NO: 239), (GGGS)ₙ (SEQ ID NO: 240), (GGGGS)ₙ (SEQ ID NO: 241), and (GSSGGS)ₙ (SEQ ID NO: 242), wherein n is an integer from 1 to 3. In some embodiments, the linker has a formula selected from the group consisting of (G₂S)ₙ (SEQ ID NO: 243), (GS)ₙ (SEQ ID NO: 244), (GSGGS)ₙ (SEQ ID NO: 245), (GGGS)ₙ (SEQ ID NO: 246), (GGGGS)ₙ (SEQ ID NO: 247), and (GSSGGS)ₙ (SEQ ID NO: 248), wherein n is an integer from 1 to 10.

In some embodiments, the linker has a formula of (G₂S)ₙ, wherein n is an integer of least 1. In some embodiments, the linker has a formula of (GS)ₙ, wherein n is an integer of least 1. In some embodiments, the linker has a formula of (GSGGS)ₙ (SEQ ID NO: 58), wherein n is an integer of least 1. In some embodiments, the linker has a formula of (GGGS)ₙ (SEQ ID NO: 59), wherein n is an integer of least 1. In some embodiments, the linker has a formula of (GGGGS)ₙ (SEQ ID NO: 60), wherein n is an integer of least 1. In some embodiments, the linker has a formula of (GSSGGS)ₙ (SEQ ID NO: 61), wherein n is an integer of least 1.

In some embodiments, the linker has a formula of (G₂S)ₙ, wherein n is an integer of 1. In some embodiments, L₁ has a formula of (GS)ₙ, wherein n is an integer of 1. In some embodiments, the linker has a formula of (GSGGS)ₙ (SEQ ID NO: 234), wherein n is an integer of 1. In some embodiments, the linker has a formula of (GGGS)ₙ (SEQ ID NO: 235), wherein n is an integer of 1. In some embodiments, the linker has a formula of (GGGGS)ₙ (SEQ ID NO: 236), wherein n is an integer of 1. In some embodiments, the linker has a formula of (GSSGGS)ₙ (SEQ ID NO: 237), wherein n is an integer of 1.

In some embodiments, the linker has a formula of (G₂S)ₙ (SEQ ID NO: 233), wherein n is an integer from 1 to 3. In some embodiments, the linker has a formula of (GS)ₙ (SEQ ID NO: 238), wherein n is an integer from 1 to 3. In some embodiments, the linker has a formula of (GSGGS)ₙ (SEQ ID NO: 239), wherein n is an integer from 1 to 3. In some embodiments, the linker has a formula of (GGGS)ₙ (SEQ ID NO: 240), wherein n is an integer from 1 to 3. In some embodiments, the linker has a formula of (GGGGS)ₙ (SEQ ID NO: 241), wherein n is an integer from 1 to 3. In some embodiments, the linker has a formula of (GSSGGS)ₙ (SEQ ID NO: 242), wherein n is an integer from 1 to 3.

In some embodiments, the linker has a formula of (G₂S)ₙ (SEQ ID NO: 243), wherein n is an integer from 1 to 10. In some embodiments, the linker has a formula of (GS)ₙ (SEQ ID NO: 244), wherein n is an integer from 1 to 10. In some embodiments, the linker has a formula of (GSGGS)ₙ (SEQ ID NO: 245), wherein n is an integer from 1 to 10. In some embodiments, the linker has a formula of (GGGS)ₙ (SEQ ID NO: 246), wherein n is an integer from 1 to 10. In some embodiments, the linker has a formula of (GGGGS)ₙ (SEQ ID NO: 247), wherein n is an integer from 1 to 10. In some embodiments, the linker has a formula of (GSSGGS)ₙ (SEQ ID NO: 248), wherein n is an integer from 1 to 10.

TABLE 7
Linker sequences

| Construct | Amino Acid Sequence | SEQ ID |
|---|---|---|
| Description | (N to C) | NO: |
| Linker 1 | GGGGSGGGGSGGGGS | 18 |
| Linker 2 | GGGGS | 19 |

In some embodiments, the linker connects the C-terminus of A to an N-terminus of B. In some embodiments, the linker connects the N-terminus of A to a C-terminus of B. In some embodiments, the linker connects the C-terminus of A to the N-terminus of the Fab heavy chain polypeptide. In some embodiments, the linker connects the N-terminus of A to the C-terminus of the Fab heavy chain polypeptide.

In some embodiments, the linker connects the C-terminus of A to the N-terminus of the Fab light chain polypeptide. In some embodiments, the linker connects the N-terminus of A to the C-terminus of the Fab light chain polypeptide. In some embodiments, the linker connects the Fab light chain polypeptide to the scFv light chain variable domain. In some embodiments, the linker connects the Fab light chain polypeptide to the scFv heavy chain variable domain. In some embodiments, the linker connects the Fab heavy chain polypeptide to the scFv light chain variable domain. In some embodiments, the linker connects the Fab heavy chain polypeptide to the scFv heavy chain variable domain. In some embodiments, the linker connects the Fab light chain polypeptide to the N-terminus of the scFv light chain variable domain. In some embodiments, the linker connects the Fab light chain polypeptide to the C-terminus of the scFv light chain variable domain. In some embodiments, the linker connects the Fab light chain polypeptide to the N-terminus of the scFv heavy chain variable domain. In some embodiments, the linker connects the Fab light chain polypeptide to the C-terminus of the scFv heavy chain variable domain. In some embodiments, the linker connects the Fab heavy chain polypeptide to the N-terminus of the scFv light chain variable domain.

In some embodiments, the linker connects the Fab heavy chain polypeptide to the C-terminus of the scFv light chain variable domain. In some embodiments, the linker connects the Fab heavy chain polypeptide to the N-terminus of the scFv heavy chain variable domain. In some embodiments, the linker connects the Fab heavy chain polypeptide to the C-terminus of the scFv heavy chain variable domain.

Multispecific Antibodies that Bind to CD28 and PD-L1

In some embodiments, the multispecific antibody comprising a CD28 binding domain and a PD-L1 binding domain comprises a variety of multispecific antibody formats. In some embodiments, the multispecific antibody comprises a CD28 binding domain and a PD-L1 binding domain, wherein when the CD28 binding domain is a single chain variable fragment (scFv), then the PD-L1 binding domain is not a scFv.

In some embodiments, the multispecific antibody further comprises a fragment crystallizable (Fc) region. In some embodiments, the Fc region comprises an IgG CH2 domain and an IgG CH3 domain. In some embodiments, the Fc region comprises a heterodimeric Fc region. In some embodiments, the Fc region comprises at least one amino acid modification that increases the half-life of the multispecific antibody. In some embodiments, the Fc region comprises at least one amino acid modification that modulates its interaction with an Fc receptor. In some embodiments, the Fc region comprises at least one amino acid modification that increases binding of the Fc region to an Fc receptor. In some embodiments, the Fc region comprises at least one amino acid modification that decreases glycosylation of the Fc region. In some embodiments, the modification is an amino acid substitution, deletion, or addition. In some embodiments, the modification is an amino acid substitution. In some embodiments, the at least one amino acid modification that decreases glycosylation of the Fc region comprises an amino acid substitution at a position corresponding to position N297 of human IgG1, wherein the numbering is according to the EU index of Kabat. In some embodiments, the Fc region is afucosylated.

In some embodiments, the multispecific antibody is assembled from at least two different heavy and light chains expressed in the same producer cell. In some embodiments, the multispecific antibody is produced using knobs-into-holes technology to force heavy-chain heterodimerization in which mutations are introduced into the two CH3 domains.

In some embodiments, the multispecific antibody lacks a fragment crystallizable (Fc) region. In some embodiments, two or more different antibodies are linked together to form the multispecific antibody.

In some embodiments, two different antibodies are linked together to form the multispecific antibody. For example, the PD-L1 binding domain is a Fab or Fab′ and is linked to the CD28 binding domain that is a scFv, another Fab or Fab′, or a single domain antibody. In some embodiments, the CD28 binding domain is a scFv and is linked to the PD-L1 binding domain that is Fab or Fab′ or a single domain antibody. In some embodiments, the PD-L1 binding domain is a single domain antibody and is linked to the CD28 binding domain that is another single domain antibody. In some embodiments, the PD-L1 binding domain is a Fab or Fab′ and is linked to the CD28 binding domain that is another Fab or Fab′. In some embodiments, the PD-L1 binding domain is a Fab or Fab′ and is linked to the CD28 binding domain that is a single domain antibody. In some embodiments, the PD-L1 binding domain is a Fab or Fab′ and is linked to the CD28 binding domain that is a scFv. In some embodiments, the PD-L1 binding domain is a single domain antibody and is linked to the CD28 binding domain that is a Fab or Fab′. In some embodiments, the PD-L1 binding domain is a single domain antibody and is linked to the CD28 binding domain that is a scFv. In some embodiments, the PD-L1 binding domain is a scFv and is linked to the CD28 binding domain that is a Fab or Fab′. In some embodiments, the PD-L1 binding domain is a scFv and is linked to the CD28 binding domain that is a single domain antibody. In some embodiments, the PD-L1 binding domain is a scFv and is linked to the CD28 binding domain that is a scFv.

In some embodiments, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21. In some embodiments, the linker connects the Fab heavy chain polypeptide to the C-terminus of the scFv light chain variable domain and wherein the Fab light chain polypeptide comprises an amino acid sequence that has at least 90% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 90% sequence identity to the amino acid sequence according to SEQ ID NO: 21. In some embodiments, the linker connects the Fab heavy chain polypeptide to the C-terminus of the scFv light chain variable domain and wherein the Fab light chain polypeptide comprises an amino acid sequence that has at least 95% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 95% sequence identity to the amino acid sequence according to SEQ ID NO: 21. In some embodiments, the linker connects the Fab heavy chain polypeptide to the C-terminus of the scFv light chain variable domain and wherein the Fab light chain polypeptide comprises an amino acid sequence that has at least 96% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 96% sequence identity to the amino acid sequence according to SEQ ID NO: 21.

In some embodiments, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21. In some embodiments, the linker connects the Fab heavy chain polypeptide to the C-terminus of the scFv light chain variable domain and wherein the Fab light chain polypeptide comprises an amino acid sequence that has at least 97% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 97% sequence identity to the amino acid sequence according to SEQ ID NO: 21. In some embodiments, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21. In some embodiments, the linker connects the Fab heavy chain polypeptide to the C-terminus of the scFv light chain variable domain and wherein the Fab light chain polypeptide comprises an amino acid sequence that has at least 98% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 98% sequence identity to the amino acid sequence according to SEQ ID NO: 21. In some embodiments, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21. In some embodiments, the linker connects the Fab heavy chain polypeptide to the C-terminus of the scFv light chain variable domain and wherein the Fab light chain polypeptide comprises an amino acid sequence that has at least 99% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 99% sequence identity to the amino acid sequence according to SEQ ID NO: 21.

In some embodiments, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 210 consecutive amino acid residues of SEQ ID NO: 20, and an amino acid sequence of the Fab heavy chain polypeptide that is connected to the C-terminus of the scFv light chain variable domain comprises an amino acid sequence of at least 470 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 90% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 90% sequence identity to the at least 450 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 95% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 95% sequence identity to the at least 450 consecutive amino acid residues of SEQ ID NO: 21.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 99% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 99% sequence identity to the at least 450 consecutive amino acid residues of SEQ ID NO: 21.

In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 90% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 90% sequence identity to the amino acid sequence according to SEQ ID NO: 22.

In some embodiments, the linker connects the Fab light chain polypeptide to the C-terminus of the scFv light chain variable domain and wherein the Fab heavy chain polypeptide comprises an amino acid sequence that has at least 95% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 95% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 96% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 96% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 97% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 97% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 98% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 98% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence that has at least 99% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 99% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22. In some embodiments, the linker connects the Fab light chain polypeptide to the C-terminus of the scFv light chain variable domain and wherein the Fab heavy chain polypeptide comprises an amino acid sequence of at least 220 consecutive amino acid residues of SEQ ID NO: 23, and an amino acid sequence of the Fab light chain polypeptide that is connected to the C-terminus of the scFv light chain variable domain comprises an amino acid sequence of at least 460 consecutive amino acid residues of SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 90% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 90% sequence identity to the at least 450 consecutive amino acid residues of SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 95% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 95% sequence identity to the at least 450 consecutive amino acid residues of SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 96% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 96% sequence identity to the at least 450 consecutive amino acid residues of SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 97% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 97% sequence identity to the at least 450 consecutive amino acid residues of SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 98% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 98% sequence identity to the at least 450 consecutive amino acid residues of SEQ ID NO: 22.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 99% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 99% sequence identity to the at least 450 consecutive amino acid residues of SEQ ID NO: 22.

In some embodiments, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 42, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 48. In some embodiments, the linker connects the Fab heavy chain polypeptide to the C-terminus of the scFv light chain variable domain and wherein the Fab light chain polypeptide comprises an amino acid sequence that has at least 90% sequence identity to the amino acid sequence according to SEQ ID NO: 42, and an amino acid sequence of the Fab heavy chain polypeptide that is connected to the C-terminus of the scFv light chain variable domain comprises an amino acid sequence that has at least 90% sequence identity to the amino acid sequence according to SEQ ID NO: 48. In some embodiments, the linker connects the Fab heavy chain polypeptide to the C-terminus of the scFv light chain variable domain and wherein the Fab light chain polypeptide comprises an amino acid sequence that has at least 95% sequence identity to the amino acid sequence according to SEQ ID NO: 42, and an amino acid sequence of the Fab heavy chain polypeptide that is connected to the C-terminus of the scFv light chain variable domain comprises an amino acid sequence that has at least 95% sequence identity to the amino acid sequence according to SEQ ID NO: 48. In some embodiments, the linker connects the Fab heavy chain polypeptide to the C-terminus of the scFv light chain variable domain and wherein the Fab light chain polypeptide comprises an amino acid sequence that has at least 96% sequence identity to the amino acid sequence according to SEQ ID NO: 42, and an amino acid sequence of the Fab heavy chain polypeptide that is connected to the C-terminus of the scFv light chain variable domain comprises an amino acid sequence that has at least 96% sequence identity to the amino acid sequence according to SEQ ID NO: 48.

In some embodiments, the linker connects the Fab heavy chain polypeptide to the C-terminus of the scFv light chain variable domain and wherein the Fab light chain polypeptide comprises an amino acid sequence according to SEQ ID NO: 42, and an amino acid sequence of the Fab heavy chain polypeptide that is connected to the C-terminus of the scFv light chain variable domain comprises an amino acid sequence to SEQ ID NO: 48. In some embodiments, the linker connects the Fab heavy chain polypeptide to the C-terminus of the scFv light chain variable domain and wherein the Fab light chain polypeptide comprises an amino acid sequence that has at least 97% sequence identity to the amino acid sequence according to SEQ ID NO: 42, and an amino acid sequence of the Fab heavy chain polypeptide that is connected to the C-terminus of the scFv light chain variable domain comprises an amino acid sequence that has at least 97% sequence identity to the amino acid sequence according to SEQ ID NO: 48. In some embodiments, the linker connects the Fab heavy chain polypeptide to the C-terminus of the scFv light chain variable domain and wherein the Fab light chain polypeptide comprises an amino acid sequence according to SEQ ID NO: 42, and an amino acid sequence of the Fab heavy chain polypeptide that is connected to the C-terminus of the scFv light chain variable domain comprises an amino acid sequence to SEQ ID NO: 48. In some embodiments, the linker connects the Fab heavy chain polypeptide to the C-terminus of the scFv light chain variable domain and wherein the Fab light chain polypeptide comprises an amino acid sequence that has at least 98% sequence identity to the amino acid sequence according to SEQ ID NO: 42, and an amino acid sequence of the Fab heavy chain polypeptide that is connected to the C-terminus of the scFv light chain variable domain comprises an amino acid sequence that has at least 98% sequence identity to the amino acid sequence according to SEQ ID NO: 48. In some embodiments, the linker connects the Fab heavy chain polypeptide to the C-terminus of the scFv light chain variable domain and wherein the Fab light chain polypeptide comprises an amino acid sequence according to SEQ ID NO: 42, and an amino acid sequence of the Fab heavy chain polypeptide that is connected to the C-terminus of the scFv light chain variable domain comprises an amino acid sequence to SEQ ID NO: 48. In some embodiments, the linker connects the Fab heavy chain polypeptide to the C-terminus of the scFv light chain variable domain and wherein the Fab light chain polypeptide comprises an amino acid sequence that has at least 99% sequence identity to the amino acid sequence according to SEQ ID NO: 42, and an amino acid sequence of the Fab heavy chain polypeptide that is connected to the C-terminus of the scFv light chain variable domain comprises an amino acid sequence that has at least 99% sequence identity to the amino acid sequence according to SEQ ID NO: 48.

In some embodiments, the linker connects the Fab heavy chain polypeptide to the C-terminus of the scFv light chain variable domain and wherein the Fab light chain polypeptide comprises an amino acid sequence according to SEQ ID NO: 42, and an amino acid sequence of the Fab heavy chain polypeptide that is connected to the C-terminus of the scFv light chain variable domain comprises an amino acid sequence to SEQ ID NO: 48.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 42 and has at least 95% sequence identity to the at least 200 consecutive amino acid residues of SEQ ID NO: 42 and an amino acid sequence of the Fab heavy chain polypeptide that is connected to the C-terminus of the scFv light chain variable domain comprises an amino acid sequence of at least 450 consecutive amino acid residues of SEQ ID NO: 48 and has at least 95% sequence identity to the at least 450 consecutive amino acid residues of SEQ ID NO: 48.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 42 and has at least 99% sequence identity to the at least 200 consecutive amino acid residues of SEQ ID NO: 42 and an amino acid sequence of the Fab heavy chain polypeptide that is connected to the C-terminus of the scFv light chain variable domain comprises an amino acid sequence of at least 450 consecutive amino acid residues of SEQ ID NO: 48 and has at least 99% sequence identity to the at least 450 consecutive amino acid residues of SEQ ID NO: 48.

In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 43, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 49. In some embodiments, the linker connects the Fab light chain polypeptide to the C-terminus of the scFv light chain variable domain and wherein the Fab heavy chain polypeptide comprises an amino acid sequence that has at least 90% sequence identity to the amino acid sequence according to SEQ ID NO: 43, and an amino acid sequence of the Fab light chain polypeptide that is connected to the C-terminus of the scFv light chain variable domain comprises an amino acid sequence that has at least 90% sequence identity to the amino acid sequence according to SEQ ID NO: 49.

In some embodiments, the linker connects the Fab light chain polypeptide to the C-terminus of the scFv light chain variable domain and wherein the Fab heavy chain polypeptide comprises an amino acid sequence that has at least 95% sequence identity to the amino acid sequence according to SEQ ID NO: 43, and an amino acid sequence of the Fab light chain polypeptide that is connected to the C-terminus of the scFv light chain variable domain comprises an amino acid sequence that has at least 95% sequence identity to the amino acid sequence according to SEQ ID NO: 49. In some embodiments, the linker connects the Fab light chain polypeptide to the C-terminus of the scFv light chain variable domain and wherein the Fab heavy chain polypeptide comprises an amino acid sequence that has at least 96% sequence identity to the amino acid sequence according to SEQ ID NO: 43, and an amino acid sequence of the Fab light chain polypeptide that is connected to the C-terminus of the scFv light chain variable domain comprises an amino acid sequence that has at least 96% sequence identity to the amino acid sequence according to SEQ ID NO: 49. In some embodiments, the linker connects the Fab light chain polypeptide to the C-terminus of the scFv light chain variable domain and wherein the Fab heavy chain polypeptide comprises an amino acid sequence that has at least 97% sequence identity to the amino acid sequence according to SEQ ID NO: 43, and an amino acid sequence of the Fab light chain polypeptide that is connected to the C-terminus of the scFv light chain variable domain comprises an amino acid sequence that has at least 97% sequence identity to the amino acid sequence according to SEQ ID NO: 49. In some embodiments, the linker connects the Fab light chain polypeptide to the C-terminus of the scFv light chain variable domain and wherein the Fab heavy chain polypeptide comprises an amino acid sequence that has at least 98% sequence identity to the amino acid sequence according to SEQ ID NO: 43, and an amino acid sequence of the Fab light chain polypeptide that is connected to the C-terminus of the scFv light chain variable domain comprises an amino acid sequence that has at least 98% sequence identity to the amino acid sequence according to SEQ ID NO: 49. In some embodiments, the linker connects the Fab light chain polypeptide to the C-terminus of the scFv light chain variable domain and wherein the Fab heavy chain polypeptide comprises an amino acid sequence that has at least 99% sequence identity to the amino acid sequence according to SEQ ID NO: 43, and an amino acid sequence of the Fab light chain polypeptide that is connected to the C-terminus of the scFv light chain variable domain comprises an amino acid sequence that has at least 99% sequence identity to the amino acid sequence according to SEQ ID NO: 49. In some embodiments, the linker connects the Fab light chain polypeptide to the C-terminus of the scFv light chain variable domain and wherein the Fab heavy chain polypeptide comprises an amino acid sequence according to SEQ ID NO: 43, and an amino acid sequence of the Fab light chain polypeptide that is connected to the C-terminus of the scFv light chain variable domain comprises an amino acid sequence to SEQ ID NO: 49.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 43 and has at least 95% sequence identity to the at least 200 consecutive amino acid residues of SEQ ID NO: 43 and an amino acid sequence of the Fab light chain polypeptide that is connected to the C-terminus of the scFv light chain variable domain comprises an amino acid sequence of at least 450 consecutive amino acid residues of SEQ ID NO: 49 and has at least 95% sequence identity to the at least 450 consecutive amino acid residues of SEQ ID NO: 49.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 43 and has at least 99% sequence identity to the at least 200 consecutive amino acid residues of SEQ ID NO: 43 and an amino acid sequence of the Fab light chain polypeptide that is connected to the C-terminus of the scFv light chain variable domain comprises an amino acid sequence of at least 450 consecutive amino acid residues of SEQ ID NO: 49 and has at least 99% sequence identity to the at least 450 consecutive amino acid residues of SEQ ID NO: 49.

In some embodiments, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 44, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 50. In some embodiments, the linker connects the Fab heavy chain polypeptide to the C-terminus of the scFv light chain variable domain and wherein the Fab light chain polypeptide comprises an amino acid sequence that has at least 90% sequence identity to the amino acid sequence according to SEQ ID NO: 44, and an amino acid sequence of the Fab heavy chain polypeptide that is connected to the C-terminus of the scFv light chain variable domain comprises an amino acid sequence that has at least 90% sequence identity to the amino acid sequence according to SEQ ID NO: 50. In some embodiments, the linker connects the Fab heavy chain polypeptide to the C-terminus of the scFv light chain variable domain and wherein the Fab light chain polypeptide comprises an amino acid sequence that has at least 95% sequence identity to the amino acid sequence according to SEQ ID NO: 44, and an amino acid sequence of the Fab heavy chain polypeptide that is connected to the C-terminus of the scFv light chain variable domain comprises an amino acid sequence that has at least 95% sequence identity to the amino acid sequence according to SEQ ID NO: 50. In some embodiments, the linker connects the Fab heavy chain polypeptide to the C-terminus of the scFv light chain variable domain and wherein the Fab light chain polypeptide comprises an amino acid sequence that has at least 96% sequence identity to the amino acid sequence according to SEQ ID NO: 44, and an amino acid sequence of the Fab heavy chain polypeptide that is connected to the C-terminus of the scFv light chain variable domain comprises an amino acid sequence that has at least 96% sequence identity to the amino acid sequence according to SEQ ID NO: 50.

In some embodiments, the linker connects the Fab heavy chain polypeptide to the C-terminus of the scFv light chain variable domain and wherein the Fab light chain polypeptide comprises an amino acid sequence according to SEQ ID NO: 44, and an amino acid sequence of the Fab heavy chain polypeptide that is connected to the C-terminus of the scFv light chain variable domain comprises an amino acid sequence to SEQ ID NO: 50. In some embodiments, the linker connects the Fab heavy chain polypeptide to the C-terminus of the scFv light chain variable domain and wherein the Fab light chain polypeptide comprises an amino acid sequence that has at least 97% sequence identity to the amino acid sequence according to SEQ ID NO: 44, and an amino acid sequence of the Fab heavy chain polypeptide that is connected to the C-terminus of the scFv light chain variable domain comprises an amino acid sequence that has at least 97% sequence identity to the amino acid sequence according to SEQ ID NO: 50. In some embodiments, the linker connects the Fab heavy chain polypeptide to the C-terminus of the scFv light chain variable domain and wherein the Fab light chain polypeptide comprises an amino acid sequence according to SEQ ID NO: 44, and an amino acid sequence of the Fab heavy chain polypeptide that is connected to the C-terminus of the scFv light chain variable domain comprises an amino acid sequence to SEQ ID NO: 50. In some embodiments, the linker connects the Fab heavy chain polypeptide to the C-terminus of the scFv light chain variable domain and wherein the Fab light chain polypeptide comprises an amino acid sequence that has at least 98% sequence identity to the amino acid sequence according to SEQ ID NO: 44, and an amino acid sequence of the Fab heavy chain polypeptide that is connected to the C-terminus of the scFv light chain variable domain comprises an amino acid sequence that has at least 98% sequence identity to the amino acid sequence according to SEQ ID NO: 50. In some embodiments, the linker connects the Fab heavy chain polypeptide to the C-terminus of the scFv light chain variable domain and wherein the Fab light chain polypeptide comprises an amino acid sequence according to SEQ ID NO: 44, and an amino acid sequence of the Fab heavy chain polypeptide that is connected to the C-terminus of the scFv light chain variable domain comprises an amino acid sequence to SEQ ID NO: 50. In some embodiments, the linker connects the Fab heavy chain polypeptide to the C-terminus of the scFv light chain variable domain and wherein the Fab light chain polypeptide comprises an amino acid sequence that has at least 99% sequence identity to the amino acid sequence according to SEQ ID NO: 44, and an amino acid sequence of the Fab heavy chain polypeptide that is connected to the C-terminus of the scFv light chain variable domain comprises an amino acid sequence that has at least 99% sequence identity to the amino acid sequence according to SEQ ID NO: 50.

In some embodiments, the linker connects the Fab heavy chain polypeptide to the C-terminus of the scFv light chain variable domain and wherein the Fab light chain polypeptide comprises an amino acid sequence according to SEQ ID NO: 44, and an amino acid sequence of the Fab heavy chain polypeptide that is connected to the C-terminus of the scFv light chain variable domain comprises an amino acid sequence to SEQ ID NO: 50.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 44 and has at least 95% sequence identity to the at least 200 consecutive amino acid residues of SEQ ID NO: 44 and an amino acid sequence of the Fab heavy chain polypeptide that is connected to the C-terminus of the scFv light chain variable domain comprises an amino acid sequence of at least 450 consecutive amino acid residues of SEQ ID NO: 50 and has at least 95% sequence identity to the at least 450 consecutive amino acid residues of SEQ ID NO: 50.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 44 and has at least 99% sequence identity to the at least 200 consecutive amino acid residues of SEQ ID NO: 44 and an amino acid sequence of the Fab heavy chain polypeptide that is connected to the C-terminus of the scFv light chain variable domain comprises an amino acid sequence of at least 450 consecutive amino acid residues of SEQ ID NO: 50 and has at least 99% sequence identity to the at least 450 consecutive amino acid residues of SEQ ID NO: 50.

In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 45, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 51. In some embodiments, the linker connects the Fab light chain polypeptide to the C-terminus of the scFv light chain variable domain and wherein the Fab heavy chain polypeptide comprises an amino acid sequence that has at least 90% sequence identity to the amino acid sequence according to SEQ ID NO: 45, and an amino acid sequence of the Fab light chain polypeptide that is connected to the C-terminus of the scFv light chain variable domain comprises an amino acid sequence that has at least 90% sequence identity to the amino acid sequence according to SEQ ID NO: 51.

In some embodiments, the linker connects the Fab light chain polypeptide to the C-terminus of the scFv light chain variable domain and wherein the Fab heavy chain polypeptide comprises an amino acid sequence that has at least 95% sequence identity to the amino acid sequence according to SEQ ID NO: 45, and an amino acid sequence of the Fab light chain polypeptide that is connected to the C-terminus of the scFv light chain variable domain comprises an amino acid sequence that has at least 95% sequence identity to the amino acid sequence according to SEQ ID NO: 51. In some embodiments, the linker connects the Fab light chain polypeptide to the C-terminus of the scFv light chain variable domain and wherein the Fab heavy chain polypeptide comprises an amino acid sequence that has at least 96% sequence identity to the amino acid sequence according to SEQ ID NO: 45, and an amino acid sequence of the Fab light chain polypeptide that is connected to the C-terminus of the scFv light chain variable domain comprises an amino acid sequence that has at least 96% sequence identity to the amino acid sequence according to SEQ ID NO: 51. In some embodiments, the linker connects the Fab light chain polypeptide to the C-terminus of the scFv light chain variable domain and wherein the Fab heavy chain polypeptide comprises an amino acid sequence that has at least 97% sequence identity to the amino acid sequence according to SEQ ID NO: 45, and an amino acid sequence of the Fab light chain polypeptide that is connected to the C-terminus of the scFv light chain variable domain comprises an amino acid sequence that has at least 97% sequence identity to the amino acid sequence according to SEQ ID NO: 51. In some embodiments, the linker connects the Fab light chain polypeptide to the C-terminus of the scFv light chain variable domain and wherein the Fab heavy chain polypeptide comprises an amino acid sequence that has at least 98% sequence identity to the amino acid sequence according to SEQ ID NO: 45, and an amino acid sequence of the Fab light chain polypeptide that is connected to the C-terminus of the scFv light chain variable domain comprises an amino acid sequence that has at least 98% sequence identity to the amino acid sequence according to SEQ ID NO: 51. In some embodiments, the linker connects the Fab light chain polypeptide to the C-terminus of the scFv light chain variable domain and wherein the Fab heavy chain polypeptide comprises an amino acid sequence that has at least 99% sequence identity to the amino acid sequence according to SEQ ID NO: 45, and an amino acid sequence of the Fab light chain polypeptide that is connected to the C-terminus of the scFv light chain variable domain comprises an amino acid sequence that has at least 99% sequence identity to the amino acid sequence according to SEQ ID NO: 51. In some embodiments, the linker connects the Fab light chain polypeptide to the C-terminus of the scFv light chain variable domain and wherein the Fab heavy chain polypeptide comprises an amino acid sequence according to SEQ ID NO: 45, and an amino acid sequence of the Fab light chain polypeptide that is connected to the C-terminus of the scFv light chain variable domain comprises an amino acid sequence to SEQ ID NO: 51.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 45 and has at least 95% sequence identity to the at least 200 consecutive amino acid residues of SEQ ID NO: 45 and an amino acid sequence of the Fab light chain polypeptide that is connected to the C-terminus of the scFv light chain variable domain comprises an amino acid sequence of at least 450 consecutive amino acid residues of SEQ ID NO: 51 and has at least 95% sequence identity to the at least 450 consecutive amino acid residues of SEQ ID NO: 51.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 45 and has at least 99% sequence identity to the at least 200 consecutive amino acid residues of SEQ ID NO: 45 and an amino acid sequence of the Fab light chain polypeptide that is connected to the C-terminus of the scFv light chain variable domain comprises an amino acid sequence of at least 450 consecutive amino acid residues of SEQ ID NO: 51 and has at least 99% sequence identity to the at least 450 consecutive amino acid residues of SEQ ID NO: 51.

In some embodiments, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 46, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 52. In some embodiments, the linker connects the Fab heavy chain polypeptide to the C-terminus of the scFv light chain variable domain and wherein the Fab light chain polypeptide comprises an amino acid sequence that has at least 90% sequence identity to the amino acid sequence according to SEQ ID NO: 46, and an amino acid sequence of the Fab heavy chain polypeptide that is connected to the C-terminus of the scFv light chain variable domain comprises an amino acid sequence that has at least 90% sequence identity to the amino acid sequence according to SEQ ID NO: 52. In some embodiments, the linker connects the Fab heavy chain polypeptide to the C-terminus of the scFv light chain variable domain and wherein the Fab light chain polypeptide comprises an amino acid sequence that has at least 95% sequence identity to the amino acid sequence according to SEQ ID NO: 46, and an amino acid sequence of the Fab heavy chain polypeptide that is connected to the C-terminus of the scFv light chain variable domain comprises an amino acid sequence that has at least 95% sequence identity to the amino acid sequence according to SEQ ID NO: 52. In some embodiments, the linker connects the Fab heavy chain polypeptide to the C-terminus of the scFv light chain variable domain and wherein the Fab light chain polypeptide comprises an amino acid sequence that has at least 96% sequence identity to the amino acid sequence according to SEQ ID NO: 46, and an amino acid sequence of the Fab heavy chain polypeptide that is connected to the C-terminus of the scFv light chain variable domain comprises an amino acid sequence that has at least 96% sequence identity to the amino acid sequence according to SEQ ID NO: 52.

In some embodiments, the linker connects the Fab heavy chain polypeptide to the C-terminus of the scFv light chain variable domain and wherein the Fab light chain polypeptide comprises an amino acid sequence according to SEQ ID NO: 46, and an amino acid sequence of the Fab heavy chain polypeptide that is connected to the C-terminus of the scFv light chain variable domain comprises an amino acid sequence to SEQ ID NO: 52. In some embodiments, the linker connects the Fab heavy chain polypeptide to the C-terminus of the scFv light chain variable domain and wherein the Fab light chain polypeptide comprises an amino acid sequence that has at least 97% sequence identity to the amino acid sequence according to SEQ ID NO: 46, and an amino acid sequence of the Fab heavy chain polypeptide that is connected to the C-terminus of the scFv light chain variable domain comprises an amino acid sequence that has at least 97% sequence identity to the amino acid sequence according to SEQ ID NO: 52. In some embodiments, the linker connects the Fab heavy chain polypeptide to the C-terminus of the scFv light chain variable domain and wherein the Fab light chain polypeptide comprises an amino acid sequence according to SEQ ID NO: 46, and an amino acid sequence of the Fab heavy chain polypeptide that is connected to the C-terminus of the scFv light chain variable domain comprises an amino acid sequence to SEQ ID NO: 52. In some embodiments, the linker connects the Fab heavy chain polypeptide to the C-terminus of the scFv light chain variable domain and wherein the Fab light chain polypeptide comprises an amino acid sequence that has at least 98% sequence identity to the amino acid sequence according to SEQ ID NO: 46, and an amino acid sequence of the Fab heavy chain polypeptide that is connected to the C-terminus of the scFv light chain variable domain comprises an amino acid sequence that has at least 98% sequence identity to the amino acid sequence according to SEQ ID NO: 52. In some embodiments, the linker connects the Fab heavy chain polypeptide to the C-terminus of the scFv light chain variable domain and wherein the Fab light chain polypeptide comprises an amino acid sequence according to SEQ ID NO: 46, and an amino acid sequence of the Fab heavy chain polypeptide that is connected to the C-terminus of the scFv light chain variable domain comprises an amino acid sequence to SEQ ID NO: 52. In some embodiments, the linker connects the Fab heavy chain polypeptide to the C-terminus of the scFv light chain variable domain and wherein the Fab light chain polypeptide comprises an amino acid sequence that has at least 99% sequence identity to the amino acid sequence according to SEQ ID NO: 46, and an amino acid sequence of the Fab heavy chain polypeptide that is connected to the C-terminus of the scFv light chain variable domain comprises an amino acid sequence that has at least 99% sequence identity to the amino acid sequence according to SEQ ID NO: 52.

In some embodiments, the linker connects the Fab heavy chain polypeptide to the C-terminus of the scFv light chain variable domain and wherein the Fab light chain polypeptide comprises an amino acid sequence according to SEQ ID NO: 46, and an amino acid sequence of the Fab heavy chain polypeptide that is connected to the C-terminus of the scFv light chain variable domain comprises an amino acid sequence to SEQ ID NO: 52.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 46 and has at least 95% sequence identity to the at least 200 consecutive amino acid residues of SEQ ID NO: 46 and an amino acid sequence of the Fab heavy chain polypeptide that is connected to the C-terminus of the scFv light chain variable domain comprises an amino acid sequence of at least 450 consecutive amino acid residues of SEQ ID NO: 52 and has at least 95% sequence identity to the at least 450 consecutive amino acid residues of SEQ ID NO: 52.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 46 and has at least 99% sequence identity to the at least 200 consecutive amino acid residues of SEQ ID NO: 46 and an amino acid sequence of the Fab heavy chain polypeptide that is connected to the C-terminus of the scFv light chain variable domain comprises an amino acid sequence of at least 450 consecutive amino acid residues of SEQ ID NO: 52 and has at least 99% sequence identity to the at least 450 consecutive amino acid residues of SEQ ID NO: 52.

In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 47, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 53. In some embodiments, the linker connects the Fab light chain polypeptide to the C-terminus of the scFv light chain variable domain and wherein the Fab heavy chain polypeptide comprises an amino acid sequence that has at least 90% sequence identity to the amino acid sequence according to SEQ ID NO: 47, and an amino acid sequence of the Fab light chain polypeptide that is connected to the C-terminus of the scFv light chain variable domain comprises an amino acid sequence that has at least 90% sequence identity to the amino acid sequence according to SEQ ID NO: 53.

In some embodiments, the linker connects the Fab light chain polypeptide to the C-terminus of the scFv light chain variable domain and wherein the Fab heavy chain polypeptide comprises an amino acid sequence that has at least 95% sequence identity to the amino acid sequence according to SEQ ID NO: 47, and an amino acid sequence of the Fab light chain polypeptide that is connected to the C-terminus of the scFv light chain variable domain comprises an amino acid sequence that has at least 95% sequence identity to the amino acid sequence according to SEQ ID NO: 53. In some embodiments, the linker connects the Fab light chain polypeptide to the C-terminus of the scFv light chain variable domain and wherein the Fab heavy chain polypeptide comprises an amino acid sequence that has at least 96% sequence identity to the amino acid sequence according to SEQ ID NO: 47, and an amino acid sequence of the Fab light chain polypeptide that is connected to the C-terminus of the scFv light chain variable domain comprises an amino acid sequence that has at least 96% sequence identity to the amino acid sequence according to SEQ ID NO: 53. In some embodiments, the linker connects the Fab light chain polypeptide to the C-terminus of the scFv light chain variable domain and wherein the Fab heavy chain polypeptide comprises an amino acid sequence that has at least 97% sequence identity to the amino acid sequence according to SEQ ID NO: 47, and an amino acid sequence of the Fab light chain polypeptide that is connected to the C-terminus of the scFv light chain variable domain comprises an amino acid sequence that has at least 97% sequence identity to the amino acid sequence according to SEQ ID NO: 53. In some embodiments, the linker connects the Fab light chain polypeptide to the C-terminus of the scFv light chain variable domain and wherein the Fab heavy chain polypeptide comprises an amino acid sequence that has at least 98% sequence identity to the amino acid sequence according to SEQ ID NO: 47, and an amino acid sequence of the Fab light chain polypeptide that is connected to the C-terminus of the scFv light chain variable domain comprises an amino acid sequence that has at least 98% sequence identity to the amino acid sequence according to SEQ ID NO: 53. In some embodiments, the linker connects the Fab light chain polypeptide to the C-terminus of the scFv light chain variable domain and wherein the Fab heavy chain polypeptide comprises an amino acid sequence that has at least 99% sequence identity to the amino acid sequence according to SEQ ID NO: 47, and an amino acid sequence of the Fab light chain polypeptide that is connected to the C-terminus of the scFv light chain variable domain comprises an amino acid sequence that has at least 99% sequence identity to the amino acid sequence according to SEQ ID NO: 53. In some embodiments, the linker connects the Fab light chain polypeptide to the C-terminus of the scFv light chain variable domain and wherein the Fab heavy chain polypeptide comprises an amino acid sequence according to SEQ ID NO: 47, and an amino acid sequence of the Fab light chain polypeptide that is connected to the C-terminus of the scFv light chain variable domain comprises an amino acid sequence to SEQ ID NO: 53.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 47 and has at least 95% sequence identity to the at least 200 consecutive amino acid residues of SEQ ID NO: 47 and an amino acid sequence of the Fab light chain polypeptide that is connected to the C-terminus of the scFv light chain variable domain comprises an amino acid sequence of at least 450 consecutive amino acid residues of SEQ ID NO: 53 and has at least 95% o sequence identity to the at least 450 consecutive amino acid residues of SEQ ID NO: 53.

In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 47 and has at least 9900 sequence identity to the at least 200 consecutive amino acid residues of SEQ ID NO: 47 and an amino acid sequence of the Fab light chain polypeptide that is connected to the C-terminus of the scFv light chain variable domain comprises an amino acid sequence of at least 450 consecutive amino acid residues of SEQ ID NO: 53 and has at least 99% o sequence identity to the at least 450 consecutive amino acid residues of SEQ ID NO: 53.

TABLE 8
Antibody sequences that bind to CD28 and PD-L1. CDR sequences are
underlined and were determined using IMGT definition

| | Amino Acid Sequence | SEQ ID |
|---|---|---|
| Construct Description | (N to C) | NO: |
| Ab-1 LC | EIVLTQSPATLSLSPGERATLSC | 20 |
| Anti-PDL1 Fab LC | RASQSVSSYLAWYQQKPGQA | |
| | PRLLIYDASNRATGIPARFSGS | |
| | GSGTDFTLTISSLEPEDFAVYY | |
| | CQQRSNWPTFGQGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| Ab-1 HC | QVQLVQSGAEVKKPGASVKV | 21 |
| Anti-CD28 scFv-Linker 2- | SCKASGYTFTSYYIHWVRQAP | |
| anti-PDL1 Fab HC | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSQVQ | |
| | LVQSGAEVKKPGSSVKVSCKT | |
| | SGDTESTYAISWVRQAPGQGL | |
| | EWMGGIIPIFGKAHYAQKFQ | |
| | GRVTITADESTSTAYMELSSLR | |
| | SEDTAVYFCARKFHFVSGSPF | |
| | GMDVWGQGTTVTVSSASTKG | |
| | PSVFPLAPSSKSTSGGTAALGC | |
| | LVKDYFPEPVTVSWNSGALTS | |
| | GVHTFPAVLQSSGLYSLSSVV | |
| | TVPSSSLGTQTYICNVNHKPSN | |
| | TKVDKKVEPKSC | |
| Ab-2 LC | QVQLVQSGAEVKKPGASVKV | 22 |
| Anti-CD28 scFv-Linker 2- | SCKASGYTFTSYYIHWVRQAP | |
| Anti-PDL1 Fab LC | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSEIV | |
| | LTQSPATLSLSPGERATLSCRA | |
| | SQSVSSYLAWYQQKPGQAPR | |
| | LLIYDASNRATGIPARFSGSGS | |
| | GTDFTLTISSLEPEDFAVYYCQ | |
| | QRSNWPTFGQGTKVEIKRTV | |
| | AAPSVFIFPPSDEQLKSGTASV | |
| | VCLLNNFYPREAKVQWKVDN | |
| | ALQSGNSQESVTEQDSKDSTY | |
| | SLSSTLTLSKADYEKHKVYAC | |
| | EVTHQGLSSPVTKSFNRGEC | |
| Ab-2 HC | QVQLVQSGAEVKKPGSSVKVS | 23 |
| Anti-PDL1 Fab HC | CKTSGDTFSTYAISWVRQAPG | |
| | QGLEWMGGIIPIFGKAHYAQ | |
| | KFQGRVTITADESTSTAYMEL | |
| | SSLRSEDTAVYFCARKFHFVS | |
| | GSPFGMDVWGQGTTVTVSSA | |
| | STKGPSVFPLAPSSKSTSGGTA | |
| | ALGCLVKDYFPEPVTVSWNSG | |
| | ALTSGVHTFPAVLQSSGLYSLS | |
| | SVVTVPSSSLGTQTYICNVNH | |
| | KPSNTKVDKKVEPKSC | |
| Ab-3 LC | DIQMTQSPSSLSASVGDRVTIT | 42 |
| Anti-PDL1 Fab 2 LC | CRASQDVSTAVAWYQQKPGK | |
| | APKLLIYSASFLYSGVPSRFSG | |
| | SGSGTDFTLTISSLQPEDFATY | |
| | YCQQYLYHPATFGQGTKVEI | |
| | KRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| Ab-3 HC | QVQLVQSGAEVKKPGASVKV | 48 |
| Anti-CD28 scFv-Linker 2- | SCKASGYTFTSYYIHWVRQAP | |
| anti-PDL1 Fab 2 HC | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSEVQ | |
| | LVESGGGLVQPGGSLRLSCAA | |
| | SGFTFSDSWIHWVRQAPGKG | |
| | LEWVAWISPYGGSTYYADSV | |
| | KGRFTISADTSKNTAYLQMNS | |
| | LRAEDTAVYYCARRHWPGG | |
| | FDYWGQGTLVTVSSASTKGPS | |
| | VFPLAPSSKSTSGGTAALGCLV | |
| | KDYFPEPVTVSWNSGALTSGV | |
| | HTFPAVLQSSGLYSLSSVVTVP | |
| | SSSLGTQTYICNVNHKPSNTK | |
| | VDKKVEPKSC | |
| Ab-4 LC | QVQLVQSGAEVKKPGASVKV | 49 |
| Anti-CD28 scFv-Linker 2- | SCKASGYTFTSYYIHWVRQAP | |
| Anti-PDL1 Fab 2 LC | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSDIQ | |
| | MTQSPSSLSASVGDRVTITCRA | |
| | SQDVSTAVAWYQQKPGKAPK | |
| | LLIYSASFLYSGVPSRFSGSGS | |
| | GTDFTLTISSLQPEDFATYYCQ | |
| | QYLYHPATFGQGTKVEIKRTV | |
| | AAPSVFIFPPSDEQLKSGTASV | |
| | VCLLNNFYPREAKVQWKVDN | |
| | ALQSGNSQESVTEQDSKDSTY | |
| | SLSSTLTLSKADYEKHKVYAC | |
| | EVTHQGLSSPVTKSFNRGEC | |
| Ab-4 HC | EVQLVESGGGLVQPGGSLRLS | 43 |
| Anti-PDL1 Fab 2 HC | CAASGFTFSDSWIHWVRQAP | |
| | GKGLEWVAWISPYGGSTYYA | |
| | DSVKGRFTISADTSKNTAYLQ | |
| | MNSLRAEDTAVYYCARRHW | |
| | PGGFDYWGQGTLVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKKVEPKSC | |
| Ab-5 LC | QSALTQPASVSGSPGQSITISCT | 44 |
| Anti-PDL1 Fab 3 LC | GTSSDVGGYNYVSWYQQHPG | |
| | KAPKLMIYDVSNRPSGVSNRF | |
| | SGSKSGNTASLTISGLQAEDEA | |
| | DYYCSSYTSSSTRVFGTGTKV | |
| | TVLGQPKANPTVTLFPPSSEEL | |
| | QANKATLVCLISDFYPGAVTV | |
| | AWKADGSPVKAGVETTKPSK | |
| | QSNNKYAASSYLSLTPEQWKS | |
| | HRSYSCQVTHEGSTVEKTVAP | |
| | TECS | |
| Ab-5 HC | QVQLVQSGAEVKKPGASVKV | 50 |
| Anti-CD28 scFv-Linker 2- | SCKASGYTFTSYYIHWVRQAP | |
| anti-PDL1 Fab 3 HC | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSEVQ | |
| | LLESGGGLVQPGGSLRLSCAA | |
| | SGFTFSSYIMMWVRQAPGKG | |
| | LEWVSSIYPSGGITFYADTVK | |
| | GRFTISRDNSKNTLYLQMNSL | |
| | RAEDTAVYYCARIKLGTVTT | |
| | VDYWGQGTLVTVSSASTKGPS | |
| | VFPLAPSSKSTSGGTAALGCLV | |
| | KDYFPEPVTVSWNSGALTSGV | |
| | HTFPAVLQSSGLYSLSSVVTVP | |
| | SSSLGTQTYICNVNHKPSNTK | |
| | VDKKVEPKSC | |
| Ab-6 LC | QVQLVQSGAEVKKPGASVKV | 51 |
| Anti-CD28 scFv-Linker 2- | SCKASGYTFTSYYIHWVRQAP | |
| Anti-PDL1 Fab 3 | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSQSA | |
| | LTQPASVSGSPGQSITISCTGTS | |
| | SDVGGYNYVSWYQQHPGKAP | |
| | KLMIYDVSNRPSGVSNRFSGS | |
| | KSGNTASLTISGLQAEDEADY | |
| | YCSSYTSSSTRVFGTGTKVTV | |
| | LGQPKANPTVTLFPPSSEELQ | |
| | ANKATLVCLISDFYPGAVTVA | |
| | WKADGSPVKAGVETTKPSKQ | |
| | SNNKYAASSYLSLTPEQWKSH | |
| | RSYSCQVTHEGSTVEKTVAPT | |
| | ECS | |
| Ab-6 HC | EVQLLESGGGLVQPGGSLRLS | 45 |
| Anti-PDL1 Fab 3 HC | CAASGFTFSSYIMMWVRQAP | |
| | GKGLEWVSSIYPSGGITFYAD | |
| | TVKGRFTISRDNSKNTLYLQM | |
| | NSLRAEDTAVYYCARIKLGT | |
| | VTTVDYWGQGTLVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKKVEPKSC | |
| Ab-7 LC | EIVLTQSPGTLSLSPGERATLSC | 46 |
| Anti-PDL1 Fab 4 LC | RASQRVSSSYLAWYQQKPGQ | |
| | APRLLIYDASSRATGIPDRFSG | |
| | SGSGTDFTLTISRLEPEDFAVY | |
| | YCQQYGSLPWTFGQGTKVEI | |
| | KRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| Ab-7 HC | QVQLVQSGAEVKKPGASVKV | 52 |
| Anti-CD28 scFv-Linker 2- | SCKASGYTFTSYYIHWVRQAP | |
| anti-PDL1 Fab 4 HC | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSEVQ | |
| | LVESGGGLVQPGGSLRLSCAA | |
| | SGFTFSRYWMSWVRQAPGK | |
| | GLEWVANIKQDGSEKYYVDS | |
| | VKGRFTISRDNAKNSLYLQMN | |
| | SLRAEDTAVYYCAREGGWFG | |
| | ELAFDYWGQGTLVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKRVEPKSC | |
| Ab-8 LC | QVQLVQSGAEVKKPGASVKV | 53 |
| Anti-CD28 scFv-Linker 2- | SCKASGYTFTSYYIHWVRQAP | |
| Anti-PDL1 Fab 4 LC | GQGLEWIGSIYPGNVNTNYNE | |
| | KFKDRATLTVDTSISTAYMEL | |
| | SRLRSDDTAVYFCTRSHYGLD | |
| | WNFDVWGQGTTVTVSSGGG | |
| | GSGGGGSGGGGSDIQMTQSPS | |
| | SLSASVGDRVTITCHASQNIYV | |
| | WLNWYQQKPGKAPKLLIYKA | |
| | SNLHTGVPSRFSGSGSGTDFTL | |
| | TISSLQPEDFATYYCQQGQTY | |
| | PYTFGGGTKVEIKGGGGSEIV | |
| | LTQSPGTLSLSPGERATLSCRA | |
| | SQRVSSSYLAWYQQKPGQAP | |
| | RLLIYDASSRATGIPDRFSGSG | |
| | SGTDFTLTISRLEPEDFAVYYC | |
| | QQYGSLPWTFGQGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| Ab-8 HC | EVQLVESGGGLVQPGGSLRLS | 47 |
| Anti-PDL1 Fab 4 HC | CAASGFTFSRYWMSWVRQAP | |
| | GKGLEWVANIKQDGSEKYYV | |
| | DSVKGRFTISRDNAKNSLYLQ | |
| | MNSLRAEDTAVYYCAREGG | |
| | WFGELAFDYWGQGTLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKRVEPKSC | |

In some embodiments, the multispecific antibodies described herein comprise improved activity in tumor cell killing. In some embodiments, the multispecific antibodies comprise an IC50 of less than about 300 picomolar (pM) in a cell killing assay. In some embodiments, the multispecific antibodies comprise an IC50 of less than about 200 picomolar (pM) in a cell killing assay. In some embodiments, the multispecific antibodies comprise an IC50 of less than about 100 picomolar (pM) in a cell killing assay. In some embodiments, the multispecific antibodies comprise an IC50 of less than about 75 picomolar (pM) in a cell killing assay. In some embodiments, the multispecific antibodies comprise an IC50 of less than about 50 picomolar (pM) in a cell killing assay. In some embodiments, the multispecific antibodies comprise an IC50 of less than about 25 picomolar (pM) in a cell killing assay. In some embodiments, the multispecific antibodies comprise an IC50 of less than about 10 picomolar (pM) in a cell killing assay.

In some embodiments, the multispecific antibodies described herein trigger little or no non-specific activation of immune cells. In some embodiments, the multispecific antibodies described herein trigger little or no cytokine release. In some embodiments, the multispecific antibodies described herein trigger little or no IFNy, TNF-alpha, or IL-2 release from immune cells. In some embodiments, the multispecific antibodies described herein trigger little or no proliferation of immune cells.

Multispecific Antibodies that Bind to CD28 and PD-L1: Formats for Selective Activation in Tumor Microenvironments

In some embodiments, the multispecific antibodies described herein are selectively activated in tumor microenvironments.

In some embodiments, the multispecific antibody is according to the following subformula: P₁-L₁-A-L-B (Formula Ia) wherein A comprises the CD28 binding domain; B comprises the PD-L1 binding domain; L comprises the linker that connects A to B; P₁ comprises a peptide that binds to A and L1 comprises a linking moiety that connects A to P₁ and is a substrate for a tumor specific protease.

In some embodiments, the multispecific antibody is according to the following subformula: A-L-B-L₂-P₂ (Formula Ib) wherein A comprises the CD28 binding domain; B comprises the PD-L1 binding domain; L comprises the linker that connects A to B; P₂ comprises a peptide that binds to B and L₂ comprises a linking moiety that connects B to P₂ and is a substrate for a tumor specific protease.

In some embodiments, the multispecific antibody is according to the following subformula: P₁-L₁-A-L-B-L₂-P₂ (Formula Ic) wherein A comprises the CD28 binding domain; B comprises the PD-L1 binding domain; L comprises the linker that connects A to B; P₁ comprises a peptide that binds to A and L1 comprises a linking moiety that connects A to P₁ and is a substrate for a tumor specific protease; P₂ comprises a peptide that binds to B and L₂ comprises a linking moiety that connects B to P₂ and is a substrate for a tumor specific protease.

### Half-Life Extending Molecule (H₁)

In some embodiments, the multispecific antibodies of Formula Ia, Formula Ib, Formula Ic further comprise a half-life extending molecule (H₁). In some embodiments, H₁ is connected to P₁. In some embodiments, H₁ is connected to P₂. In some embodiments, H₁ does not block A binding to CD28. In some embodiments, H₁ comprises a linking moiety (L₅) that connects H₁ to P₁ or H₁ to P₂. In some embodiments, half-life extending molecule (H₁) does not have binding affinity to PD-L1. In some embodiments, the half-life extending molecule (H₁) does not have binding affinity to CD28. In some embodiments, the half-life extending molecule (H₁) does not shield the multispecific antibody from CD28.

In some embodiments, H₁ comprises a sequence as disclosed in Table 9 or a sequence substantially identical thereto (e.g., a sequence that has at least 90%, 95%, 96%, 97%, 98%, or 99% sequence identity).

TABLE 9
H1 Sequences

| | Amino Acid Sequence | |
|---|---|---|
| Construct Description | (N to C) | SEQ ID NO: |
| Anti-Albumin: CDR-H1 | GSTFYTAV | 54 |
| Anti-Albumin: CDR-H2 | IRWTALTT | 55 |
| Anti-Albumin: CDR-H3 | AARGTLGLFTTADSYDY | 56 |
| Anti-albumin | EVQLVESGGGLVQPGGSLRLSCAASGSTF | 57 |
| | YTAVMGWVRQAPGKGLEWVAAIRWTA | |
| | LTTSYADSVKGRFTISRDGAKTTLYLQM | |
| | NSLRPEDTAVYYCAARGTLGLFTTADSY | |
| | DYWGQGTLVTVSS | |

In some embodiments, H₁ comprises an amino acid sequence that has repetitive sequence motifs. In some embodiments, H₁ comprises an amino acid sequence that has highly ordered secondary structure. “Highly ordered secondary structure,” as used in this context, means that at least about 50%, or about 70%, or about 80%, or about 90%, of amino acid residues of H₁ contribute to secondary structure, as measured or determined by means, including, but not limited to, spectrophotometry (e.g. by circular dichroism spectroscopy in the “far-UV” spectral region (190-250 nm), and computer programs or algorithms, such as the Chou-Fasman algorithm and the Garier-Osguthorpe-Robson (“GOR”) algorithm.

In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab.

In some embodiments, the single domain antibody comprises a single domain antibody that binds to albumin. In some embodiments, the single domain antibody is a human or humanized antibody. In some embodiments, the single domain antibody is selected from the group consisting of 645gHlgL1, 645dsgH5gL4, 23-13-A01-sc02, A10m3 or a fragment thereof, DOM7r-31, DOM7h-11-15, Alb-1, Alb-8, Alb-23, 10G, 10E and SA21. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3.

In some embodiments, H₁ comprises an amino acid sequence according to SEQ ID NO: 57. In some embodiments, H₁ comprises an amino acid sequence that has at least 80% sequence identity to SEQ ID NO: 57. In some embodiments, H₁ comprises an amino acid sequence that has at least 85% sequence identity to SEQ ID NO: 57. In some embodiments, H₁ comprises an amino acid sequence that has at least 90% sequence identity to SEQ ID NO: 57. In some embodiments, H₁ comprises an amino acid sequence that has at least 95% sequence identity to SEQ ID NO: 57. In some embodiments, H₁ comprises an amino acid sequence that has at least 99% sequence identity to SEQ ID NO: 57.

In some embodiments, H₁ comprise a modified amino acid or non-natural amino acid, or a modified non-natural amino acid, or a combination thereof. In some embodiments, the modified amino acid or a modified non-natural amino acid comprises a post-translational modification. In some embodiments H₁ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to H₁ including the peptide backbone, the amino acid side chains, and the terminus.

In some embodiments, H₁ comprises a linking moiety (L₅) that connects H₁ to P₁ or P₂.In some embodiments, L₅ is a peptide sequence having at least 5 to no more than 50 amino acids. In some embodiments, L₅ is a peptide sequence having at least 10 to no more than 30 amino acids. In some embodiments, L₅ is a peptide sequence having at least 10 amino acids. In some embodiments, L₅ is a peptide sequence having at least 18 amino acids. In some embodiments, L₅ is a peptide sequence having at least 26 amino acids. In some embodiments, L₅ has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Linking Moiety (L₁ or L₂)

In some embodiments, L₁ or L₂ is a peptide sequence having at least 5 to no more than 50 amino acids. In some embodiments L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments, L₁ or L₂ has a formula comprising (G₂S)ₙ (SEQ ID NO: 233), wherein n is an integer from 1 to 3. In some embodiments, L₁ or L₂ has a formula comprising (G₂S)ₙ, wherein n is an integer of at least 1. In some embodiments, L₁ or L₂ has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments L₁ or L₂ comprises a urokinase cleavable amino acid sequence, a matriptase cleavable amino acid sequence, a legumain cleavable amino acid sequence, or a matrix metalloprotease cleavable amino acid sequence.

In some embodiments, L₁ or L₂ comprises a sequence as disclosed in Table 10 or a sequence substantially identical thereto (e.g., a sequence that has 0, 1, or 2 amino acid modifications).

TABLE 10
L1 or L2

| Construct | Amino Acid Sequence | SEQ ID |
|---|---|---|
| Description | (N to C) | NO: |
| Linker 1 | GGGGSGGGGSGGGGS | 18 |
| Linker 2 | GGGGS | 19 |
| Linker 3 | GGGGSGGGS | 62 |
| Cleavable linker | GGGGSGGGLSGRSDAGSPLGL | 63 |
| | AGSGGGS | |
| Linker 4 | GGGGSLSGRSDNHGSSGT | 64 |
| Linker 5 | GGGGSSGGSGGSGLSGRSDNH | 65 |
| | GSSGT | |
| Linker 6 | ASGRSDNH | 66 |
| Linker 7 | LAGRSDNH | 67 |
| Linker 8 | ISSGLASGRSDNH | 68 |
| Linker 9 | ISSGLLAGRSDNH | 69 |
| Linker 10 | LSGRSDNH | 70 |
| Linker 11 | ISSGLLSGRSDNP | 71 |
| Linker 12 | ISSGLLSGRSDNH | 72 |
| Linker 13 | LSGRSDNHSPLGLAGS | 73 |
| Linker 14 | SPLGLAGSLSGRSDNH | 74 |
| Linker 15 | SPLGLSGRSDNH | 75 |
| Linker 16 | LAGRSDNHSPLGLAGS | 76 |
| Linker 17 | LSGRSDNHVPLSLKMG | 77 |
| Linker 18 | LSGRSDNHVPLSLSMG | 78 |
| Linker 19 | GSSGGSGGSGGSGISSGLLSGR | 79 |
| | SDNHGSSGT | |
| Linker 20 | GSSGGSGGSGGISSGLLSGRSD | 80 |
| | NHGGGS | |
| Linker 21 | ASGRSDNH | 81 |
| Linker 22 | LAGRSDNH | 82 |
| Linker 23 | ISSGLASGRSDNH | 83 |
| Linker 24 | LSGRSDAG | 84 |
| Linker 25 | ISSGLLSGRSDAG | 85 |
| Linker 26 | AAGLLAPPGGLSGRSDAG | 86 |
| Linker 27 | SPLGLSGRSDAG | 87 |
| Linker 28 | LSGRSDAGSPLGLAG | 88 |

In some embodiments, L₁ is bound to N-terminus of A. In some embodiments, L₁ is bound to C-terminus of A. In some embodiments, L₂ is bound to N-terminus of B. In some embodiments, L₂ is bound to C-terminus of B. In some embodiments, P₁ becomes unbound from A when L₁ is cleaved by the tumor specific protease thereby exposing A to CD28. In some embodiments, P₂ becomes unbound from B when L₂ is cleaved by the tumor specific protease thereby exposing B to PD-L1.

In some embodiments, L₁ or L₂, comprise a modified amino acid or non-natural amino acid, or a modified non-natural amino acid, or a combination thereof. In some embodiments, the modified amino acid or a modified non-natural amino acid comprises a post-translational modification. In some embodiments, L₁ or L₂ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₁ or L₂ including the peptide backbone, or the amino acid side chains.

Peptide (P₁ and P₂)

P₁ or P₂ is designed to be optimized for a specific antigen-binding domain through an iterative process of phage display and quantitative binding assays designed to select for those peptides that are able to prevent binding to CD28 or PD-L1. A directed evolution-based process that includes phage libraries is used for identifying P₁ or P₂. Multiple cycles of selection and amplification of potential inhibitory peptides that are capable of blocking the antigen binding domain from binding to its target of CD28 or PD-L1 with the goal of optimizing masked PD-L1 x CD28 antibodies in serum and limiting cleavage, thereby reducing toxicity. Discovery of P₁ or P₂ is depicted in FIG. 18.

In some embodiments, P₁ impairs binding of A to CD28. In some embodiments, P₁ is bound to A through ionic interactions, electrostatic interactions, hydrophobic interactions, Pi-stacking interactions, and H-bonding interactions, or a combination thereof. In some embodiments, P₁ is bound to A at or near an antigen binding site. In some embodiments, P₁ becomes unbound from A when L₁ is cleaved by the tumor specific protease thereby exposing A to CD28. In some embodiments, P₁ has less than 70% sequence identity to CD28. In some embodiments, P₁ has less than 75% sequence identity to CD28. In some embodiments, P₁ has less than 80% sequence identity to CD28. In some embodiments, P₁ has less than 85% sequence identity to CD28. In some embodiments, P₁ has less than 90% sequence identity to CD28. In some embodiments, P₁ has less than 95% sequence identity to CD28. In some embodiments, P₁ has less than 98% sequence identity to CD28. In some embodiments, P₁ has less than 99% sequence identity to CD28. In some embodiments, P₁ comprises a de novo amino acid sequence that shares less than 10% sequence identity to CD28.

In some embodiments, P₂ impairs binding of B to PD-L1. In some embodiments, P₂ is bound to B through ionic interactions, electrostatic interactions, hydrophobic interactions, Pi-stacking interactions, and H-bonding interactions, or a combination thereof. In some embodiments, P₂ is bound to B at or near an antigen binding site. In some embodiments, P₂ becomes unbound from B when L₂ is cleaved by the tumor specific protease thereby exposing B to the PD-L1. In some embodiments, P₂ has less than 70% sequence identity to the PD-L1. In some embodiments, P₂ has less than 75% sequence identity to the PD-L1. In some embodiments, P₂ has less than 80% sequence identity to the PD-L1. In some embodiments, P₂ has less than 85% sequence identity to the PD-L1. In some embodiments, P₂ has less than 90% sequence identity to the PD-L1. In some embodiments, P₂ has less than 95% sequence identity to the PD-L1. In some embodiments, P₂ has less than 98% sequence identity to the PD-L1. In some embodiments, P₂ has less than 99% sequence identity to the PD-L1. In some embodiments, P₂ comprises a de novo amino acid sequence that shares less than 10% sequence identity to the PD-L1.

In some embodiments, P₁ or P₂ comprises a peptide sequence of at least 5 amino acids in length. In some embodiments, P₁ or P₂ comprises a peptide sequence of at least 6 amino acids in length. In some embodiments, P₁ or P₂ comprises a peptide sequence of at least 10 amino acids in length. In some embodiments, P₁ or P₂ comprises a peptide sequence of at least 10 amino acids in length and no more than 20 amino acids in length. In some embodiments, P₁ or P₂ comprises a peptide sequence of at least 16 amino acids in length. In some embodiments, P₁ or P₂ comprises a peptide sequence of no more than 40 amino acids in length. In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide.

In some embodiments, P₁ or P₂ comprise a modified amino acid or non-natural amino acid, or a modified non-natural amino acid, or a combination thereof. In some embodiments, the modified amino acid or a modified non-natural amino acid comprises a post-translational modification. In some embodiments P₁ or P₂ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁ or P₂ including the peptide backbone, the amino acid side chains, and the terminus.

In some embodiments, P₁ or P₂ does not comprise albumin or an albumin fragment. In some embodiments, P₁ or P₂ does not comprise an albumin binding domain.

Polynucleotides Encoding Multispecific Antibodies that Bind to CD28 and PD-L1

Disclosed herein, in some embodiments, are isolated recombinant nucleic acid molecules encoding the multispecific antibodies disclosed herein. Described herein, in some embodiments, are isolated recombinant nucleic acid molecules encoding multispecific antibodies that comprise a CD28 binding domain and a PD-L1 binding domain, wherein when the CD28 binding domain is a single chain variable fragment (scFv), then the PD-L1 binding domain is not a scFv.

In some embodiments, the isolated recombinant nucleic acid molecules encode multispecific antibodies according to the formula:

A-L-B (Formula I)

wherein A comprises the CD28 binding domain; B comprises the PD-L1 binding domain; and L comprises a linker that connects A to B. In some embodiments, the isolated recombinant nucleic acid molecules encode multispecific antibodies comprising the formula:

A-L-B (Formula I)

wherein A comprises the CD28 binding domain; B comprises the PD-L1 binding domain; and L comprises a linker that connects A to B. In some embodiments, the isolated recombinant nucleic acid molecules encode multispecific antibodies comprising the formula:

A-L-B (Formula I)

wherein A is the CD28 binding domain; B is the PD-L1 binding domain; and L is a linker that connects A to B. In some embodiments, the isolated recombinant nucleic acid molecules encode multispecific antibodies according to the formula:

A-L-B (Formula I)

wherein A is the CD28 binding domain; B is the PD-L1 binding domain; and L is a linker that connects A to B.

Disclosed herein, in some embodiments, are isolated recombinant nucleic acid molecules encoding an isolated polypeptide comprising an amino acid according to SEQ ID NO: 20.

Disclosed herein, in some embodiments, are isolated recombinant nucleic acid molecules encoding an isolated polypeptide comprising an amino acid according to SEQ ID NO: 21.

Disclosed herein, in some embodiments, are isolated recombinant nucleic acid molecules encoding an isolated polypeptide comprising an amino acid according to SEQ ID NO: 22.

Disclosed herein, in some embodiments, are isolated recombinant nucleic acid molecules encoding an isolated polypeptide comprising an amino acid according to SEQ ID NO: 23.

Disclosed herein, in some embodiments, are isolated recombinant nucleic acid molecules encoding an isolated polypeptide comprising an amino acid according to SEQ ID NO: 42.

Disclosed herein, in some embodiments, are isolated recombinant nucleic acid molecules encoding an isolated polypeptide comprising an amino acid according to SEQ ID NO: 48.

Disclosed herein, in some embodiments, are isolated recombinant nucleic acid molecules encoding an isolated polypeptide comprising an amino acid according to SEQ ID NO: 49.

Disclosed herein, in some embodiments, are isolated recombinant nucleic acid molecules encoding an isolated polypeptide comprising an amino acid according to SEQ ID NO: 43.

Disclosed herein, in some embodiments, are isolated recombinant nucleic acid molecules encoding an isolated polypeptide comprising an amino acid according to SEQ ID NO: 44.

Disclosed herein, in some embodiments, are isolated recombinant nucleic acid molecules encoding an isolated polypeptide comprising an amino acid according to SEQ ID NO: 50.

Disclosed herein, in some embodiments, are isolated recombinant nucleic acid molecules encoding an isolated polypeptide comprising an amino acid according to SEQ ID NO: 51.

Disclosed herein, in some embodiments, are isolated recombinant nucleic acid molecules encoding an isolated polypeptide comprising an amino acid according to SEQ ID NO: 45.

Disclosed herein, in some embodiments, are isolated recombinant nucleic acid molecules encoding an isolated polypeptide comprising an amino acid according to SEQ ID NO: 46.

Disclosed herein, in some embodiments, are isolated recombinant nucleic acid molecules encoding an isolated polypeptide comprising an amino acid according to SEQ ID NO: 52.

Disclosed herein, in some embodiments, are isolated recombinant nucleic acid molecules encoding an isolated polypeptide comprising an amino acid according to SEQ ID NO: 53.

Disclosed herein, in some embodiments, are isolated recombinant nucleic acid molecules encoding an isolated polypeptide comprising an amino acid according to SEQ ID NO: 47.

Pharmaceutical Compositions

Disclosed herein, in some embodiments, are pharmaceutical compositions comprising: (a) multispecific antibodies as disclosed herein; and (b) a pharmaceutically acceptable excipient.

In some embodiments, the pharmaceutical composition comprises (a) multispecific antibodies that comprise a CD28 binding domain and a PD-L1 binding domain, wherein when the CD28 binding domain is a single chain variable fragment (scFv), then the PD-L1 binding domain is not a scFv; and (b) a pharmaceutically acceptable excipient. In some embodiments, the pharmaceutical composition comprises (a) multispecific antibodies according to the formula: A-L-B (Formula I) wherein A comprises the CD28 binding domain; B comprises the PD-L1 binding domain; and L comprises a linker that connects A to B; and (b) a pharmaceutically acceptable excipient. In some embodiments, the pharmaceutical composition comprises (a) a multispecific antibodies comprising the formula: A-L-B (Formula I) wherein A comprises the CD28 binding domain; B comprises the PD-L1 binding domain; and L comprises a linker that connects A to B; and (b) a pharmaceutically acceptable excipient. In some embodiments, the pharmaceutical composition comprises (a) a multispecific antibodies comprising the formula: A-L-B (Formula I) wherein A is the CD28 binding domain; B is the PD-L1 binding domain; and L is a linker that connects A to B; and (b) a pharmaceutically acceptable excipient. In some embodiments, the pharmaceutical composition comprises (a) multispecific antibodies according to the formula: A-L-B (Formula I) wherein A is the CD28 binding domain; B is the PD-L1 binding domain; and L is a linker that connects A to B; and (b) a pharmaceutically acceptable excipient.

In some embodiments, the multispecific antibody further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the multispecific antibody as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Use

In some embodiments, are methods of treating cancer in a subject in need thereof comprising administering to the subject a multispecific antibody as disclosed herein that binds to CD28 and PD-L1.

In some embodiments, the cancer comprises cancer cells that express PD-L1.

In some embodiments, the cancer is a hematological malignancy. In some embodiments, wherein the cancer is leukemia or lymphoma. In some embodiments, the cancer is lymphoma, and wherein the lymphoma is B-cell lymphoma. In some embodiments, the cancer is a solid tumor. In some embodiments, the solid tumor is sarcoma, breast cancer, lung cancer, or carcinoma. In some embodiments, the solid tumor is lung cancer, and wherein the lung cancer is non-small cell lung cancer.

In some embodiments, the multispecific antibody induces T cell mediated cytotoxicity of tumor cells. In some embodiments, the administering to the subject of the multispecific antibody is sufficient to reduce or eliminate the cancer as compared to a baseline measurement of the cancer taken from the subject prior to the administering of the multispecific antibody. In some embodiments, the reduction is at least about 1-fold, 5-fold, 10-fold, 20-fold, 40-fold, 60-fold, 80-fold, or up to about 100 fold.

In some embodiments, the multispecific antibody is administered to the subject as a single agent therapy.

In some embodiments, the subject is refractory to checkpoint inhibitor therapy.

In some embodiments, the subject has relapsed from checkpoint inhibitor therapy.

In some embodiments, the multispecific antibody is not administered as a part of a treatment regimen with a second therapeutic agent. In some embodiments, the multispecific antibody is not administered as a part of a treatment regimen with a second therapeutic agent comprising a tumor binding domain. In some embodiments, the multispecific antibody is not administered as a part of a treatment regimen with a second therapeutic agent comprising an anti-CD19 antibody. In some embodiments, the multispecific antibody is not administered as a part of a treatment regimen with a second therapeutic agent comprising an antibody that has an anti-CD19 binding domain and an anti-CD3 binding domain.

In some embodiments, the multispecific antibody is any multispecific antibody as disclosed herein that binds to CD28 and PD-L1.

Disclosed herein are methods of treating cancer in a subject in need thereof comprising administering to the subject a multispecific antibody that comprises a CD28 binding domain and a PD-L1 binding domain wherein the multispecific antibody that comprises the CD28 binding domain and the PD-L1 binding domain is not administered as part of a treatment regimen with another multispecific antibody that targets a cancer antigen different from PD-L1 or CD28. In some embodiments, the multispecific antibody comprises a CD28 binding domain and a PD-L1 binding domain, wherein when the CD28 binding domain is a single chain variable fragment (scFv), then the PD-L1 binding domain is not a scFv. In some embodiments, the multispecific antibody is according to the following formula: A-L-B (Formula I) wherein A comprises the CD28 binding domain; B comprises the PD-L1 binding domain; and L comprises a linker that connects A to B. In some embodiments, the CD28 binding domain comprises a single chain variable fragment, a single domain antibody, a Fab, or a Fab′. In some embodiments, the CD28 binding domain comprises the single chain variable fragment. In some embodiments, the CD28 binding domain comprises the single domain antibody. In some embodiments, the CD28 binding domain comprises the Fab or the Fab′. In some embodiments, the PD-L1 binding domain comprises a single domain antibody, a Fab, or a Fab′. In some embodiments, the PD-L1 binding domain comprises the Fab or the Fab′. In some embodiments, the PD-L1 binding domain comprises the Fab or the Fab′ and the CD28 binding domain comprises the single chain variable fragment. In some embodiments, the PD-L1 binding domain that comprises the Fab or the Fab′ comprises a Fab heavy chain polypeptide comprising a Fab heavy chain variable domain and a Fab light chain polypeptide comprising a Fab light chain variable domain. In some embodiments, the CD28 binding domain that comprises the single chain variable fragment comprises a scFv heavy chain variable domain and a scFv light chain variable domain.

In some embodiments, the subject is refractory to checkpoint inhibitor therapy.

In some embodiments, the subject has relapsed from checkpoint inhibitor therapy.

In some embodiments, the linker connects the C-terminus of A to an N-terminus of B. In some embodiments, the linker connects the N-terminus of A to a C-terminus of B. In some embodiments, the linker connects the C-terminus of A to the N-terminus of the Fab heavy chain polypeptide. In some embodiments, the linker connects the N-terminus of A to the C-terminus of the Fab heavy chain polypeptide. In some embodiments, the linker connects the C-terminus of A to the N-terminus of the Fab light chain polypeptide. In some embodiments, the linker connects the N-terminus of A to the C-terminus of the Fab light chain polypeptide. In some embodiments, the linker connects the Fab light chain polypeptide to the scFv light chain variable domain. In some embodiments, the linker connects the Fab light chain polypeptide to the scFv heavy chain variable domain. In some embodiments, the linker connects the Fab heavy chain polypeptide to the scFv light chain variable domain. In some embodiments, the linker connects the Fab heavy chain polypeptide to the scFv heavy chain variable domain. In some embodiments, the linker connects the Fab light chain polypeptide to the N-terminus of the scFv light chain variable domain. In some embodiments, the linker connects the Fab light chain polypeptide to the C-terminus of the scFv light chain variable domain. In some embodiments, the linker connects the Fab light chain polypeptide to the N-terminus of the scFv heavy chain variable domain. In some embodiments, the linker connects the Fab light chain polypeptide to the C-terminus of the scFv heavy chain variable domain. In some embodiments, the linker connects the Fab heavy chain polypeptide to the N-terminus of the scFv light chain variable domain.

In some embodiments, the linker connects the Fab heavy chain polypeptide to the C-terminus of the scFv light chain variable domain. In some embodiments, the linker connects the Fab heavy chain polypeptide to the N-terminus of the scFv heavy chain variable domain. In some embodiments, the linker connects the Fab heavy chain polypeptide to the C-terminus of the scFv heavy chain variable domain.

In some embodiments, the linker is at least 5 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker is 15 amino acids in length. In some embodiments, the linker is selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, L₁ or L₂ has a formula comprising (G₂S)ₙ (SEQ ID NO: 233), wherein n is an integer from 1 to 3. In some embodiments, the linker comprises an amino acid sequence of SEQ ID NO: 18 (GGGGSGGGGSGGGGS) or SEQ ID NO: 19 (GGGGS).

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3. In some embodiments, the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3. In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise:LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of said LC-CDR1, LC-CDR2, or LC-CDR3.

In some embodiments, the scFv heavy chain variable domain comprises an amino acid sequence that has at least 80% sequence identity to the amino acid sequence according to SEQ ID NO: 7. In some embodiments, the scFv heavy chain variable domain comprises an amino acid sequence of at least 75 consecutive amino acid residues of SEQ ID NO: 7. In some embodiments, the scFv heavy chain variable domain comprises an amino acid sequence of at least 110 consecutive amino acid residues of SEQ ID NO: 7.

In some embodiments, the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7. In some embodiments, the scFv heavy chain variable domain comprises an amino acid sequence according to SEQ ID NO: 7. In some embodiments, the scFv light chain variable domain comprises an amino acid sequence that has at least 80% sequence identity to the amino acid sequence according to SEQ ID NO: 8. In some embodiments, the scFv light chain variable domain comprises an amino acid sequence of at least 75 consecutive amino acid residues of SEQ ID NO: 8. In some embodiments, the scFv light chain variable domain comprises an amino acid sequence of at least 100 consecutive amino acid residues of SEQ ID NO: 8. In some embodiments, the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8. In some embodiments, the scFv light chain variable domain comprises an amino acid sequence according to SEQ ID NO: 8. In some embodiments, the scFv comprises an amino acid sequence that has at least 80% sequence identity to the amino acid sequence according to SEQ ID NO: 9. In some embodiments, the scFv comprises an amino acid sequence of at least 175 consecutive amino acid residues of SEQ ID NO: 9. In some embodiments, the scFv comprises an amino acid sequence of at least 210 consecutive amino acid residues of SEQ ID NO: 9. In some embodiments, the scFv comprises an amino acid sequence of at least 210 consecutive amino acid residues of SEQ ID NO: 9 and has at least 80% sequence identity to the at least 210 consecutive amino acid residues of SEQ ID NO: 9. In some embodiments, the scFv comprises an amino acid sequence according to SEQ ID NO: 9. In some embodiments, the Fab heavy chain polypeptide comprises an amino acid sequence that has at least 80% sequence identity to the amino acid sequence according to SEQ ID NO: 17. In some embodiments, the Fab heavy chain polypeptide comprises an amino acid sequence of at least 175 consecutive amino acid residues of SEQ ID NO: 17.In some embodiments, the Fab heavy chain polypeptide comprises an amino acid sequence of at least 215 consecutive amino acid residues of SEQ ID NO: 17. In some embodiments, the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17.

In some embodiments, the Fab heavy chain polypeptide comprises an amino acid sequence according to SEQ ID NO: 17. In some embodiments, the Fab light chain polypeptide comprises an amino acid sequence that has at least 80% sequence identity to the amino acid sequence according to SEQ ID NO: 16. In some embodiments, the Fab light chain polypeptide comprises an amino acid sequence of at least 175 consecutive amino acid residues of SEQ ID NO: 16. In some embodiments, the Fab light chain polypeptide comprises an amino acid sequence of at least 200 consecutive amino acid residues of SEQ ID NO: 16. In some embodiments, the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16. In some embodiments, the Fab light chain polypeptide comprises an amino acid sequence according to SEQ ID NO: 16. In some embodiments, the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21. In some embodiments, the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21. In some embodiments, the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22. In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22. In some embodiments, the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22. In some embodiments, the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

Disclosed herein are methods of treating cancer in a subject in need thereof comprising administering to the subject a multispecific antibody that comprises a CD28 binding domain and a PD-L1 binding domain wherein the multispecific antibody that comprises the CD28 binding domain and the PD-L1 binding domain is not administered as part of a treatment regimen with another multispecific antibody that targets a cancer antigen different from PD-L1 or CD28. In some embodiments, the multispecific antibody that comprises the CD28 binding domain and the PD-L1 binding domain is administered to the subject as a single agent therapy. In some embodiments, the multispecific antibody that comprises the CD28 binding domain and the PD-L1 binding domain comprises an IgG framework, an IgA framework, an IgE framework, or an IgM framework. In some embodiments, the CD28 binding domain comprises a single chain variable fragment, a single domain antibody, a Fab, or a Fab′. In some embodiments, the PD-L1 binding domain comprises a single chain variable fragment, a single domain antibody, a Fab, or a Fab′. In some embodiments, the PD-L1 binding domain comprises a single chain variable fragment and the CD28 binding domain comprises a single chain variable fragment. In some embodiments, the CD28 binding domain comprises an anti-CD28 light chain polypeptide. In some embodiments, the anti-CD28 light chain polypeptide comprises a variable domain of an IgG1, IgG2, IgG3, or IgG4 light chain. In some embodiments, the CD28 binding domain comprises an anti-CD28 heavy chain polypeptide. In some embodiments, the anti-CD28 heavy chain polypeptide comprises a variable domain of an IgG1, IgG2, IgG3, or IgG4 heavy chain. In some embodiments, the PD-L1 binding domain comprises an anti-PD-L1 light chain polypeptide. In some embodiments, the anti-PD-L1 light chain polypeptide comprises a variable domain of an IgG1, IgG2, IgG3, or IgG4 light chain. In some embodiments, the PD-L1 binding domain comprises an anti-PD-L1 heavy chain polypeptide. In some embodiments, the anti-PD-L1 heavy chain polypeptide comprises a variable domain of an IgG1, IgG2, IgG3, or IgG4 heavy chain. In some embodiments, the multispecific antibody further comprises a fragment crystallizable (Fc) region. In some embodiments, the Fc region comprises an IgG CH2 domain and an IgG CH3 domain. In some embodiments, the Fc region comprises a heterodimeric Fc region. In some embodiments, the Fc region comprises at least one amino acid modification that increases the half-life of the multispecific antibody. In some embodiments, the Fc region comprises at least one amino acid modification that modulates its interaction with an Fc receptor. In some embodiments, the Fc region comprises at least one amino acid modification that increases binding of the Fc region to an Fc receptor. In some embodiments, the Fc region comprises at least one amino acid modification that decreases glycosylation of the Fc region. In some embodiments, the modification is an amino acid substitution, deletion, or addition. In some embodiments, the modification is an amino acid substitution. In some embodiments, the at least one amino acid modification that decreases glycosylation of the Fc region comprises an amino acid substitution at a position corresponding to position N297 of human IgG1, wherein the numbering is according to the EU index of Kabat. In some embodiments, the Fc region is afucosylated.

In some embodiments, the anti-CD28 light chain polypeptide comprises complementarity determining regions (CDRs): LC-CDR1, LC-CDR2, and LC-CDR3, wherein the LC-CDR1, the LC-CDR2, and the LC-CDR3 the anti-CD28 light chain polypeptide: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3. In some embodiments, the anti-CD28 heavy chain polypeptide comprises complementarity determining regions (CDRs): HC-CDR1, HC-CDR2, and HC-CDR3, wherein the HC-CDR1, the HC-CDR2, and the HC-CDR3 of the anti-CD28 heavy chain polypeptide comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 13; LC-CDR2: SEQ ID NO: 14; and LC-CDR3: SEQ ID NO: 15, and wherein the CDRs comprise from 0-2 amino acid modifications in at least one of said LC-CDR1, LC-CDR2, or LC-CDR3. In some embodiments, the anti-PD-L1 heavy chain polypeptide the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 10; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26, and wherein said CDRs comprise from 0-2 amino acid modifications in at least one of said HC-CDR1, HC-CDR2, or HC-CDR3. In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 33; LC-CDR2: SEQ ID NO: 34; and LC-CDR3: SEQ ID NO: 35, and wherein the CDRs comprise from 0-2 amino acid modifications in at least one of said LC-CDR1, LC-CDR2, or LC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 33; LC-CDR2: SEQ ID NO: 34; and LC-CDR3: SEQ ID NO: 35.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26, and wherein said CDRs comprise from 0-2 amino acid modifications in at least one of said HC-CDR1, HC-CDR2, or HC-CDR3; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 33; LC-CDR2: SEQ ID NO: 34; and LC-CDR3: SEQ ID NO: 35, and wherein the CDRs comprise from 0-2 amino acid modifications in at least one of said LC-CDR1, LC-CDR2, or LC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 24; HC-CDR2: SEQ ID NO: 25; HC-CDR3: SEQ ID NO: 26; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 33; LC-CDR2: SEQ ID NO: 34; and LC-CDR3: SEQ ID NO: 35.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29, and wherein said CDRs comprise from 0-2 amino acid modifications in at least one of said HC-CDR1, HC-CDR2, or HC-CDR3. In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 36; LC-CDR2: SEQ ID NO: 37; and LC-CDR3: SEQ ID NO: 38, and wherein the CDRs comprise from 0-2 amino acid modifications in at least one of said LC-CDR1, LC-CDR2, or LC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 36; LC-CDR2: SEQ ID NO: 37; and LC-CDR3: SEQ ID NO: 38.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29, and wherein said CDRs comprise from 0-2 amino acid modifications in at least one of said HC-CDR1, HC-CDR2, or HC-CDR3; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 36; LC-CDR2: SEQ ID NO: 37; and LC-CDR3: SEQ ID NO: 38, and wherein the CDRs comprise from 0-2 amino acid modifications in at least one of said LC-CDR1, LC-CDR2, or LC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 27; HC-CDR2: SEQ ID NO: 28; HC-CDR3: SEQ ID NO: 29; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 36; LC-CDR2: SEQ ID NO: 37; and LC-CDR3: SEQ ID NO: 38.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32, and wherein said CDRs comprise from 0-2 amino acid modifications in at least one of said HC-CDR1, HC-CDR2, or HC-CDR3. In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32.

In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 39; LC-CDR2: SEQ ID NO: 40; and LC-CDR3: SEQ ID NO: 41, and wherein the CDRs comprise from 0-2 amino acid modifications in at least one of said LC-CDR1, LC-CDR2, or LC-CDR3. In some embodiments, the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 39; LC-CDR2: SEQ ID NO: 40; and LC-CDR3: SEQ ID NO: 41.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32, and wherein said CDRs comprise from 0-2 amino acid modifications in at least one of said HC-CDR1, HC-CDR2, or HC-CDR3; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 39; LC-CDR2: SEQ ID NO: 40; and LC-CDR3: SEQ ID NO: 41, and wherein the CDRs comprise from 0-2 amino acid modifications in at least one of said LC-CDR1, LC-CDR2, or LC-CDR3.

In some embodiments, the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 30; HC-CDR2: SEQ ID NO: 31; HC-CDR3: SEQ ID NO: 32; and the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise: LC-CDR1: SEQ ID NO: 39; LC-CDR2: SEQ ID NO: 40; and LC-CDR3: SEQ ID NO: 41.

Combination Therapy

In some embodiments, are methods of treating cancer in a subject in need thereof comprising administering to the subject the multispecific antibodies comprising a CD28 binding domain and a PD-L1 binding domain as described herein in combination with an anti-cancer therapy.

In some embodiments, the subject is refractory to checkpoint inhibitor therapy.

In some embodiments, the subject has relapsed from checkpoint inhibitor therapy.

In some embodiments, the anti-cancer therapy comprises a small molecule, a cell-based therapy, or an antibody-based therapy.

In some embodiments, the antibody-based therapy is a T cell engager.

In some embodiments, the T cell engager comprises a formula according to: D-L₀-E (Formula II), wherein D comprises an effector cell binding domain that binds to an effector cell antigen, E comprises a tumor antigen binding domain that binds to a tumor antigen, and L₀ comprises a linker that connects D to E.

In some embodiments, D comprises a single chain variable fragment, a single domain antibody, or a Fab fragment. In some embodiments, D comprises the single chain variable fragment. In some embodiments, E comprises a single chain variable fragment, a single domain antibody, or a Fab fragment. In some embodiments, E comprises the Fab fragment. In some embodiments, the effector cell antigen comprises CD3. In some embodiments, the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 1 ID8, XIII-141, XIII-46, XIII-87, 12F6, T3/RW2-8C8, T3/RW2-4B6, OKT31D, M-T301, SMC2, FlOlOl1, UCHT-1, WT-31, 15865, 15865v12, 15865v16, and 15865v19. In some embodiments, the effector cell binding domain comprises an amino acid sequence as disclosed in Table 11.

TABLE 11
Effector cell binding domain comprises an amino acid sequences
(CDRs as determined by IMGT numbering system).

| | Amino Acid Sequence | SEQ ID |
|---|---|---|
| Construct Description | (N to C) | NO: |
| SP34.185 CD3: HC: CDR1 | GFTFNKYA | 89 |
| SP34.185 CD3: HC: CDR2 | IRSKYNNYAT | 90 |
| SP34.185 CD3: HC: CDR3 | VRHGNFGNSYISYWAY | 91 |
| SP34.185 CD3: LC: CDR1 | TGAVTSGNY | 92 |
| SP34.185 CD3: LC: CDR2 | GTK | 93 |
| SP34.185 CD3: LC: CDR3 | VLWYSNRWV | 94 |
| SP34.194 CD3: HC: CDR1 | GFTFNTYA | 95 |
| SP34.194 CD3: HC: CDR2 | IRSKYNNYAT | 90 |
| SP34.194 CD3: HC: CDR3 | VRHGNFGNSYVSWFAY | 96 |
| SP34.194 CD3: LC: CDR1 | TGAVTTSNY | 97 |
| SP34.194 CD3: LC: CDR2 | GT | 98 |
| SP34.194 CD3: LC: CDR3 | ALWYSNLWV | 99 |
| SP34.185 scFv | EVQLVESGGGLVQPGGSLKLS | 100 |
| (VH-linker 1-VL) | CAASGFTFNKYAMNWVRQA | |
| | PGKGLEWVARIRSKYNNYAT | |
| | YYADSVKDRFTISRDDSKNTA | |
| | YLQMNNLKTEDTAVYYCVRH | |
| | GNFGNSYISYWAYWGQGTLV | |
| | TVSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKL | |
| | TVL | |
| SP34.194 scFv | QTVVTQEPSLTVSPGGTVTLT | 101 |
| (VL-linker 1-VH) | CRSSTGAVTTSNYANWVQQK | |
| | PGQAPRGLIGGTNKRAPGTPA | |
| | RFSGSLLGGKAALTLSGVQPE | |
| | DEAEYYCALWYSNLWVFGG | |
| | GTKLTVLGGGGSGGGGSGGG | |
| | GSEVQLVESGGGLVQPGGSLK | |
| | LSCAASGFTFNTYAMNWVRQ | |
| | APGKGLEWVARIRSKYNNYA | |
| | TYYADSVKDRFTISRDDSKNT | |
| | AYLQMNNLKTEDTAVYYCVR | |
| | HGNFGNSYVSWFAYWGQGT | |
| | LVTVSS | |

In some embodiments, the tumor antigen comprises epidermal growth factor receptor (EGFR), prostate-specific membrane antigen (PSMA), or tumor-associated calcium signal transducer 2 (referred to herein after as TROP2).

In some embodiments, the tumor antigen comprises EGFR. In some embodiments, the cancer has cells that express EGFR. In some embodiments, the cancer comprises colorectal cancer (CRC), squamous cell carcinoma of the head and Neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarnan cancer, bladder cancer, kidney cancer, or pancreatic cancer. In some embodiments, the tumor antigen binding domain comprises an amino acid sequence as disclosed in Table 12.

TABLE 12
Tumor antigen binding domain amino acid sequences-anti-EGFR (CDRS
defined by IMGT)

| Construct | Amino Acid Sequence | |
|---|---|---|
| Description | (N to C) | SEQ ID NO: |
| EGFR: LC: CDR1 | QSIGTN | 102 |
| EGFR: LC: CDR2 | YAS | 103 |
| EGFR: LC: CDR3 | QQNNNWPTT | 104 |
| EGFR: HC: CDR1 | GFSLTNYG | 105 |
| EGFR: HC: CDR2 | IWSGGNT | 106 |
| EGFR: HC: CDR3 | ARALTYYDYEFAY | 107 |
| EGFR Fab LC v1 | QILLTQSPVILSVSPGERVSFSCRASQ | 108 |
| | SIGTNIHWYQQRTNGSPRLLIKYAS | |
| | ESISGIPSRFSGSGSGTDFTLSINSVES | |
| | EDIADYYCQQNNNWPTTFGAGTKL | |
| | ELKRTVAAPSVFIFPPSDEQLKSGTA | |
| | SVVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR Fab LC v2 | DILLTQSPVILSVSPGERVSFSCRASQ | 109 |
| | SIGTNIHWYQQRTNGSPRLLIKYAS | |
| | ESISGIPSRFSGSGSGTDFTLSINSVES | |
| | EDIADYYCQQNNNWPTTFGAGTKL | |
| | ELKRTVAAPSVFIFPPSDEQLKSGTA | |
| | SVVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR Fab HC | QVQLKQSGPGLVQPSQSLSITCTVS | 110 |
| | GFSLTNYGVHWVRQSPGKGLEWL | |
| | GVIWSGGNTDYNTPFTSRLSINKDN | |
| | SKSQVFFKMNSLQSNDTAIYYCARA | |
| | LTYYDYEFAYWGQGTLVTVSAAST | |
| | KGPSVFPLAPSSKSTSGGTAALGCL | |
| | VKDYFPEPVTVSWNSGALTSGVHTF | |
| | PAVLQSSGLYSLSSVVTVPSSSLGTQ | |
| | TYICNVNHKPSNTKVDKKVEPKSC | |
| EGFR Fab HC | QVQLKQSGPGLVQPSQSLSITCTVS | 111 |
| (N88Q) | GFSLTNYGVHWVRQSPGKGLEWL | |
| | GVIWSGGNTDYNTPFTSRLSINKDN | |
| | SKSQVFFKMNSLQSQDTAIYYCARA | |
| | LTYYDYEFAYWGQGTLVTVSAAST | |
| | KGPSVFPLAPSSKSTSGGTAALGCL | |
| | VKDYFPEPVTVSWNSGALTSGVHTF | |
| | PAVLQSSGLYSLSSVVTVPSSSLGTQ | |
| | TYICNVNHKPSNTKVDKKVEPKSC | |

In some embodiments, the tumor antigen comprises TROP2. In some embodiments, the cancer has cells that express TROP2. In some instances, the cancer is a solid tumor cancer. In some embodiments, the cancer is lung, breast (e.g. HER2+; ER/PR+; TNBC), cervical, ovarian, colorectal, pancreatic gastric, urothelial, endometrial, head and neck, or glioma. In some embodiments, the tumor antigen binding domain comprises an amino acid sequence as disclosed in Table 13

TABLE 13
Tumor antigen binding domain amino acid sequences-anti-TROP2
(CDRS defined by IMGT)

| | Amino Acid Sequence | SEQ ID |
|---|---|---|
| Construct Description | (N to C) | NO: |
| TROP2: HC: CDR1 | GYTFTNYG | 112 |
| TROP2: HC: CDR2 | INTYTGEP | 113 |
| TROP2: HC: CDR3 | ARGGFGSSYWYFDV | 114 |
| TROP2: LC: CDR1 | QDVSIA | 115 |
| TROP2: LC: CDR2 | SAS | 116 |
| TROP2: LC: CDR3 | QQHYITPLT | 117 |
| TROP2 Fab LC | DIQLTQSPSSLSASVGDRVSITC | 118 |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 Fab HC | QVQLQQSGSELKKPGASVKVS | 119 |
| | CKASGYTFTNYGMNWVKQA | |
| | PGQGLKWMGWINTYTGEPTY | |
| | TDDFKGRFAFSLDTSVSTAYL | |
| | QISSLKADDTAVYFCARGGFG | |
| | SSYWYFDVWGQGSLVTVSSA | |
| | STKGPSVFPLAPSSKSTSGGTA | |
| | ALGCLVKDYFPEPVTVSWNSG | |
| | ALTSGVHTFPAVLQSSGLYSLS | |
| | SVVTVPSSSLGTQTYICNVNH | |
| | KPSNTKVDKKVEPKSC | |

In some embodiments, the tumor antigen comprises PSMA. In some embodiments, the cancer comprises prostate cancer. In some embodiments, the cancer comprises metastatic castrate-resistant prostate cancer (mCRPC). In some embodiments, the tumor antigen binding domain comprises an amino acid sequence as disclosed in Table 14.

TABLE 14
Tumor antigen binding domain amino acid sequences-anti-PSMA
(CDRS defined by IMGT)

| | Amino Acid Sequence | SEQ ID |
|---|---|---|
| Construct Description | (N to C) | NO: |
| PSMA: HC: CDR1 | GFAFSRYG | 120 |
| PSMA: HC: CDR2 | IWYDGSNK | 121 |
| PSMA: HC: CDR3 | ARGGDFLYYYYYGMDV | 122 |
| PSMA: LC: CDR1 | QGISNY | 123 |
| PSMA: LC: CDR2 | EA | 124 |
| PSMA: LC: CDR3 | QNYNSAPFT | 125 |
| 006 PSMA Fab LC | DIQMTQSPSSLSASVGDRVTIT | 126 |
| | CRASQGISNYLAWYQQKTGK | |
| | VPKFLIYEASTLQSGVPSRFSG | |
| | GGSGTDFTLTISSLQPEDVATY | |
| | YCQNYNSAPFTFGPGTKVDIK | |
| | RTVAAPSVFIFPPSDEQLKSGT | |
| | ASVVCLLNNFYPREAKVQWK | |
| | VDNALQSGNSQESVTEQDSKD | |
| | STYSLSSTLTLSKADYEKHKV | |
| | YACEVTHQGLSSPVTKSFNRG | |
| | EC | |
| 006 PSMA Fab HC | QVQLVESGGGVVQPGRSLRLS | 127 |
| | CAASGFAFSRYGMHWVRQAP | |
| | GKGLEWVAVIWYDGSNKYY | |
| | ADSVKGRFTISRDNSKNTQYL | |
| | QMNSLRAEDTAVYYCARGGD | |
| | FLYYYYYGMDVWGQGTTVT | |
| | VSSASTKGPSVFPLAPSSKSTS | |
| | GGTAALGCLVKDYFPEPVTVS | |
| | WNSGALTSGVHTFPAVLQSSG | |
| | LYSLSSVVTVPSSSLGTQTYIC | |
| | NVNHKPSNTKVDKKVEPKSC | |

In some embodiments, the T cell engager molecule is selectively activated in tumor microenvironments.

In some embodiments, the multispecific antibodies described herein are selectively activated in tumor microenvironments.

In some embodiments, the T cell engager is according to the following subformula: P₃-L₃-D-L₀-E (Formula IIa) wherein D comprises the CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease.

In some embodiments, the T cell engager is according to the following subformula: D-L₀-E-L₄-P₄ (Formula IIb) wherein D comprises the CD3 binding domain; E comprises the tumor antigen binding domain; L₀ comprises the linker that connects D to E; P₄ comprises a peptide that binds to E and L₄ comprises a linking moiety that connects E to P₄ and is a substrate for a tumor specific protease.

In some embodiments, the T cell engager is according to the following subformula: P₃-L₃-D-L₀-E-L₄-P₄ (Formula IIc) wherein D comprises the CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease; P₄ comprises a peptide that binds to E and L₄ comprises a linking moiety that connects E to P₄ and is a substrate for a tumor specific protease.

In some embodiments, the T cell engager comprises the half-life extending molecule (H₁).

Linking Moiety (L₃ or L₄)

In some embodiments, L₃ or L₄ is a peptide sequence having at least 5 to no more than 50 amino acids. In some embodiments L₃ or L₄ is a peptide sequence having at least 10 to no more than 30 amino acids. In some embodiments, L₃ or L₄ is a peptide sequence having at least 10 amino acids. In some embodiments, L₃ or L₄ is a peptide sequence having at least 18 amino acids. In some embodiments, L₃ or L4 is a peptide sequence having at least 26 amino acids. In some embodiments, L₃ or L₄ has a formula comprising (G₂S)ₙ (SEQ ID NO: 233), wherein n is an integer from 1 to 3. In some embodiments, L₃ or L4 has a formula comprising (G₂S)ₙ, wherein n is an integer of at least 1. In some embodiments, L₃ or L₄ has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1. In some embodiments, the tumor specific protease is selected from the group consisting of metalloprotease, serine protease, cysteine protease, threonine protease, and aspartic protease. In some embodiments L₃ or L₄ comprises a urokinase cleavable amino acid sequence, a matriptase cleavable amino acid sequence, a legumain cleavable amino acid sequence, or a matrix metalloprotease cleavable amino acid sequence. In some embodiments, L₃ or L₄ comprises a sequence as disclosed in Table 10 or a sequence substantially identical thereto (e.g., a sequence that has 0, 1, or 2 amino acid modifications).

In some embodiments, L₃ is bound to N-terminus of D. In some embodiments, L₃ is bound to C-terminus of D. In some embodiments, L₄ is bound to N-terminus of E. In some embodiments, L₄ is bound to C-terminus of E. In some embodiments, P₃ becomes unbound from D when L₃ is cleaved by the tumor specific protease thereby exposing D to CD3. In some embodiments, P₄ becomes unbound from E when L₄ is cleaved by the tumor specific protease thereby exposing E to the tumor antigen.

In some embodiments, L₃ or L₄, comprise a modified amino acid or non-natural amino acid, or a modified non-natural amino acid, or a combination thereof. In some embodiments, the modified amino acid or a modified non-natural amino acid comprises a post-translational modification. In some embodiments, L₃ or L₄ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₃ or L₄ including the peptide backbone, or the amino acid side chains.

Peptide (P₃ and P₄)

P₃ or P₄ is designed to be optimized for a specific antigen-binding domain through an iterative process of phage display and quantitative binding assays designed to select for those peptides that are able to prevent binding to CD3 or the tumor antigen. A directed evolution-based process that includes phage libraries is used for identifying P₃ or P₄. Multiple cycles of selection and amplification of potential inhibitory peptides that are capable of blocking the antigen binding domain from binding to its target of CD3 or the tumor antigen with the goal of optimizing masked T cell engager antibodies in serum and limiting cleavage, thereby reducing toxicity. Discovery of P₃ or P₄ is depicted in FIG. 18.

In some embodiments, P₃ impairs binding of D to CD3. In some embodiments, P₃ is bound to D through ionic interactions, electrostatic interactions, hydrophobic interactions, Pi-stacking interactions, and H-bonding interactions, or a combination thereof. In some embodiments, P₃ is bound to D at or near an antigen binding site. In some embodiments, P₃ becomes unbound from D when L₃ is cleaved by the tumor specific protease thereby exposing D to CD3. In some embodiments, P₃ has less than 70% sequence identity to CD3. In some embodiments, P₃ has less than 750% sequence identity to CD3. In some embodiments, P₃ has less than 80% sequence identity to CD3. In some embodiments, P₃ has less than 85% sequence identity to CD3. In some embodiments, P₃ has less than 90% sequence identity to CD3. In some embodiments, P₃ has less than 95% sequence identity to CD3. In some embodiments, P₃ has less than 98% sequence identity to CD3. In some embodiments, P₃ has less than 99% sequence identity to CD3. In some embodiments, P₃ comprises a de novo amino acid sequence that shares less than 10% sequence identity to CD3.

In some embodiments, P₄ impairs binding of E to the tumor antigen antigen. In some embodiments, P₄ is bound to E through ionic interactions, electrostatic interactions, hydrophobic interactions, Pi-stacking interactions, and H-bonding interactions, or a combination thereof. In some embodiments, P₄ is bound to E at or near an antigen binding site. In some embodiments, P₄ becomes unbound from E when L₄ is cleaved by the tumor specific protease thereby exposing E to the tumor antigen. In some embodiments, P₄ has less than 70% sequence identity to the the tumor antigen. In some embodiments, P₄ has less than 75% sequence identity to the tumor antigen. In some embodiments, P₄ has less than 80% sequence identity to the tumor antigen. In some embodiments, P₄ has less than 85% sequence identity to the tumor antigen. In some embodiments, P₄ has less than 90% sequence identity to the tumor antigen. In some embodiments, P₄ has less than 95% sequence identity to the tumor antigen. In some embodiments, P₄ has less than 98% sequence identity to the tumor antigen. In some embodiments, P₄ has less than 99% sequence identity to the tumor antigen. In some embodiments, P₄ comprises a de novo amino acid sequence that shares less than 10% sequence identity to the tumor antigen.

In some embodiments, P₃ or P₄ comprises a peptide sequence of at least 5 amino acids in length. In some embodiments, P₃ or P₄ comprises a peptide sequence of at least 6 amino acids in length. In some embodiments, P₃ or P₄ comprises a peptide sequence of at least 10 amino acids in length. In some embodiments, P₃ or P₄ comprises a peptide sequence of at least 10 amino acids in length and no more than 20 amino acids in length. In some embodiments, P₃ or P₄ comprises a peptide sequence of at least 16 amino acids in length. In some embodiments, P₃ or P₄ comprises a peptide sequence of no more than 40 amino acids in length. In some embodiments, P₃ or P₄ comprises at least two cysteine amino acid residues. In some embodiments, P₃ or P₄ comprises a cyclic peptide or a linear peptide. In some embodiments, P₃ or P₄ comprises a cyclic peptide. In some embodiments, P₃ or P₄ comprises a linear peptide.

In some embodiments, P₃ or P₄ comprise a modified amino acid or non-natural amino acid, or a modified non-natural amino acid, or a combination thereof. In some embodiments, the modified amino acid or a modified non-natural amino acid comprises a post-translational modification. In some embodiments P₃ or P₄ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₃ or P₄ including the peptide backbone, the amino acid side chains, and the terminus.

In some embodiments, P₃ or P₄ does not comprise albumin or an albumin fragment. In some embodiments, P₃ or P₄ does not comprise an albumin binding domain.

### Production of Antibodies

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBacl, pFastBac DUAL, pFastBac ET, pFastBac HTa, pFastBac HTb, pFastBac HTc, pFastBac M3Oa, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway@ pDEST™ 15 vector, Gateway@ pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™ TOP10, DH5α, DH10Bac™, OmniMax™, MegaX™, DH12S™, INV110, TOP10F′, INVaF, TOP10/P3, ccdB Survival, PIR1, PIR2, Stbl2™, Stbl3™, or Stbl4™

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVScl.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic.

The label or package insert indicates that the composition is used for treating the condition of choice. The article of manufacture in this embodiment of the disclosure may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Embodiments

Embodiment 1 comprises a multispecific antibody comprising a CD28 binding domain and a PD-L1 binding domain.

Embodiment 2 comprises a multispecific antibody of embodiment 1, wherein when the CD28 binding domain is a single chain variable fragment (scFv), then the PD-L1 binding domain is not a scFv.

Embodiment 3 comprises a multispecific antibody of any one of embodiments 1-2, wherein the multispecific antibody is according to the following formula: A-L-B (Formula I) wherein A comprises the CD28 binding domain; B comprises the PD-L1 binding domain; and L comprises a linker that connects A to B.

Embodiment 4 comprises a multispecific antibody of any one of embodiments 1-3, wherein the CD28 binding domain comprises a single chain variable fragment, a single domain antibody, a Fab, or a Fab′.

Embodiment 5 comprises a multispecific antibody of embodiment 4, wherein the CD28 binding domain comprises the single chain variable fragment.

Embodiment 6 comprises a multispecific antibody of embodiment 4, wherein the CD28 binding domain comprises the single domain antibody.

Embodiment 7 comprises a multispecific antibody of embodiment 4, wherein the CD28 binding domain comprises the Fab or the Fab′.

Embodiment 8 comprises a multispecific antibody of any one of embodiments 1-7, wherein the PD-L1 binding domain comprises a single chain variable fragment, a single domain antibody, a Fab, or a Fab′.

Embodiment 9 comprises a multispecific antibody of embodiment 8, wherein the PD-L1 binding domain comprises the Fab or the Fab′.

Embodiment 10 comprises a multispecific antibody of embodiment 8, wherein the PD-L1 binding domain comprises the Fab or the Fab′ and the CD28 binding domain comprises the single chain variable fragment.

Embodiment 11 comprises a multispecific antibody of embodiment 9, wherein the PD-L1 binding domain that comprises the Fab or the Fab′ comprises a Fab heavy chain polypeptide comprising a Fab heavy chain variable domain and a Fab light chain polypeptide comprising a Fab light chain variable domain.

Embodiment 12 comprises a multispecific antibody of embodiment 10, wherein the CD28 binding domain that comprises the single chain variable fragment comprises a scFv heavy chain variable domain and a scFv light chain variable domain.

Embodiment 13 comprises a multispecific antibody of any one of embodiments 1-12, wherein the linker connects the C-terminus of A to an N-terminus of B.

Embodiment 14 comprises a multispecific antibody of any one of embodiments 1-12, wherein the linker connects the N-terminus of A to a C-terminus of B.

Embodiment 15 comprises a multispecific antibody of any one of embodiments 11 or 12, wherein the linker connects the C-terminus of A to the N-terminus of the Fab heavy chain polypeptide.

Embodiment 16 comprises a multispecific antibody of any one of embodiments 11 or 12, wherein the linker connects the N-terminus of A to the C-terminus of the Fab heavy chain polypeptide.

Embodiment 17 comprises a multispecific antibody of any one of embodiments 11 or 12, wherein the linker connects the C-terminus of A to the N-terminus of the Fab light chain polypeptide.

Embodiment 18 comprises a multispecific antibody of any one of embodiments 11 or 12, wherein the linker connects the N-terminus of A to the C-terminus of the Fab light chain polypeptide.

Embodiment 19 comprises a multispecific antibody of embodiment 12, wherein the linker connects the Fab light chain polypeptide to the scFv light chain variable domain.

Embodiment 20 comprises a multispecific antibody of embodiment 12, wherein the linker connects the Fab light chain polypeptide to the scFv heavy chain variable domain.

Embodiment 21 comprises a multispecific antibody of embodiment 12, wherein the linker connects the Fab heavy chain polypeptide to the scFv light chain variable domain.

Embodiment 22 comprises a multispecific antibody of embodiment 12, wherein the linker connects the Fab heavy chain polypeptide to the scFv heavy chain variable domain.

Embodiment 23 comprises a multispecific antibody of embodiment 12, wherein the linker connects the Fab light chain polypeptide to the N-terminus of the scFv light chain variable domain.

Embodiment 24 comprises a multispecific antibody of embodiment 12, wherein the linker connects the Fab light chain polypeptide to the C-terminus of the scFv light chain variable domain.

Embodiment 25 comprises a multispecific antibody of embodiment 12, wherein the linker connects the Fab light chain polypeptide to the N-terminus of the scFv heavy chain variable domain.

Embodiment 26 comprises a multispecific antibody of embodiment 12, wherein the linker connects the Fab light chain polypeptide to the C-terminus of the scFv heavy chain variable domain.

Embodiment 27 comprises a multispecific antibody of embodiment 12, wherein the linker connects the Fab heavy chain polypeptide to the N-terminus of the scFv light chain variable domain.

Embodiment 28 comprises a multispecific antibody of embodiment 12, wherein the linker connects the Fab heavy chain polypeptide to the C-terminus of the scFv light chain variable domain.

Embodiment 29 comprises a multispecific antibody of embodiment 12, wherein the linker connects the Fab heavy chain polypeptide to the N-terminus of the scFv heavy chain variable domain.

Embodiment 30 comprises a multispecific antibody of embodiment 12, wherein the linker connects the Fab heavy chain polypeptide to the C-terminus of the scFv heavy chain variable domain.

Embodiment 31 comprises a multispecific antibody of any one of embodiments 3-30, wherein the linker is at least 5 amino acids in length.

Embodiment 32 comprises a multispecific antibody of any one of embodiments 3-31, wherein the linker is no more than 30 amino acids in length.

Embodiment 33 comprises a multispecific antibody of any one of embodiments 3-32, wherein the linker is at least 5 amino acids and no more than 30 amino acids in length.

Embodiment 34 comprises a multispecific antibody of any one of embodiments 3-33, wherein the linker is 5 amino acids in length.

Embodiment 35 comprises a multispecific antibody of any one of embodiments 3-33, wherein the linker is 15 amino acids in length.

Embodiment 36 comprises a multispecific antibody of any one of embodiments 3-30, wherein the linker is selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 37 comprises a multispecific antibody of any one of embodiments 3-30, wherein L has a formula comprising (G₂S)ₙ (SEQ ID NO: 233), wherein n is an integer from 1 to 3.

Embodiment 38 comprises a multispecific antibody of any one of embodiments 3-30, wherein the L comprises an amino acid sequence of SEQ ID NO: 18 (GGGGSGGGGSGGGGS) or SEQ ID NO: 19 (GGGGS).

Embodiment 39 comprises a multispecific antibody of any one of embodiments 12-38, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

Embodiment 40 comprises a multispecific antibody of any one of embodiments 12-38, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3.

Embodiment 41 comprises a multispecific antibody of any one of embodiments 12-38, wherein the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, HC-CDR1 SEQ ID NO: 24; HC-CDR2: SEQ ID NO: 25; HC-CDR3: SEQ ID NO: 26; or HC-CDR1 SEQ ID NO: 27; HC-CDR2: SEQ ID NO: 28; HC-CDR3: SEQ ID NO: 29; or HC-CDR1: SEQ ID NO: 30; HC-CDR2: SEQ ID NO: 31; HC-CDR3: SEQ ID NO: 32; and wherein said CDRs comprise from 0-2 amino acid modifications in at least one of said HC-CDR1, HC-CDR2, or HC-CDR3.

Embodiment 42 comprises a multispecific antibody of any one of embodiments 12-38, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise:LC-CDR1: SEQ ID NO: 13; LC-CDR2: SEQ ID NO: 14; and LC-CDR3: SEQ ID NO: 15; LC-CDR1 SEQ ID NO: 33; LC-CDR2: SEQ ID NO: 34; and LC-CDR3: SEQ ID NO: 35; LC-CDR1 SEQ ID NO: 36; LC-CDR2: SEQ ID NO: 37; and LC-CDR3: SEQ ID NO: 38; or LC-CDR1: SEQ ID NO: 39; LC-CDR2: SEQ ID NO: 40; and LC-CDR3: SEQ ID NO: 41; and wherein the CDRs comprise from 0-2 amino acid modifications in at least one of said LC-CDR1, LC-CDR2, or LC-CDR3.

Embodiment 43 comprises a multispecific antibody of any one of embodiments 12-38, wherein the scFv heavy chain variable domain comprises an amino acid sequence that has at least 80% sequence identity to the amino acid sequence according to SEQ ID NO: 7.

Embodiment 44 comprises a multispecific antibody of any one of embodiments 12-38, wherein the scFv heavy chain variable domain comprises an amino acid sequence of at least 75 consecutive amino acid residues of SEQ ID NO: 7

Embodiment 45 comprises a multispecific antibody of any one of embodiments 12-38, wherein the scFv heavy chain variable domain comprises an amino acid sequence of at least 110 consecutive amino acid residues of SEQ ID NO: 7.

Embodiment 46 comprises a multispecific antibody of any one of embodiments 12-38, wherein the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7.

Embodiment 47 comprises a multispecific antibody of any one of embodiments 12-38, wherein the scFv heavy chain variable domain comprises an amino acid sequence according to SEQ ID NO: 7.

Embodiment 48 comprises a multispecific antibody of any one of embodiments 12-38, wherein the scFv light chain variable domain comprises an amino acid sequence that has at least 80% sequence identity to the amino acid sequence according to SEQ ID NO: 8.

Embodiment 49 comprises a multispecific antibody of any one of embodiments 12-38, wherein the scFv light chain variable domain comprises an amino acid sequence of at least 75 consecutive amino acid residues of SEQ ID NO: 8.

Embodiment 50 comprises a multispecific antibody of any one of embodiments 12-38, wherein the scFv light chain variable domain comprises an amino acid sequence of at least 100 consecutive amino acid residues of SEQ ID NO: 8.

Embodiment 51 comprises a multispecific antibody of any one of embodiments 12-38, wherein the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8.

Embodiment 52 comprises a multispecific antibody of any one of embodiments 12-38, wherein the scFv light chain variable domain comprises an amino acid sequence according to SEQ ID NO: 8.

Embodiment 53 comprises a multispecific antibody of any one of embodiments 12-38, wherein the scFv comprises an amino acid sequence that has at least 80% sequence identity to the amino acid sequence according to SEQ ID NO: 9.

Embodiment 54 comprises a multispecific antibody of any one of embodiments 12-38, wherein the scFv comprises an amino acid sequence of at least 175 consecutive amino acid residues of SEQ ID NO: 9.

Embodiment 55 comprises a multispecific antibody of any one of embodiments 12-38, wherein the scFv comprises an amino acid sequence of at least 210 consecutive amino acid residues of SEQ ID NO: 9.

Embodiment 56 comprises a multispecific antibody of any one of embodiments 12-38, wherein the scFv comprises an amino acid sequence of at least 210 consecutive amino acid residues of SEQ ID NO: 9 and has at least 80% sequence identity to the at least 210 consecutive amino acid residues of SEQ ID NO: 9.

Embodiment 57 comprises a multispecific antibody of any one of embodiments 11-38, wherein the scFv comprises an amino acid sequence according to SEQ ID NO: 9.

Embodiment 58 comprises a multispecific antibody of any one of embodiments 11-57, wherein the Fab heavy chain polypeptide comprises an amino acid sequence that has at least 80% sequence identity to the amino acid sequence according to SEQ ID NO: 17, 43, 45, or 47.

Embodiment 59 comprises a multispecific antibody of any one of embodiments 11-57, wherein the Fab heavy chain polypeptide comprises an amino acid sequence of at least 175 consecutive amino acid residues of SEQ ID NO: 17, 43, 45, or 47.

Embodiment 60 comprises a multispecific antibody of any one of embodiments 11-57, wherein the Fab heavy chain polypeptide comprises an amino acid sequence of at least 215 consecutive amino acid residues of SEQ ID NO: 17, 43, 45, or 47.

Embodiment 61 comprises a multispecific antibody of any one of embodiments 11-57, wherein the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17, 43, 45, or 47.

Embodiment 62 comprises a multispecific antibody of any one of embodiments 11-57, wherein the Fab heavy chain polypeptide comprises an amino acid sequence according to SEQ ID NO: 17, 43, 45, or 47.

Embodiment 63 comprises a multispecific antibody of any one of embodiments 11-57, wherein the Fab light chain polypeptide comprises an amino acid sequence that has at least 80% sequence identity to the amino acid sequence according to SEQ ID NO: 16, 42, 44, or 46.

Embodiment 64 comprises a multispecific antibody of any one of embodiments 11-57, wherein the Fab light chain polypeptide comprises an amino acid sequence of at least 175 consecutive amino acid residues of SEQ ID NO: 16, 42, 44, or 46.

Embodiment 65 comprises a multispecific antibody of any one of embodiments 11-57, wherein the Fab light chain polypeptide comprises an amino acid sequence of at least 200 consecutive amino acid residues of SEQ ID NO: 16, 42, 44, or 46.

Embodiment 66 comprises a multispecific antibody of any one of embodiments 11-57, wherein the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16, 42, 44, or 46.

Embodiment 67 comprises a multispecific antibody of any one of embodiments 11-57, wherein the Fab light chain polypeptide comprises an amino acid sequence according to SEQ ID NO: 16, 42, 44, or 46.

Embodiment 68 comprises a multispecific antibody of any one of embodiments 12-38, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

Embodiment 69 comprises a multispecific antibody of any one of embodiments 12-38, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21.

Embodiment 70 comprises a multispecific antibody of any one of embodiments 12-38, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21.

Embodiment 71 comprises a multispecific antibody of any one of embodiments 12-38, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

Embodiment 72 comprises a multispecific antibody of any one of embodiments 12-38, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

Embodiment 73 comprises a multispecific antibody of any one of embodiments 12-38, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22.

Embodiment 74 comprises a multispecific antibody of any one of embodiments 12-38, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22.

Embodiment 75 comprises a multispecific antibody of any one of embodiments 12-38, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

Embodiment 76 comprises a multispecific antibody comprising a CD28 binding domain and a PD-L1 binding domain wherein the multispecific antibody is selectively activated in a tumor microenvironment.

Embodiment 77 comprises a multispecific antibody of embodiment 76, wherein the CD28 binding domain comprises a single chain variable fragment, a single domain antibody, a Fab, or a Fab′.

Embodiment 78 comprises a multispecific antibody of embodiment 77, wherein the CD28 binding domain comprises the single chain variable fragment.

Embodiment 79 comprises a multispecific antibody of embodiment 77, wherein the CD28 binding domain comprises the single domain antibody.

Embodiment 80 comprises a multispecific antibody of embodiment 77, wherein the CD28 binding domain comprises the Fab or the Fab′.

Embodiment 81 comprises a multispecific antibody of any one of embodiments 76-81, wherein the PD-L1 binding domain comprises a single chain variable fragment, single domain antibody, a Fab, or a Fab′.

Embodiment 82 comprises a multispecific antibody of embodiment 81, wherein the PD-L1 binding domain comprises the Fab or the Fab′.

Embodiment 83 comprises a multispecific antibody of embodiment 81, wherein the PD-L1 binding domain comprises the Fab or the Fab′ and the CD28 binding domain comprises the single chain variable fragment.

Embodiment 84 comprises a multispecific antibody of any one of embodiments 81-83, wherein the PD-L1 binding domain that comprises the Fab or the Fab′ comprises a Fab heavy chain polypeptide comprising a Fab heavy chain variable domain and a Fab light chain polypeptide comprising a Fab light chain variable domain.

Embodiment 85 comprises a multispecific antibody of any one of embodiments 77-78, or 80-84, wherein the CD28 binding domain that comprises the single chain variable fragment comprises a scFv heavy chain variable domain and a scFv light chain variable domain.

Embodiment 86 comprises a multispecific antibody of any one of embodiments 76-85, wherein the multispecific antibody is according to the following formula: P₁-L₁-A-L-B (Formula Ia) wherein A comprises the CD28 binding domain; B comprises the PD-L1 binding domain; L comprises a linker that connects A to B; P₁ comprises a peptide that binds to A and L₁ comprises a linking moiety that connects A to P₁ and is a substrate for a tumor specific protease.

Embodiment 87 comprises a multispecific antibody of any one of embodiments 76-85, wherein the multispecific antibody is according to the following formula: A-L-B-L₂-P₂ (Formula Ib) wherein A comprises the CD28 binding domain; B comprises the PD-L1 binding domain; L comprises a linker that connects A to B; P₂ comprises a peptide that binds to B and L₂ comprises a linking moiety that connects B to P₂ and is a substrate for a tumor specific protease.

Embodiment 88 comprises a multispecific antibody of any one of embodiments 76-85, wherein the multispecific antibody is according to the following formula: P₁-L₁-A-L-B-L₂-P₂ (Formula Ic) wherein A comprises the CD28 binding domain; B comprises the PD-L1 binding domain; L comprises a linker that connects A to B; P1 comprises a peptide that binds to A and L₁ comprises a linking moiety that connects A to P₁ and is a substrate for a tumor specific protease; P₂ comprises a peptide that binds to B and L₂ comprises a linking moiety that connects B to P₂ and is a substrate for a tumor specific protease.

Embodiment 89 comprises a multispecific antibody of any one of embodiments 86-88, wherein the linker connects the C-terminus of A to an N-terminus of B.

Embodiment 90 comprises a multispecific antibody of any one of embodiments 86-88, wherein the linker connects the N-terminus of A to a C-terminus of B.

Embodiment 91 comprises a multispecific antibody of any one of embodiments 86-88, wherein the linker connects the C-terminus of A to the N-terminus of the Fab heavy chain polypeptide.

Embodiment 92 comprises a multispecific antibody of any one of embodiments 86-88, wherein the linker connects the N-terminus of A to the C-terminus of the Fab heavy chain polypeptide.

Embodiment 93 comprises a multispecific antibody of any one of embodiments 86-88, wherein the linker connects the C-terminus of A to the N-terminus of the Fab light chain polypeptide.

Embodiment 94 comprises a multispecific antibody of any one of embodiments 86-88, wherein the linker connects the N-terminus of A to the C-terminus of the Fab light chain polypeptide.

Embodiment 95 comprises a multispecific antibody of any one of embodiments 86-88, wherein the linker connects the Fab light chain polypeptide to the scFv light chain variable domain.

Embodiment 96 comprises a multispecific antibody of any one of embodiments 86-88, wherein the linker connects the Fab light chain polypeptide to the scFv heavy chain variable domain.

Embodiment 97 comprises a multispecific antibody of any one of embodiments 86-88, wherein the linker connects the Fab heavy chain polypeptide to the scFv light chain variable domain.

Embodiment 98 comprises a multispecific antibody of any one of embodiments 86-88, wherein the linker connects the Fab heavy chain polypeptide to the scFv heavy chain variable domain.

Embodiment 99 comprises a multispecific antibody of any one of embodiments 86-88, wherein the linker connects the Fab light chain polypeptide to the N-terminus of the scFv light chain variable domain.

Embodiment 100 comprises a multispecific antibody of any one of embodiments 86-88, wherein the linker connects the Fab light chain polypeptide to the C-terminus of the scFv light chain variable domain.

Embodiment 101 comprises a multispecific antibody of any one of embodiments 86-88, wherein the linker connects the Fab light chain polypeptide to the N-terminus of the scFv heavy chain variable domain.

Embodiment 102 comprises a multispecific antibody of any one of embodiments 86-88, wherein the linker connects the Fab light chain polypeptide to the C-terminus of the scFv heavy chain variable domain.

Embodiment 103 comprises a multispecific antibody of any one of embodiments 86-88, wherein the linker connects the Fab heavy chain polypeptide to the N-terminus of the scFv light chain variable domain.

Embodiment 104 comprises a multispecific antibody of any one of embodiments 86-88, wherein the linker connects the Fab heavy chain polypeptide to the C-terminus of the scFv light chain variable domain.

Embodiment 105 comprises a multispecific antibody of any one of embodiments 86-88, wherein the linker connects the Fab heavy chain polypeptide to the N-terminus of the scFv heavy chain variable domain.

Embodiment 106 comprises a multispecific antibody of any one of embodiments 86-88, wherein the linker connects the Fab heavy chain polypeptide to the C-terminus of the scFv heavy chain variable domain.

Embodiment 107 comprises a multispecific antibody of any one of embodiments 86-106, wherein the linker is at least 5 amino acids in length.

Embodiment 108 comprises a multispecific antibody of any one of embodiments 86-107, wherein the linker is no more than 30 amino acids in length.

Embodiment 109 comprises a multispecific antibody of any one of embodiments 86-108, wherein the linker is at least 5 amino acids and no more than 30 amino acids in length.

Embodiment 110 comprises a multispecific antibody of any one of embodiments 86-109, wherein the linker is 5 amino acids in length.

Embodiment 111 comprises a multispecific antibody of any one of embodiments 86-109, wherein the linker is 15 amino acids in length.

Embodiment 112 comprises a multispecific antibody of any one of embodiments 86-109, wherein the linker is selected from the group consisting of (G₂S)ₙ, (GS)ₙ, (GSGGS)ₙ (SEQ ID NO: 58), (GGGS)˜(SEQ ID NO: 59), (GGGGS)ₙ (SEQ ID NO: 60), and (GSSGGS)ₙ (SEQ ID NO: 61), wherein n is an integer of at least 1.

Embodiment 113 comprises a multispecific antibody of any one of embodiments 86-109, wherein L has a formula comprising (G₂S)ₙ (SEQ ID NO: 233), wherein n is an integer from 1 to 3.

Embodiment 114 comprises a multispecific antibody of any one of embodiments 86-109, wherein the L comprises an amino acid sequence of SEQ ID NO: 18 (GGGGSGGGGSGGGGS) or SEQ ID NO: 19 (GGGGS).

Embodiment 115 comprises a multispecific antibody of any one of embodiments 86-114, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

Embodiment 116 comprises a multispecific antibody of any one of embodiments 86-114, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3.

Embodiment 117 comprises a multispecific antibody of any one of embodiments 86-114, wherein the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, HC-CDR1: SEQ ID NO: 24; HC-CDR2: SEQ ID NO: 25; HC-CDR3: SEQ ID NO: 26; or HC-CDR1: SEQ ID NO: 27; HC-CDR2: SEQ ID NO: 28; HC-CDR3: SEQ ID NO: 29; or HC-CDR1: SEQ ID NO: 30; HC-CDR2: SEQ ID NO: 31; HC-CDR3: SEQ ID NO: 32; and wherein said CDRs comprise from 0-2 amino acid modifications in at least one of said HC-CDR1, HC-CDR2, or HC-CDR3.

Embodiment 118 comprises a multispecific antibody of any one of embodiments 86-114, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise:LC-CDR1: SEQ ID NO: 13; LC-CDR2: SEQ ID NO: 14; and LC-CDR3: SEQ ID NO: 15; LC-CDR1 SEQ ID NO: 33; LC-CDR2: SEQ ID NO: 34; and LC-CDR3: SEQ ID NO: 35; LC-CDR1 SEQ ID NO: 36; LC-CDR2: SEQ ID NO: 37; and LC-CDR3: SEQ ID NO: 38; or LC-CDR1: SEQ ID NO: 39; LC-CDR2: SEQ ID NO: 40; and LC-CDR3: SEQ ID NO: 41; and wherein the CDRs comprise from 0-2 amino acid modifications in at least one of said LC-CDR1, LC-CDR2, or LC-CDR3.

Embodiment 119 comprises a multispecific antibody of any one of embodiments 86-114, wherein the scFv heavy chain variable domain comprises an amino acid sequence that has at least 80% sequence identity to the amino acid sequence according to SEQ ID NO: 7.

Embodiment 120 comprises a multispecific antibody of any one of embodiments 86-114, wherein the scFv heavy chain variable domain comprises an amino acid sequence of at least 75 consecutive amino acid residues of SEQ ID NO: 7

Embodiment 121 comprises a multispecific antibody of any one of embodiments 86-114, wherein the scFv heavy chain variable domain comprises an amino acid sequence of at least 110 consecutive amino acid residues of SEQ ID NO: 7.

Embodiment 122 comprises a multispecific antibody of any one of embodiments 86-114, wherein the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7.

Embodiment 123 comprises a multispecific antibody of any one of embodiments 86-114, wherein the scFv heavy chain variable domain comprises an amino acid sequence according to SEQ ID NO: 7.

Embodiment 124 comprises a multispecific antibody of any one of embodiments 86-114, wherein the scFv light chain variable domain comprises an amino acid sequence that has at least 80% sequence identity to the amino acid sequence according to SEQ ID NO: 8.

Embodiment 125 comprises a multispecific antibody of any one of embodiments 86-114, wherein the scFv light chain variable domain comprises an amino acid sequence of at least 75 consecutive amino acid residues of SEQ ID NO: 8.

Embodiment 126 comprises a multispecific antibody of any one of embodiments 86-114, wherein the scFv light chain variable domain comprises an amino acid sequence of at least 100 consecutive amino acid residues of SEQ ID NO: 8.

Embodiment 127 comprises a multispecific antibody of any one of embodiments 86-114, wherein the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8.

Embodiment 128 comprises a multispecific antibody of any one of embodiments 86-114, wherein the scFv light chain variable domain comprises an amino acid sequence according to SEQ ID NO: 8.

Embodiment 129 comprises a multispecific antibody of any one of embodiments 86-114, wherein the scFv comprises an amino acid sequence that has at least 80% sequence identity to the amino acid sequence according to SEQ ID NO: 9.

Embodiment 130 comprises a multispecific antibody of any one of embodiments 86-114, wherein the scFv comprises an amino acid sequence of at least 175 consecutive amino acid residues of SEQ ID NO: 9.

Embodiment 131 comprises a multispecific antibody of any one of embodiments 86-114, wherein the scFv comprises an amino acid sequence of at least 210 consecutive amino acid residues of SEQ ID NO: 9.

Embodiment 132 comprises a multispecific antibody of any one of embodiments 86-114, wherein the scFv comprises an amino acid sequence of at least 210 consecutive amino acid residues of SEQ ID NO: 9 and has at least 80% sequence identity to the at least 210 consecutive amino acid residues of SEQ ID NO: 9.

Embodiment 133 comprises a multispecific antibody of any one of embodiments 86-114, wherein the scFv comprises an amino acid sequence according to SEQ ID NO: 9.

Embodiment 134 comprises a multispecific antibody of any one of embodiments 86-114, wherein the Fab heavy chain polypeptide comprises an amino acid sequence that has at least 80% sequence identity to the amino acid sequence according to SEQ ID NO: 17, 43, 45, or 47.

Embodiment 135 comprises a multispecific antibody of any one of embodiments 86-114, wherein the Fab heavy chain polypeptide comprises an amino acid sequence of at least 175 consecutive amino acid residues of SEQ ID NO: 17, 43, 45, or 47.

Embodiment 136 comprises a multispecific antibody of any one of embodiments 86-114, wherein the Fab heavy chain polypeptide comprises an amino acid sequence of at least 215 consecutive amino acid residues of SEQ ID NO: 17, 43, 45, or 47.

Embodiment 137 comprises a multispecific antibody of any one of embodiments 86-114, wherein the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17, 43, 45, or 47.

Embodiment 138 comprises a multispecific antibody of any one of embodiments 86-114, wherein the Fab heavy chain polypeptide comprises an amino acid sequence according to SEQ ID NO: 17, 43, 45, or 47.

Embodiment 139 comprises a multispecific antibody of any one of embodiments 86-114, wherein the Fab light chain polypeptide comprises an amino acid sequence that has at least 80% sequence identity to the amino acid sequence according to SEQ ID NO: 16, 42, 44, or 46.

Embodiment 140 comprises a multispecific antibody of any one of embodiments 86-114, wherein the Fab light chain polypeptide comprises an amino acid sequence of at least 175 consecutive amino acid residues of SEQ ID NO: 16, 42, 44, or 46.

Embodiment 141 comprises a multispecific antibody of any one of embodiments 86-114, wherein the Fab light chain polypeptide comprises an amino acid sequence of at least 200 consecutive amino acid residues of SEQ ID NO: 16, 42, 44, or 46.

Embodiment 142 comprises a multispecific antibody of any one of embodiments 86-114, wherein the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16, 42, 44, or 46.

Embodiment 143 comprises a multispecific antibody of any one of embodiments 86-114, wherein the Fab light chain polypeptide comprises an amino acid sequence according to SEQ ID NO: 16, 42, 44, or 46.

Embodiment 144 comprises a multispecific antibody of any one of embodiments 86-114, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

Embodiment 145 comprises a multispecific antibody of any one of embodiments 86-114, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21.

Embodiment 146 comprises a multispecific antibody of any one of embodiments 86-114, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21.

Embodiment 147 comprises a multispecific antibody of any one of embodiments 86-114, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

Embodiment 148 comprises a multispecific antibody of any one of embodiments 86-114, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

Embodiment 149 comprises a multispecific antibody of any one of embodiments 86-114, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22.

Embodiment 150 comprises a multispecific antibody of any one of embodiments 86-114, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22.

Embodiment 151 comprises a multispecific antibody of any one of embodiments 86-114, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

Embodiment 152 comprises a multispecific antibody of any one of embodiments 86-151, wherein the multispecific antibodies of Formula Ia, Formula Ib, Formula Ic further comprise a half-life extending molecule (H1).

Embodiment 153 comprises a multispecific antibody of embodiment 152, wherein H₁ is connected to P₁.

Embodiment 154 comprises a multispecific antibody of embodiment 152, wherein H₁ is connected to P₂.

Embodiment 155 comprises a multispecific antibody of any one of embodiments 152-154, wherein H₁ does not block A binding to CD28.

Embodiment 156 comprises a multispecific antibody of any one of embodiments 152-155, wherein H₁ does not block B binding to PD-L1.

Embodiment 157 comprises a multispecific antibody of any one of embodiments 152-156, H₁ comprises a linking moiety (L₅) that connects H₁ to P₁ or H₁ to P₂.

Embodiment 158 comprises a multispecific antibody of any one of embodiments 152-157, wherein the half-life extending molecule (H₁) does not have binding affinity to PD-L1.

Embodiment 159 comprises a multispecific antibody of any one of embodiments 152-158, wherein the half-life extending molecule (H₁) does not have binding affinity to CD28.

Embodiment 160 comprises a multispecific antibody of any one of embodiments 152-159, wherein the half-life extending molecule (H₁) does not shield the multispecific antibody from CD28.

Embodiment 161 comprises a multispecific antibody of any one of embodiments 152-160, wherein H₁ comprises a sequence according to SEQ ID NOs: 54-57.

Embodiment 162 comprises a multispecific antibody of any one of embodiments 152-161, wherein H₁ comprises an amino acid sequence that has repetitive sequence motifs.

Embodiment 163 comprises a multispecific antibody of any one of embodiments 152-162, wherein H₁ comprises an amino acid sequence that has highly ordered secondary structure.

Embodiment 164 comprises a multispecific antibody of any one of embodiments 152-163, wherein H₁ comprises a polymer.

Embodiment 165 comprises a multispecific antibody of embodiment 164, wherein the polymer is polyethylene glycol (PEG).

Embodiment 166 comprises a multispecific antibody of any one of embodiments of embodiments 152-163, wherein H₁ comprises albumin.

Embodiment 167 comprises a multispecific antibody of any one of embodiments of embodiments 152-163, wherein H₁ comprises an Fc domain.

Embodiment 168 comprises a multispecific antibody of any one of embodiments of embodiment 166, wherein the albumin is serum albumin.

Embodiment 169 comprises a multispecific antibody of embodiment 168, wherein the albumin is human serum albumin.

Embodiment 170 comprises a multispecific antibody of any one of embodiments of embodiments 152-163, wherein H₁ comprises a polypeptide, a ligand, or a small molecule.

Embodiment 171 comprises a multispecific antibody of embodiment 170, wherein the polypeptide, the ligand or the small molecule binds serum protein or a fragment thereof, a circulating immunoglobulin or a fragment thereof, or CD35/CR1.

Embodiment 172 comprises a multispecific antibody of embodiment 170, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 173 comprises a multispecific antibody of embodiment 171, wherein the circulating immunoglobulin molecule comprises IgG1, IgG2, IgG3, IgG4, slgA, IgM or IgD.

Embodiment 174 comprises a multispecific antibody of embodiment 171, wherein the serum protein is albumin.

Embodiment 175 comprises a multispecific antibody of embodiment 170, wherein the polypeptide is an antibody.

Embodiment 176 comprises a multispecific antibody of embodiment 175, wherein the antibody comprises a single domain antibody, a single chain variable fragment or a Fab.

Embodiment 177 comprises a multispecific antibody of embodiment 176, wherein the single domain antibody comprises a single domain antibody that binds to albumin.

Embodiment 178 comprises a multispecific antibody of embodiment 177, wherein the single domain antibody is a human or humanized antibody.

Embodiment 179 comprises a multispecific antibody of any one of embodiments 176-178, wherein the single domain antibody is selected from the group consisting of 645gHlgL1, 645dsgH5gL4, 23-13-A01-sc02, A10m3 or a fragment thereof, DOM7r-31, DOM7h-11-15, Alb-1, Alb-8, Alb-23, 10G, 10E and SA21.

Embodiment 180 comprises a multispecific antibody of embodiment 176, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3.

Embodiment 181 comprises a multispecific antibody of embodiment 176, wherein H₁ comprises an amino acid sequence according to SEQ ID NO: 57.

Embodiment 182 comprises a multispecific antibody of embodiment 176, wherein H₁ comprises an amino acid sequence that has at least 80% sequence identity to SEQ ID NO: 57.

Embodiment 183 comprises a multispecific antibody of embodiment 176, wherein H₁ comprises an amino acid sequence that has at least 85% sequence identity to SEQ ID NO: 57.

Embodiment 184 comprises a multispecific antibody of embodiment 176, wherein H₁ comprises an amino acid sequence that has at least 90% sequence identity to SEQ ID NO: 57.

Embodiment 185 comprises a multispecific antibody of embodiment 176, wherein H₁ comprises an amino acid sequence that has at least 95% sequence identity to SEQ ID NO: 57.

Embodiment 186 comprises a multispecific antibody of embodiment 176, wherein H₁ comprises an amino acid sequence that has at least 99% sequence identity to SEQ ID NO: 57.

Embodiment 187 comprises a multispecific antibody of any one of embodiments 152-186, wherein H₁ comprise a modified amino acid or non-natural amino acid, or a modified non-natural amino acid, or a combination thereof.

Embodiment 188 comprises a multispecific antibody of embodiment 187, wherein the modified amino acid or a modified non-natural amino acid comprises a post-translational modification.

Embodiment 189 comprises a multispecific antibody of any one of embodiments 152-188, wherein H₁ comprises a linking moiety (L₅) that connects H₁ to P₁ or P₂.

Embodiment 190 comprises a multispecific antibody of embodiment 189, wherein L₅ is a peptide sequence having at least 5 to no more than 50 amino acids.

Embodiment 191 comprises a multispecific antibody of embodiment 190, wherein L₅ is a peptide sequence having at least 10 to no more than 30 amino acids.

Embodiment 192 comprises a multispecific antibody of embodiment 191, wherein L₅ is a peptide sequence having at least 10 amino acids.

Embodiment 193 comprises a multispecific antibody of embodiment 192, wherein L₅ is a peptide sequence having at least 18 amino acids.

Embodiment 194 comprises a multispecific antibody of embodiment 193, wherein L₅ is a peptide sequence having at least 26 amino acids.

Embodiment 195 comprises a multispecific antibody of embodiment 189, wherein L₅ has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 196 comprises a multispecific antibody of any one of embodiments 86-195, wherein L₁ or L₂ is a peptide sequence having at least 5 to no more than 50 amino acids.

Embodiment 197 comprises a multispecific antibody of any one of embodiments 86-195, wherein L₁ or L₂ is a peptide sequence having at least 10 to no more than 30 amino acids.

Embodiment 198 comprises a multispecific antibody of any one of embodiments 86-195, wherein L₁ or L₂ is a peptide sequence having at least 10 amino acids.

Embodiment 199 comprises a multispecific antibody of any one of embodiments 86-195, wherein L₁ or L₂ is a peptide sequence having at least 18 amino acids.

Embodiment 200 comprises a multispecific antibody of any one of embodiments 86-195, wherein L₁ or L₂ is a peptide sequence having at least 26 amino acids.

Embodiment 201 comprises a multispecific antibody of any one of embodiments 86-195, wherein L₁ or L₂ has a formula comprising (G2S)ₙ (SEQ ID NO: 233), wherein n is an integer from 1 to 3.

Embodiment 202 comprises a multispecific antibody of any one of embodiments 86-195, wherein L₁ or L₂ has a formula comprising (G2S)ₙ, wherein n is an integer of at least 1.

Embodiment 203 comprises a multispecific antibody of any one of embodiments 86-195, wherein L₁ or L₂ has a formula selected from the group consisting of (G₂S)ₙ, (GS)ₙ, (GSGGS)y (SEQ ID NO: 58), (GGGS)˜(SEQ ID NO: 59), (GGGGS)ₙ (SEQ ID NO: 60), and (GSSGGS)ₙ (SEQ ID NO: 61), wherein n is an integer of at least 1.

Embodiment 204 comprises a multispecific antibody of any one of embodiments 86-203, wherein the tumor specific protease is selected from the group consisting of metalloprotease, serine protease, cysteine protease, threonine protease, and aspartic protease.

Embodiment 205 comprises a multispecific antibody of any one of embodiments 86-203, wherein L₁ or L₂ comprises a urokinase cleavable amino acid sequence, a matriptase cleavable amino acid sequence, a legumain cleavable amino acid sequence, or a matrix metalloprotease cleavable amino acid sequence.

Embodiment 206 comprises a multispecific antibody of any one of embodiments 86-205, wherein L₁ or L₂ comprises a sequence according to SEQ ID NOs: 18-19, 62-88.

Embodiment 207 comprises a multispecific antibody of any one of embodiments 86-206, wherein L₁ is bound to N-terminus of A.

Embodiment 208 comprises a multispecific antibody of any one of embodiments 86-206, wherein L₁ is bound to C-terminus of A.

Embodiment 209 comprises a multispecific antibody of any one of embodiments 86-206, wherein L₂ is bound to N-terminus of B.

Embodiment 210 comprises a multispecific antibody of any one of embodiments 86-206, wherein L₂ is bound to C-terminus of B.

Embodiment 211 comprises a multispecific antibody of any one of embodiments 86-206, wherein P₁ becomes unbound from A when L₁ is cleaved by the tumor specific protease thereby exposing A to CD28.

Embodiment 212 comprises a multispecific antibody of any one of embodiments 86-206, wherein P₂ becomes unbound from B when L₂ is cleaved by the tumor specific protease thereby exposing B to PD-L1.

Embodiment 213 comprises a multispecific antibody of any one of embodiments 86-213, wherein L₁ or L₂, comprise a modified amino acid or non-natural amino acid, or a modified non-natural amino acid, or a combination thereof.

Embodiment 214 comprises a multispecific antibody of embodiment 213, wherein the modified amino acid or a modified non-natural amino acid comprises a post-translational modification.

Embodiment 215 comprises a multispecific antibody of any one of embodiments 86-214, wherein P₁ impairs binding of A to CD28.

Embodiment 216 comprises a multispecific antibody of any one of embodiments 86-215, wherein P₁ is bound to A through ionic interactions, electrostatic interactions, hydrophobic interactions, Pi-stacking interactions, and H-bonding interactions, or a combination thereof.

Embodiment 217 comprises a multispecific antibody of any one of embodiments 86-216, wherein P₁ is bound to A at or near an antigen binding site.

Embodiment 218 comprises a multispecific antibody of any one of embodiments 86-217, wherein P₁ becomes unbound from A when L1 is cleaved by the tumor specific protease thereby exposing A to CD28.

Embodiment 219 comprises a multispecific antibody of any one of embodiments 86-218, wherein P₁ has less than 75% sequence identity to CD28.

Embodiment 220 comprises a multispecific antibody of any one of embodiments 86-218, wherein P₁ has less than 80% sequence identity to CD28.

Embodiment 221 comprises a multispecific antibody of any one of embodiments 86-218, wherein P₁ has less than 85% sequence identity to CD28.

Embodiment 222 comprises a multispecific antibody of any one of embodiments 86-218, wherein P₁ has less than 90% sequence identity to CD28.

Embodiment 223 comprises a multispecific antibody of any one of embodiments 86-218, wherein P₁ has less than 95% sequence identity to CD28.

Embodiment 224 comprises a multispecific antibody of any one of embodiments 86-218, wherein P₁ comprises a de novo amino acid sequence that shares less than 10% sequence identity to CD28.

Embodiment 225 comprises a multispecific antibody of any one of embodiments 87-224, wherein P₂ impairs binding of B to PD-L1.

Embodiment 226 comprises a multispecific antibody of any one of embodiments 87-224, wherein P₂ is bound to B through ionic interactions, electrostatic interactions, hydrophobic interactions, Pi-stacking interactions, and H-bonding interactions, or a combination thereof.

Embodiment 227 comprises a multispecific antibody of any one of embodiments 87-224, wherein P₂ is bound to B at or near an antigen binding site.

Embodiment 228 comprises a multispecific antibody of any one of embodiments 87-224, wherein P₂ becomes unbound from B when L₂ is cleaved by the tumor specific protease thereby exposing B to the PD-L1.

Embodiment 229 comprises a multispecific antibody of any one of embodiments 87-224, wherein P₂ has less than 70% sequence identity to the PD-L1.

Embodiment 230 comprises a multispecific antibody of any one of embodiments 87-224, wherein P₂ has less than 75% sequence identity to the PD-L1.

Embodiment 231 comprises a multispecific antibody of any one of embodiments 87-224, wherein P₂ has less than 80% sequence identity to the PD-L1.

Embodiment 232 comprises a multispecific antibody of any one of embodiments 87-224, wherein P₂ has less than 85% sequence identity to the PD-L1.

Embodiment 233 comprises a multispecific antibody of any one of embodiments 87-224, wherein P₂ has less than 90% sequence identity to the PD-L1.

Embodiment 234 comprises a multispecific antibody of any one of embodiments 87-224, wherein P₂ has less than 95% sequence identity to the PD-L1.

Embodiment 235 comprises a multispecific antibody of any one of embodiments 87-224, wherein P₂ comprises a de novo amino acid sequence that shares less than 10% sequence identity to the PD-L1.

Embodiment 236 comprises a multispecific antibody of any one of embodiments 86-235, wherein P₁ or P₂ comprises a peptide sequence of at least 5 amino acids in length.

Embodiment 237 comprises a multispecific antibody of any one of embodiments 86-235, wherein P₁ or P₂ comprises a peptide sequence of at least 6 amino acids in length.

Embodiment 238 comprises a multispecific antibody of any one of embodiments 86-235, wherein P₁ or P₂ comprises a peptide sequence of at least 10 amino acids in length.

Embodiment 239 comprises a multispecific antibody of any one of embodiments 86-235, wherein P₁ or P₂ comprises a peptide sequence of at least 10 amino acids in length and no more than 20 amino acids in length.

Embodiment 240 comprises a multispecific antibody of any one of embodiments 86-235, wherein P₁ or P₂ comprises a peptide sequence of at least 16 amino acids in length.

Embodiment 241 comprises a multispecific antibody of any one of embodiments 86-235, wherein P₁ or P₂ comprises a peptide sequence of no more than 40 amino acids in length.

Embodiment 242 comprises a multispecific antibody of any one of embodiments 86-241, wherein P₁ or P₂ comprises at least two cysteine amino acid residues.

Embodiment 243 comprises a multispecific antibody of any one of embodiments 86-242, wherein P₁ or P₂ comprises a cyclic peptide or a linear peptide.

Embodiment 244 comprises a multispecific antibody of any one of embodiments 86-242, wherein P₁ or P₂ comprises a cyclic peptide.

Embodiment 245 comprises a multispecific antibody of any one of embodiments 86-242, wherein P₁ or P₂ comprises a linear peptide.

Embodiment 246 comprises a multispecific antibody of any one of embodiments 86-245, wherein P₁ or P₂ comprise a modified amino acid or non-natural amino acid, or a modified non-natural amino acid, or a combination thereof.

Embodiment 247 comprises a multispecific antibody of any one of embodiments 86-246, wherein P1 or P₂ does not comprise albumin or an albumin fragment.

Embodiment 248 comprises a multispecific antibody of any one of embodiments 86-247, wherein P1 or P₂ does not comprise an albumin binding domain.

Embodiment 249 comprises an isolated recombinant nucleic acid molecule encoding a polypeptide of the multispecific antibody of any one of embodiments 1-248.

Embodiment 250 comprises a pharmaceutical composition comprising: (a) the multispecific antibody of any one of embodiments 1-248; and (b) a pharmaceutically acceptable excipient.

Embodiment 251 comprises a pharmaceutical composition comprising: (a) the multispecific antibody of any one of embodiments 1-248, (b) an anti-cancer therapy, and (c) a pharmaceutically acceptable excipient.

Embodiment 252 comprises a pharmaceutical composition of embodiment 251, wherein the anti-cancer therapy comprises a small molecule, a cell-based therapy, or an antibody-based therapy.

Embodiment 253 comprises a pharmaceutical composition of embodiment 252, wherein the antibody-based therapy is a T cell engager.

Embodiment 254 comprises a pharmaceutical composition of embodiment 253, wherein the T cell engager comprises a formula according to: D-L₀-E (Formula II), wherein D comprises an effector cell binding domain that binds to an effector cell antigen, E comprises a tumor antigen binding domain that binds to a tumor antigen, and L₀ comprises a linker that connects D to E.

Embodiment 255 comprises a pharmaceutical composition of embodiment 254, wherein D comprises a single chain variable fragment, a single domain antibody, or a Fab fragment.

Embodiment 256 comprises a pharmaceutical composition of embodiment 255, wherein D comprises the single chain variable fragment.

Embodiment 257 comprises a pharmaceutical composition of any one of embodiments 254-256, wherein E comprises a single chain variable fragment, a single domain antibody, or a Fab fragment.

Embodiment 258 comprises a pharmaceutical composition of embodiment 257, wherein E comprises the Fab fragment.

Embodiment 259 comprises a pharmaceutical composition of any one of embodiments 254-258, wherein the effector cell antigen comprises CD3.

Embodiment 260 comprises a pharmaceutical composition of embodiment 259, wherein the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 261 comprises a pharmaceutical composition of embodiment 259, wherein the effector cell binding domain comprises an amino acid sequence according to SEQ ID NOs: 89-101.

Embodiment 262 comprises a pharmaceutical composition of any one of embodiments 254-261, wherein the tumor antigen comprises epidermal growth factor receptor (EGFR), prostate-specific membrane antigen (PSMA), or tumor-associated calcium signal transducer 2 (referred to herein after as TROP2).

Embodiment 263 comprises a pharmaceutical composition of embodiment 262, wherein the tumor antigen comprises EGFR.

Embodiment 264 comprises a pharmaceutical composition of embodiment 263, wherein the tumor antigen binding domain comprises an amino acid sequence according to SEQ ID NOs: 102-111.

Embodiment 265 comprises a pharmaceutical composition of embodiment 263, wherein the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise:LC-CDR1 SEQ ID NO: 102; LC-CDR2: SEQ ID NO: 103; and LC-CDR3: SEQ ID NO: 104.

Embodiment 266 comprises a pharmaceutical composition of embodiment 263, wherein the tumor antigen comprises EGFR, and the T cell engager comprises amino acid sequences with at least 95% sequence identity according to SEQ ID NOs: 139-142.

Embodiment 267 comprises a pharmaceutical composition of embodiment 263, wherein the tumor antigen comprises EGFR, and the T cell engager comprises amino acid sequences according to SEQ ID NOs: 139-142.

Embodiment 268 comprises a pharmaceutical composition of embodiment 262, wherein the tumor antigen comprises TROP2.

Embodiment 269 comprises a pharmaceutical composition of embodiment 268, wherein the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise:LC-CDR1 SEQ ID NO: 115; LC-CDR2: SEQ ID NO: 116; and LC-CDR3: SEQ ID NO: 117.

Embodiment 270 comprises a pharmaceutical composition of embodiment 268, wherein the tumor antigen comprises TROP2, and the T cell engager comprises amino acid sequences with at least 95% sequence identity according to SEQ ID NOs: 143-150.

Embodiment 271 comprises a pharmaceutical composition of embodiment 268, Wherein the tumor antigen comprises TROP2, and the T cell engager comprises amino acid sequences according to SEQ ID NOs: 143-150.

Embodiment 272 comprises a pharmaceutical composition of embodiment 268, wherein the tumor antigen binding domain comprises an amino acid sequence according to SEQ ID NOs: 112-119.

Embodiment 273 comprises a pharmaceutical composition of embodiment 262, wherein the tumor antigen comprises PSMA.

Embodiment 274 comprises a pharmaceutical composition of embodiment 273, wherein the tumor antigen binding domain comprises an amino acid sequence according to SEQ ID NOs: 120-127.

Embodiment 275 comprises a pharmaceutical composition of embodiment 273, wherein the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise:LC-CDR1 SEQ ID NO: 123; LC-CDR2: SEQ ID NO: 124; and LC-CDR3: SEQ ID NO: 125.

Embodiment 276 comprises a pharmaceutical composition of embodiment 273, wherein the tumor antigen comprises PSMA, and the T cell engager comprises amino acid sequences with at least 95% sequence identity according to SEQ ID NOs: 151-160.

Embodiment 277 comprises a pharmaceutical composition of embodiment 273, wherein the tumor antigen comprises PSMA, and the T cell engager comprises amino acid sequences according to SEQ ID NOs: 151-160.

Embodiment 278 comprises a pharmaceutical composition of any one of embodiments 253-277, wherein the T cell engager molecule is selectively activated in tumor microenvironments.

Embodiment 279 comprises a pharmaceutical composition of any one of embodiments 253-278, wherein the T cell engager is according to the following subformula: P₃-L₃-D-L₀-E (Formula IIa) wherein D comprises the CD3 binding domain; E comprises the tumor antigen binding domain; L0 comprises the linker that connects D to E; P₃ comprises a peptide that binds to D and L₃ comprises a linking moiety that connects D to P₃ and is a substrate for a tumor specific protease.

Embodiment 280 comprises a pharmaceutical composition of any one of embodiments 253-278, wherein the T cell engager is according to the following subformula: D-L₀-E-L₄-P₄ (Formula IIb) wherein D comprises the CD3 binding domain; E comprises the tumor antigen binding domain; L0 comprises the linker that connects D to E; P4 comprises a peptide that binds to E and L4 comprises a linking moiety that connects E to P4 and is a substrate for a tumor specific protease.

Embodiment 281 comprises a pharmaceutical composition of any one of embodiments 253-278, wherein the T cell engager is according to the following subformula: P₃-L₃-D-L₀-E-L₄-P₄ (Formula IIc) wherein D comprises the CD3 binding domain; E comprises the tumor antigen binding domain; L0 comprises the linker that connects D to E; P₃ comprises a peptide that binds to D and L₃ comprises a linking moiety that connects D to P₃ and is a substrate for a tumor specific protease; P₄ comprises a peptide that binds to E and L₄ comprises a linking moiety that connects E to P₄ and is a substrate for a tumor specific protease.

Embodiment 282 comprises a pharmaceutical composition of any one of embodiments 253-281, wherein the T cell engager comprises H₁.

Embodiment 283 comprises a pharmaceutical composition of embodiment 282, wherein H₁ comprises a sequence according to SEQ ID NO: 54-57.

Embodiment 284 comprises a pharmaceutical composition of any one of embodiments 282-283, wherein H₁ comprises a single domain antibody.

Embodiment 285 comprises a pharmaceutical composition of any one of embodiments 282-284, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56.

Embodiment 286 comprises a pharmaceutical composition of any one of embodiments 279-285, wherein L₃ or L₄ is a peptide sequence having at least 5 to no more than 50 amino acids.

Embodiment 287 comprises a pharmaceutical composition of any one of embodiments 279-285, wherein L₃ or L₄ is a peptide sequence having at least 10 to no more than 30 amino acids.

Embodiment 288 comprises a pharmaceutical composition of any one of embodiments 279-285, wherein L₃ or L₄ is a peptide sequence having at least 10 amino acids.

Embodiment 289 comprises a pharmaceutical composition of any one of embodiments 279-285, wherein L₃ or L₄ is a peptide sequence having at least 18 amino acids.

Embodiment 290 comprises a pharmaceutical composition of any one of embodiments 279-285, wherein L₃ or L₄ is a peptide sequence having at least 26 amino acids.

Embodiment 291 comprises a pharmaceutical composition of any one of embodiments 279-285, wherein L₃ or L₄ has a formula comprising (G₂S)ₙ (SEQ ID NO: 233), wherein n is an integer from 1 to 3.

Embodiment 292 comprises a pharmaceutical composition of any one of embodiments 279-285, wherein L₃ or L₄ has a formula comprising (G₂S)ₙ, wherein n is an integer of at least 1.

Embodiment 293 comprises a pharmaceutical composition of any one of embodiments 279-285, wherein L₃ or L₄ has a formula selected from the group consisting of (G₂S)ₙ, (GS)ₙ, (GSGGS)ₙ (SEQ ID NO: 58), (GGGS)ₙ (SEQ ID NO: 59), (GGGGS)˜(SEQ ID NO: 60), and (GSSGGS)ₙ (SEQ ID NO: 61), wherein n is an integer of at least 1.

Embodiment 294 comprises a pharmaceutical composition of any one of embodiments 279-293, wherein the tumor specific protease is selected from the group consisting of metalloprotease, serine protease, cysteine protease, threonine protease, and aspartic protease.

Embodiment 295 comprises a pharmaceutical composition of any one of embodiments 279-293, wherein L₃ or L₄ comprises a urokinase cleavable amino acid sequence, a matriptase cleavable amino acid sequence, a legumain cleavable amino acid sequence, or a matrix metalloprotease cleavable amino acid sequence.

Embodiment 296 comprises a pharmaceutical composition of any one of embodiments 279-295, wherein L₃ or L₄ comprises a sequence according to SEQ ID NOs: 18-19, 62-88.

Embodiment 297 comprises a pharmaceutical composition of any one of embodiments 279-296, wherein L₃ is bound to N-terminus of D.

Embodiment 298 comprises a pharmaceutical composition of any one of embodiments 279-296, wherein L₃ is bound to C-terminus of D.

Embodiment 299 comprises a pharmaceutical composition of any one of embodiments 279-296, wherein L₄ is bound to N-terminus of E.

Embodiment 300 comprises a pharmaceutical composition of any one of embodiments 279-296, wherein L₄ is bound to C-terminus of E.

Embodiment 301 comprises a pharmaceutical composition of any one of embodiments 279-300, wherein P₃ becomes unbound from D when L₃ is cleaved by the tumor specific protease thereby exposing D to CD3.

Embodiment 302 comprises a pharmaceutical composition of any one of embodiments 279-301, wherein P₄ becomes unbound from E when L₄ is cleaved by the tumor specific protease thereby exposing E to the tumor antigen.

Embodiment 303 comprises a pharmaceutical composition of any one of embodiments 279-302, wherein P₃ impairs binding of D to CD3.

Embodiment 304 comprises a pharmaceutical composition of any one of embodiments 279-303, wherein P₃ is bound to D through ionic interactions, electrostatic interactions, hydrophobic interactions, Pi-stacking interactions, and H-bonding interactions, or a combination thereof.

Embodiment 305 comprises a pharmaceutical composition of any one of embodiments 279-304, wherein P₃ is bound to D at or near an antigen binding site.

Embodiment 306 comprises a pharmaceutical composition of any one of embodiments 279-305, wherein P₃ becomes unbound from D when L₃ is cleaved by the tumor specific protease thereby exposing D to CD3.

Embodiment 307 comprises a pharmaceutical composition of any one of embodiments 279-306, wherein P₃ has less than 70% sequence identity to CD3.

Embodiment 308 comprises a pharmaceutical composition of any one of embodiments 279-306, wherein P₃ has less than 85% sequence identity to CD3.

Embodiment 309 comprises a pharmaceutical composition of any one of embodiments 279-306, wherein P₃ has less than 90% sequence identity to CD3.

Embodiment 310 comprises a pharmaceutical composition of any one of embodiments 279-306, wherein P₃ has less than 95% sequence identity to CD3.

Embodiment 311 comprises a pharmaceutical composition of any one of embodiments 279-306, wherein P₃ has less than 98% sequence identity to CD3.

Embodiment 312 comprises a pharmaceutical composition of any one of embodiments 279-306, wherein P₃ has less than 99% sequence identity to CD3.

Embodiment 313 comprises a pharmaceutical composition of any one of embodiments 279-306, wherein P₃ comprises the amino acid sequence according to SEQ ID NO: 161 (GSQCLGPEWEVCPY) or SEQ ID NO: 162 (VYCGPEFDESVGCM).

Embodiment 314 comprises a pharmaceutical composition of any one of embodiments 279-306, wherein P₃ comprises a de novo amino acid sequence that shares less than 10% sequence identity to CD3.

Embodiment 315 comprises a pharmaceutical composition of any one of embodiments 280-314, wherein P₄ impairs binding of E to the tumor antigen.

Embodiment 316 comprises a pharmaceutical composition of any one of embodiments 280-315, wherein P₄ is bound to E through ionic interactions, electrostatic interactions, hydrophobic interactions, Pi-stacking interactions, and H-bonding interactions, or a combination thereof.

Embodiment 317 comprises a pharmaceutical composition of any one of embodiments 280-316, wherein P₄ is bound to E at or near an antigen binding site.

Embodiment 318 comprises a pharmaceutical composition of any one of embodiments 280-317, wherein P₄ becomes unbound from E when L4 is cleaved by the tumor specific protease thereby exposing E to the tumor antigen.

Embodiment 319 comprises a pharmaceutical composition of any one of embodiments 280-318, wherein P₄ has less than 70% sequence identity to the tumor antigen.

Embodiment 320 comprises a pharmaceutical composition of any one of embodiments 280-318, wherein P₄ has less than 80% sequence identity to the tumor antigen.

Embodiment 321 comprises a pharmaceutical composition of any one of embodiments 280-318, wherein P₄ has less than 85% sequence identity to the tumor antigen.

Embodiment 322 comprises a pharmaceutical composition of any one of embodiments 280-318, wherein P₄ has less than 90% sequence identity to the tumor antigen.

Embodiment 323 comprises a pharmaceutical composition of any one of embodiments 280-318, wherein P₄ has less than 95% sequence identity to the tumor antigen.

Embodiment 324 comprises a pharmaceutical composition of any one of embodiments 280-318, wherein P₄ comprises a de novo amino acid sequence that shares less than 10% sequence identity to the tumor antigen.

Embodiment 325 comprises a pharmaceutical composition of any one of embodiments 279-324, wherein P₃ or P₄ comprises a peptide sequence of at least 5 amino acids in length.

Embodiment 326 comprises a pharmaceutical composition of any one of embodiments 279-324, wherein P₃ or P₄ comprises a peptide sequence of at least 6 amino acids in length.

Embodiment 327 comprises a pharmaceutical composition of any one of embodiments 279-324, wherein P₃ or P₄ comprises a peptide sequence of at least 10 amino acids in length.

Embodiment 328 comprises a pharmaceutical composition of any one of embodiments 279-324, wherein P₃ or P₄ comprises a peptide sequence of at least 10 amino acids in length and no more than 20 amino acids in length.

Embodiment 329 comprises a pharmaceutical composition of any one of embodiments 279-328, wherein P₃ or P₄ comprises a peptide sequence of at least 16 amino acids in length.

Embodiment 330 comprises a pharmaceutical composition of any one of embodiments 279-329, wherein P₃ or P₄ comprises a peptide sequence of no more than 40 amino acids in length.

Embodiment 331 comprises a pharmaceutical composition of any one of embodiments 279-330, wherein P₃ or P₄ comprises at least two cysteine amino acid residues.

Embodiment 332 comprises a pharmaceutical composition of any one of embodiments 279-331, wherein P₃ or P₄ comprises a cyclic peptide or a linear peptide.

Embodiment 333 comprises a pharmaceutical composition of any one of embodiments 279-332, wherein P₃ or P₄ comprises a cyclic peptide.

Embodiment 334 comprises a pharmaceutical composition of any one of embodiments 279-332, wherein P₃ or P₄ comprises a linear peptide.

Embodiment 335 comprises a pharmaceutical composition of any one of embodiments 280-334, wherein P4 comprises the amino acid sequence according to SEQ ID NO: 163 (PCRSHIDVAKPICV).

Embodiment 336 comprises a pharmaceutical composition of any one of embodiments 279-335, wherein the tumor antigen comprises EGFR, and the T cell engager comprises the amino acid sequence SEQ ID NOs: 164-178.

Embodiment 337 comprises a pharmaceutical composition of any one of embodiments 280-335, wherein P4 comprises the amino acid sequence according to SEQ ID NO: 179 (SVLFCVKNLYCWVT), SEQ ID NO: 180 (VDFCKIYSWPVCHQ), SEQ ID NO: 181 (IDFCMLYNWPICAG).

Embodiment 338 comprises a pharmaceutical composition of any one of embodiments 279-335, wherein the tumor antigen comprises TROP2, and the T cell engager comprises the amino acid sequence SEQ ID NOs: 182-219.

Embodiment 339 comprises a pharmaceutical composition of any one of embodiments 279-335, wherein the tumor antigen comprises PSMA, and the T cell engager comprises the amino acid sequence SEQ ID NOs: 220-231.

Embodiment 340 comprises a method of treating cancer in a subject in need thereof comprising administering to the subject the multispecific antibody of any one of embodiments 1-248 or the pharmaceutical composition of embodiment 249.

Embodiment 341 comprises a method of embodiment 340, wherein the multispecific antibody induces T cell mediated cytotoxicity of tumor cells.

Embodiment 342 comprises a method of any one of embodiments 340-341, wherein the cancer is a hematological malignancy.

Embodiment 343 comprises a method of any one of embodiments 340-342, wherein the cancer is leukemia or lymphoma.

Embodiment 344 comprises a method of any one of embodiments 340-343, wherein the cancer is lymphoma, and wherein the lymphoma is B-cell lymphoma.

Embodiment 345 comprises a method of any one of embodiments 340-341, wherein the cancer is a solid tumor.

Embodiment 346 comprises a method of embodiment 345, wherein the solid tumor expresses PD-L1.

Embodiment 347 comprises a method of any one of embodiments 345-346, wherein the solid tumor is sarcoma, breast cancer, lung cancer, or carcinoma.

Embodiment 348 comprises a method of any one of embodiments 345-347, wherein the solid tumor is lung cancer, and wherein the lung cancer is non-small cell lung cancer.

Embodiment 349 comprises a method of any one of embodiments 340-348, wherein the multispecific antibody is administered in combination with an anti-cancer therapy.

Embodiment 350 comprises a method of embodiment 349, wherein the multispecific antibody and the anti-cancer therapy are administered in the same pharmaceutical composition.

Embodiment 351 comprises a method of embodiment 349, wherein the multispecific antibody and the anti-cancer therapy are administered as separate pharmaceutical compositions.

Embodiment 352 comprises a method of any one of embodiments 340-351, wherein the subject is refractory to checkpoint inhibitor therapy.

Embodiment 353 comprises a method of any one of embodiments 340-352, wherein the subject has relapsed from checkpoint inhibitor therapy.

Embodiment 354 comprises a method of any one of embodiments 349-353, wherein the anti-cancer therapy comprises a small molecule, a cell-based therapy, or an antibody-based therapy.

Embodiment 355 comprises a method of embodiment 354, wherein the antibody-based therapy is a T cell engager.

Embodiment 356 comprises a method of embodiment 355, wherein the T cell engager comprises a formula according to: D-L₀-E (Formula II), wherein D comprises an effector cell binding domain that binds to an effector cell antigen, E comprises a tumor antigen binding domain that binds to a tumor antigen, and L0 comprises a linker that connects D to E.

Embodiment 357 comprises a method of embodiment 356, wherein D comprises a single chain variable fragment, a single domain antibody, or a Fab fragment.

Embodiment 358 comprises a method of embodiment 357, wherein D comprises the single chain variable fragment.

Embodiment 359 comprises a method of any one of embodiments 356-358, wherein E comprises a single chain variable fragment, a single domain antibody, or a Fab fragment.

Embodiment 360 comprises a method of embodiment 359, wherein E comprises the Fab fragment.

Embodiment 361 comprises a method of any one of embodiments 356-360, wherein the effector cell antigen comprises CD3.

Embodiment 362 comprises a method of any one of embodiments 356-361, wherein the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 363 comprises a method of any one of embodiments 356-362, wherein the effector cell binding domain comprises an amino acid sequence according to SEQ ID NOs: 89-101.

Embodiment 364 comprises a method of any one of embodiments 356-363, wherein the tumor antigen comprises epidermal growth factor receptor (EGFR), prostate-specific membrane antigen (PSMA), or tumor-associated calcium signal transducer 2 (referred to herein after as TROP2).

Embodiment 365 comprises a method of embodiment 364, wherein the tumor antigen comprises EGFR.

Embodiment 366 comprises a method of any one of embodiments 364-365, wherein the cancer has cells that express EGFR.

Embodiment 367 comprises a method of any one of embodiments 364-367, wherein the cancer comprises colorectal cancer (CRC), squamous cell carcinoma of the head and Neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarian cancer, bladder cancer, kidney cancer, or pancreatic cancer.

Embodiment 368 comprises a method of any one of embodiments 364-367, wherein the tumor antigen binding domain comprises an amino acid sequence according to SEQ ID NOs: 102-111.

Embodiment 369 comprises a method of any one of embodiments 364-368, wherein the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 105; HC-CDR2: SEQ ID NO: 106; HC-CDR3: SEQ ID NO: 107; and wherein the LC-CDR1, the LC-CDR2, and the LC-CDR3 comprise:LC-CDR1 SEQ ID NO: 102; LC-CDR2: SEQ ID NO: 103; and LC-CDR3: SEQ ID NO: 104.

Embodiment 370 comprises a method of any one of embodiments 364-369, wherein the tumor antigen comprises EGFR, and the T cell engager comprises amino acid sequences with at least 95% sequence identity according to SEQ ID NOs: 139-142.

Embodiment 371 comprises a method of any one of embodiments 364-369, wherein the tumor antigen comprises EGFR, and the T cell engager comprises amino acid sequences according to SEQ ID NOs: 139-142.

Embodiment 372 comprises a method of embodiment 364, wherein the tumor antigen comprises TROP2.

Embodiment 373 comprises a method of embodiment 372, wherein the cancer has cells that express TROP2.

Embodiment 374 comprises a method of any one of embodiments 372-373, wherein the cancer is a solid tumor cancer.

Embodiment 375 comprises a method of any one of embodiments 372-374, wherein the cancer is lung, breast (e.g. HER2+; ER/PR+; TNBC), cervical, ovarian, colorectal, pancreatic gastric, urothelial, endometrial, head and neck, or glioma.

Embodiment 376 comprises a method of any one of embodiments 372-375, wherein the tumor antigen binding domain comprises an amino acid sequence according to SEQ ID NOs: 112-119.

Embodiment 377 comprises a method of any one of embodiments 340-376, wherein the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 112; HC-CDR2: SEQ ID NO: 113; HC-CDR3: SEQ ID NO: 114; and wherein the LC-CDR1, the LC-CDR2, and the LC-CDR3 comprise:LC-CDR1 SEQ ID NO: 115; LC-CDR2: SEQ ID NO: 116; and LC-CDR3: SEQ ID NO: 117.

Embodiment 378 comprises a method of any one of embodiments 372-377, wherein the tumor antigen comprises TROP2, and the T cell engager comprises amino acid sequences with at least 95% sequence identity according to SEQ ID NOs: 143-150.

Embodiment 379 comprises a method of any one of embodiments 372-378, wherein the tumor antigen comprises TROP2, and the T cell engager comprises amino acid sequences according to SEQ ID NOs: 143-150.

Embodiment 380 comprises a method of embodiment 364, wherein the tumor antigen comprises PSMA.

Embodiment 381 comprises a method of embodiment 380, wherein the cancer comprises prostate cancer.

Embodiment 382 comprises a method of any one of embodiments 380-381, wherein the cancer comprises metastatic castrate-resistant prostate cancer (mCRPC).

Embodiment 383 comprises a method of any one of embodiments 380-382, wherein the tumor antigen binding domain comprises an amino acid sequence according to SEQ ID NOs: 120-127.

Embodiment 384 comprises a method of any one of embodiments 380-383, wherein the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 120; HC-CDR2: SEQ ID NO: 121; HC-CDR3: SEQ ID NO: 122; and wherein the LC-CDR1, the LC-CDR2, and the LC-CDR3 comprise:LC-CDR1: SEQ ID NO: 123; LC-CDR2: SEQ ID NO: 124; and LC-CDR3: SEQ ID NO: 125.

Embodiment 385 comprises a method of any one of embodiments 380-384, wherein the tumor antigen comprises PSMA, and the T cell engager comprises amino acid sequences with at least 95% sequence identity according to SEQ ID NOs: 151-160.

Embodiment 386 comprises a method of any one of embodiments 380-385, wherein the tumor antigen comprises PSMA, and the T cell engager comprises amino acid sequences according to SEQ ID NOs: 151-160.

Embodiment 387 comprises a method of any one of embodiments 356-386, wherein the T cell engager molecule is selectively activated in tumor microenvironments.

Embodiment 388 comprises a method of any one of embodiments 356-387, wherein the T cell engager is according to the following subformula: P₃-L₃-D-L₀-E (Formula IIa) wherein D comprises the CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease.

Embodiment 389 comprises a method of any one of embodiments 356-387, wherein the T cell engager is according to the following subformula: D-L₀-E-L₄-P₄ (Formula IIb) wherein D comprises the CD3 binding domain; E comprises the tumor antigen binding domain; L₀ comprises the linker that connects D to E; P₄ comprises a peptide that binds to E and L₄ comprises a linking moiety that connects E to P₄ and is a substrate for a tumor specific protease.

Embodiment 390 comprises a method of any one of embodiments 356-387, wherein the T cell engager is according to the following subformula: P₃-L₃-D-L₀-E-L₄-P₄ (Formula IIc) wherein D comprises the CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease; P4 comprises a peptide that binds to E and L₄ comprises a linking moiety that connects E to P₄ and is a substrate for a tumor specific protease.

Embodiment 391 comprises a method of any one of embodiments 356-390, wherein the T cell engager comprises H₁.

Embodiment 392 comprises a method of embodiment 391, wherein H₁ comprises a sequence according to SEQ ID NO: 54-57.

Embodiment 393 comprises a method of embodiment 391, wherein H₁ comprises a single domain antibody.

Embodiment 394 comprises a method of embodiment 393, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56.

Embodiment 395 comprises a method of any one of embodiments 388-394, wherein L₃ or L₄ is a peptide sequence having at least 5 to no more than 50 amino acids.

Embodiment 396 comprises a method of any one of embodiments 388-394, wherein L₃ or L₄ is a peptide sequence having at least 10 to no more than 30 amino acids.

Embodiment 397 comprises a method of any one of embodiments 388-394, wherein L₃ or L₄ is a peptide sequence having at least 10 amino acids.

Embodiment 398 comprises a method of any one of embodiments 388-394, wherein L₃ or L₄ is a peptide sequence having at least 18 amino acids.

Embodiment 399 comprises a method of any one of embodiments 388-394, wherein L₃ or L₄ is a peptide sequence having at least 26 amino acids.

Embodiment 400 comprises a method of any one of embodiments 388-394, wherein L₃ or L₄ has a formula comprising (G₂S)ₙ (SEQ ID NO: 233), wherein n is an integer from 1 to 3.

Embodiment 401 comprises a method of any one of embodiments 388-394, wherein L₃ or L₄ has a formula comprising (G₂S)ₙ, wherein n is an integer of at least 1.

Embodiment 402 comprises a method of any one of embodiments 388-394, wherein L3 or L4 has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 403 comprises a method of any one of embodiments 388-402, wherein the tumor specific protease is selected from the group consisting of metalloprotease, serine protease, cysteine protease, threonine protease, and aspartic protease.

Embodiment 404 comprises a method of any one of embodiments 388-402, wherein L₃ or L₄ comprises a urokinase cleavable amino acid sequence, a matriptase cleavable amino acid sequence, a legumain cleavable amino acid sequence, or a matrix metalloprotease cleavable amino acid sequence.

Embodiment 405 comprises a method of any one of embodiments 388-394, wherein L₃ or L₄ comprises a sequence according to SEQ ID NOs: 18-19, 62-88.

Embodiment 406 comprises a method of any one of embodiments 388-394, wherein L₃ is bound to N-terminus of D.

Embodiment 407 comprises a method of any one of embodiments 388-394, wherein L₃ is bound to C-terminus of D.

Embodiment 408 comprises a method of any one of embodiments 388-394, wherein L₄ is bound to N-terminus of E.

Embodiment 409 comprises a method of any one of embodiments 388-394, wherein L₄ is bound to C-terminus of E.

Embodiment 410 comprises a method of any one of embodiments 388-409, wherein P₃ becomes unbound from D when L₃ is cleaved by the tumor specific protease thereby exposing D to CD3.

Embodiment 411 comprises a method of any one of embodiments 388-410, wherein P₄ becomes unbound from E when L₄ is cleaved by the tumor specific protease thereby exposing E to the tumor antigen.

Embodiment 412 comprises a method of any one of embodiments 388-411, wherein P₃ impairs binding of D to CD3.

Embodiment 413 comprises a method of any one of embodiments 388-412, wherein P₃ is bound to D through ionic interactions, electrostatic interactions, hydrophobic interactions, Pi-stacking interactions, and H-bonding interactions, or a combination thereof.

Embodiment 414 comprises a method of any one of embodiments 388-413, wherein P₃ is bound to D at or near an antigen binding site.

Embodiment 415 comprises a method of any one of embodiments 388-414, wherein P₃ becomes unbound from D when L3 is cleaved by the tumor specific protease thereby exposing D to CD3.

Embodiment 416 comprises a method of any one of embodiments 388-415, wherein P₃ has less than 70% sequence identity to CD3.

Embodiment 417 comprises a method of any one of embodiments 388-415, wherein P₃ has less than 85% sequence identity to CD3.

Embodiment 418 comprises a method of any one of embodiments 388-415, wherein P₃ has less than 90% sequence identity to CD3.

Embodiment 419 comprises a method of any one of embodiments 388-415, wherein P₃ has less than 95% sequence identity to CD3.

Embodiment 420 comprises a method of any one of embodiments 388-415, wherein P₃ has less than 98% sequence identity to CD3.

Embodiment 421 comprises a method of any one of embodiments 388-415, wherein P₃ has less than 99% sequence identity to CD3.

Embodiment 422 comprises a method of any one of embodiments 388-421, wherein P₃ comprises the amino acid sequence according to SEQ ID NO: 161 (GSQCLGPEWEVCPY) or SEQ ID NO: 162 (VYCGPEFDESVGCM).

Embodiment 423 comprises a method of any one of embodiments 388-422, wherein P₃ comprises a de novo amino acid sequence that shares less than 10% sequence identity to CD3.

Embodiment 424 comprises a method of any one of embodiments 388-423, wherein P₄ impairs binding of E to the tumor antigen.

Embodiment 425 comprises a method of any one of embodiments 388-424, wherein P₄ is bound to E through ionic interactions, electrostatic interactions, hydrophobic interactions, Pi-stacking interactions, and H-bonding interactions, or a combination thereof.

Embodiment 426 comprises a method of any one of embodiments 388-425, wherein P₄ is bound to E at or near an antigen binding site.

Embodiment 427 comprises a method of any one of embodiments 388-426, wherein P₄ becomes unbound from E when L4 is cleaved by the tumor specific protease thereby exposing E to the tumor antigen.

Embodiment 428 comprises a method of any one of embodiments 388-427, wherein P₄ has less than 70% sequence identity to the tumor antigen.

Embodiment 429 comprises a method of any one of embodiments 388-428, wherein P₄ has less than 80% sequence identity to the tumor antigen.

Embodiment 430 comprises a method of any one of embodiments 388-429, wherein P₄ has less than 85% sequence identity to the tumor antigen.

Embodiment 431 comprises a method of any one of embodiments 388-430, wherein P₄ has less than 90% sequence identity to the tumor antigen.

Embodiment 432 comprises a method of any one of embodiments 388-431, wherein P₄ has less than 95% sequence identity to the tumor antigen.

Embodiment 433 comprises a method of any one of embodiments 388-432, wherein P₄ comprises a de novo amino acid sequence that shares less than 10% sequence identity to the tumor antigen.

Embodiment 434 comprises a method of any one of embodiments 388-433, wherein P₃ or P₄ comprises a peptide sequence of at least 5 amino acids in length.

Embodiment 435 comprises a method of any one of embodiments 388-434, wherein P₃ or P₄ comprises a peptide sequence of at least 6 amino acids in length.

Embodiment 436 comprises a method of any one of embodiments 388-435, wherein P₃ or P₄ comprises a peptide sequence of at least 10 amino acids in length.

Embodiment 437 comprises a method of any one of embodiments 388-436, wherein P₃ or P₄ comprises a peptide sequence of at least 10 amino acids in length and no more than 20 amino acids in length.

Embodiment 438 comprises a method of any one of embodiments 388-437, wherein P₃ or P₄ comprises a peptide sequence of at least 16 amino acids in length.

Embodiment 439 comprises a method of any one of embodiments 388-438, wherein P₃ or P₄ comprises a peptide sequence of no more than 40 amino acids in length.

Embodiment 440 comprises a method of any one of embodiments 388-439, wherein P₃ or P₄ comprises at least two cysteine amino acid residues.

Embodiment 441 comprises a method of any one of embodiments 388-440, wherein P₃ or P₄ comprises a cyclic peptide or a linear peptide.

Embodiment 442 comprises a method of any one of embodiments 388-440, wherein P₃ or P₄ comprises a cyclic peptide.

Embodiment 443 comprises a method of any one of embodiments 388-440, wherein P₃ or P₄ comprises a linear peptide.

Embodiment 444 comprises a method of any one of embodiments 388-440, wherein P₄ comprises the amino acid sequence according to SEQ ID NO: 163 (PCRSHIDVAKPICV).

Embodiment 445 comprises a method of any one of embodiments 364-390 wherein the tumor antigen comprises EGFR, and the T cell engager comprises the amino acid sequence SEQ ID NOs: 164-178.

Embodiment 446 comprises a method of any one of embodiments 364-390, wherein P4 comprises the amino acid sequence according to SEQ ID NO: 179 (SVLFCVKNLYCWVT), SEQ ID NO: 180 (VDFCKIYSWPVCHQ), SEQ ID NO: 181 (IDFCMLYNWPICAG).

Embodiment 447 comprises a method of any one of embodiments 364-390, wherein the tumor antigen comprises TROP2, and the T cell engager comprises the amino acid sequence SEQ ID NOs: 182-219.

Embodiment 448 comprises a method of any one of embodiments 364-390, wherein the tumor antigen comprises PSMA, and the T cell engager comprises the amino acid sequence SEQ ID NOs: 220-231.

Embodiment 449 comprises a method of treating cancer in a subject in need thereof comprising administering to the subject a multispecific antibody that comprises a CD28 binding domain and a PD-L1 binding domain wherein the multispecific antibody that comprises the CD28 binding domain and the PD-L1 binding domain is not administered as part of a treatment regimen with another multispecific antibody that targets a cancer antigen different from PD-L1 or CD28.

Embodiment 450 comprises a method of embodiment 449, wherein the multispecific antibody that comprises the CD28 binding domain and the PD-L1 binding domain is administered to the subject as a single agent therapy.

Embodiment 451 comprises a method of any one of embodiments 449-450, wherein the multispecific antibody that comprises the CD28 binding domain and the PD-L1 binding domain comprises an IgG framework, an IgA framework, an IgE framework, or an IgM framework.

Embodiment 452 comprises a method of any one of embodiments 449-451, wherein the CD28 binding domain comprises a single chain variable fragment, a single domain antibody, a Fab, or a Fab′.

Embodiment 453 comprises a method of any one of embodiments 449-452, wherein the PD-L1 binding domain comprises a single chain variable fragment, a single domain antibody, a Fab, or a Fab′.

Embodiment 454 comprises a method of any one of embodiments 449-453, wherein the PD-L1 binding domain comprises a single chain variable fragment and the CD28 binding domain comprises a single chain variable fragment.

Embodiment 455 comprises a method of any one of embodiments 449-454, wherein the CD28 binding domain comprises an anti-CD28 light chain polypeptide.

Embodiment 456 comprises a method of any one of embodiments 449-455, wherein the anti-CD28 light chain polypeptide comprises a variable domain of an IgG1, IgG2, IgG3, or IgG4 light chain.

Embodiment 457 comprises a method of any one of embodiments 449-456, wherein the CD28 binding domain comprises an anti-CD28 heavy chain polypeptide.

Embodiment 458 comprises a method of any one of embodiments 449-457, wherein the anti-CD28 heavy chain polypeptide comprises a variable domain of an IgG1, IgG2, IgG3, or IgG4 heavy chain.

Embodiment 459 comprises a method of any one of embodiments 449-458, wherein the PD-L1 binding domain comprises an anti-PD-L1 light chain polypeptide.

Embodiment 460 comprises a method of any one of embodiments 449-459, the anti-PD-L1 light chain polypeptide comprises a variable domain of an IgG1, IgG2, IgG3, or IgG4 light chain.

Embodiment 461 comprises a method of any one of embodiments 449-460, wherein the PD-L1 binding domain comprises an anti-PD-L1 heavy chain polypeptide.

Embodiment 462 comprises a method of any one of embodiments 449-461, the anti-PD-L1 heavy chain polypeptide comprises a variable domain of an IgG1, IgG2, IgG3, or IgG4 heavy chain.

Embodiment 463 comprises a method of any one of embodiments 449-462, wherein the multispecific antibody further comprises a fragment crystallizable (Fc) region.

Embodiment 464 comprises a method of embodiment 463, wherein the Fc region comprises an IgG CH2 domain and an IgG CH3 domain.

Embodiment 465 comprises a method of any one of embodiments 464-465, wherein the Fc region comprises a heterodimeric Fc region.

Embodiment 466 comprises a method of any one of embodiments 463-465, wherein the Fc region comprises at least one amino acid modification that increases the half-life of the multispecific antibody.

Embodiment 467 comprises a method of any one of embodiments 463-466, wherein the Fc region comprises at least one amino acid modification that modulates its interaction with an Fc receptor.

Embodiment 468 comprises a method of any one of embodiments 463-467, wherein the Fc region comprises at least one amino acid modification that increases binding of the Fc region to an Fc receptor.

Embodiment 469 comprises a method of any one of embodiments 463-468, wherein the Fc region comprises at least one amino acid modification that decreases glycosylation of the Fc region.

Embodiment 470 comprises a method of embodiment 469, wherein the modification is an amino acid substitution, deletion, or addition.

Embodiment 471 comprises a method of embodiment 470, wherein the modification is an amino acid substitution.

Embodiment 472 comprises a method of any one of embodiments 469-471, wherein the at least one amino acid modification that decreases glycosylation of the Fc region comprises an amino acid substitution at a position corresponding to position N297 of human IgG1, wherein the numbering is according to the EU index of Kabat.

Embodiment 473 comprises a method of any one of embodiments 463-472, wherein the Fc region is afucosylated.

Embodiment 474 comprises a method of any one of embodiments 455-473, wherein the anti-CD28 light chain polypeptide comprises complementarity determining regions (CDRs): LC-CDR1, LC-CDR2, and LC-CDR3, wherein the LC-CDR1, the LC-CDR2, and the LC-CDR3 the anti-CD28 light chain polypeptide: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3.

Embodiment 475 comprises a method of any one of embodiments 457-474, wherein the anti-CD28 heavy chain polypeptide comprises complementarity determining regions (CDRs): HC-CDR1, HC-CDR2, and HC-CDR3, wherein the HC-CDR1, the HC-CDR2, and the HC-CDR3 of the anti-CD28 heavy chain polypeptide comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

Embodiment 476 comprises a method of any one of embodiments 459-475, wherein the anti-PD-L1 light chain polypeptide comprises complementarity determining regions (CDRs): LC-CDR1, LC-CDR2, and LC-CDR3, wherein the LC-CDR1, the LC-CDR2, and the LC-CDR3 of the anti-PD-L1 light chain polypeptide comprise:LC-CDR1: SEQ ID NO: 13; LC-CDR2: SEQ ID NO: 14; LC-CDR1: SEQ ID NO: 33; LC-CDR2: SEQ ID NO: 34; LC-CDR3: SEQ ID NO: 35; LC-CDR1 SEQ ID NO: 36; LC-CDR2: SEQ ID NO: 37; and LC-CDR3: SEQ ID NO: 38; or LC-CDR1: SEQ ID NO: 39; LC-CDR2: SEQ ID NO: 40; and LC-CDR3: SEQ ID NO: 41; and LC-CDR3: SEQ ID NO: 15, and wherein the CDRs comprise from 0-2 amino acid modifications in at least one of said LC-CDR1, LC-CDR2, or LC-CDR3.

Embodiment 477 comprises a method of any one of embodiments 457-476, wherein the anti-PD-L1 heavy chain polypeptide the anti-PD-L1 heavy chain polypeptide comprises complementarity determining region (CDRs): HC-CDR1, HC-CDR2, and HC-CDR3, wherein the HC-CDR1, the HC-CDR2, and the HC-CDR3 of the anti-PD-L1 heavy chain polypeptide comprises: HC-CDR1: SEQ ID NO: 10; HC-CDR1: SEQ ID NO: 24; HC-CDR2: SEQ ID NO: 25; HC-CDR3: SEQ ID NO: 26; HC-CDR1: SEQ ID NO: 27; HC-CDR2: SEQ ID NO: 28; HC-CDR3: SEQ ID NO: 29; or HC-CDR1 SEQ ID NO: 30; HC-CDR2: SEQ ID NO: 31; HC-CDR3: SEQ ID NO: 32; HC-CDR2: SEQ ID NO: 11; HC-CDR3: SEQ ID NO: 12, and wherein said CDRs comprise from 0-2 amino acid modifications in at least one of said HC-CDR1, HC-CDR2, or HC-CDR3.

Embodiment 478 comprises a method of any one of embodiments 449-477, wherein the multispecific antibody induces T cell mediated cytotoxicity of tumor cells.

Embodiment 479 comprises a method of any one of embodiments 449-478, wherein the administering to the subject of the multispecific antibody is sufficient to reduce or eliminate the cancer as compared to a baseline measurement of the cancer taken from the subject prior to the administering of the multispecific antibody.

Embodiment 480 comprises a method of embodiment 479, wherein the reduction is at least about 1-fold, 5-fold, 10-fold, 20-fold, 40-fold, 60-fold, 80-fold, or up to about 100-fold.

Embodiment 481 comprises a method of any one of embodiments 449-480, wherein the cancer is a hematological malignancy.

Embodiment 482 comprises a method of any one of embodiments 449-480, wherein the cancer is leukemia or lymphoma.

Embodiment 483 comprises a method of any one of embodiments 449-480, wherein the cancer is lymphoma, and wherein the lymphoma is B-cell lymphoma.

Embodiment 484 comprises a method of any one of embodiments 449-480, wherein the cancer is a solid tumor.

Embodiment 485 comprises a method of embodiment 484, wherein the solid tumor expresses PD-L1.

Embodiment 486 comprises a method of any one of embodiments 484-485, wherein the solid tumor is sarcoma, breast cancer, lung cancer, or carcinoma.

Embodiment 487 comprises a method of embodiment 486, wherein the solid tumor is lung cancer, and wherein the lung cancer is non-small cell lung cancer.

Embodiment 488 comprises a method of any one of embodiments 449-487, wherein the multispecific antibody is selectively activated in tumor microenvironments.

Embodiment 489 comprises a method of any one of embodiments 449-488, wherein the multispecific antibody is according to the following formula: P₁-L₁-A-L-B (Formula Ia) wherein A comprises the CD28 binding domain; B comprises the PD-L1 binding domain; L comprises a linker that connects A to B; P₁ comprises a peptide that binds to A and L₁ comprises a linking moiety that connects A to P₁ and is a substrate for a tumor specific protease.

Embodiment 490 comprises a method of any one of embodiments 449-488, wherein the multispecific antibody is according to the following formula: A-L-B-L₂-P₂ (Formula Ib) wherein A comprises the CD28 binding domain; B comprises the PD-L1 binding domain; L comprises a linker that connects A to B; P₂ comprises a peptide that binds to B and L₂ comprises a linking moiety that connects B to P₂ and is a substrate for a tumor specific protease.

Embodiment 491 comprises a method of any one of embodiments 449-488, wherein the multispecific antibody is according to the following formula: P₁-L₁-A-L-B-L₂-P₂ (Formula Ic) wherein A comprises the CD28 binding domain; B comprises the PD-L1 binding domain; L comprises a linker that connects A to B; P₁ comprises a peptide that binds to A and L₁ comprises a linking moiety that connects A to P₁ and is a substrate for a tumor specific protease; P₂ comprises a peptide that binds to B and L₂ comprises a linking moiety that connects B to P₂ and is a substrate for a tumor specific protease.

Embodiment 492 comprises a method of any one of embodiments 489-491, wherein the linker connects the C-terminus of A to an N-terminus of B.

Embodiment 493 comprises a method of any one of embodiments 489-491, wherein the linker connects the N-terminus of A to a C-terminus of B.

Embodiment 494 comprises a method of any one of embodiments 489-491, wherein the linker connects the C-terminus of A to the N-terminus of the Fab heavy chain polypeptide.

Embodiment 495 comprises a method of any one of embodiments 489-491, wherein the linker connects the N-terminus of A to the C-terminus of the Fab heavy chain polypeptide.

Embodiment 496 comprises a method of any one of embodiments 489-491, wherein the linker connects the C-terminus of A to the N-terminus of the Fab light chain polypeptide.

Embodiment 497 comprises a method of any one of embodiments 489-491, wherein the linker connects the N-terminus of A to the C-terminus of the Fab light chain polypeptide.

Embodiment 498 comprises a method of any one of embodiments 489-491, wherein the linker connects the Fab light chain polypeptide to the scFv light chain variable domain.

Embodiment 499 comprises a method of any one of embodiments 489-491, wherein the linker connects the Fab light chain polypeptide to the scFv heavy chain variable domain.

Embodiment 500 comprises a method of any one of embodiments 489-491, wherein the linker connects the Fab heavy chain polypeptide to the scFv light chain variable domain.

Embodiment 501 comprises a method of any one of embodiments 489-491, wherein the linker connects the Fab heavy chain polypeptide to the scFv heavy chain variable domain.

Embodiment 502 comprises a method of any one of embodiments 489-491, wherein the linker connects the Fab light chain polypeptide to the N-terminus of the scFv light chain variable domain.

Embodiment 503 comprises a method of any one of embodiments 489-491, wherein the linker connects the Fab light chain polypeptide to the C-terminus of the scFv light chain variable domain.

Embodiment 504 comprises a method of any one of embodiments 489-491, wherein the linker connects the Fab light chain polypeptide to the N-terminus of the scFv heavy chain variable domain.

Embodiment 505 comprises a method of any one of embodiments 489-491, wherein the linker connects the Fab light chain polypeptide to the C-terminus of the scFv heavy chain variable domain.

Embodiment 506 comprises a method of any one of embodiments 489-491, wherein the linker connects the Fab heavy chain polypeptide to the N-terminus of the scFv light chain variable domain.

Embodiment 507 comprises a method of any one of embodiments 489-491, wherein the linker connects the Fab heavy chain polypeptide to the C-terminus of the scFv light chain variable domain.

Embodiment 508 comprises a method of any one of embodiments 489-491, wherein the linker connects the Fab heavy chain polypeptide to the N-terminus of the scFv heavy chain variable domain.

Embodiment 509 comprises a method of any one of embodiments 489-491, wherein the linker connects the Fab heavy chain polypeptide to the C-terminus of the scFv heavy chain variable domain.

Embodiment 510 comprises a method of any one of embodiments 489-509, wherein the linker is at least 5 amino acids in length.

Embodiment 511 comprises a method of any one of embodiments 489-509, wherein the linker is no more than 30 amino acids in length.

Embodiment 512 comprises a method of any one of embodiments 489-509, wherein the linker is at least 5 amino acids and no more than 30 amino acids in length.

Embodiment 513 comprises a method of any one of embodiments 489-509, wherein the linker is 5 amino acids in length.

Embodiment 514 comprises a method of any one of embodiments 489-509, wherein the linker is 15 amino acids in length.

Embodiment 515 comprises a method of any one of embodiments 489-509, wherein the linker is selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 516 comprises a method of any one of embodiments 489-509, wherein L has a formula comprising (G₂S)ₙ (SEQ ID NO: 233), wherein n is an integer from 1 to 3.

Embodiment 517 comprises a method of any one of embodiments 489-509, wherein the L comprises an amino acid sequence of SEQ ID NO: 18 (GGGGSGGGGSGGGGS) or SEQ ID NO: 19 (GGGGS).

Embodiment 518 comprises a method of any one of embodiments 489-517, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

Embodiment 519 comprises a method of any one of embodiments 489-517, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3.

Embodiment 520 comprises a method of any one of embodiments 489-517, wherein the Fab heavy chain variable domain comprises complementarity determining region (CDRs): HC-CDR1, HC-CDR2, and HC-CDR3, wherein the HC-CDR1, the HC-CDR2, and the HC-CDR3 of the Fab heavy chain variable domain comprise: HC-CDR1: SEQ ID NO: 10; HC-CDR2: SEQ ID NO: 11; HC-CDR3: SEQ ID NO: 12, HC-CDR1 SEQ ID NO: 24; HC-CDR2: SEQ ID NO: 25; HC-CDR3: SEQ ID NO: 26; or HC-CDR1 SEQ ID NO: 27; HC-CDR2: SEQ ID NO: 28; HC-CDR3: SEQ ID NO: 29; or HC-CDR1 SEQ ID NO: 30; HC-CDR2: SEQ ID NO: 31; HC-CDR3: SEQ ID NO: 32; and wherein said CDRs comprise from 0-2 amino acid modifications in at least one of said HC-CDR1, HC-CDR2, or HC-CDR3.

Embodiment 521 comprises a method of any one of embodiments 489-518, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise:LC-CDR1 SEQ ID NO: 13; LC-CDR2: SEQ ID NO: 14; and LC-CDR3: SEQ ID NO: 15; LC-CDR1 SEQ ID NO: 33; LC-CDR2: SEQ ID NO: 34; and LC-CDR3: SEQ ID NO: 35; LC-CDR1 SEQ ID NO: 36; LC-CDR2: SEQ ID NO: 37; and LC-CDR3: SEQ ID NO: 38; or LC-CDR1 SEQ ID NO: 39; LC-CDR2: SEQ ID NO: 40; and LC-CDR3: SEQ ID NO: 41; and wherein the CDRs comprise from 0-2 amino acid modifications in at least one of said LC-CDR1, LC-CDR2, or LC-CDR3.

Embodiment 522 comprises a method of any one of embodiments 489-517, wherein the scFv heavy chain variable domain comprises an amino acid sequence that has at least 80% sequence identity to the amino acid sequence according to SEQ ID NO: 7.

Embodiment 523 comprises a method of any one of embodiments 489-517, wherein the scFv heavy chain variable domain comprises an amino acid sequence of at least 75 consecutive amino acid residues of SEQ ID NO: 7

Embodiment 524 comprises a method of any one of embodiments 489-517, wherein the scFv heavy chain variable domain comprises an amino acid sequence of at least 110 consecutive amino acid residues of SEQ ID NO: 7.

Embodiment 525 comprises a method of any one of embodiments 489-517, wherein the scFv heavy chain variable domain comprises an amino acid sequence of at least 110 consecutive amino acid residues of SEQ ID NO: 7 and has at least 80% sequence identity to the at least 110 consecutive amino acid residues of SEQ ID NO: 7.

Embodiment 526 comprises a method of any one of embodiments 489-517, wherein the scFv heavy chain variable domain comprises an amino acid sequence according to SEQ ID NO: 7.

Embodiment 527 comprises a method of any one of embodiments 489-518, wherein the scFv light chain variable domain comprises an amino acid sequence that has at least 80% sequence identity to the amino acid sequence according to SEQ ID NO: 8.

Embodiment 528 comprises a method of any one of embodiments 489-517, wherein the scFv light chain variable domain comprises an amino acid sequence of at least 75 consecutive amino acid residues of SEQ ID NO: 8.

Embodiment 529 comprises a method of any one of embodiments 489-517, wherein the scFv light chain variable domain comprises an amino acid sequence of at least 100 consecutive amino acid residues of SEQ ID NO: 8.

Embodiment 530 comprises a method of any one of embodiments 489-517, wherein the scFv light chain variable domain comprises an amino acid sequence of at least 100 consecutive amino acid residues of SEQ ID NO: 8 and has at least 80% sequence identity to the at least 100 consecutive amino acid residues of SEQ ID NO: 8.

Embodiment 531 comprises a method of any one of embodiments 489-517, wherein the scFv light chain variable domain comprises an amino acid sequence according to SEQ ID NO: 8.

Embodiment 532 comprises a method of any one of embodiments 489-517, wherein the scFv comprises an amino acid sequence that has at least 80% sequence identity to the amino acid sequence according to SEQ ID NO: 9.

Embodiment 533 comprises a method of any one of embodiments 489-517, wherein the scFv comprises an amino acid sequence of at least 175 consecutive amino acid residues of SEQ ID NO: 9.

Embodiment 534 comprises a method of any one of embodiments 489-517, wherein the scFv comprises an amino acid sequence of at least 210 consecutive amino acid residues of SEQ ID NO: 9.

Embodiment 535 comprises a method of any one of embodiments 489-517, wherein the scFv comprises an amino acid sequence of at least 210 consecutive amino acid residues of SEQ ID NO: 9 and has at least 80% sequence identity to the at least 210 consecutive amino acid residues of SEQ ID NO: 9.

Embodiment 536 comprises a method of any one of embodiments 489-517, wherein the scFv comprises an amino acid sequence according to SEQ ID NO: 9.

Embodiment 537 comprises a method of any one of embodiments 489-517, wherein the Fab heavy chain polypeptide comprises an amino acid sequence that has at least 80% sequence identity to the amino acid sequence according to SEQ ID NO: 17, 43, 45, or 47.

Embodiment 538 comprises a method of any one of embodiments 489-517, wherein the Fab heavy chain polypeptide comprises an amino acid sequence of at least 175 consecutive amino acid residues of SEQ ID NO: 17, 43, 45, or 47.

Embodiment 539 comprises a method of any one of embodiments 489-517, wherein the Fab heavy chain polypeptide comprises an amino acid sequence of at least 215 consecutive amino acid residues of SEQ ID NO: 17, 43, 45, or 47.

Embodiment 540 comprises a method of any one of embodiments 489-517, wherein the Fab heavy chain polypeptide comprises an amino acid sequence of at least 215 consecutive amino acid residues of SEQ ID NO: 17 and has at least 80% sequence identity to the at least 215 consecutive amino acid residues of SEQ ID NO: 17, 43, 45, or 47.

Embodiment 541 comprises a method of any one of embodiments 489-517, wherein the Fab heavy chain polypeptide comprises an amino acid sequence according to SEQ ID NO: 17, 43, 45, or 47.

Embodiment 542 comprises a method of any one of embodiments 489-517, wherein the Fab light chain polypeptide comprises an amino acid sequence that has at least 80% sequence identity to the amino acid sequence according to SEQ ID NO: 16, 42, 44, or 46.

Embodiment 543 comprises a method of any one of embodiments 489-517, wherein the Fab light chain polypeptide comprises an amino acid sequence of at least 175 consecutive amino acid residues of SEQ ID NO: 16, 42, 44, or 46.

Embodiment 544 comprises a method of any one of embodiments 489-517, wherein the Fab light chain polypeptide comprises an amino acid sequence of at least 200 consecutive amino acid residues of SEQ ID NO: 16, 42, 44, or 46.

Embodiment 545 comprises a method of any one of embodiments 489-517, wherein the Fab light chain polypeptide comprises an amino acid sequence of at least 200 consecutive amino acid residues of SEQ ID NO: 16 and has at least 80% sequence identity to the at least 200 consecutive amino acid residues of SEQ ID NO: 16, 42, 44, or 46.

Embodiment 546 comprises a method of any one of embodiments 489-517, wherein the Fab light chain polypeptide comprises an amino acid sequence according to SEQ ID NO: 16, 42, 44, or 46.

Embodiment 547 comprises a method of any one of embodiments 489-517, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

Embodiment 548 comprises a method of any one of embodiments 489-517, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21.

Embodiment 549 comprises a method of any one of embodiments 489-517, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21.

Embodiment 550 comprises a method of any one of embodiments 489-517, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

Embodiment 551 comprises a method of any one of embodiments 489-517, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

Embodiment 552 comprises a method of any one of embodiments 489-517, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22.

Embodiment 553 comprises a method of any one of embodiments 489-517, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22.

Embodiment 554 comprises a method of any one of embodiments 489-517, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

Embodiment 555 comprises a method of any one of embodiments 489-554, wherein the multispecific antibodies of Formula Ia, Formula Ib, Formula Ic further comprise a half-life extending molecule (H₁).

Embodiment 556 comprises a method of embodiment 555, wherein H₁ is connected to P1.

Embodiment 557 comprises a method of embodiment 555, wherein H₁ is connected to P₂.

Embodiment 558 comprises a method of any one of embodiments 555-557, wherein H₁ does not block A binding to CD28.

Embodiment 559 comprises a method of any one of embodiments 555-558, wherein H₁ does not block B binding to PD-L1.

Embodiment 560 comprises a method of any one of embodiments 555-559, H₁ comprises a linking moiety (L₅) that connects H₁ to P₁ or H₁ to P₂.

Embodiment 561 comprises a method of any one of embodiments 555-560, wherein the half-life extending molecule (H₁) does not have binding affinity to PD-L1.

Embodiment 562 comprises a method of any one of embodiments 555-5561, wherein the half-life extending molecule (H₁) does not have binding affinity to CD28.

Embodiment 563 comprises a method of any one of embodiments 555-562, wherein the half-life extending molecule (H₁) does not shield the multispecific antibody from CD28.

Embodiment 564 comprises a method of any one of embodiments 555-563, wherein H₁ comprises a sequence according to SEQ ID NOs: 54-57.

Embodiment 565 comprises a method of any one of embodiments 555-564, wherein H₁ comprises an amino acid sequence that has repetitive sequence motifs.

Embodiment 566 comprises a method of any one of embodiments 555-565, wherein H₁ comprises an amino acid sequence that has highly ordered secondary structure.

Embodiment 567 comprises a method of any one of embodiments 555-566, wherein H₁ comprises a polymer.

Embodiment 568 comprises a method of embodiment 567, wherein the polymer is polyethylene glycol (PEG).

Embodiment 569 comprises a method of any one of embodiments 555-566, wherein H₁ comprises albumin.

Embodiment 570 comprises a method of any one of embodiments 555-566, wherein H₁ comprises an Fc domain.

Embodiment 571 comprises a method of embodiment 569, wherein the albumin is serum albumin.

Embodiment 572 comprises a method of embodiment 571, wherein the albumin is human serum albumin.

Embodiment 573 comprises a method of any one of embodiments 555-566, wherein H₁ comprises a polypeptide, a ligand, or a small molecule.

Embodiment 574 comprises a method of embodiment 573, wherein the polypeptide, the ligand or the small molecule binds serum protein or a fragment thereof, a circulating immunoglobulin or a fragment thereof, or CD35/CR1.

Embodiment 575 comprises a method of embodiment 574, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 576 comprises a method of embodiment 574, wherein the circulating immunoglobulin molecule comprises IgG1, IgG2, IgG3, IgG4, slgA, IgM or IgD.

Embodiment 577 comprises a method of embodiment 574, wherein the serum protein is albumin.

Embodiment 578 comprises a method of embodiment 573, wherein the polypeptide is an antibody.

Embodiment 579 comprises a method of embodiment 578, wherein the antibody comprises a single domain antibody, a single chain variable fragment or a Fab.

Embodiment 580 comprises a method of embodiment 579, wherein the single domain antibody comprises a single domain antibody that binds to albumin.

Embodiment 581 comprises a method of embodiment 579, wherein the single domain antibody is a human or humanized antibody.

Embodiment 582 comprises a method of embodiment 579, wherein the single domain antibody is selected from the group consisting of 645gHlgL1, 645dsgH5gL4, 23-13-A01-sc02, A10m3 or a fragment thereof, DOM7r-31, DOM7h-11-15, Alb-1, Alb-8, Alb-23, 10G, 10E and SA21.

Embodiment 583 comprises a method of embodiment 579, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 54, HC-CDR2: SEQ ID NO: 55, and HC-CDR3: SEQ ID NO: 56; and wherein the CDRs comprise from 0-2 amino acid modifications in at least one of the HC-CDR1, HC-CDR2, or HC-CDR3.

Embodiment 584 comprises a method of any one of embodiments 555-566, wherein H₁ comprises an amino acid sequence according to SEQ ID NO: 57.

Embodiment 585 comprises a method of any one of embodiments 555-566, wherein H₁ comprises an amino acid sequence that has at least 80% sequence identity to SEQ ID NO: 57.

Embodiment 586 comprises a method of any one of embodiments 555-566, wherein H₁ comprises an amino acid sequence that has at least 85% sequence identity to SEQ ID NO: 57.

Embodiment 587 comprises a method of any one of embodiments 555-566, wherein H₁ comprises an amino acid sequence that has at least 90% sequence identity to SEQ ID NO: 57.

Embodiment 588 comprises a method of any one of embodiments 555-566, wherein H₁ comprises an amino acid sequence that has at least 95% sequence identity to SEQ ID NO: 57.

Embodiment 589 comprises a method of any one of embodiments 555-566, wherein H₁ comprises an amino acid sequence that has at least 99% sequence identity to SEQ ID NO: 57.

Embodiment 590 comprises a method of any one of embodiments 555-566, wherein H₁ comprise a modified amino acid or non-natural amino acid, or a modified non-natural amino acid, or a combination thereof.

Embodiment 591 comprises a method of embodiment 590, wherein the modified amino acid or a modified non-natural amino acid comprises a post-translational modification.

Embodiment 592 comprises a method of any one of embodiments 555-591, wherein H₁ comprises a linking moiety (L5) that connects H₁ to P₁ or P₂.

Embodiment 593 comprises a method of embodiment 592, wherein L₅ is a peptide sequence having at least 5 to no more than 50 amino acids.

Embodiment 594 comprises a method of embodiment 592, wherein L₅ is a peptide sequence having at least 10 to no more than 30 amino acids.

Embodiment 595 comprises a method of embodiment 592, wherein L₅ is a peptide sequence having at least 10 amino acids.

Embodiment 596 comprises a method of embodiment 592, wherein L₅ is a peptide sequence having at least 18 amino acids.

Embodiment 597 comprises a method of embodiment 592, wherein L₅ is a peptide sequence having at least 26 amino acids.

Embodiment 598 comprises a method of embodiment 592, wherein L₅ has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 599 comprises a method of any one of embodiments 489-598, wherein L₁ or L₂ is a peptide sequence having at least 5 to no more than 50 amino acids.

Embodiment 600 comprises a method of any one of embodiments 489-598, wherein L₁ or L₂ is a peptide sequence having at least 10 to no more than 30 amino acids.

Embodiment 601 comprises a method of any one of embodiments 489-598, wherein L₁ or L₂ is a peptide sequence having at least 10 amino acids.

Embodiment 602 comprises a method of any one of embodiments 489-598, wherein L₁ or L₂ is a peptide sequence having at least 18 amino acids.

Embodiment 603 comprises a method of any one of embodiments 489-598, wherein L₁ or L₂ is a peptide sequence having at least 26 amino acids.

Embodiment 604 comprises a method of any one of embodiments 489-598, wherein L₁ or L₂ has a formula comprising (G₂S)ₙ (SEQ ID NO: 233), wherein n is an integer from 1 to 3.

Embodiment 605 comprises a method of any one of embodiments 489-598, wherein L₁ or L₂ has a formula comprising (G₂S)ₙ, wherein n is an integer of at least 1.

Embodiment 606 comprises a method of any one of embodiments 489-598, wherein L₁ or L₂ has a formula selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

Embodiment 607 comprises a method of any one of embodiments 489-606, wherein the tumor specific protease is selected from the group consisting of metalloprotease, serine protease, cysteine protease, threonine protease, and aspartic protease.

Embodiment 608 comprises a method of any one of embodiments 489-606, wherein L₁ or L₂ comprises a urokinase cleavable amino acid sequence, a matriptase cleavable amino acid sequence, a legumain cleavable amino acid sequence, or a matrix metalloprotease cleavable amino acid sequence.

Embodiment 609 comprises a method of any one of embodiments 489-606, wherein L₁ or L₂ comprises a sequence according to SEQ ID NOs: 18-19, 62-88.

Embodiment 610 comprises a method of any one of embodiments 489-609, wherein L₁ is bound to N-terminus of A.

Embodiment 611 comprises a method of any one of embodiments 489-609, wherein L₁ is bound to C-terminus of A.

Embodiment 612 comprises a method of any one of embodiments 489-609, wherein L₂ is bound to N-terminus of B.

Embodiment 613 comprises a method of any one of embodiments 489-609, wherein L₂ is bound to C-terminus of B.

Embodiment 614 comprises a method of any one of embodiments 489-613, wherein P₁ becomes unbound from A when L1 is cleaved by the tumor specific protease thereby exposing A to CD28.

Embodiment 615 comprises a method of any one of embodiments 489-614, wherein P2 becomes unbound from B when L2 is cleaved by the tumor specific protease thereby exposing B to PD-L1.

Embodiment 616 comprises a method of any one of embodiments 489-615, wherein L₁ or L₂, comprise a modified amino acid or non-natural amino acid, or a modified non-natural amino acid, or a combination thereof.

Embodiment 617 comprises a method of embodiment 616, wherein the modified amino acid or a modified non-natural amino acid comprises a post-translational modification.

Embodiment 618 comprises a method of any one of embodiments 489-617, wherein P₁ impairs binding of A to CD28.

Embodiment 619 comprises a method of any one of embodiments 489-618, wherein P₁ is bound to A through ionic interactions, electrostatic interactions, hydrophobic interactions, Pi-stacking interactions, and H-bonding interactions, or a combination thereof.

Embodiment 620 comprises a method of any one of embodiments 489-619, wherein P₁ is bound to A at or near an antigen binding site.

Embodiment 621 comprises a method of any one of embodiments 489-620, wherein P₁ becomes unbound from A when L1 is cleaved by the tumor specific protease thereby exposing A to CD28.

Embodiment 622 comprises a method of any one of embodiments 489-621, wherein P₁ has less than 75% sequence identity to CD28.

Embodiment 623 comprises a method of any one of embodiments 489-622, wherein P₁ has less than 80% sequence identity to CD28.

Embodiment 624 comprises a method of any one of embodiments 489-623, wherein P₁ has less than 85% sequence identity to CD28.

Embodiment 625 comprises a method of any one of embodiments 489-624, wherein P₁ has less than 90% sequence identity to CD28.

Embodiment 626 comprises a method of any one of embodiments 489-625, wherein P₁ has less than 95% sequence identity to CD28.

Embodiment 627 comprises a method of any one of embodiments 489-626, wherein P₁ comprises a de novo amino acid sequence that shares less than 10% sequence identity to CD28.

Embodiment 628 comprises a method of any one of embodiments 489-627, wherein P₂ impairs binding of B to PD-L1.

Embodiment 629 comprises a method of any one of embodiments 489-628, wherein P₂ is bound to B through ionic interactions, electrostatic interactions, hydrophobic interactions, Pi-stacking interactions, and H-bonding interactions, or a combination thereof.

Embodiment 630 comprises a method of any one of embodiments 489-629, wherein P₂ is bound to B at or near an antigen binding site.

Embodiment 631 comprises a method of any one of embodiments 489-630, wherein P₂ becomes unbound from B when L2 is cleaved by the tumor specific protease thereby exposing B to the PD-L1.

Embodiment 632 comprises a method of any one of embodiments 489-631, wherein P₂ has less than 70% sequence identity to the PD-L1.

Embodiment 633 comprises a method of any one of embodiments 489-632, wherein P₂ has less than 75% sequence identity to the PD-L1.

Embodiment 634 comprises a method of any one of embodiments 489-633, wherein P₂ has less than 80% sequence identity to the PD-L1.

Embodiment 635 comprises a method of any one of embodiments 489-634, wherein P₂ has less than 85% sequence identity to the PD-L1.

Embodiment 636 comprises a method of any one of embodiments 489-635, wherein P₂ has less than 90% sequence identity to the PD-L1.

Embodiment 637 comprises a method of any one of embodiments 489-636, wherein P₂ has less than 95% sequence identity to the PD-L1.

Embodiment 638 comprises a method of any one of embodiments 489-637, wherein P₂ comprises a de novo amino acid sequence that shares less than 10% sequence identity to the PD-L1.

Embodiment 639 comprises a method of any one of embodiments 489-638, wherein P₁ or P₂ comprises a peptide sequence of at least 5 amino acids in length.

Embodiment 640 comprises a method of any one of embodiments 489-639, wherein P₁ or P₂ comprises a peptide sequence of at least 6 amino acids in length.

Embodiment 641 comprises a method of any one of embodiments 489-640, wherein P₁ or P₂ comprises a peptide sequence of at least 10 amino acids in length.

Embodiment 642 comprises a method of any one of embodiments 489-641, wherein P₁ or P₂ comprises a peptide sequence of at least 10 amino acids in length and no more than 20 amino acids in length.

Embodiment 643 comprises a method of any one of embodiments 489-642, wherein P₁ or P₂ comprises a peptide sequence of at least 16 amino acids in length.

Embodiment 644 comprises a method of any one of embodiments 489-641, wherein P₁ or P₂ comprises a peptide sequence of no more than 40 amino acids in length.

Embodiment 645 comprises a method of any one of embodiments 489-644, wherein P₁ or P₂ comprises at least two cysteine amino acid residues.

Embodiment 646 comprises a method of any one of embodiments 489-644, wherein P₁ or P₂ comprises a cyclic peptide or a linear peptide.

Embodiment 647 comprises a method of any one of embodiments 489-644, wherein P₁ or P₂ comprises a cyclic peptide.

Embodiment 648 comprises a method of any one of embodiments 489-644, wherein P₁ or P₂ comprises a linear peptide.

Embodiment 649 comprises a method of any one of embodiments 489-644, wherein P₁ or P₂ comprise a modified amino acid or non-natural amino acid, or a modified non-natural amino acid, or a combination thereof.

Embodiment 650 comprises a method of any one of embodiments 489-644, wherein P₁ or P₂ does not comprise albumin or an albumin fragment.

Embodiment 651 comprises a method of any one of embodiments 489-644, wherein P₁ or P₂ does not comprise an albumin binding domain.

### EXAMPLES

### Example 1. Immune Cell Activation Assays

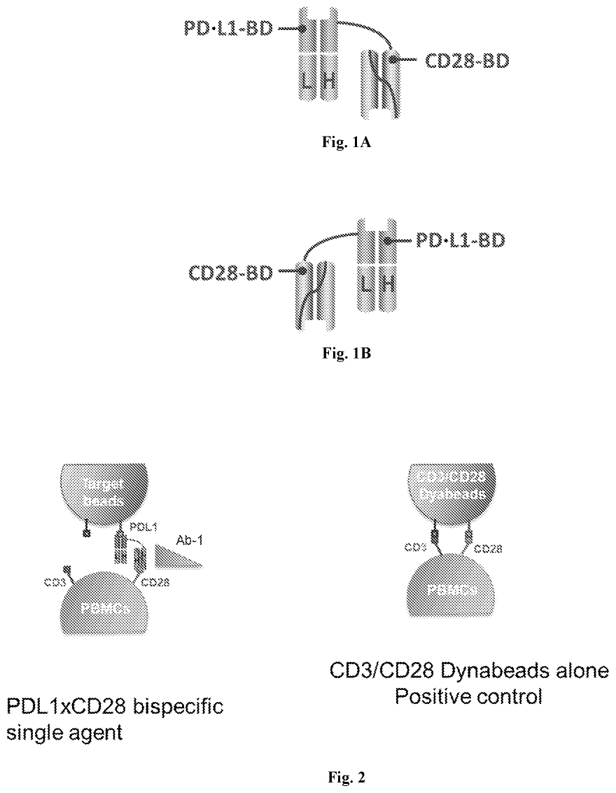

This Example assesses PDL1 x CD28 multispecific antibody Ab-1 in in vitro immune cell activation assays using target coated beads. An exemplary schema of the PDL1 x CD28 multispecific antibodies is seen in FIGS. 1A-1B.

Immune cell activation was measured via cytokine release and immune cell proliferation after co-culture of target coated beads and PBMCs. Briefly, M280 magnetic streptavidin beads were treated with soluble biotinylated PDL1. M280 beads were washed and seeded in a 96 well plate at 200,000 beads per well. Compounds were then titrated as single agents and added to the wells followed by 100,000 PBMCs. Human T cell activator CD3/CD28 beads (Invitrogen) were used as a positive control in the absence of compound. Wells lacking M280 target coated beads were used as negative controls. After 48 hours of co-culture, cytokines were measured in the supernatant using Cytometric Bead Array (CBA) Human Th1/Th2/Th17 Cytokine Kit from BD Biosciences. The concentration of cytokines was calculated using a standard curve per manufacturer's instructions. Co-culture of PBMCs, M280 target coated beads, and compounds continued for a total of 8 days where medium and compounds were replaced on day 4. On days 1, 4, and 8, magnetic beads were removed using a magnet, cells harvested, and stained using LIVE/DEAD, anti-CD3, anti-CD4, and anti-CD8 fluorescent markers. The total amount of live cells, live CD3+ cells, live CD4+ cells, or live CD8+ cells were measured via flow cytometry and plotted versus logarithmic compound concentration. Proliferation was determined by the increase in live cells and associated T cell populations. An exemplary schema of the assay is seen in FIG. 2.

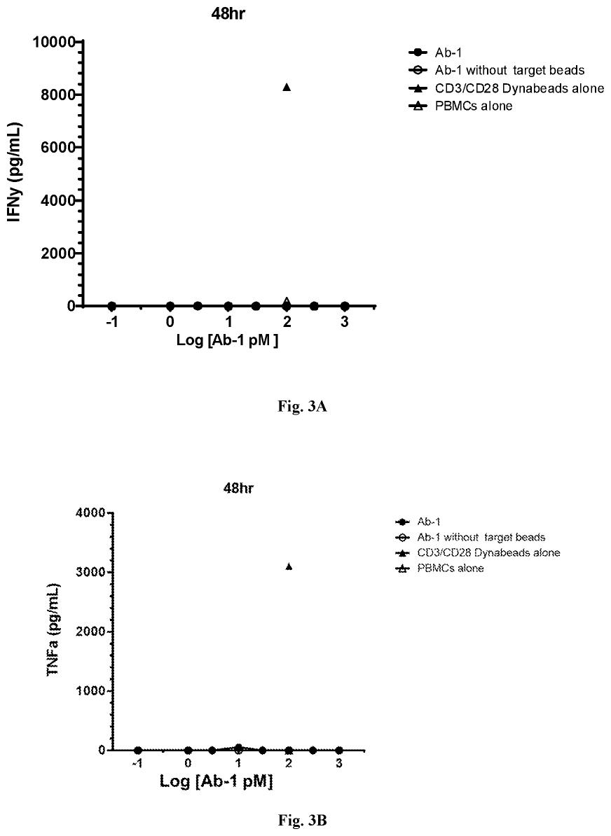

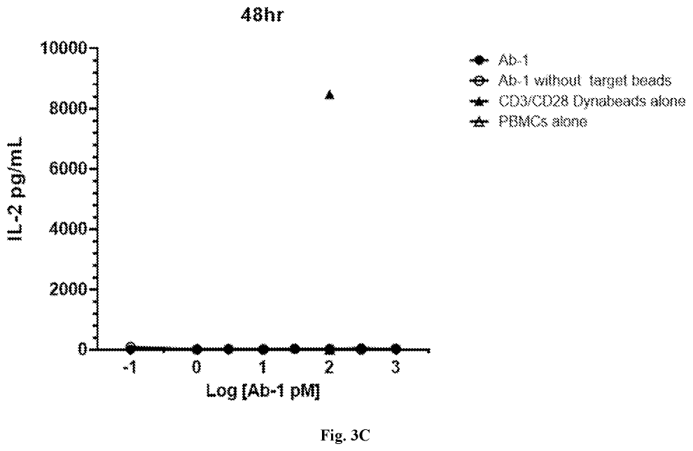

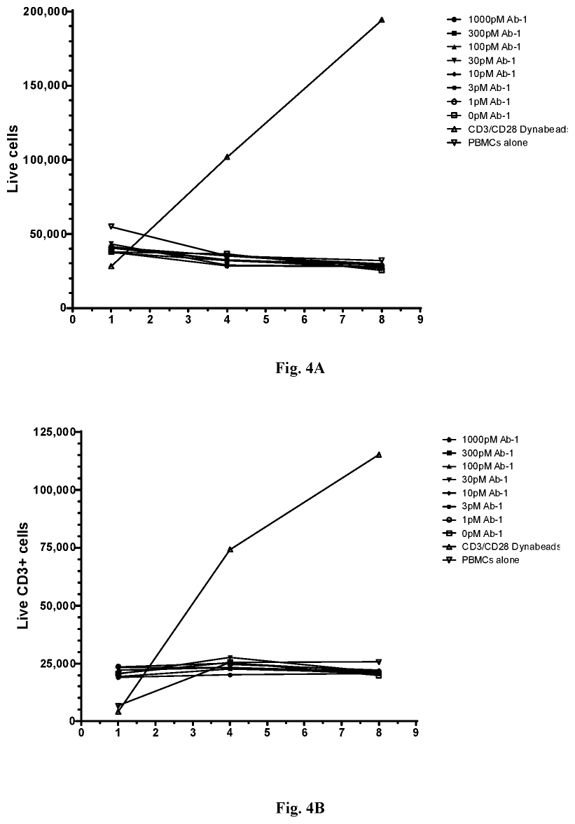

Cytokine release was measured from PBMCs cultured with single agent PDL1 x CD28 multispecific antibody Ab-1. FIG. 3A shows data for IFNy. FIG. 3B shows data for TNFα. FIG. 3C shows data for IL-2. The data shows Ab-1 does not trigger non-specific IFNy, TNFα, and IL-2 release from PBMC's and that Ab-1 requires immune cells to recognize an antigen to trigger activation.

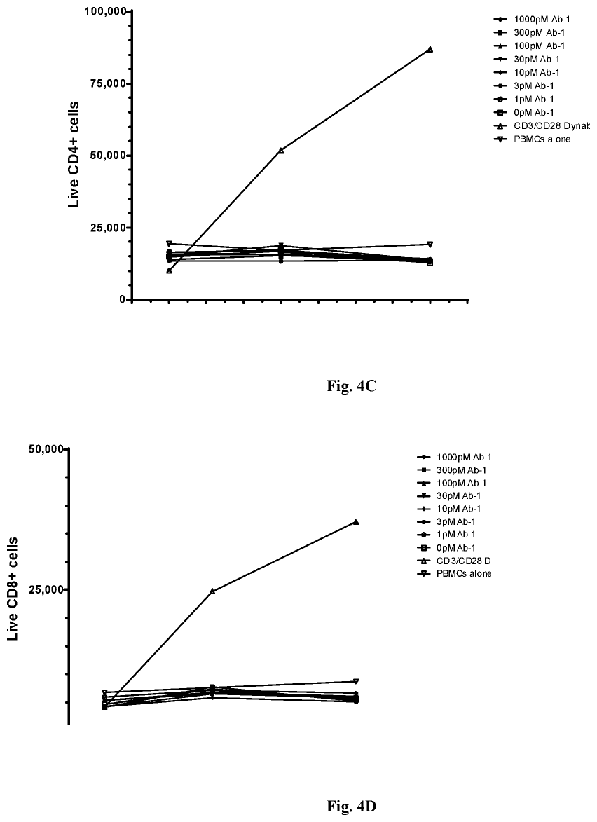

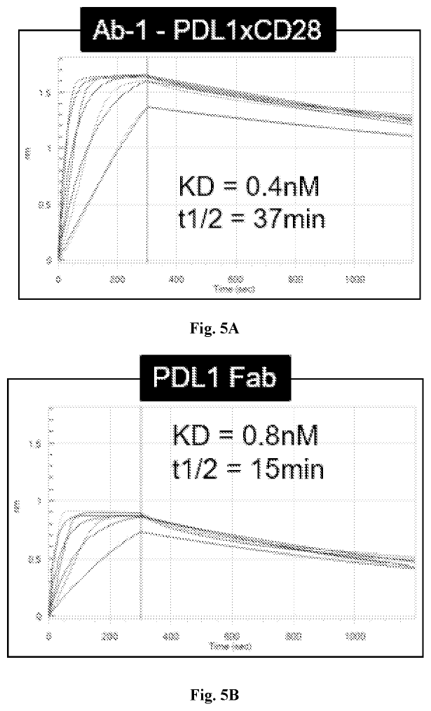

FIGS. 4A-4D show data for the number of live immune cells (FIG. 4A), CD3+ cells (FIG. 4B), CD4+ cells (FIG. 4C), and CD8+ cells (FIG. 4D) overtime in response to PBMC co-cultured with PDL1 target coated beads and Ab-1. The data shows that Ab-1 does not trigger non-specific immune cell proliferation and that Ab-1 requires immune cells to recognize an antigen to trigger proliferation.

### Example 2. Kinetic Binding Assays Against Human PD-L1

This Example assesses binding of Ab-1 and anti-PD-L1 Fab 1 to human PD-L1 in an in vitro assay.

Kinetic binding of Ab-1 and anti-PD-L1 Fab 1 to biotinylated human PDL1 was evaluated by bio-layer interferometry using an Octet RED96 instrument. Briefly, biosensors were loaded with antigen and baselined in buffer. Polypeptide molecules were titrated in solution at 50 nM, 25 nM, 12.5 nM, and 6.25 nM then associated onto the antigen loaded sensors. After a short association period, sensors were transferred into buffer and the dissociation of bound polypeptide molecules was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. Off rate constants were converted to half-life as shown in FIGS. 5A and 5B.

TABLE 15
Timing and Steps of Assay

| Step | Time |
|---|---|
| Baseline: Octet buffer | 60 sec |
| Load: | |
| 10 nM Human PDL1-biotin | 300 sec |
| Biocytin quench (100 uM) | 300 sec |
| Baseline: Octet buffer | 300 sec |
| Association: Octet buffer | |
| 50 nM Ab-1 | 300 sec |
| 25 nM Ab-1 | |
| 12.5 nM Ab-1 | |
| 6.25 nM Ab-1 | |
| 50 nM anti-PD-L1 Fab 1 | |
| 25 nM anti-PD-L1 Fab 1 | |
| 12.5 nM anti-PD-L1 Fab 1 | |
| 6.25 nM anti-PD-L1 Fab 1 | |
| Dissociation: Octet Buffer | 900 sec |

### Example 3. Kinetic Binding Assays Against Human CD28

This Example assesses binding of Ab-1 and Ab-2 to human PD-L1 in an in vitro assay.

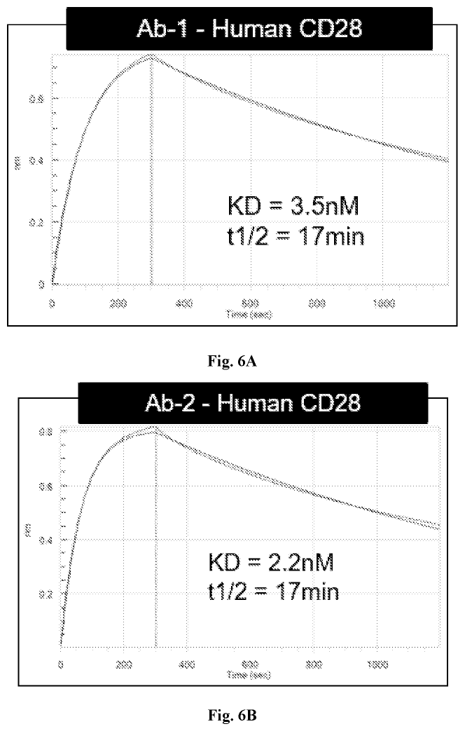

Kinetic binding of Ab-1 and Ab-2 to biotinylated human CD28 was evaluated by bio-layer interferometry using an Octet RED96 instrument. Briefly, biosensors were loaded with antigen and baselined in buffer. Polypeptide molecules were titrated in solution at 50 nM then associated onto the antigen loaded sensors. After a short association period, sensors were transferred into buffer and the dissociation of bound polypeptide molecules was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. Off rate constants were converted to half-life shown in FIGS. 6A and 6B.

TABLE 16
Timing and Steps of Assay

| Step | Time |
|---|---|
| Baseline: Octet buffer | 60 sec |
| Load: | |
| 10 nM Human CD28-Biotin | 300 sec |
| Biocytin quench (100 uM) | 300 sec |
| Baseline: Octet buffer | 300 sec |
| Association in octet buffer | |
| 50 nM Ab-2 | 300 sec |
| 50 nM Ab-1 | |
| Dissociation: Octet Buffer | 900 sec |

### Example 4. Kinetic Binding Assays Against Human and Cynomolgus Monkey PD-L1

This Example assesses binding of Ab-1 to human PD-L1 Fe and cynomolgus monkey PD-L1 Fc in an in vitro assay.

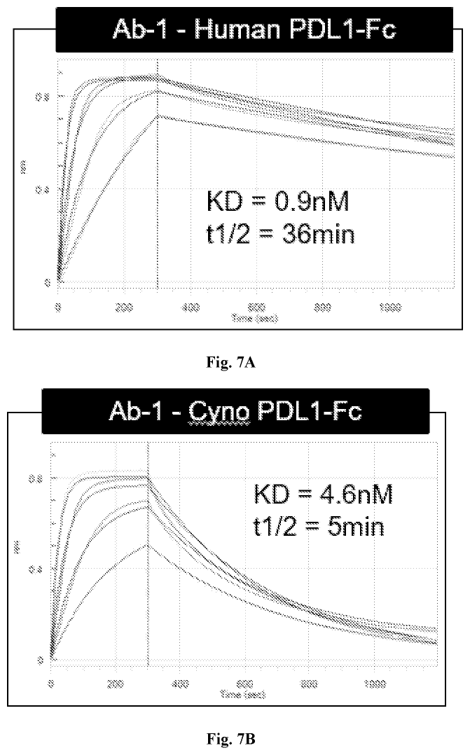

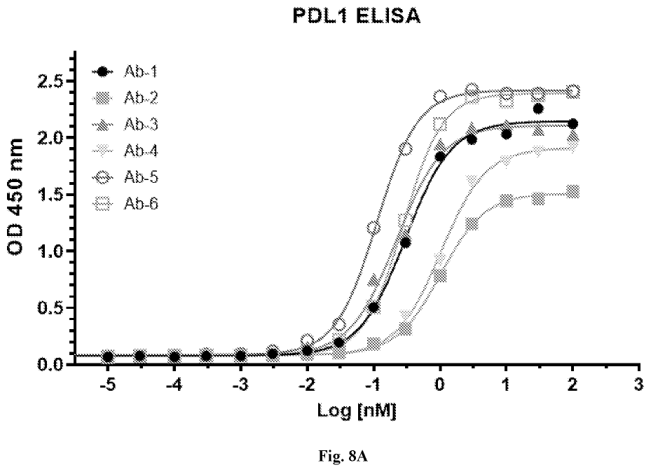

Kinetic binding of Ab-1 to human PD-L1 Fe and cynomolgus monkey PD-L1 Fe was evaluated by bio-layer interferometry using an Octet RED96 instrument. Briefly, biosensors were loaded with antigen and baselined in buffer. Polypeptide molecules were titrated in solution at 50 nM then associated onto the antigen loaded sensors. After a short association period, sensors were transferred into buffer and the dissociation of bound polypeptide molecules was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. Off rate constants were converted to half-life shown in FIGS. 7A and 7B.

TABLE 17
Timing and Steps of Assay

| Step | Time |
|---|---|
| Baseline: Octet buffer | 60 sec |
| Load: | |
| 15 nM human PDL1-Fc | 300 sec |
| 15 nM cyno PDL1-Fc | |
| Baseline: Octet buffer | 300 sec |
| Association in octet buffer | |
| 50 nM Ab-1 | 300 sec |
| 25 nM Ab-1 | |
| 12.5 nM Ab-1 | |
| 6.25 nM Ab-1 | |
| Dissociation: Octet Buffer | 900 sec |

### Example 5. ELISA Binding Assays of Polypeptide Complex Molecules to Human PD-L1, CD28 and EGFR

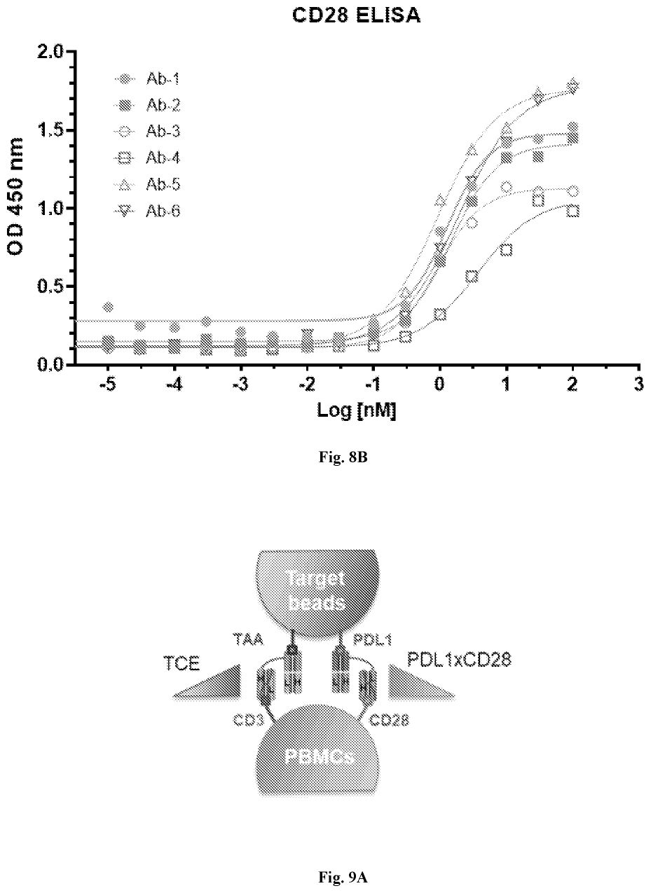

The polypeptide complex molecules were evaluated for their ability to bind human PDL1 or CD28 in a standard enzyme linked immunosorbent assay (ELISA) format. Briefly, biotinylated antigen was captured on neutravidin coated plates. Polypeptide complex molecules diluted in buffer were then added to the antigen coated plates. Bound polypeptide complex was detected using a standard horse radish peroxidase conjugate secondary antibody. The concentration of polypeptide complex required to achieve 50% maximal signal (EC50) was calculated using Graphpad Prism software. FIG. 8A illustrates binding of Ab-1, Ab-2, Ab-3, Ab-4, Ab-5, and Ab-6 to PD-L1 and is summarized in Table 18. FIG. 8B illustrates binding of Ab-1, Ab-2, Ab-3, Ab-4, Ab-5, and Ab-6 to CD28 and is summarized in Table 19.

TABLE 18
Summary of binding of Ab-1, Ab-2,
Ab-3, Ab-4, Ab-5, and Ab-6 to PD-L1

PD-L1 ELISA

| | Ab-1 | Ab-2 | Ab-3 | Ab-4 | Ab-5 | Ab-6 |
|---|---|---|---|---|---|---|
| EC50 | 0.30 | 1.02 | 0.22 | 1.03 | 0.11 | 0.28 |
| nM | | | | | | |

TABLE 19
Summary of binding of Ab-1, Ab-2,
Ab-3, Ab-4, Ab-5, and Ab-6 to CD28

CD28 ELISA

| | Ab-1 | Ab-2 | Ab-3 | Ab-4 | Ab-5 | Ab-6 |
|---|---|---|---|---|---|---|
| EC50 | 1.27 | 1.44 | 0.92 | 3.64 | 0.91 | 1.89 |
| nM | | | | | | |

### Example 6. T Cell Activation Assays of Ab-1 in Combination with an Anti-TROP2 x CD3 T Cell Engager (TCE-1)

Immune cell activation was measured via cytokine release and immune cell proliferation after co-culture of non-immunogenic target coated beads and PBMCs. Briefly, M280 magnetic streptavidin beads were treated with soluble biotinylated PDL1 and soluble biotinylated TROP2. M280 beads were washed and seeded in a 96 well plate at 200,000 beads per well. Compounds were then titrated as single agents and in combination then added to the wells followed by 100,000 PBMCs. Human T cell activator CD3/CD28 beads (Invitrogen) were used as a positive control in the absence of compound. Wells lacking M280 target coated beads were used as negative controls. After 48 hours of co-culture, cytokines were measured in the supernatant using Cytometric Bead Array (CBA) Human Th1/Th2/Th17 Cytokine Kit from BD Biosciences. The concentration of cytokines was calculated using a standard curve per manufacturer's instructions. Co-culture of PBMCs, M280 target coated beads, and compounds continued for a total of 8 days where medium and compounds were replaced on day 4. On days 1, 4, and 8, magnetic beads were removed using a magnet, cells harvested, and stained using LIVE/DEAD, anti-CD3, anti-CD4, and anti-CD8 fluorescent markers. The total amount of live cells, live CD3+ cells, live CD4+ cells, or live CD8+ cells were measured via flow cytometry and plotted versus logarithmic compound concentration. Proliferation was determined by the increase in live cells and associated T cell populations. FIGS. 9B-9D illustrate that PBMC activation measured by cytokine release require both Ab-1 and TCE-1, an anti-TROP2 x CD3 T cell engager. FIGS. 9E-9H illustrate immune cell proliferation after co-culture of target coated beads and PBMCs and administration of Ab-1 and the combination of Ab-1 and TCE-1, an anti-TROP2 x CD3 T cell engager. FIGS. 9B-9D illustrate that immune cell proliferation from PBMCs activation measured by flow cytometry against non-immunogenic beads coated with TAA and PDL1 requires both Ab-1 and TCE-1, an anti-TROP2 x CD3 T cell engager. The sequences of TCE-1 is provided in Table 20.

TABLE 20
Amino acid sequences of TCE-1

| | Amino Acid Sequence | SEQ ID |
|---|---|---|
| Construct Description | (N to C) | NO: |
| TCE-1 LC | DIQLTQSPSSLSASVGDRVSITC | 118 |
| TROP2 Fab LC | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TCE-1 HC | QTVVTQEPSLTVSPGGTVTLT | 128 |
| SP34.194 scFv (VH- | CRSSTGAVTTSNYANWVQQK | |
| linker 1-VL) + | PGQAPRGLIGGTNKRAPGTPA | |
| Linker 2 + TROP2 Fab | RFSGSLLGGKAALTLSGVQPE | |
| HC | DEAEYYCALWYSNLWVFGG | |
| | GTKLTVLGGGGSGGGGSGGG | |
| | GSEVQLVESGGGLVQPGGSLK | |
| | LSCAASGFTFNTYAMNWVRQ | |
| | APGKGLEWVARIRSKYNNYA | |
| | TYYADSVKDRFTISRDDSKNT | |
| | AYLQMNNLKTEDTAVYYCVR | |
| | HGNFGNSYVSWFAYWGQGT | |
| | LVTVSSGGGGSQVQLQQSGSE | |
| | LKKPGASVKVSCKASGYTFT | |
| | NYGMNWVKQAPGQGLKWM | |
| | GWINTYTGEPTYTDDFKGRF | |
| | AFSLDTSVSTAYLQISSLKADD | |
| | TAVYFCARGGFGSSYWYFDV | |
| | WGQGSLVTVSSASTKGPSVFP | |
| | LAPSSKSTSGGTAALGCLVKD | |
| | YFPEPVTVSWNSGALTSGVHT | |
| | FPAVLQSSGLYSLSSVVTVPSS | |
| | SLGTQTYICNVNHKPSNTKVD | |
| | KKVEPKSC | |

### Example 7. T Cell Activation Assays of Ab-1 Through Ab-8 in Combination with an Anti-TROP2 x CD3 T Cell Engager (TCE-3)

Immune cell activation was measured via cytokine release and immune cell proliferation after co-culture of target coated beads and PBMCs. Briefly, M280 magnetic streptavidin beads were treated with soluble biotinylated PDL1 and soluble biotinylated TROP2. M280 beads were washed and seeded in a 96 well plate at 200,000 beads per well. Compounds were then titrated as single agents and in combination then added to the wells followed by 100,000 PBMCs. Human T cell activator CD3/CD28 beads (Invitrogen) were used as a positive control in the absence of compound. Wells lacking M280 target coated beads were used as negative controls. After 48 hours of co-culture, cytokines were measured in the supernatant using Cytometric Bead Array (CBA) Human Th/Th2/Th17 Cytokine Kit from BD Biosciences. The concentration of cytokines was calculated using a standard curve per manufacturer's instructions. Co-culture of PBMCs, M280 target coated beads, and compounds continued for a total of 8 days where medium and compounds were replaced on day 4. On days 1, 4, and 8, magnetic beads were removed using a magnet, cells harvested, and stained using LIVE/DEAD, anti-CD3, anti-CD4, and anti-CD8 fluorescent markers. The total amount of live cells, live CD3+ cells, live CD4+ cells, or live CD8+ cells were measured via flow cytometry and plotted versus logarithmic compound concentration. Proliferation was determined by the increase in live cells and associated T cell populations.

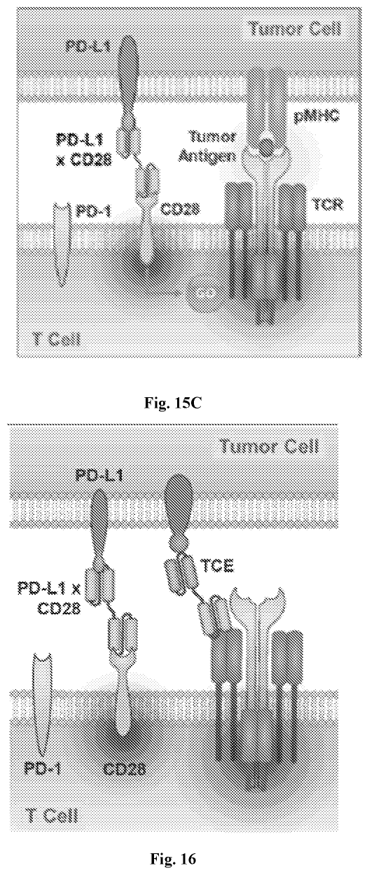

FIGS. 91-9K illustrate polypeptide complexes of different orientation harboring different PD-L1 binding domains (Ab-1 through Ab-8) are able to activate PBMCs as measured by cytokine release in combination with a T cell engager (TCE-3) against non-immunogenic beads coated with TAA and PD-L1. The sequences of TCE-3 is provided in Table 21. FIG. 9L illustrates that polypeptide complex mediated activation of PBMCs is dependent on PD-L1 surface density and the combination with a T cell engager. FIGS. 9M-9S illustrate polypeptide complex mediated activation of PBMCs is dependent on PD-L1 surface density and the combination with a T cell engager.

TABLE 21
Amino acid sequences of TCE-3

| Construct | Amino Acid Sequence | SEQ ID |
|---|---|---|
| Description | (N to C) | NO: |
| TCE-3 HC | QVQLQQSGSELKKPGASVKVS | 129 |
| TROP2 Fab HC | CKASGYTFTNYGMNWVKQAP | |
| | GQGLKWMGWINTYTGEPTYT | |
| | DDFKGRFAFSLDTSVSTAYLQI | |
| | SSLKADDTAVYFCARGGFGSS | |
| | YWYFDVWGQGSLVTVSSAST | |
| | KGPSVFPLAPSSKSTSGGTAAL | |
| | GCLVKDYFPEPVTVSWNSGAL | |
| | TSGVHTFPAVLQSSGLYSLSSV | |
| | VTVPSSSLGTQTYICNVNHKPS | |
| | NTKVDKKVEPKSC | |
| TCE-3 LC | QTVVTQEPSLTVSPGGTVTLT | 130 |
| SP34.185 scFv + | CRSSTGAVTTSNYANWVQQK | |
| linker + TROP2 Fab LC | PGQAPRGLIGGTNKRAPGTPA | |
| | RFSGSLLGGKAALTLSGVQPE | |
| | DEAEYYCALWYSNLWVFGGG | |
| | TKLTVLGGGGSGGGGSGGGG | |
| | SEVQLVESGGGLVQPGGSLKL | |
| | SCAASGFTFNTYAMNWVRQA | |
| | PGKGLEWVARIRSKYNNYAT | |
| | YYADSVKDRFTISRDDSKNTA | |
| | YLQMNNLKTEDTAVYYCVRH | |
| | GNFGNSYVSWFAYWGQGTLV | |
| | TVSSGGGGSDIQLTQSPSSLSA | |
| | SVGDRVSITCKASQDVSIAVA | |
| | WYQQKPGKAPKLLIYSASYRY | |
| | TGVPDRFSGSGSGTDFTLTISS | |
| | LQPEDFAVYYCQQHYITPLTF | |
| | GAGTKVEIKRTVAAPSVFIFPP | |
| | SDEQLKSGTASVVCLLNNFYP | |
| | REAKVQWKVDNALQSGNSQE | |
| | SVTEQDSKDSTYSLSSTLTLSK | |
| | ADYEKHKVYACEVTHQGLSS | |
| | PVTKSFNRGEC | |

### Example 8. Tumor Cell Killing Assays with of Ab-1 in Combination with an Anti-TROP2 X CD3 T Cell Engager (TCE-2)

Polypeptide complexes were evaluated in a functional in vitro tumor cell killing and cytokine release assays using the PDL 1 positive tumor cell line, LNCaP. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. Tumor cells were added and allowed to adhere overnight on a 96 well E-Plate. The following day polypeptide complexes as single agents or in combination with a TCE, TRACTr, or pre-cleaved TRACTr were titrated in human serum supplemented medium along with PBMCs and added to the wells. Cell index measurements were taken every 10 minutes for an additional 120 hours. The cell index times number of hours (tumor cell growth kinetics) was then plotted versus concentration of polypeptide complex where the concentration required to reduce the tumor growth 50% o (IC50) was calculated using Graphpad Prism software. Cytokines were measured at study endpoint using the Th1/Th2/Th17 cytometric bead array from BD Biosciences.

FIGS. 10A-10C illustrate results of an in vitro tumor cell killing assay using the LNCaP PDL 1 positive tumor cell line in which Ab-1 and TCE-2 are co-administered in the presence of human PBMCs. In vitro tumor cell killing is synergized when Ab-1 is combined with an anti-PSMA x CD3 T cell engager, TCE-2. The sequences of TCE-2 is provided in Table 22.

TABLE 22
Amino acid sequences of TCE-2

| Construct | Amino Acid Sequence | SEQ ID |
|---|---|---|
| Description | (N to C) | NO: |
| TCE-2 LC | EVQLVESGGGLVQPGGSLKLS | 232 |
| | CAASGFTFNKYAMNWVRQA | |
| | PGKGLEWVARIRSKYNNYAT | |
| | YYADSVKDRFTISRDDSKNTA | |
| | YLQMNNLKTEDTAVYYCVRH | |
| | GNFGNSYISYWAYWGQGTLV | |
| | TVSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKL | |
| | TVLGGGGSDIQMTQSPSSLSAS | |
| | VGDRVTITCRASQGISNYLAW | |
| | YQQKTGKVPKFLIYEASTLQS | |
| | GVPSRFSGGGSGTDFTLTISSL | |
| | QPEDVATYYCQNYNSAPFTF | |
| | GPGTKVDIKRTVAAPSVFIFPP | |
| | SDEQLKSGTASVVCLLNNFYP | |
| | REAKVQWKVDNALQSGNSQE | |
| | SVTEQDSKDSTYSLSSTLTLSK | |
| | ADYEKHKVYACEVTHQGLSS | |
| | PVTKSFNRGEC | |
| TCE-2 HC | QVQLVESGGGVVQPGRSLRLS | 127 |
| | CAASGFAFSRYGMHWVRQAP | |
| | GKGLEWVAVIWYDGSNKYY | |
| | ADSVKGRFTISRDNSKNTQYL | |
| | QMNSLRAEDTAVYYCARGGD | |
| | FLYYYYYGMDVWGQGTTVT | |
| | VSSASTKGPSVFPLAPSSKSTS | |
| | GGTAALGCLVKDYFPEPVTVS | |
| | WNSGALTSGVHTFPAVLQSSG | |
| | LYSLSSVVTVPSSSLGTQTYIC | |
| | NVNHKPSNTKVDKKVEPKSC | |

FIG. 10D illustrates a graph of killing H292 tumor cells using Ab-1 and TCE-4. FIG. 10E illustrates a graph of killing H292 tumor cells using Ab-1 and TRACTr-1. FIG. 10F illustrates a graph of killing H292 tumor cells using Ab-1, TRACTr-1, and MTSP1. Polypeptide complexes enhance TRACTr tumor cell killing in the presence of human PBMCs. FIG. 10G illustrates a graph of IL-2 cytokine release from PBMCs cultured with Ab-1 and TCE-4. FIG. 10H illustrates a graph of IL-2 cytokine release from PBMCs cultured with Ab-1 and TRACTr-1. FIG. 10I illustrates a graph of IL-2 cytokine release from PBMCs cultured with Ab-1, TRACTr-1, and MTSP1. Polypeptide complexes enhance TRACTr tumor cell killing in the presence of human PBMCs.

The sequences of TCE-4 and TRACTr-1 are provided in Table 23.

TABLE 23
Amino acid sequences of TCE-4 and TRACTr-1

| | Amino Acid Sequence | SEQ ID |
|---|---|---|
| Construct Description | (N to C) | NO: |
| TCE-4 LC | DIQLTQSPSSLSASVGDRVSITC | 131 |
| TROP2 Fab LC | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TCE-4 HC | EVQLVESGGGLVQPGGSLKLS | 132 |
| SP34.185 scFv + | CAASGFTFNKYAMNWVRQAP | |
| linker + TROP2 Fab HC | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYFDVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TRACTr-1 LC | GGVDFCKIYSWPVCHQGGGG | 133 |
| TROP2 TRACTr | SGGLSGRSDAGSPLGLAGSGG | |
| Mask + linker + | SDIQLTQSPSSLSASVGDRVSIT | |
| cleavable linker + | CKASQDVSIAVAWYQQKPGK | |
| linker + TROP2 Fab LC | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TRACTr-1 HC | EVQLVESGGGLVQPGGSLRLS | 134 |
| TROP2 TRACTr | CAASGSTFYTAVMGWVRQAP | |
| Albumin binding domain + | GKGLEWVAAIRWTALTTSYA | |
| linker + mask + linker + | DSVKGRFTISRDGAKTTLYLQ | |
| cleavable linker + | MNSLRPEDTAVYYCAARGTL | |
| linker + SP34.185 scFv + | GLFTTADSYDYWGQGTLVTV | |
| linker + TROP2 Fab HC | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYFDVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |

### Example 9. Pharmacokinetics of Ab-1 in Cynomolgus Monkey

Pharmacokinetics and exploratory safety of Ab-1 were evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Concentration of polypeptide molecules in plasma was measured via standard ELISA techniques relative to a reference standard diluted in control cyno plasma. Plasma concentration curves were fit to a standard two phase exponential equation representing distribution and elimination phases. Fitting of pharmacokinetics enabled the calculation of Cmax, half-life, volume of distribution, clearance, and 7 day area under the curve (AUC) shown in Table 24. FIG. 11 illustrates pharmacokinetics of Ab-1 in cynomolgus monkey after a single IV bolus injection.

TABLE 24
FIG. 11 pharmacokinetic summary of Ab-1

| | Ab-1 30 ug/kg | Units |
|---|---|---|
| CMAX | 10.73 | nM |
| t1/2 | 2.15 | hr |
| Vd | 0.11 | L |
| VSS | 1.17 | L |
| CL | 12.09 | mL/hr/kg |
| BW | 3.00 | kg |
| 7 day | 843 | nM · min |
| AUC | | |

### Example 10. Cytokine Release in Cynomolgus Monkey after Single IV Bolus Injection of Ab-1

Cytokine release after Ab-1 administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for cytokines using a non-human primate cytometric Th1/Th2 bead array kit from BD biosciences following the manufacturers instructions. Interferon gamma, tumor necrosis factor alpha, interleukin 6, interleukin 5, interleukin 4, and interleukin 2 levels in plasma were calculated relative to reference standards provided with the bead array kit. FIG. 12A-12F illustrates cytokine release in cynomolgus monkey after a single IV bolus injection of Ab-1.

### Example 11. Analysis of Liver Enzymes in Cynomolgus Monkey after Single IV Bolus Injection of Ab-1

Systemic liver enzymes after polypeptide molecule administration by IV bolus was evaluated in cynomolgus monkeys. Briefly, cynomolgus monkeys of approximately 3 kg bodyweight were administered polypeptides as an IV bolus and observed daily for signs of adverse events. No in-life adverse events were observed. After dosing, blood was collected in K2 EDTA tubes at specific timepoints and processed to plasma. Plasma was stored frozen until analysis. Plasma samples were analyzed for the presence of liver enzymes aspartate transaminase (AST) and alanine aminotransferase (ALT) as signs of potential liver toxicity. AST and ALT levels remained within the normal ranges for all timepoints tested after dosing suggesting a lack of liver toxicity. AST and ALT were quantified following the instructions provided in a commercially available kit from Millipore. AST and ALT levels were calculated according to manufacturers instructions relative to a positive control reference standard. FIG. 13A-13B illustrates serum liver enzymes in cynomolgus monkey after a single IV bolus injection of Ab-1.

What is claimed is:

1. A multispecific antibody comprising a cluster of differentiation 28 (CD28) binding domain and a programmed death-ligand 1 (PD-L1) binding domain.

2. The multispecific antibody of claim 1, wherein when the CD28 binding domain is a single chain variable fragment (scFv), then the PD-L1 binding domain is not a scFv.

3. The multispecific antibody of claim 1, wherein the multispecific antibody is according to the following formula:
A-L-B (Formula I)
wherein
A comprises the CD28 binding domain;
B comprises the PD-L1 binding domain; and
L comprises a linker that connects A to B.

4. The multispecific antibody of claim 3, wherein the CD28 binding domain comprises a single chain variable fragment, a single domain antibody, a Fab, or a Fab′.

5. The multispecific antibody of claim 4, wherein the CD28 binding domain comprises the single chain variable fragment.

6. The multispecific antibody of claim 4, wherein the CD28 binding domain comprises the single domain antibody.

7. The multispecific antibody of claim 4, wherein the CD28 binding domain comprises the Fab or the Fab′.

8. The multispecific antibody of claim 1, wherein the PD-L1 binding domain comprises a single domain antibody, a Fab, or a Fab′.

9. The multispecific antibody of claim 8, wherein the PD-L1 binding domain comprises the Fab or the Fab′.

10. The multispecific antibody of claim 9, wherein the PD-L1 binding domain comprises the Fab or the Fab′ and the CD28 binding domain comprises the single chain variable fragment.

11. The multispecific antibody of claim 10, wherein the PD-L1 binding domain that comprises the Fab or the Fab′ comprises a Fab heavy chain polypeptide comprising a Fab heavy chain variable domain and a Fab light chain polypeptide comprising a Fab light chain variable domain.

12. The multispecific antibody of claim 11, wherein the CD28 binding domain that comprises the single chain variable fragment comprises a scFv heavy chain variable domain and a scFv light chain variable domain.

13. The multispecific antibody of claim 3, wherein the linker connects the C-terminus of A to an N-terminus of B.

14. The multispecific antibody of claim 3, wherein the linker connects the N-terminus of A to a C-terminus of B.

15. The multispecific antibody of claim 12, wherein the linker connects the C-terminus of A to the N-terminus of the Fab heavy chain polypeptide.

16. The multispecific antibody of claim 12, wherein the linker connects the N-terminus of A to the C-terminus of the Fab heavy chain polypeptide.

17. The multispecific antibody of claim 12, wherein the linker connects the C-terminus of A to the N-terminus of the Fab light chain polypeptide.

18. The multispecific antibody of claim 12, wherein the linker connects the N-terminus of A to the C-terminus of the Fab light chain polypeptide.

19. The multispecific antibody of claim 12, wherein the linker connects the Fab light chain polypeptide to the scFv light chain variable domain.

20. The multispecific antibody of claim 12, wherein the linker connects the Fab light chain polypeptide to the scFv heavy chain variable domain.

21. The multispecific antibody of claim 12, wherein the linker connects the Fab heavy chain polypeptide to the scFv light chain variable domain.

22. The multispecific antibody of claim 12, wherein the linker connects the Fab heavy chain polypeptide to the scFv heavy chain variable domain.

23. The multispecific antibody of claim 12, wherein the linker connects the Fab light chain polypeptide to the N-terminus of the scFv light chain variable domain.

24. The multispecific antibody of claim 12, wherein the linker connects the Fab light chain polypeptide to the C-terminus of the scFv light chain variable domain.

25. The multispecific antibody of claim 12, wherein the linker connects the Fab light chain polypeptide to the N-terminus of the scFv heavy chain variable domain.

26. The multispecific antibody of claim 12, wherein the linker connects the Fab light chain polypeptide to the C-terminus of the scFv heavy chain variable domain.

27. The multispecific antibody of claim 12, wherein the linker connects the Fab heavy chain polypeptide to the N-terminus of the scFv light chain variable domain.

28. The multispecific antibody of claim 12, wherein the linker connects the Fab heavy chain polypeptide to the C-terminus of the scFv light chain variable domain.

29. The multispecific antibody of claim 12, wherein the linker connects the Fab heavy chain polypeptide to the N-terminus of the scFv heavy chain variable domain.

30. The multispecific antibody of claim 12, wherein the linker connects the Fab heavy chain polypeptide to the C-terminus of the scFv heavy chain variable domain.

31. The multispecific antibody of claim 3, wherein the linker is at least 5 amino acids in length.

32. The multispecific antibody of claim 31, wherein the linker is no more than 30 amino acids in length.

33. The multispecific antibody of claim 32, wherein the linker is at least 5 amino acids and no more than 30 amino acids in length.

34. The multispecific antibody of claim 33, wherein the linker is 5 amino acids in length.

35. The multispecific antibody of claim 33, wherein the linker is 15 amino acids in length.

36. The multispecific antibody of claim 3, wherein the linker is selected from the group consisting of (G₂S)ₙ, (GS)ₙ, (GSGGS)ₙ (SEQ ID NO: 58), (GGGS)ₙ (SEQ ID NO: 59), (GGGGS)ₙ (SEQ ID NO: 60), and (GSSGGS)ₙ (SEQ ID NO: 61), wherein n is an integer of at least 1.

37. The multispecific antibody of claim 3, wherein L has a formula comprising (G₂S)ₙ (SEQ ID NO: 233), wherein n is an integer from 1 to 3.

38. The multispecific antibody of claim 3, wherein the L comprises an amino acid sequence of SEQ ID NO: 18 (GGGGSGGGGSGGGGS) or SEQ ID NO: 19 (GGGGS).

39. The multispecific antibody of claim 1, wherein the CD28 binding domain comprises heavy chain variable domain complementarity determining regions (CDRs): HC-CDR1, HC-CDR2, and HC-CDR3, wherein the HC-CDR1, the HC-CDR2, and the HC-CDR3 comprise: HC-CDR1: SEQ ID NO: 1; HC-CDR2: SEQ ID NO: 2; HC-CDR3: SEQ ID NO: 3, and wherein said CDRs comprise from 0-2 amino acid modifications in at least one of said HC-CDR1, HC-CDR2, or HC-CDR3.

40. The multispecific antibody of claim 1, wherein the CD28 binding domain comprises light chain variable domain complementarity determining regions (CDRs): LC-CDR1, LC-CDR2, and LC-CDR3, wherein the LC-CDR1, the LC-CDR2, and the LC-CDR3 comprise: LC-CDR1: SEQ ID NO: 4; LC-CDR2: SEQ ID NO: 5; and LC-CDR3: SEQ ID NO: 6, and wherein the CDRs comprise from 0-2 amino acid modifications in at least one of said LC-CDR1, LC-CDR2, or LC-CDR3.

41. The multispecific antibody of claim 1, wherein the PD-L1 binding domain comprises heavy chain variable domain complementarity determining region (CDRs): HC-CDR1, HC-CDR2, and HC-CDR3, wherein the HC-CDR1, the HC-CDR2, and the HC-CDR3 comprise: HC-CDR1: SEQ ID NO: 10; HC-CDR2: SEQ ID NO: 11; HC-CDR3: SEQ ID NO: 12, HC-CDR1: SEQ ID NO: 24; HC-CDR2: SEQ ID NO: 25; HC-CDR3: SEQ ID NO: 26; or HC-CDR1 SEQ ID NO: 27; HC-CDR2: SEQ ID NO: 28; HC-CDR3: SEQ ID NO: 29; or HC-CDR1: SEQ ID NO: 30; HC-CDR2: SEQ ID NO: 31; HC-CDR3: SEQ ID NO: 32; and wherein said CDRs comprise from 0-2 amino acid modifications in at least one of said HC-CDR1, HC-CDR2, or HC-CDR3.

42. The multispecific antibody of claim 1, wherein the PD-L1 binding domain comprises light chain variable domain complementarity determining regions (CDRs): LC-CDR1, LC-CDR2, and LC-CDR3, wherein the LC-CDR1, the LC-CDR2, and the LC-CDR3 comprise:LC-CDR1: SEQ ID NO: 13; LC-CDR2: SEQ ID NO: 14; and LC-CDR3: SEQ ID NO: 15; LC-CDR1: SEQ ID NO: 33; LC-CDR2: SEQ ID NO: 34; and LC-CDR3: SEQ ID NO: 35;
LC-CDR1: SEQ ID NO: 36; LC-CDR2: SEQ ID NO: 37; and LC-CDR3: SEQ ID NO: 38; or
LC-CDR1: SEQ ID NO: 39; LC-CDR2: SEQ ID NO: 40; and LC-CDR3: SEQ ID NO: 41; and wherein the CDRs comprise from 0-2 amino acid modifications in at least one of said LC-CDR1, LC-CDR2, or LC-CDR3.

43. The multispecific antibody of claim 1, wherein the CD28 binding domain comprises an amino acid sequence that has at least 80% sequence identity to the amino acid sequence according to SEQ ID NO: 7.

44. The multispecific antibody of claim 1, wherein the CD28 binding domain comprises an amino acid sequence of at least 75 consecutive amino acid residues of SEQ ID NO: 7

45. The multispecific antibody of claim 1, wherein the CD28 binding domain comprises an amino acid sequence of at least 110 consecutive amino acid residues of SEQ ID NO: 7.

46. The multispecific antibody of claim 1, wherein the CD28 binding domain comprises an amino acid sequence of at least 110 consecutive amino acid residues of SEQ ID NO: 7 and has at least 80% sequence identity to the at least 110 consecutive amino acid residues of SEQ ID NO: 7.

47. The multispecific antibody of claim 1, wherein the CD28 binding domain comprises an amino acid sequence according to SEQ ID NO: 7.

48. The multispecific antibody of claim 1, wherein the CD28 binding domain comprises an amino acid sequence that has at least 80% sequence identity to the amino acid sequence according to SEQ ID NO: 8.

49. The multispecific antibody of claim 1, wherein the CD28 binding domain comprises an amino acid sequence of at least 75 consecutive amino acid residues of SEQ ID NO: 8.

50. The multispecific antibody of claim 1, wherein the CD28 binding domain comprises an amino acid sequence of at least 100 consecutive amino acid residues of SEQ ID NO: 8.

51. The multispecific antibody of claim 1, wherein the CD28 binding domain comprises an amino acid sequence of at least 100 consecutive amino acid residues of SEQ ID NO: 8 and has at least 80% sequence identity to the at least 100 consecutive amino acid residues of SEQ ID NO: 8.

52. The multispecific antibody of claim 1, wherein the CD28 binding domain comprises an amino acid sequence according to SEQ ID NO: 8.

53. The multispecific antibody of claim 1, wherein the CD28 binding domain comprises an amino acid sequence that has at least 80% sequence identity to the amino acid sequence according to SEQ ID NO: 9.

54. The multispecific antibody of claim 1, wherein the CD28 binding domain comprises an amino acid sequence of at least 175 consecutive amino acid residues of SEQ ID NO: 9.

55. The multispecific antibody of claim 1, wherein the CD28 binding domain comprises an amino acid sequence of at least 210 consecutive amino acid residues of SEQ ID NO: 9.

56. The multispecific antibody of claim 1, wherein the CD28 binding domain comprises an amino acid sequence of at least 210 consecutive amino acid residues of SEQ ID NO: 9 and has at least 80% sequence identity to the at least 210 consecutive amino acid residues of SEQ ID NO: 9.

57. The multispecific antibody of claim 1, wherein the CD28 binding domain comprises an amino acid sequence according to SEQ ID NO: 9.

58. The multispecific antibody of claim 1, wherein the PD-L1 binding domain comprises an amino acid sequence that has at least 80% sequence identity to the amino acid sequence according to SEQ ID NO: 17, 43, 45, or 47.

59. The multispecific antibody of claim 1, wherein the PD-L1 binding domain comprises an amino acid sequence of at least 175 consecutive amino acid residues of SEQ ID NO: 17, 43, 45, or 47.

60. The multispecific antibody of claim 1, wherein the PD-L1 binding domain comprises an amino acid sequence of at least 215 consecutive amino acid residues of SEQ ID NO: 17, 43, 45, or 47.

61. The multispecific antibody of claim 1, wherein the PD-L1 binding domain comprises an amino acid sequence of at least 215 consecutive amino acid residues of SEQ ID NO: 17 and has at least 80% sequence identity to the at least 215 consecutive amino acid residues of SEQ ID NO: 17, 43, 45, or 47.

62. The multispecific antibody of claim 1, wherein the PD-L1 binding domain comprises an amino acid sequence according to SEQ ID NO: 17, 43, 45, or 47.

63. The multispecific antibody of claim 1, wherein the PD-L1 binding domain comprises an amino acid sequence that has at least 80% sequence identity to the amino acid sequence according to SEQ ID NO: 16, 42, 44, or 46.

64. The multispecific antibody of claim 1, wherein the PD-L1 binding domain comprises an amino acid sequence of at least 175 consecutive amino acid residues of SEQ ID NO: 16, 42, 44, or 46.

65. The multispecific antibody of claim 1, wherein the PD-L1 binding domain comprises an amino acid sequence of at least 200 consecutive amino acid residues of SEQ ID NO: 16, 42, 44, or 46.

66. The multispecific antibody of claim 1, wherein the PD-L1 binding domain comprises an amino acid sequence of at least 200 consecutive amino acid residues of SEQ ID NO: 16 and has at least 80% sequence identity to the at least 200 consecutive amino acid residues of SEQ ID NO: 16, 42, 44, or 46.

67. The multispecific antibody of claim 1, wherein PD-L1 binding domain comprises an amino acid sequence according to SEQ ID NO: 16, 42, 44, or 46.

68. The multispecific antibody of claim 12, wherein the linker connects the Fab heavy chain polypeptide to the C-terminus of the scFv light chain variable domain and wherein the Fab light chain polypeptide comprises an amino acid sequence that has at least 80% sequence identity to the amino acid sequence according to SEQ ID NO: 20, and an amino acid sequence of the Fab heavy chain polypeptide that is connected to the C-terminus of the scFv light chain variable domain comprises an amino acid sequence that has at least 80% sequence identity to the amino acid sequence according to SEQ ID NO: 21.

69. The multispecific antibody of claim 12, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20, and an amino acid sequence of the Fab heavy chain polypeptide that is connected to the C-terminus of the scFv light chain variable domain comprises an amino acid sequence of at least 450 consecutive amino acid residues of SEQ ID NO: 21.

70. The multispecific antibody of claim 12, wherein the linker connects the Fab heavy chain polypeptide to the C-terminus of the scFv light chain variable domain and wherein the Fab light chain polypeptide comprises an amino acid sequence of at least 200 consecutive amino acid residues of SEQ ID NO: 20 and has at least 80% sequence identity to the at least 200 consecutive amino acid residues of SEQ ID NO: 20 and an amino acid sequence of the Fab heavy chain polypeptide that is connected to the C-terminus of the scFv light chain variable domain comprises an amino acid sequence of at least 450 consecutive amino acid residues of SEQ ID NO: 21 and has at least 80% sequence identity to the at least 450 consecutive amino acid residues of SEQ ID NO: 21.

71. The multispecific antibody of claim 12, wherein the linker connects the Fab heavy chain polypeptide to the C-terminus of the scFv light chain variable domain and wherein the Fab light chain polypeptide comprises an amino acid sequence according to SEQ ID NO: 20, and an amino acid sequence of the Fab heavy chain polypeptide that is connected to the C-terminus of the scFv light chain variable domain comprises an amino acid sequence to SEQ ID NO: 21.

72. The multispecific antibody of claim 12, wherein the linker connects the Fab light chain polypeptide to the C-terminus of the scFv light chain variable domain and wherein the Fab heavy chain polypeptide comprises an amino acid sequence that has at least 80% sequence identity to the amino acid sequence according to SEQ ID NO: 23, and an amino acid sequence of the Fab light chain polypeptide that is connected to the C-terminus of the scFv light chain variable domain comprises an amino acid sequence that has at least 80% sequence identity to the amino acid sequence according to SEQ ID NO: 22.

73. The multispecific antibody of claim 12, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23, and an amino acid sequence of the Fab light chain polypeptide that is connected to the C-terminus of the scFv light chain variable domain comprises an amino acid sequence of at least 450 consecutive amino acid residues of SEQ ID NO: 22.

74. The multispecific antibody of claim 12, wherein the linker connects the Fab light chain polypeptide to the C-terminus of the scFv light chain variable domain and wherein the Fab heavy chain polypeptide comprises an amino acid sequence of at least 200 consecutive amino acid residues of SEQ ID NO: 23 and has at least 80% sequence identity to the at least 200 consecutive amino acid residues of SEQ ID NO: 23 and an amino acid sequence of the Fab light chain polypeptide that is connected to the C-terminus of the scFv light chain variable domain comprises an amino acid sequence of at least 450 consecutive amino acid residues of SEQ ID NO: 22 and has at least 80% sequence identity to the at least 450 consecutive amino acid residues of SEQ ID NO: 22.

75. The multispecific antibody of claim 12, wherein the linker connects the Fab light chain polypeptide to the C-terminus of the scFv light chain variable domain and wherein the Fab heavy chain polypeptide comprises an amino acid sequence according to SEQ ID NO: 23, and an amino acid sequence of the Fab light chain polypeptide that is connected to the C-terminus of the scFv light chain variable domain comprises an amino acid sequence to SEQ ID NO: 22.

76. A pharmaceutical composition comprising:
(i) the multispecific antibody of any one of claims 1-75; and
(ii) a pharmaceutically acceptable excipient.

77. An isolated recombinant nucleic acid molecule encoding a polypeptide of the multispecific antibody of any one of claims 1-75.


SEQUENCE LISTING (250 sequences)

SEQ ID NO: 1 - protein, 8 aa, Artificial Sequence
  Description of Artificial Sequence Synthetic peptide
GYTFTSYY

SEQ ID NO: 2 - protein, 8 aa, Artificial Sequence
  Description of Artificial Sequence Synthetic peptide
IYPGNVNT

SEQ ID NO: 3 - protein, 13 aa, Artificial Sequence
  Description of Artificial Sequence Synthetic peptide
TRSHYGLDWNFDV

SEQ ID NO: 4 - protein, 6 aa, Artificial Sequence
  Description of Artificial Sequence Synthetic peptide
QNIYVW

SEQ ID NO: 5 - protein, 2 aa, Artificial Sequence
  Description of Artificial Sequence Synthetic peptide
KA

SEQ ID NO: 6 - protein, 9 aa, Artificial Sequence
  Description of Artificial Sequence Synthetic peptide
QQGQTYPYT

SEQ ID NO: 7 - protein, 120 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 8 - protein, 107 aa, Artificial Sequence
  Description of Artificial Sequence Synthetic polypeptide
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 9 - protein, 242 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 10 - protein, 8 aa, Artificial Sequence
  Description of Artificial Sequence Synthetic peptide
GDTFSTYA

SEQ ID NO: 11 - protein, 8 aa, Artificial Sequence
  Description of Artificial Sequence Synthetic peptide
IIPIFGKA

SEQ ID NO: 12 - protein, 16 aa, Artificial Sequence
  Description of Artificial Sequence Synthetic peptide
ARKFHFVSGSPFGMDV

SEQ ID NO: 13 - protein, 6 aa, Artificial Sequence
  Description of Artificial Sequence Synthetic peptide
QSVSSY

SEQ ID NO: 14 - protein, 2 aa, Artificial Sequence
  Description of Artificial Sequence Synthetic peptide
DA

SEQ ID NO: 15 - protein, 8 aa, Artificial Sequence
  Description of Artificial Sequence Synthetic peptide
QQRSNWPT

SEQ ID NO: 16 - protein, 213 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 226 aa, Artificial Sequence
  Description of Artificial Sequence Synthetic polypeptide
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 15 aa, Artificial Sequence
  Description of Artificial Sequence Synthetic peptide
GGGGSGGGGSGGGGS

SEQ ID NO: 19 - protein, 5 aa, Artificial Sequence
  Description of Artificial Sequence Synthetic peptide
GGGGS

SEQ ID NO: 20 - protein, 213 aa, Artificial Sequence; identical to SEQ ID NO: 16
  Description of Artificial Sequence Synthetic polypeptide

SEQ ID NO: 21 - protein, 473 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 22 - protein, 460 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 226 aa, Artificial Sequence; identical to SEQ ID NO: 17
  Description of Artificial Sequence Synthetic polypeptide

SEQ ID NO: 24 - protein, 8 aa, Artificial Sequence
  Description of Artificial Sequence Synthetic peptide
GFTFSDSW

SEQ ID NO: 25 - protein, 8 aa, Artificial Sequence
  Description of Artificial Sequence Synthetic peptide
ISPYGGST

SEQ ID NO: 26 - protein, 11 aa, Artificial Sequence
  Description of Artificial Sequence Synthetic peptide
ARRHWPGGFDY

SEQ ID NO: 27 - protein, 8 aa, Artificial Sequence
  Description of Artificial Sequence Synthetic peptide
GFTFSSYI

SEQ ID NO: 28 - protein, 8 aa, Artificial Sequence
  Description of Artificial Sequence Synthetic peptide
IYPSGGIT

SEQ ID NO: 29 - protein, 13 aa, Artificial Sequence
  Description of Artificial Sequence Synthetic peptide
ARIKLGTVTTVDY

SEQ ID NO: 30 - protein, 8 aa, Artificial Sequence
  Description of Artificial Sequence Synthetic peptide
GFTFSRYW

SEQ ID NO: 31 - protein, 8 aa, Artificial Sequence
  Description of Artificial Sequence Synthetic peptide
IKQDGSEK

SEQ ID NO: 32 - protein, 14 aa, Artificial Sequence
  Description of Artificial Sequence Synthetic peptide
AREGGWFGELAFDY

SEQ ID NO: 33 - protein, 6 aa, Artificial Sequence
  Description of Artificial Sequence Synthetic peptide
QDVSTA

SEQ ID NO: 34 - protein, 2 aa, Artificial Sequence
  Description of Artificial Sequence Synthetic peptide
SA

SEQ ID NO: 35 - protein, 9 aa, Artificial Sequence
  Description of Artificial Sequence Synthetic peptide
QQYLYHPAT

SEQ ID NO: 36 - protein, 9 aa, Artificial Sequence
  Description of Artificial Sequence Synthetic peptide
SSDVGGYNY

SEQ ID NO: 37 - protein, 2 aa, Artificial Sequence
  Description of Artificial Sequence Synthetic peptide
DV

SEQ ID NO: 38 - protein, 13 aa, Artificial Sequence
  Description of Artificial Sequence Synthetic peptide
FGTGTKVTVLGQP

SEQ ID NO: 39 - protein, 7 aa, Artificial Sequence
  Description of Artificial Sequence Synthetic peptide
QRVSSSY

SEQ ID NO: 40 - protein, 2 aa, Artificial Sequence
  Description of Artificial Sequence Synthetic peptide
DA

SEQ ID NO: 41 - protein, 9 aa, Artificial Sequence
  Description of Artificial Sequence Synthetic peptide
QQYGSLPWT

SEQ ID NO: 42 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 221 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 44 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 45 - protein, 223 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 215 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 224 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 48 - protein, 468 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 49 - protein, 461 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 470 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - protein, 463 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 52 - protein, 471 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 53 - protein, 462 aa, Artificial Sequence
  Description of Artificial Sequence Synthetic polypeptide
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 8 aa, Artificial Sequence
  Description of Artificial Sequence Synthetic peptide
GSTFYTAV

SEQ ID NO: 55 - protein, 8 aa, Artificial Sequence
  Description of Artificial Sequence Synthetic peptide
IRWTALTT

SEQ ID NO: 56 - protein, 17 aa, Artificial Sequence
  Description of Artificial Sequence Synthetic peptide
AARGTLGLFTTADSYDY

SEQ ID NO: 57 - protein, 124 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSS

SEQ ID NO: 58 - protein, 5 aa, Artificial Sequence
  Description of Artificial Sequence Synthetic peptide
  See specification as filed for detailed description of substitutions and preferred embodiments
GSGGS

SEQ ID NO: 59 - protein, 4 aa, Artificial Sequence
  Description of Artificial Sequence Synthetic peptide
  See specification as filed for detailed description of substitutions and preferred embodiments
GGGS

SEQ ID NO: 60 - protein, 5 aa, Artificial Sequence
  Description of Artificial Sequence Synthetic peptide
  See specification as filed for detailed description of substitutions and preferred embodiments
GGGGS

SEQ ID NO: 61 - protein, 6 aa, Artificial Sequence
  Description of Artificial Sequence Synthetic peptide
  See specification as filed for detailed description of substitutions and preferred embodiments
GSSGGS

SEQ ID NO: 62 - protein, 9 aa, Artificial Sequence
  Description of Artificial Sequence Synthetic peptide
GGGGSGGGS

SEQ ID NO: 63 - protein, 28 aa, Artificial Sequence
  Description of Artificial Sequence Synthetic peptide
GGGGSGGGLSGRSDAGSPLGLAGSGGGS

SEQ ID NO: 64 - protein, 18 aa, Artificial Sequence
  Description of Artificial Sequence Synthetic peptide
GGGGSLSGRSDNHGSSGT

SEQ ID NO: 65 - protein, 26 aa, Artificial Sequence
  Description of Artificial Sequence Synthetic peptide
GGGGSSGGSGGSGLSGRSDNHGSSGT

SEQ ID NO: 66 - protein, 8 aa, Artificial Sequence
  Description of Artificial Sequence Synthetic peptide
ASGRSDNH

SEQ ID NO: 67 - protein, 8 aa, Artificial Sequence
  Description of Artificial Sequence Synthetic peptide
LAGRSDNH

SEQ ID NO: 68 - protein, 13 aa, Artificial Sequence
  Description of Artificial Sequence Synthetic peptide
ISSGLASGRSDNH

SEQ ID NO: 69 - protein, 13 aa, Artificial Sequence
  Description of Artificial Sequence Synthetic peptide
ISSGLLAGRSDNH

SEQ ID NO: 70 - protein, 8 aa, Artificial Sequence
  Description of Artificial Sequence Synthetic peptide
LSGRSDNH

SEQ ID NO: 71 - protein, 13 aa, Artificial Sequence
  Description of Artificial Sequence Synthetic peptide
ISSGLLSGRSDNP

SEQ ID NO: 72 - protein, 13 aa, Artificial Sequence
  Description of Artificial Sequence Synthetic peptide
ISSGLLSGRSDNH

SEQ ID NO: 73 - protein, 16 aa, Artificial Sequence
  Description of Artificial Sequence Synthetic peptide
LSGRSDNHSPLGLAGS

SEQ ID NO: 74 - protein, 16 aa, Artificial Sequence
  Description of Artificial Sequence Synthetic peptide
SPLGLAGSLSGRSDNH

SEQ ID NO: 75 - protein, 12 aa, Artificial Sequence
  Description of Artificial Sequence Synthetic peptide
SPLGLSGRSDNH

SEQ ID NO: 76 - protein, 16 aa, Artificial Sequence
  Description of Artificial Sequence Synthetic peptide
LAGRSDNHSPLGLAGS

SEQ ID NO: 77 - protein, 16 aa, Artificial Sequence
  Description of Artificial Sequence Synthetic peptide
LSGRSDNHVPLSLKMG

SEQ ID NO: 78 - protein, 16 aa, Artificial Sequence
  Description of Artificial Sequence Synthetic peptide
LSGRSDNHVPLSLSMG

SEQ ID NO: 79 - protein, 31 aa, Artificial Sequence
  Description of Artificial Sequence Synthetic polypeptide
GSSGGSGGSGGSGISSGLLSGRSDNHGSSGT

SEQ ID NO: 80 - protein, 28 aa, Artificial Sequence
  Description of Artificial Sequence Synthetic peptide
GSSGGSGGSGGISSGLLSGRSDNHGGGS

SEQ ID NO: 81 - protein, 8 aa, Artificial Sequence
  Description of Artificial Sequence Synthetic peptide
ASGRSDNH

SEQ ID NO: 82 - protein, 8 aa, Artificial Sequence
  Description of Artificial Sequence Synthetic peptide
LAGRSDNH

SEQ ID NO: 83 - protein, 13 aa, Artificial Sequence
  Description of Artificial Sequence Synthetic peptide
ISSGLASGRSDNH

SEQ ID NO: 84 - protein, 8 aa, Artificial Sequence
  Description of Artificial Sequence Synthetic peptide
LSGRSDAG

SEQ ID NO: 85 - protein, 13 aa, Artificial Sequence
  Description of Artificial Sequence Synthetic peptide
ISSGLLSGRSDAG

SEQ ID NO: 86 - protein, 18 aa, Artificial Sequence
  Description of Artificial Sequence Synthetic peptide
AAGLLAPPGGLSGRSDAG

SEQ ID NO: 87 - protein, 12 aa, Artificial Sequence
  Description of Artificial Sequence Synthetic peptide
SPLGLSGRSDAG

SEQ ID NO: 88 - protein, 15 aa, Artificial Sequence
  Description of Artificial Sequence Synthetic peptide
LSGRSDAGSPLGLAG

SEQ ID NO: 89 - protein, 8 aa, Artificial Sequence
  Description of Artificial Sequence Synthetic peptide
GFTFNKYA

SEQ ID NO: 90 - protein, 10 aa, Artificial Sequence
  Description of Artificial Sequence Synthetic peptide
IRSKYNNYAT

SEQ ID NO: 91 - protein, 16 aa, Artificial Sequence
  Description of Artificial Sequence Synthetic peptide
VRHGNFGNSYISYWAY

SEQ ID NO: 92 - protein, 9 aa, Artificial Sequence
  Description of Artificial Sequence Synthetic peptide
TGAVTSGNY

SEQ ID NO: 93 - protein, 3 aa, Artificial Sequence
  Description of Artificial Sequence Synthetic peptide
GTK

SEQ ID NO: 94 - protein, 9 aa, Artificial Sequence
  Description of Artificial Sequence Synthetic peptide
VLWYSNRWV

SEQ ID NO: 95 - protein, 8 aa, Artificial Sequence
  Description of Artificial Sequence Synthetic peptide
GFTFNTYA

SEQ ID NO: 96 - protein, 16 aa, Artificial Sequence
  Description of Artificial Sequence Synthetic peptide
VRHGNFGNSYVSWFAY

SEQ ID NO: 97 - protein, 9 aa, Artificial Sequence
  Description of Artificial Sequence Synthetic peptide
TGAVTTSNY

SEQ ID NO: 98 - protein, 2 aa, Artificial Sequence
  Description of Artificial Sequence Synthetic peptide
GT

SEQ ID NO: 99 - protein, 9 aa, Artificial Sequence
  Description of Artificial Sequence Synthetic peptide
ALWYSNLWV

SEQ ID NO: 100 - protein, 249 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 101 - protein, 249 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 102 - protein, 6 aa, Artificial Sequence
  Description of Artificial Sequence Synthetic peptide
QSIGTN

SEQ ID NO: 103 - protein, 3 aa, Artificial Sequence
  Description of Artificial Sequence Synthetic peptide
YAS

SEQ ID NO: 104 - protein, 9 aa, Artificial Sequence
  Description of Artificial Sequence Synthetic peptide
QQNNNWPTT

SEQ ID NO: 105 - protein, 8 aa, Artificial Sequence
  Description of Artificial Sequence Synthetic peptide
GFSLTNYG

SEQ ID NO: 106 - protein, 7 aa, Artificial Sequence
  Description of Artificial Sequence Synthetic peptide
IWSGGNT

SEQ ID NO: 107 - protein, 13 aa, Artificial Sequence
  Description of Artificial Sequence Synthetic peptide
ARALTYYDYEFAY

SEQ ID NO: 108 - protein, 214 aa, Artificial Sequence
  Description of Artificial Sequence Synthetic polypeptide
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 222 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 111 - protein, 222 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 112 - protein, 8 aa, Artificial Sequence
  Description of Artificial Sequence Synthetic peptide
GYTFTNYG

SEQ ID NO: 113 - protein, 8 aa, Artificial Sequence
  Description of Artificial Sequence Synthetic peptide
INTYTGEP

SEQ ID NO: 114 - protein, 14 aa, Artificial Sequence
  Description of Artificial Sequence Synthetic peptide
ARGGFGSSYWYFDV

SEQ ID NO: 115 - protein, 6 aa, Artificial Sequence
  Description of Artificial Sequence Synthetic peptide
QDVSIA

SEQ ID NO: 116 - protein, 3 aa, Artificial Sequence
  Description of Artificial Sequence Synthetic peptide
SAS

SEQ ID NO: 117 - protein, 9 aa, Artificial Sequence
  Description of Artificial Sequence Synthetic peptide
QQHYITPLT

SEQ ID NO: 118 - protein, 214 aa, Artificial Sequence
  Description of Artificial Sequence Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 224 aa, Artificial Sequence
  Description of Artificial Sequence Synthetic polypeptide
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 120 - protein, 8 aa, Artificial Sequence
  Description of Artificial Sequence Synthetic peptide
GFAFSRYG

SEQ ID NO: 121 - protein, 8 aa, Artificial Sequence
  Description of Artificial Sequence Synthetic peptide
IWYDGSNK

SEQ ID NO: 122 - protein, 16 aa, Artificial Sequence
  Description of Artificial Sequence Synthetic peptide
ARGGDFLYYYYYGMDV

SEQ ID NO: 123 - protein, 6 aa, Artificial Sequence
  Description of Artificial Sequence Synthetic peptide
QGISNY

SEQ ID NO: 124 - protein, 2 aa, Artificial Sequence
  Description of Artificial Sequence Synthetic peptide
EA

SEQ ID NO: 125 - protein, 9 aa, Artificial Sequence
  Description of Artificial Sequence Synthetic peptide
QNYNSAPFT

SEQ ID NO: 126 - protein, 214 aa, Artificial Sequence
  Description of Artificial Sequence Synthetic polypeptide
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 226 aa, Artificial Sequence
  Description of Artificial Sequence Synthetic polypeptide
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 128 - protein, 478 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 129 - protein, 224 aa, Artificial Sequence; identical to SEQ ID NO: 119
  Description of Artificial Sequence Synthetic polypeptide

SEQ ID NO: 130 - protein, 468 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 214 aa, Artificial Sequence; identical to SEQ ID NO: 118
  Description of Artificial Sequence Synthetic polypeptide

SEQ ID NO: 132 - protein, 478 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 133 - protein, 256 aa, Artificial Sequence
  Description of Artificial Sequence Synthetic polypeptide
GGVDFCKIYSWPVCHQGGGGSGGLSGRSDAGSPLGLAGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 655 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 135 - protein, 224 aa, Artificial Sequence; identical to SEQ ID NO: 119
  Description of Artificial Sequence Synthetic polypeptide

SEQ ID NO: 136 - protein, 468 aa, Artificial Sequence; identical to SEQ ID NO: 130
  Description of Artificial Sequence Synthetic polypeptide

SEQ ID NO: 137 - protein, 214 aa, Artificial Sequence; identical to SEQ ID NO: 118
  Description of Artificial Sequence Synthetic polypeptide

SEQ ID NO: 138 - protein, 478 aa, Artificial Sequence; identical to SEQ ID NO: 132
  Description of Artificial Sequence Synthetic polypeptide

SEQ ID NO: 139 - protein, 214 aa, Artificial Sequence; identical to SEQ ID NO: 108
  Description of Artificial Sequence Synthetic polypeptide

SEQ ID NO: 140 - protein, 222 aa, Artificial Sequence; identical to SEQ ID NO: 110
  Description of Artificial Sequence Synthetic polypeptide

SEQ ID NO: 141 - protein, 214 aa, Artificial Sequence; identical to SEQ ID NO: 109
  Description of Artificial Sequence Synthetic polypeptide

SEQ ID NO: 142 - protein, 222 aa, Artificial Sequence; identical to SEQ ID NO: 111
  Description of Artificial Sequence Synthetic polypeptide

SEQ ID NO: 143 - protein, 214 aa, Artificial Sequence; identical to SEQ ID NO: 118
  Description of Artificial Sequence Synthetic polypeptide

SEQ ID NO: 144 - protein, 478 aa, Artificial Sequence; identical to SEQ ID NO: 132
  Description of Artificial Sequence Synthetic polypeptide

SEQ ID NO: 145 - protein, 468 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 224 aa, Artificial Sequence; identical to SEQ ID NO: 119
  Description of Artificial Sequence Synthetic polypeptide

SEQ ID NO: 147 - protein, 214 aa, Artificial Sequence; identical to SEQ ID NO: 118
  Description of Artificial Sequence Synthetic polypeptide

SEQ ID NO: 148 - protein, 478 aa, Artificial Sequence; identical to SEQ ID NO: 128
  Description of Artificial Sequence Synthetic polypeptide

SEQ ID NO: 149 - protein, 468 aa, Artificial Sequence; identical to SEQ ID NO: 130
  Description of Artificial Sequence Synthetic polypeptide

SEQ ID NO: 150 - protein, 224 aa, Artificial Sequence; identical to SEQ ID NO: 119
  Description of Artificial Sequence Synthetic polypeptide

SEQ ID NO: 151 - protein, 214 aa, Artificial Sequence
  Description of Artificial Sequence Synthetic polypeptide
DIQMTQSPSSLSASVGDRVTITCRASQGITNYLAWFQQKPGKAPKSLIYAASSLQSGVPSKFSGSGSGTDFSLTISSLQPEDFATYYCQQYNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 481 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLVESGGGVVQPGRSLRLSCAASGFTFSNYVMHWVRQAPGKGLEWVAIIWYDGSNKYYADSVKGRFTISRDNSKNTLYLQMNSLRAEDTAVYYCAGGYNWNYEYHYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 153 - protein, 214 aa, Artificial Sequence; identical to SEQ ID NO: 126
  Description of Artificial Sequence Synthetic polypeptide

SEQ ID NO: 154 - protein, 480 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 155 - protein, 468 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 226 aa, Artificial Sequence; identical to SEQ ID NO: 127
  Description of Artificial Sequence Synthetic polypeptide

SEQ ID NO: 157 - protein, 214 aa, Artificial Sequence; identical to SEQ ID NO: 126
  Description of Artificial Sequence Synthetic polypeptide

SEQ ID NO: 158 - protein, 480 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 159 - protein, 468 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 226 aa, Artificial Sequence; identical to SEQ ID NO: 127
  Description of Artificial Sequence Synthetic polypeptide

SEQ ID NO: 161 - protein, 14 aa, Artificial Sequence
  Description of Artificial Sequence Synthetic peptide
GSQCLGPEWEVCPY

SEQ ID NO: 162 - protein, 14 aa, Artificial Sequence
  Description of Artificial Sequence Synthetic peptide
VYCGPEFDESVGCM

SEQ ID NO: 163 - protein, 14 aa, Artificial Sequence
  Description of Artificial Sequence Synthetic peptide
PCRSHIDVAKPICV

SEQ ID NO: 164 - protein, 256 aa, Artificial Sequence
  Description of Artificial Sequence Synthetic polypeptide
GGPCRSHIDVAKPICVGGGGSGGLSGRSDAGSPLGLAGSGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 653 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 166 - protein, 256 aa, Artificial Sequence; identical to SEQ ID NO: 164
  Description of Artificial Sequence Synthetic polypeptide

SEQ ID NO: 167 - protein, 644 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 168 - protein, 256 aa, Artificial Sequence
  Description of Artificial Sequence Synthetic polypeptide
GGPCRSHIDVAKPICVGGGGSGGGGGSGGGGSGGASSGAGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 644 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGSGGGGSGGASSGAGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 170 - protein, 261 aa, Artificial Sequence
  Description of Artificial Sequence Synthetic polypeptide
GGPCRSHIDVAKPICVGGGGSSGGSAAGLLAPPGGLSGRSDAGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 644 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGSSGGSAAGLLAPPGGLSGRSDAGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 172 - protein, 256 aa, Artificial Sequence; identical to SEQ ID NO: 164
  Description of Artificial Sequence Synthetic polypeptide

SEQ ID NO: 173 - protein, 256 aa, Artificial Sequence; identical to SEQ ID NO: 164
  Description of Artificial Sequence Synthetic polypeptide

SEQ ID NO: 174 - protein, 644 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 175 - protein, 468 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 222 aa, Artificial Sequence; identical to SEQ ID NO: 111
  Description of Artificial Sequence Synthetic polypeptide

SEQ ID NO: 177 - protein, 645 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 264 aa, Artificial Sequence
  Description of Artificial Sequence Synthetic polypeptide
GGPCRSHIDVAKPICVGGGGSGGLSGRSDAGSPLGLAGSGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 179 - protein, 14 aa, Artificial Sequence
  Description of Artificial Sequence Synthetic peptide
SVLFCVKNLYCWVT

SEQ ID NO: 180 - protein, 14 aa, Artificial Sequence
  Description of Artificial Sequence Synthetic peptide
VDFCKIYSWPVCHQ

SEQ ID NO: 181 - protein, 14 aa, Artificial Sequence
  Description of Artificial Sequence Synthetic peptide
IDFCMLYNWPICAG

SEQ ID NO: 182 - protein, 256 aa, Artificial Sequence; identical to SEQ ID NO: 133
  Description of Artificial Sequence Synthetic polypeptide

SEQ ID NO: 183 - protein, 655 aa, Artificial Sequence; identical to SEQ ID NO: 134
  Description of Artificial Sequence Synthetic polypeptide

SEQ ID NO: 184 - protein, 256 aa, Artificial Sequence
  Description of Artificial Sequence Synthetic polypeptide
GGSVLFCVKNLYCWVTGGGGSGGLSGRSDAGSPLGLAGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 655 aa, Artificial Sequence; identical to SEQ ID NO: 134
  Description of Artificial Sequence Synthetic polypeptide

SEQ ID NO: 186 - protein, 645 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - protein, 266 aa, Artificial Sequence
  Description of Artificial Sequence Synthetic polypeptide
GGVDFCKIYSWPVCHQGGGGSGGLSGRSDAGSPLGLAGSGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 188 - protein, 645 aa, Artificial Sequence; identical to SEQ ID NO: 186
  Description of Artificial Sequence Synthetic polypeptide

SEQ ID NO: 189 - protein, 266 aa, Artificial Sequence
  Description of Artificial Sequence Synthetic polypeptide
GGSVLFCVKNLYCWVTGGGGSGGLSGRSDAGSPLGLAGSGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 190 - protein, 655 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGGSQCLGPEWEVCPYGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 191 - protein, 645 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGGSQCLGPEWEVCPYGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 266 aa, Artificial Sequence; identical to SEQ ID NO: 187
  Description of Artificial Sequence Synthetic polypeptide

SEQ ID NO: 193 - protein, 256 aa, Artificial Sequence; identical to SEQ ID NO: 184
  Description of Artificial Sequence Synthetic polypeptide

SEQ ID NO: 194 - protein, 655 aa, Artificial Sequence; identical to SEQ ID NO: 190
  Description of Artificial Sequence Synthetic polypeptide

SEQ ID NO: 195 - protein, 645 aa, Artificial Sequence; identical to SEQ ID NO: 191
  Description of Artificial Sequence Synthetic polypeptide

SEQ ID NO: 196 - protein, 266 aa, Artificial Sequence; identical to SEQ ID NO: 189
  Description of Artificial Sequence Synthetic polypeptide

SEQ ID NO: 197 - protein, 256 aa, Artificial Sequence; identical to SEQ ID NO: 133
  Description of Artificial Sequence Synthetic polypeptide

SEQ ID NO: 198 - protein, 646 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 199 - protein, 256 aa, Artificial Sequence; identical to SEQ ID NO: 184
  Description of Artificial Sequence Synthetic polypeptide

SEQ ID NO: 200 - protein, 646 aa, Artificial Sequence; identical to SEQ ID NO: 198
  Description of Artificial Sequence Synthetic polypeptide

SEQ ID NO: 201 - protein, 636 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 266 aa, Artificial Sequence; identical to SEQ ID NO: 187
  Description of Artificial Sequence Synthetic polypeptide

SEQ ID NO: 203 - protein, 636 aa, Artificial Sequence; identical to SEQ ID NO: 201
  Description of Artificial Sequence Synthetic polypeptide

SEQ ID NO: 204 - protein, 266 aa, Artificial Sequence; identical to SEQ ID NO: 189
  Description of Artificial Sequence Synthetic polypeptide

SEQ ID NO: 205 - protein, 261 aa, Artificial Sequence
  Description of Artificial Sequence Synthetic polypeptide
GGIDFCMLYNWPICAGGGGGSSGGSAAGLLAPPGGLSGRSDAGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 646 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGSSGGSAAGLLAPPGGLSGRSDAGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 207 - protein, 261 aa, Artificial Sequence; identical to SEQ ID NO: 205
  Description of Artificial Sequence Synthetic polypeptide

SEQ ID NO: 208 - protein, 655 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGSSGGSAAGLLAPPGGLSGRSDAGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 209 - protein, 645 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGSSGGSAAGLLAPPGGLSGRSDAGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 271 aa, Artificial Sequence
  Description of Artificial Sequence Synthetic polypeptide
GGIDFCMLYNWPICAGGGGGSSGGSAAGLLAPPGGLSGRSDAGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 211 - protein, 636 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGSSGGSAAGLLAPPGGLSGRSDAGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 271 aa, Artificial Sequence; identical to SEQ ID NO: 210
  Description of Artificial Sequence Synthetic polypeptide

SEQ ID NO: 213 - protein, 655 aa, Artificial Sequence; identical to SEQ ID NO: 208
  Description of Artificial Sequence Synthetic polypeptide

SEQ ID NO: 214 - protein, 256 aa, Artificial Sequence
  Description of Artificial Sequence Synthetic polypeptide
GGIDFCMLYNWPICAGGGGGSGGLSGRSDAGSPLGLAGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 655 aa, Artificial Sequence; identical to SEQ ID NO: 134
  Description of Artificial Sequence Synthetic polypeptide

SEQ ID NO: 216 - protein, 256 aa, Artificial Sequence; identical to SEQ ID NO: 214
  Description of Artificial Sequence Synthetic polypeptide

SEQ ID NO: 217 - protein, 646 aa, Artificial Sequence; identical to SEQ ID NO: 198
  Description of Artificial Sequence Synthetic polypeptide

SEQ ID NO: 218 - protein, 259 aa, Artificial Sequence
  Description of Artificial Sequence Synthetic polypeptide
GGIDFCMLYNWPICAGGGGGSGGGGGSGGGGSGGASSGAGGSGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 646 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGSGGGGSGGASSGAGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 220 - protein, 214 aa, Artificial Sequence; identical to SEQ ID NO: 126
  Description of Artificial Sequence Synthetic polypeptide

SEQ ID NO: 221 - protein, 657 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGGSQCLGPEWEVCPYGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 222 - protein, 645 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGGSQCLGPEWEVCPYGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 226 aa, Artificial Sequence; identical to SEQ ID NO: 127
  Description of Artificial Sequence Synthetic polypeptide

SEQ ID NO: 224 - protein, 214 aa, Artificial Sequence; identical to SEQ ID NO: 126
  Description of Artificial Sequence Synthetic polypeptide

SEQ ID NO: 225 - protein, 657 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 226 - protein, 645 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 226 aa, Artificial Sequence; identical to SEQ ID NO: 127
  Description of Artificial Sequence Synthetic polypeptide

SEQ ID NO: 228 - protein, 636 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 226 aa, Artificial Sequence; identical to SEQ ID NO: 127
  Description of Artificial Sequence Synthetic polypeptide

SEQ ID NO: 230 - protein, 636 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGVYCGPEFDESVGCMGGGGSGGGSGGGGSGGASSGAGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 226 aa, Artificial Sequence; identical to SEQ ID NO: 127
  Description of Artificial Sequence Synthetic polypeptide

SEQ ID NO: 232 - protein, 468 aa, Artificial Sequence; identical to SEQ ID NO: 159
  Description of Artificial Sequence Synthetic polypeptide

SEQ ID NO: 233 - protein, 9 aa, Artificial Sequence
  Description of Artificial Sequence Synthetic peptide
  SITE (1)..(9): This sequence may encompass 1-3 “Gly Gly Ser” repeating units
GGSGGSGGS

SEQ ID NO: 234 - protein, 5 aa, Artificial Sequence
  Description of Artificial Sequence Synthetic peptide
GSGGS

SEQ ID NO: 235 - protein, 4 aa, Artificial Sequence
  Description of Artificial Sequence Synthetic peptide
GGGS

SEQ ID NO: 236 - protein, 5 aa, Artificial Sequence
  Description of Artificial Sequence Synthetic peptide
GGGGS

SEQ ID NO: 237 - protein, 6 aa, Artificial Sequence
  Description of Artificial Sequence Synthetic peptide
GSSGGS

SEQ ID NO: 238 - protein, 6 aa, Artificial Sequence
  Description of Artificial Sequence Synthetic peptide
  SITE (1)..(6): This sequence may encompass 1-3 “Gly Ser” repeating units
GSGSGS

SEQ ID NO: 239 - protein, 15 aa, Artificial Sequence
  Description of Artificial Sequence Synthetic peptide
  SITE (1)..(15): This sequence may encompass 1-3 “Gly Ser Gly Gly Ser” repeating units
GSGGSGSGGSGSGGS

SEQ ID NO: 240 - protein, 12 aa, Artificial Sequence
  Description of Artificial Sequence Synthetic peptide
  SITE (1)..(12): This sequence may encompass 1-3 “Gly Gly Gly Ser” repeating units
GGGSGGGSGGGS

SEQ ID NO: 241 - protein, 15 aa, Artificial Sequence
  Description of Artificial Sequence Synthetic peptide
  SITE (1)..(15): This sequence may encompass 1-3 “Gly Gly Gly Gly Ser” repeating units
GGGGSGGGGSGGGGS

SEQ ID NO: 242 - protein, 18 aa, Artificial Sequence
  Description of Artificial Sequence Synthetic peptide
  SITE (1)..(18): This sequence may encompass 1-3 “Gly Ser Ser Gly Gly Ser” repeating units
GSSGGSGSSGGSGSSGGS

SEQ ID NO: 243 - protein, 30 aa, Artificial Sequence
  Description of Artificial Sequence Synthetic polypeptide
  SITE (1)..(30): This sequence may encompass 1-10 “Gly Gly Ser” repeating units
GGSGGSGGSGGSGGSGGSGGSGGSGGSGGS

SEQ ID NO: 244 - protein, 20 aa, Artificial Sequence
  Description of Artificial Sequence Synthetic peptide
  SITE (1)..(20): This sequence may encompass 1-10 “Gly Ser” repeating units
GSGSGSGSGSGSGSGSGSGS

SEQ ID NO: 245 - protein, 50 aa, Artificial Sequence
  Description of Artificial Sequence Synthetic polypeptide
  SITE (1)..(50): This sequence may encompass 1-10 “Gly Ser Gly Gly Ser” repeating units
GSGGSGSGGSGSGGSGSGGSGSGGSGSGGSGSGGSGSGGSGSGGSGSGGS

SEQ ID NO: 246 - protein, 40 aa, Artificial Sequence
  Description of Artificial Sequence Synthetic polypeptide
  SITE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 247 - protein, 50 aa, Artificial Sequence
  Description of Artificial Sequence Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 248 - protein, 60 aa, Artificial Sequence
  Description of Artificial Sequence Synthetic polypeptide
  SITE (1)..(60): This sequence may encompass 1-10 “Gly Ser Ser Gly Gly Ser” repeating units
GSSGGSGSSGGSGSSGGSGSSGGSGSSGGSGSSGGSGSSGGSGSSGGSGSSGGSGSSGGS

SEQ ID NO: 249 - protein, 261 aa, Artificial Sequence
  Description of Artificial Sequence Synthetic polypeptide
GGVDFCKIYSWPVCHQGGGGSSGGSAAGLLAPPGGLSGRSDAGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 653 aa, Artificial Sequence
  Description of Artificial Sequence Synthetic polypeptide
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC